(19) United States
(12) Patent Application Publication
(10) Pub. No.: US 2025/0101421 A1
(43) Pub. Date: Mar. 27, 2025

(54) OLIGONUCLEOTIDE MODULATORS ACTIVATING UTROPHIN EXPRESSION

(71) Applicant: Ractigen Therapeutics, Nantong Jiangsu (CN)

(72) Inventors: Moorim KANG, Nantong Jiangsu (CN); Robert PLACE, Nantong Jiangsu (CN); Longcheng LI, Nantong Jiangsu (CN)

(21) Appl. No.: 18/730,577

(22) PCT Filed: Jan. 13, 2023
(86) PCT No.: PCT/CN2023/072076

§ 371 Date: Jul. 19, 2024

(30) Foreign Application Priority Data
Jan. 20, 2022 (WO) PCT/CN2022/072958

(51) Int. Cl. C12N 15/113; C12N 15/11

(52) U.S. Cl. CPC C12N 15/113; C12N 15/111; C12N 2310/11

(57) ABSTRACT

Provided is an oligonucleotide modulator for preventing or treating dystrophin-deficient-related disorders (DDD) including Duchenne muscular dystrophy (DMD) and Becker muscular dystrophy (BMD). The oligonucleotide modulator comprises a sense nucleic acid strand and an antisense nucleic acid strand, wherein the sense nucleic acid strand and the antisense nucleic acid strand are independently an oligonucleotide strand of 16 to 35 nucleotides in length, in which one nucleotide strand has at least 75% base homology or complementarity to a target selected from a promoter region of a target gene UTRN.

### TECHNICAL FIELD

The present disclosure relates to the technical field of nucleic acids, specifically as it relates to an oligonucleotide modulator associated with gene activation and pharmaceutical use thereof.

### BACKGROUND

Duchenne muscular dystrophy (DMD) is a recessive, X-linked genetic disease occurring at a frequency of about 1 in 3,500 to 5000 new-born males. About 20,000 children are diagnosed with DMD globally each year. DMD leads to premature death of patients in the 2nd-4th decade of life. The disease is caused by mutation in the DMD gene that encodes the dystrophin protein which is important in muscle fibers, and its absence results in muscle weakness that gets worse over time because muscle cells break down and are gradually lost. Approximately ⅓ of the children obtained DMD as a result of spontaneous mutation in the dystrophin gene and have no family history of the disease.

Dystrophin is part of the dystrophin-glycoprotein complex (DGC), which bridges the inner cytoskeleton (F-actin) and the extra-cellular matrix. In this manner it provides stability to muscle fibers during contraction. Becker muscular dystrophy (BMD) is a milder, less progressive form of the disease, and is also caused by changes in the same DMD gene.

In general, DMD patients carry mutations which yield an incomplete dystrophin protein (nonsense or frame shift mutations) that is not functional, while in BMD, internally deleted proteins of reduced molecular weight (derived from in-frame deletions) are expressed, which are partially functional.

The DMD gene is highly complex, containing at least seven independent, tissue-specific promoters and two polyadenylation sites. Furthermore, dystrophin transcripts are alternatively spliced, producing a range of different transcripts, encoding a large set of protein isoforms. Dystrophin is also expressed in brain, where it has yet unknown functions. However, lack of brain dystrophin probably underlies cognitive problems that many DMD patients experience.

Utrophin gene (UTRN) encodes the utrophin protein which shares both structural and functional similarities with the dystrophin. Utrophin contains an actin-binding N-terminus, a triple coiled-coil repeat central region, and a C-terminus that consists of protein-protein interaction motifs which interact with dystroglycan protein components. Utrophin is located at the neuromuscular synapse and myotendinous junctions, where it participates in post-synaptic membrane maintenance and acetylcholine receptor clustering.

In early human developing muscles, utrophin is found at the sarcolemma and is progressively replaced by dystrophin toward birth. In adult tissues, utrophin is expressed in a wide range of tissues such as lung, kidney, liver, spleen and is limited not only to neuromuscular and myotendinous junctions in muscles but also the sarcolemma in regenerating myofibers and blood vessels. Two utrophin promoters, A and B, differentially regulated, have been reported to drive two distinct mRNA isoforms translated into full length utrophin proteins with unique N-termini and different expression patterns.

Unlike the dystrophin tissue-specific promoters, utrophin A promoter driving the gene encoding the full-length protein is associated with a CpG island at the 5′-end of the gene. Expressed in many tissues, utrophin A is the isoform that is expressed in muscle at neuromuscular and myotendinous junctions, choroid plexus, pia mater, and renal glomerulus and found at the sarcolemma in regenerating myofibers. utrophin B, which differs from A by a slightly different N-terminal acting binding site, is confined to endothelial cells and blood vessels. Both dystrophin and utrophin have smaller transcripts driven by similar internal promoters (Dp71, Dp140, Up71, Up140).

Utrophin is an autosomal and functional paralogue of dystrophin and is able to compensate for the primary defect of dystrophin in DMD and BMD. Mouse studies have suggested that utrophin gene may serve as a functional substitute for the dystrophin gene and, therefore, may serve as a potential therapeutic target for muscular dystrophy resulted from dystrophin deficiency. Therefore, a utrophin based strategy has the potential to offer a treatment to all DMD/BMD patients irrespective of their genetic defect.

The induction of utrophin expression at both the transcriptional and post-transcriptional levels by utilizing oligonucleotides represents an attractive approach for developing novel therapies for DMD and BMD regardless of the location of mutation in DMD gene. However, such approach was limited by tools of gene upregulation [US20120122953A1 and WO2019183005A1].

### SUMMARY

In order to address the aforementioned problem, the present disclosure provides an oligonucleotide modulator such as a small activating RNA (saRNA) molecule, for treating diseases or conditions caused by the lack or insufficient level of dystrophin such as DMD and BMD by targeting UTRN gene promoter and subsequently activating UTRN gene transcription and increasing the expression level of utrophin protein to compensate the deficiency of dystrophin via the RNA activation (RNAa) mechanism.

In particular, the inventors discovered that such saRNAs capable of activating/up-regulating the expression of UTRN mRNA were not randomly distributed on the promoter but were clustered in certain specific hotspot regions. Only some regions on the promotor of UTRN gene are in favor of gene activation by saRNAs, for example, the regions −636 to −496, −351 to −294, −236 to −187 and −101 to −65 upstream of the transcription start site of UTRN gene. The inventors also discovered that optimal target sequences/sense strand of an saRNA within the UTRN promoter region include sequences having criteria of: (1) a GC content between 35% and 70%; (2) less than 5 consecutive identical nucleotides; (3) 3 or less dinucleotide repeats; and (4) 3 or less trinucleotide repeats. As a beneficial consequence of the criteria, a target sequence (e.g., an isolated nucleic acid sequence comprising the target sequence), upon interacting with the saRNA, can activate/upregulate the expression of UTRN mRNA by at least 10% as compared to a baseline level of UTRN mRNA. Based at least in part on these discoveries, the present disclosure features saRNA, compositions, and pharmaceutical compositions for activating/up-regulating the expression of UTRN mRNA by at least 10% as compared to baseline levels of UTRN gene. Also provided herein are methods for preventing or treating a disease or condition induced by insufficient expression of dystrophin, a dystrophin gene mutation, and/or low functional dystrophin levels in an individual comprising administering any of the saRNA, compositions, and/or pharmaceutical compositions described herein.

In one aspect of the present disclosure, an oligonucleotide modulator (such as saRNA molecule) capable of activating/up-regulating expression of the UTRN gene in a cell is provided, the oligonucleotide modulator (e.g., the saRNA) comprising an oligonucleotide sequence of 16 to 35 consecutive nucleotides in length, wherein the continuous oligonucleotide sequence has at least 75%, or at least 80%, or at least 85%, or at least 90% sequence homology or complementary to an equal length region of SEQ ID NO:1200, and thereby activating or up-regulating the expression of the gene by at least 10% as compared to baseline expression of the UTRN gene. In some embodiments, the equal length region of SEQ ID NO:1200 is located in the region −636 to −496 (SEQ ID NO:1207), region −351 to −294 (SEQ ID NO:1208), region −236 to −187 (SEQ ID NO:1209), or region −101 to −65 (SEQ ID NO:1210) upstream of the transcription start site of UTRN gene.

In certain embodiments, the saRNA disclosed in the present disclosure comprises a sense strand and an antisense strand, wherein the sense strand and the antisense strand each comprise complementary regions, wherein the complementary regions of the sense strand and the antisense strand form a double-stranded nucleic acid structure. In certain embodiments, the sense strand and the antisense strand disclosed in the present disclosure have a complementarity of at least 90%. In certain embodiments, the sense strand and the antisense strand disclosed in the present disclosure are located on two different nucleic acid strands. While in certain embodiments, the sense strand and the antisense strand disclosed in the present disclosure are located on a contiguous nucleic acid strand, optionally a hairpin single-stranded nucleic acid molecule, wherein the complementary regions of the sense strand and the antisense strand form a double-stranded nucleic acid structure. In certain embodiments, the sense strand and the antisense strand disclosed in the present disclosure comprises a 3′ overhang ranging from 0 to 6 nucleotides in length, alternatively, from 2 to 3 nucleotides in length. In certain embodiments, at least one of the nucleotides of the overhang is a thymine deoxyribonucleotide. In certain embodiments, the overhangs are natural overhangs which are nucleotides selected from or complementary to the corresponding position on the DNA target. In certain embodiments, the sense strand and the antisense strand disclosed in the present disclosure independently comprise a length of about 16 to about 35, about 17 to about 30, about 18 to about 25, or about 19 to about 22 consecutive nucleotides.

In certain embodiments, the sense strand disclosed in the present disclosure has at least 75% sequence homology to a nucleotide sequence selected from SEQ ID NOs: 400-797, and the antisense strand disclosed in the present disclosure has at least 75% sequence homology to a nucleotide sequence selected from SEQ ID NOs: 800-1197. In certain embodiments, the sense strand disclosed in the present disclosure comprises a nucleotide sequence selected from SEQ ID NOs: 400-797, and the antisense strand disclosed in the present disclosure comprises a nucleotide sequence selected from SEQ ID NOs: 800-1197.

In certain embodiments, the oligonucleotide sequence disclosed in the present disclosure has at least 75% sequence homology or complementarity to a nucleotide sequence selected from SEQ ID NOs: 1-398. In certain embodiments, the sense strand of the oligonucleotide sequence disclosed in the present disclosure has at least 75% sequence homology to a nucleotide sequence selected from SEQ ID NOs: 1-398. In certain embodiments, the antisense strand of the oligonucleotide sequence disclosed in the present disclosure has at least 75% sequence complementarity to a nucleotide sequence selected from SEQ ID NOs: 1-398.

In certain embodiments, at least one nucleotide of the saRNA disclosed in the present disclosure is a chemically modified nucleotide. In certain embodiments, at least one nucleotide of the antisense and/or sense strand of the saRNA disclosed in the present disclosure is chemically modified. In certain embodiments, the chemically modified nucleotide disclosed in the present disclosure is a nucleotide with at least one the following modifications:
a) modification of a phosphodiester bond connecting nucleotides in the nucleotide sequence of the saRNA;
b) modification of 2′-OH of a ribose in the nucleotide sequence of the saRNA; and
c) modification of a base in the nucleotide sequence of the saRNA.

In certain embodiments, at least one nucleotide of the saRNA disclosed in the present disclosure is a locked nucleic acid, an abasic nucleotide, a 2′-amino-modified nucleotide, a 2′-alkyl-modified nucleotide, a morpholino nucleotide, a phosphoramidate, or a non-natural base comprising nucleotide.

In certain embodiments, the chemical modification of the at least one chemically modified nucleotide disclosed in the present disclosure is an addition of a (E)-vinylphosphonate moiety at the 5′ end of the sense strand or the antisense strand.

In certain embodiments, the disclosure provides oligonucleotide modulator wherein the sense strand and/or the antisense strand of the saRNA disclosed in the present disclosure is conjugated to one or more conjugation moieties selected from a lipid, a fatty acid, a fluorophore, a ligand, a saccharide, a peptide, and an antibody.

In certain embodiments, the sense strand or the antisense strand of the saRNA disclosed in the present disclosure is conjugated to one or more conjugation moieties selected from a cell-penetrating peptide, polyethylene glycol, an alkaloid, a tryptamine, a benzimidazole, a quinolone, an amino acid, a cholesterol, glucose, and N-acetylgalactosamine.

In certain embodiments of the oligonucleotide modulator, the sense strand or the antisense strand of the saRNA disclosed in the present disclosure is conjugated to one or more conjugation moieties selected from a cell-penetrating peptide, polyethylene glycol, an alkaloid, a tryptamine, a benzimidazole, a quinolone, an amino acid, a cholesterol, glucose, and N-acetylgalactosamine.

In another aspect of the present disclosure, an isolated polynucleotide of saRNA is provided, wherein the isolated polynucleotide is a continuous nucleotide sequence having a length of 16 to 35 nucleotides in SEQ ID NO:1200. Specifically, the isolated polynucleotide is a nucleic acid sequence selected from SEQ ID NOs:1-398. In another aspect of the present disclosure, methods of using the isolated polynucleotide of saRNA is provided.

In another aspect of the present disclosure, an isolated oligonucleotide complex is provided, wherein the isolated oligonucleotide complex comprises the antisense strand of the saRNA disclosed herein and the sense strand of the isolated polynucleotide disclosed herein. In some embodiments, the isolated oligonucleotide complex activates the expression of UTRN gene by at least 10% as compared to the baseline level of the gene.

Another aspect of the present disclosure provides an isolated polynucleotide encoding the saRNA disclosed herein. In one embodiment, the saRNA disclosed herein is a small activating RNA (saRNA) molecule. In one embodiment, the polynucleotide is a DNA molecule. Another aspect of the present disclosure provides a vector comprising the isolated polynucleotide disclosed herein.

In another aspect of the present disclosure, an isolated nucleic acid complex is provided, wherein the isolated nucleic acid complex comprises the antisense strand of the saRNA disclosed herein and the sense strand of the isolated polynucleotide disclosed herein. In some embodiments, the isolated nucleic acid complex activates the expression of UTRN gene by at least 10% as compared to baseline expression of the UTRN gene in a cell.

The present disclosure is also related to an isolated polynucleotide encoding the saRNA disclosed herein in the present disclosure. A vector comprising the isolated polynucleotide disclosed herein is also disclosed.

Another aspect of the present disclosure provides a cell comprising the saRNA disclosed herein, the isolated polynucleotide encoding the saRNA disclosed herein, or the vector disclosed herein. In one embodiment, the cell is a mammalian cell, optionally a human cell. In some embodiments, the cell is a host cell. The aforementioned cell may be in vitro, such as a cell line or a cell strain, or may exist in a mammalian body, such as a human body. In some embodiments, the isolated polynucleotide is a DNA. In some embodiments, the vector is an AAV.

Another aspect of the present disclosure provides a composition, such as a pharmaceutical composition, comprising the aforementioned saRNA or isolated polynucleotide encoding the saRNA disclosed herein and optionally, a pharmaceutically acceptable carrier. In some embodiments, the pharmaceutically acceptable carrier includes an aqueous carrier, a liposome, a high-molecular polymer or a polypeptide. In some embodiments, the pharmaceutically acceptable carrier is selected from an aqueous carrier, a liposome, a high-molecular polymer and a polypeptide. In some embodiments, the aqueous carrier may be, for example, RNase-free water or RNase-free buffer. In some embodiments, the composition may comprise 0.001-200 nM (e.g., 0.001-200 nM, 0.001-100 nM, 0.001-50 nM, 0.001-20 nM, 10-100 nM, 10-50 nM, 20-50 nM, 20-100 nM or 50-150 nM), or optionally 1-150 nM of the aforementioned saRNA or isolated polynucleotide encoding the saRNA disclosed herein.

Another aspect of the present disclosure relates to use of the aforementioned saRNA, isolated polynucleotide encoding the saRNA disclosed herein or the composition comprising the aforementioned saRNA or isolated polynucleotide disclosed herein in preparing a preparation for activating/up-regulating the expression of UTRN gene in a cell.

The present disclosure also relates to a method for activating/up-regulating the expression of UTRN gene in a cell, wherein the method comprises administering the aforementioned saRNA, the isolated polynucleotide disclosed herein or the composition comprising the aforementioned saRNA or isolated polynucleotide disclosed herein to the cell. In the meantime, a method for increasing a level of utrophin in a cell or a level of functional utrophin in muscle is also provided, comprising introducing the saRNA, the nucleic acid, or the composition disclosed herein into the cell in an effective amount.

The aforementioned saRNA, the isolated polynucleotide disclosed herein or the composition comprising the aforementioned saRNA or isolated polynucleotide disclosed herein may be directly introduced into a cell or may be produced in the cell after a nucleotide sequence encoding the saRNA is introduced into the cell. The cell is for example a mammalian cell, such as a human cell. The aforementioned cell may be in vitro, such as a cell line or a cell strain, or may exist in a mammalian body, such as a human body. The human body can be a subject suffering from a disease or symptom caused by insufficient expression of dystrophin, a dystrophin gene mutation, and/or low functional dystrophin levels in an individual, and the saRNA, the isolated polynucleotide disclosed herein or the composition comprising the aforementioned saRNA or the isolated polynucleotide disclosed herein is administered in a sufficient amount to treat the disease or symptom. Specifically, the symptom caused by lack of dystrophin due to dystrophin gene mutation, and/or insufficient expression of functional dystrophin includes, for example, DMD and BMD. In one embodiment, the disease caused by insufficient expression of dystrophin, a dystrophin gene mutation, and/or low functional dystrophin levels is DMD. In another embodiment, the disease caused by insufficient expression of dystrophin, a dystrophin gene mutation, and/or low functional dystrophin levels is BMD.

Another aspect of the present disclosure relates to a method for preventing or treating a disorder caused by insufficient expression of dystrophin, a dystrophin gene mutation, and/or insufficient levels of functional dystrophin in an individual, which comprises administering a therapeutically effective dose of the saRNA disclosed herein, the isolated polynucleotide encoding the saRNA disclosed herein, the vector disclosed herein, or the composition comprising the saRNA disclosed to the individual. In certain embodiments, the disease or condition is DMD. In certain embodiments, the disease or condition is BMD. The individual may be a mammal, such as a human. In one embodiment, the individual suffers from a symptom caused by insufficient expression of dystrophin, a dystrophin gene mutation and/or low functional dystrophin levels in muscle may include, for example, BMD. In one embodiment, the disease caused by insufficient muscle levels of functional dystrophin due to dystrophin gene mutation is DMD or BMD. In one embodiment, the diseases described herein include DMD and BMD. In certain embodiments, the saRNA disclosed herein, the isolated polynucleotide disclosed herein, the vector disclosed herein, or the composition disclosed herein is administrated to an individual by an administration pathway selected from one or more of: parenteral infusions, oral administration, intranasal administration, inhaled administration, vaginal administration, and rectal administration. In certain embodiments, the administration pathway is selected from one or more of intrathecal, intramuscular, intravenous, intra-arterial, intraperitoneal, intravesical, intracerebroventricular, intravitreal and subcutaneous administrations. In certain embodiments, the method disclosed herein activates/up-regulates expression of UTRN gene or UTRN mRNA in the individual by at least 10% (e.g., by at least 15%, by at least 20%, by at least 25%, by at least 30%, by at least 35%, by at least 40%, by at least 45%, or by at least 50%) as compared to the baseline level of the gene. In certain embodiments, the method disclosed herein increases level of utrophin in the individual by at least 10% as compared to the baseline level of the gene.

Another aspect of the present disclosure relates to use of the saRNA disclosed herein, the isolated polynucleotide disclosed herein or the composition comprising the saRNA disclosed herein or the isolated polynucleotide disclosed herein in preparing a medicament for preventing or treating a disorder or condition caused insufficient expression of dystrophin, a dystrophin gene mutation, and/or low functional dystrophin levels in an individual. The individual may be a mammal, such as a human. In one embodiment, the disease or condition may include, for example, DMD or BMD. In one embodiment, the disease caused by insufficient expression of dystrophin, a dystrophin gene mutation, and/or low functional dystrophin levels is DMD. In one embodiment, the diseases described herein include DMD and BMD.

In addition, the present disclosure further provides kit for performing the method of prevention or treatment disclosed herein, wherein the kit comprises a) saRNA, b) instructions for use, and c) optionally, means for administering said saRNA to the individual. Specifically, a kit can be packed in a labeled package and the label on said package indicates that said molecule or composition can be used in preventing or treating a disorder or condition induced by insufficient expression of dystrophin, or against DMD or BMD.

A kit is provided by the present disclosure for performing the method disclosed herein, wherein the kit comprises a) saRNA disclosed herein, and b) instructions for use. In certain embodiments, the instruction for use comprising means or methods for administering the saRNA disclosed herein to an individual.

Aspects of the present disclosure include a kit comprising the saRNA disclosed herein, the isolated polynucleotide disclosed herein, the vector disclosed herein, or the composition disclosed herein in a labeled package and the label on package indicates that the saRNA, the isolated polynucleotide, the vector or the composition can be used in preventing or treating a disease or condition induced by insufficient expression of dystrophin, or against DMD or BMD.

Further to provide by the present disclosure is a kit for detecting dystrophin, utrophin, or utrophin related protein (e.g., dystroglycan) in muscle or plasma, or in a cell disclosed herein having been transfected with the saRNA aforementioned, or the nucleic acid aforementioned, or the composition aforementioned.

The saRNA activating/upregulating the expression of UTRN gene provided herein (such as an saRNA molecule) can efficiently and specifically upregulate the expression of UTRN gene and increase the expression level of UTRN mRNA with low toxic and adverse effects, and can be used in preparing a drug for preventing or treating disorders associated with insufficient expression of dystrophin and diseases or conditions caused by a dystrophin gene mutation.

### BRIEF DESCRIPTION OF THE DRAWINGS

FIG. 1 shows changes in expression level of human UTRN mRNA mediated by saRNAs. 398 human UTRN promotor-targeting saRNAs were individually transfected at a concentration of 25 nM for 3 days into human malignant embryonic rhabdomyoma cell line (RD). Sequences of saRNA strands and duplex composition are shown in Table 1. Mock was transfected in the absence of an oligonucleotide (not shown). dsCon2 duplex served as a non-specific duplex control (not shown). DS18-si8 was a duplex siRNA targeting UTRN gene and transfected as a silencing dsRNA control (not shown). mRNA levels of UTRN at day 3 were quantified by one-step RT-qPCR using a gene specific primer set (as shown in Table 3) in each of PCR reactions. Geometric means of the mRNA levels of TBP and B2M were used as an internal reference. The value (y-axis, log 2 fold change) shows the relative fold changes on UTRN mRNA expression levels by each of the 398 saRNAs relative to Mock treatment after normalized to TBP and B2M (mean±SEM of two replicate transfection wells). saRNAs are sorted on x-axis by their activity of inducing UTRN mRNA expression (log 2) in a descending order.

FIG. 2 shows saRNAs sorted by their target location and hotspot regions on human UTRN promoter. 398 human UTRN promoter-targeting saRNAs were individually transfected at 25 nM into RD cells for 3 days. Mock was transfected in the absence of an oligonucleotide (not shown). dsCon2 duplex served as a non-specific duplex control (not shown). DS18-si8 was a duplex siRNA targeting UTRN gene and transfected as a silencing dsRNA control (not shown). mRNA levels of UTRN at day 3 were quantified by one-step RT-qPCR using a gene specific primer set (as shown in Table 3) in individual PCR reactions. Geometric means of the mRNA levels of TBP and B2M were used as an internal reference. The value (y-axis, log 2 fold change) shows the relative fold changes in UTRN mRNA expression levels by each of the saRNAs relative to Mock treatment after normalized to B2M and TBP (mean±SEM of two replicate transfection wells). saRNAs are sorted on x-axis by their location on the promoter from −666 bp upstream and 334 bp downstream of UTRN transcription start site (TSS). Locations of the 4 saRNA hotspot regions were marked as H1 to H4 in rectangular dotted boxes. The numbers above the boxes indicate the boundaries of the hotspot regions relative to the UTRN TSS (+1 position) which span the very 5′ end of the first saRNA's target and the very 3′ end of the last saRNA's target within each hotspot region.

FIG. 3 shows the activating effects of lead saRNAs on the expression of human UTRN gene RD cells. Cells were treated at an saRNA concentration of 25 nM for 3 days. Mock was transfected in the absence of an oligonucleotide. dsCon2 was transfected as a non-specific duplex control. DS18-si8 was a duplex siRNA targeting UTRN gene and transfected as a silencing dsRNA control. FIG. 3 shows mRNA levels of UTRN in RD cells at day 3 were quantified by two step RT-qPCR using a gene specific primer set (as shown in Table 3). Geometric means of the mRNA levels of TBP and B2M were used as an internal reference. The values (y-axis) are presented as UTRN mRNA expression levels relative to Mock treatment after normalized to TBP and B2M (mean SEM of two replicate transfection wells).

FIG. 4 shows the activating effects of lead saRNAs on the protein expression of UTRN in RD cells. The cells were treated by the indicated saRNAs (see Table 1) at 25 nM for 5 days. Mock was transfected in the absence of an oligonucleotide. dsCon2 was transfected as a non-specific duplex control. DS18-si8 was a duplex siRNA targeting UTRN gene and transfected as a silencing dsRNA control. The utrophin protein levels in RD cells at day 5 were determined by Western blotting using a primary antibody against human utrophin protein. An antibody against α/β-Tubulin protein was also blotted to serve as a control for protein loading. FIG. 4 shows relative fold changes of utrophin protein levels derived from quantifying the band intensity. The values (y-axis) are relative band intensity of utrophin after being normalized to α/β-Tubulin. All saRNAs are sorted on x-axis by their activity of inducing utrophin protein expression (fold change) in a descending order.

FIG. 5 shows the activating effects of saRNAs on the expression of human UTRN mRNA in RD cells. The indicated saRNA (see Table 10) were transfected into RD cells at 25 nM for 3 days. Mock was transfected in the absence of an oligonucleotide. dsCon2 was transfected as a non-specific duplex control. DS18-si8 was a duplex siRNA targeting UTRN gene and transfected as a silencing dsRNA control. mRNA levels of UTRN were quantified by two step RT-qPCR using a gene specific primer set (as shown in Table 3). Geometric means of the mRNA levels of TBP and HPRT1 were used as an internal reference. The values (y-axis) are presented as UTRN mRNA expression levels relative to Mock treatment after normalized to TBP and HPRT1 (mean±SEM of two replicate transfection wells).

FIGS. 6A-6B show the activating effects of saRNAs on the expression of human utrophin protein in RD cells. The indicated saRNA (see Table 10) were transfected into RD cells at 25 nM for 3 days. Mock was transfected in the absence of an oligonucleotide. dsCon2 was transfected as a non-specific duplex control. Utrophin protein levels were determined by JESS using a primary antibody against human utrophin protein. An antibody against α/β-Tubulin protein was also detected to serve as a control for protein loading. FIG. 6A shows the protein bands for utrophin and α/β-Tubulin proteins. FIG. 6B shows relative fold changes of utrophin levels derived from quantifying the band intensity of FIG. 6A. The values (y-axis) are relative band intensity of utrophin after normalized to α/β-Tubulin (mean±SEM of two replicate transfection wells).

### DETAILED DESCRIPTION

Double-stranded RNAs (dsRNAs) targeting gene regulatory sequences, including promoters, have been shown to upregulate target genes in a sequence-specific manner at the transcriptional level via a mechanism known as RNA activation (RNAa) (Li, L. C., et al. Small dsRNAs induce transcriptional activation in human cells. PNAS (2006)). Such dsRNAs are termed small activating RNAs (saRNAs).

Embodiments of the present disclosure are based in part on the surprising discovery that an oligonucleotide modulator (for example, saRNA, also referred to as “UTRN gene saRNA” or “UTRN saRNA” herein) is capable of activating or upregulating the expression of a UTRN gene in a cell. The increase in production of functional UTRN gene mRNA following administration with an saRNA of the present disclosure can achieve a significant increase or upregulation in the level of UTRN mRNA and utrophin protein.

In particular, the inventors discovered that the functional saRNAs capable of activating/up-regulating the expression of UTRN mRNA were not randomly distributed on the promoter but were clustered in certain specific hotspot regions. Only some regions on the promotor of UTRN gene are in favor of gene activation by saRNA, for example, the regions −636 to −496, −351 to −294, −236 to −187 and −101 to −65 upstream of the transcription start site of UTRN gene. These specific promoter regions (referred to as “hotspot region” herein) identified by the present disclosure are optionally at least 37 nt in length, or alternatively have a length ranging from about 37 to about 200 nt.

The inventors also discovered that optimal target sequences/sense strand of an saRNA within the UTRN promoter region include sequences having criteria of: (1) a GC content between 35% and 70%; (2) less than 5 consecutive identical nucleotides; (3) 3 or less dinucleotide repeats; and (4) 3 or less trinucleotide repeats. As a beneficial consequence of the criteria, a target sequence (e.g., an isolated nucleic acid sequence comprising the target sequence), upon interacting with the saRNA, can activate/upregulate the expression of UTRN mRNA by at least 10% or 1.1 fold as compared to a baseline level of UTRN mRNA.

A “hotspot region” herein is defined by a nucleic acid region on the target gene of the saRNAs spanning the very 5′ end of the first saRNA's target and the very 3′ end of the last saRNA's target within each hotspot where at least 25% of the saRNAs designed according to the criteria (1), (2), (3), and (4) listed above, to target the region are turned out to be functional, i.e., can induce a 1.1-fold or more change in the mRNA expression of the target gene as compared to the baseline level of the mRNA expression. In some embodiments, at least 28%, at least 30%, about 35%, about 40%, or over 50% of the saRNAs designed to be functional, i.e., can induce a 1.1-fold or more change in the level of mRNA transcription or protein expression of the target gene as compared to a baseline level of the gene.

Based at least in part on these discoveries, the present disclosure features saRNA, compositions, and pharmaceutical compositions for activating/up-regulating the expression of UTRN mRNA by at least 10% as compared to baseline levels of UTRN mRNA. Also provided herein are methods for preventing or treating a disease or condition induced by insufficient expression of dystrophin, a dystrophin gene mutation, and/or low functional dystrophin levels in an individual comprising administering to the individual any of the saRNA, compositions, and/or pharmaceutical compositions described herein.

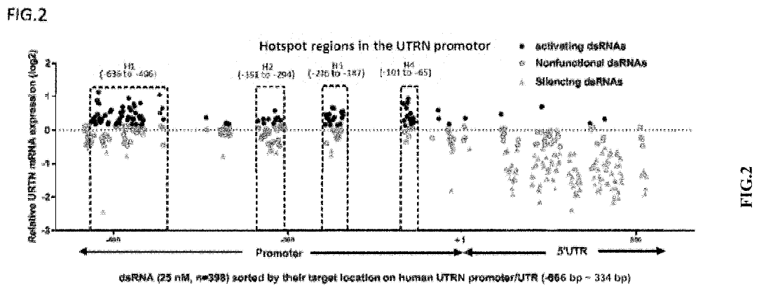

Embodiments of the present disclosure are also based in part on the surprising discovery that the saRNAs capable of activating or up-regulating the expression of UTRN gene in a cell are clustered in particular UTRN gene promoter regions, as shown in FIG. 2. The present inventors identified these clusters of UTRN gene promoter regions that were considered “hotspot” promoter regions that enrich target sites for the functional saRNAs developed (see e.g., Table 9). For example, the 4 hotspot regions of the human UTRN promoter located in regions −636 to −496 (H1), −351 to −294 (H2), −236 to −187 (H3) and −101 to −65 (H4) from the TSS of the promoter were detected and were found to be optimal target sites for saRNAs in activating UTRN gene expression by the RNA activation mechanism.

This saRNA-UTRN mRNA-utrophin pathway can provide an alternative therapeutic method different from the current treatment of dystrophin-deficiency-related disorders (DDD), e.g., for Duchenne muscular dystrophy (DMD) and Becker muscular dystrophy (BMD) patients.

In the present disclosure, the related terms are defined as follows:

The term “complementary” as used herein refers to the capability of forming base pairs between two oligonucleotide strands. The base pairs are generally formed through hydrogen bonds between nucleotides in the antiparallel oligonucleotide strands. The bases of the complementary oligonucleotide strands can be paired in the Watson-Crick manner (such as A to T, A to U, and C to G) or in any other manner allowing the formation of a duplex (such as Hoogsteen or reverse Hoogsteen base pairing).

Complementarity includes complete complementarity and incomplete complementarity. “Complete complementarity” or “100% complementarity” means that each nucleotide from the first oligonucleotide strand can form a hydrogen bond with a nucleotide at a corresponding position in the second oligonucleotide strand in the double-stranded region of the double-stranded oligonucleotide molecule, with no base pair being “mispaired”. “Incomplete complementarity” means that not all the nucleotide units of the two strands are bound with each other by hydrogen bonds. For example, for two oligonucleotide strands each of 20 nucleotides in length in the double stranded region, if only two base pairs in this double-stranded region can be formed through hydrogen bonds, the oligonucleotide strands have a complementarity of 10%. In the same example, if 18 base pairs in this double-stranded region can be formed through hydrogen bonds, the oligonucleotide strands have a complementarity of 90%. Substantial complementarity refers to at least about 75%, about 79%, about 80%, about 85%, about 90%, about 95% or 99% complementarity.

The term “oligonucleotide” or “polynucleotide” can be used interchangeably, and refers to polymers of nucleotides, and includes, but is not limited to, single-stranded or double-stranded nucleic acid molecules of DNA, RNA, or DNA/RNA hybrid, oligonucleotide strands containing regularly and irregularly alternating deoxyribosyl portions and ribosyl portions, as well as modified and naturally or unnaturally existing frameworks for such oligonucleotides. The oligonucleotide for activating target gene transcription described herein is a small activating nucleic acid molecule (saRNA).

The terms “oligonucleotide strand”, “strand” and “oligonucleotide sequence” as used herein can be used interchangeably, referring to a generic term for short nucleotide sequences having less than 35 bases (including nucleotides in deoxyribonucleic acid (DNA) or ribonucleic acid (RNA)). In a non-limiting example, the length of a strand can be any length from 16 to 35 nucleotides.

The term “target gene” as used herein can refer to nucleic acid sequences, transgenes, viral or bacterial sequences, chromosomes or extrachromosomal genes that are naturally present in organisms, and/or can be transiently or stably transfected or incorporated into cells and/or chromatins thereof. The target gene can be a protein-coding gene or a non-protein-coding gene (such as a microRNA gene and a long non-coding RNA gene). The target gene generally contains a promoter sequence, and the positive regulation for the target gene can be achieved by designing an saRNA having sequence identity (also called homology) to the promoter sequence, characterized as the up-regulation of expression of the target gene. “Target sequence” or “target site” used interchangeably herein refers to a sequence segment in the sequence of a target gene sequence, such as, a target gene promoter, which is homologous or complementary with a sense strand or an antisense strand of an saRNA according to the present disclosure. The target gene can also include one or more regulatory elements where one or more saRNA are designed to have sequence identity to a regulatory element. Non-limiting examples of one or more regulatory elements include: a promoter, an enhancer, a silencer, an insulator, a TATA box, a GC box, a CAAT box, a transcriptional start site, a DNA binding motif of a transcription factor or other protein that regulates transcription, and a 5′ untranslated region.

As used herein, the terms “sense strand” of an saRNA refers to the strand having sequence homology or sequence identity with a segment of the coding strand of the sequence of a target gene promoter in the saRNA duplex.

As used herein, the terms “antisense strand” of an saRNA refers to the strand which is complementary with the sense strand in the saRNA duplex or the target gene sequence region.

The term “coding strand” as used herein refers to a DNA strand in the target gene which cannot be used for transcription, and the nucleotide sequence of this strand is the same as that of a RNA produced from transcription (in the RNA, T in DNA is replaced by U). The coding strand of the double-stranded DNA sequence of the target gene promoter described herein refers to a promoter sequence on the same DNA strand as the DNA coding strand of the target gene.

The term “template strand” as used herein refers to the other strand complementary with the coding strand in the double-stranded DNA of the target gene, i.e., the strand that, as a template, can be transcribed into RNA, and this strand is complementary with the transcribed RNA (A to U and G to C). In the process of transcription, RNA polymerase binds to the template strand, moves along the 3′→5′ direction of the template strand, and catalyzes the synthesis of the RNA along the 5′→3′ direction. The template strand of the double-stranded DNA sequence of the target gene promoter described herein refers to a promoter sequence on the same DNA strand as the DNA template strand of the target gene.

As used herein, the term “LNA” refers to a locked nucleic acid in which the 2′-oxygen and 4′-carbon atoms are joined by an extra bridge. As used herein, the term “BNA” refers to a 2′-0 and 4′-aminoethylene bridged nucleic acid that can contain a five-membered or six-membered bridged structure with an N—O linkage. As used herein, the term “PNA” refers to a nucleic acid mimic with a pseudopeptide backbone composed of N-(2-aminoethyl) glycine units with the nucleobases attached to the glycine nitrogen via carbonyl methylene linkers.

The term “promoter” as used herein refers to a sequence which is spatially associated with a protein-coding or RNA-coding nucleic acid sequence and plays a regulatory role for the transcription of the protein-coding or RNA-coding nucleic acid sequence. Generally, a eukaryotic gene promoter contains 100 to 5000 base pairs, although this length range is not intended to limit the term “promoter” as used herein. Although the promoter sequence is generally located at the 5′ terminus of a protein-coding or RNA-coding sequence, it may also exist in exon and intron sequences.

The term “transcription start site” as used herein refers to a nucleotide marking the transcription start on the template strand of a gene. The transcription start site can appear on the template strand of the promoter region. A gene can have more than one transcription start site.

The term “identity” or “homology” as used herein means that one oligonucleotide strand (sense or antisense strand) of an saRNA has sequence similarity with a coding strand or template strand in a region of a target gene. As used herein, the “identity” or “homology” may be at least about 75%, about 79%, about 80%, about 85%, about 90%, about 95% or 99%.

The term “equal length portion” refers to a portion of a sequence that is compared with an object sequence (e.g., a continuous oligonucleotide sequence from the saRNA) and has equal length (equal number of bases) to the object sequence.

The term “sequence specific mode” as used herein means a binding or hybridization way of two nucleic acid fragments according to their nucleotide sequence, e.g., a Watson-Crick manner (such as A to T, A to U, and C to G) or any other manner allowing the formation of a duplex (such as Hoogsteen or reverse Hoogsteen base pairing).

The term “overhang” as used herein refers to non-base-paired nucleotides at the terminus (5′ or 3′) of an oligonucleotide strand, which is formed by one strand extending out of the other strand in a double-stranded oligonucleotide. A single-stranded region extending out of the 3′ terminus and/or 5′ terminus of a duplex is referred to as an overhang. The term “natural overhang” as used herein refers to an overhang which is consisted of one or more nucleotides identical or complementary to the corresponding position on the DNA target. A natural overhang on a sense strand is consisted of one or more nucleotides identical to the corresponding position on the DNA target. A natural overhang on an antisense strand is consisted of one or more nucleotides complementary to the corresponding position on the DNA target.

As used herein, the terms “gene activation” or “activating gene expression” and “gene upregulation” or “up-regulating gene expression” can be used interchangeably, and mean an increase in transcription, translation, expression or activity of a certain nucleic acid, compared with a baseline level of the nucleic acid, as determined by measuring the transcriptional level, mRNA level, protein level, enzymatic activity, methylation state, chromatin state or configuration, translation level or the activity or state in a cell or biological system of a gene. These activities or states can be determined directly or indirectly. In addition, “gene activation”, “activating gene expression”, “gene up-regulation” or “up-regulating gene expression” refers to an increase in activity associated with a nucleic acid sequence, regardless of the mechanism of such activation. For example, gene activation occurs at the transcriptional level to increase transcription into RNA and the RNA is translated into a protein, thereby increasing the expression of the protein.

The terms “baseline expression”, or “baseline level” of a nucleic acid or a gene refers to the expression level of the nucleic acid or the gene without any artificial regulation of it, for example, before or without administrating the saRNA according to the present disclosure.

As used herein, the terms “oligonucleotide modulator”, “small activating RNA”, “saRNA”, and “small activating nucleic acid molecule” can be used interchangeably, and refer to a nucleic acid molecule that can upregulate target gene expression and can be composed of a first nucleic acid fragment (sense strand) containing a nucleotide sequence having sequence identity to the non-coding nucleic acid sequence (e.g., a promoter or an enhancer) of a target gene and a second nucleic acid fragment (antisense strand) containing a nucleotide sequence complementary with the first nucleic acid fragment, wherein the first nucleic acid fragment and the second nucleic acid fragment form a duplex. The saRNA can also be comprised of a synthesized or vector-expressed single-stranded RNA molecule that can form a hairpin structure by two complementary regions (first and second regions) within the molecule, wherein the first region contains a nucleotide sequence having sequence identity to the target sequence of a promoter of a gene, and the second region contains a nucleotide sequence which is complementary with the first region. The length of the duplex region of the saRNA is typically about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45 or about 50 base pairs. In addition, the terms “oligonucleotide modulator”, “saRNA”, “small activating RNA”, and “small activating nucleic acid molecule” also contain nucleic acids other than the ribonucleotide, including, but not limited to, modified nucleotides or analogues.

As used herein, the term “functional saRNA” refers to an saRNA which activates the expression of its intended target gene by at least 10% (or at least 1.1 fold) as compared to a baseline level of the gene. The term “non-functional saRNA” refers to an saRNA which modulates the expression of UTRN gene by less than 10% (or less than 1.1 fold) as compared to a baseline level of the gene.

As used herein, the term “hotspot region” refers to a gene promoter region which contains a hotspot and a continuous target sequence spanning the very 5′ end of the first saRNA and the 3′ end of the last saRNA within the hotspot.

As used herein, the term “an isolated target site” and “an isolated polynucleotide” can be used interchangeably, and herein means a target site to which an saRNA has complementarity or hybridizes to. For example, an isolated nucleic acid sequence of a target site can include a nucleic acid sequence to which a region of saRNAs has complementarity or hybridizes to. As used herein, the term “an isolated polynucleotide” used herein means a polynucleotide which encodes an saRNA.

As used herein, the term “synthesis” refers to a method for synthesis of an oligonucleotide, including any method allowing RNA synthesis, such as chemical synthesis, in vitro transcription, and/or vector-based expression.

As used herein, the terms “disease”, “disorder”, and “condition” can be used interchangeably when referring to dystrophin-deficient-related disorders.

As used herein, the upper cased “UTRN” or “UTRN gene” refers to a human gene.

As used herein, the term “UTRN mRNA” refers to a message RNA (mRNA) generated from the expression of UTRN gene, or the transcription of UTRN gene.

As used herein, the terms “UTRN protein” and “utrophin” can be used interchangeably, and refers to a protein generated from the expression of UTRN gene, or translation of the UTRN mRNA.

saRNA

In the present disclosure, expression of the UTRN gene is upregulated by RNA activation, and a related disease (e.g., DMD) is treated by increasing the expression level of utrophin. As the UTRN gene encodes utrophin, an increase in UTRN mRNA expression results in an increase in expression of the utrophin, thereby treating the disease (e.g., DMD). Therefore, the UTRN gene, in some cases, is a target gene in the present disclosure.

Aspects of the present disclosure include an oligonucleotide modulator (e.g., saRNA) comprising an oligonucleotide sequence having a length ranging from 16 to 35 consecutive nucleotides, wherein the continuous oligonucleotide sequence has at least 75%, or at least 80%, or at least 85%, or at least 90% sequence homology or complementary to an equal length portion of SEQ ID NO: 1200, and wherein the saRNA activates/upregulates the expression of UTRN gene by at least 10% as compared to its baseline expression.

In some embodiments, the equal length portion of SEQ ID NO:1200 disclosed herein is located in the region −636 to −496 (SEQ ID NO: 1207), region −351 to −294 (SEQ ID NO:1208), region −236 to −187 (SEQ ID NO:1209), or region −101 to −65 (SEQ ID NO: 1210) upstream of the transcription start site of UTRN gene.

In some embodiments, the continuous oligonucleotide sequence of the saRNA has five or less, i.e., 5, 4, 3, 2, 1, or 0 nucleotide differences or mismatches relative to the equal length portion of SEQ ID NO:1200. In some embodiments, the differences or mismatches locate in the middle or at 3′ terminus of the oligonucleotide sequence of the saRNA. Methods and principles of saRNA molecule design are well known to those skilled in the art and are described in detail in, for example, Place et. al., Molecular Therapy—Nucleic Acids (2012) 1, e15; and Li et.al., PNAS, 2006, vol. 103, no. 46, 17337-17342, which are herein incorporated by reference in their entireties.

In some embodiments, the saRNA disclosed herein comprises a sense strand and an antisense strand. The sense strand and the antisense strand comprise complementary regions capable of forming a double-stranded nucleic acid structure that activates the expression of the UTRN gene in a cell via the RNAa mechanism. The RNAa mechanism (also known as RNA activation) used herein refers to a mechanism that a double-strand nucleic acid structure is capable of upregulating target genes in a sequence-specific manner at the transcriptional level. The sense strand and the antisense strand of the saRNA can exist either on two different nucleic acid strands or on one nucleic acid strand (e.g., a contiguous nucleic acid sequence). When the sense strand and the antisense strand are located on two different strands, at least one strand of the saRNA has a 3′ overhang of 0 to 6 nucleotides in length, such that the overhangs of 0, 1, 2, 3, 4, 5 or 6 nucleotides in length, and in some cases, both strands have a 3′ overhang of 2 or 3 nucleotides in length. The nucleotide of the overhang is, in some cases thymine deoxyribonucleotide (dT), or in some cases, natural overhangs which are nucleotides selected from or complementary to the corresponding position on the DNA target. When the sense strand and the antisense strand are located on one nucleic acid strand, in some cases, the saRNA is a hairpin single-stranded nucleic acid molecule, where the complementary regions of the sense strand and the antisense strand form a double stranded nucleic acid structure with each other. In the aforementioned saRNA, in some embodiments, the sense strand and the antisense strand have a length ranging from 16 to 35 nucleotides, respectively. For example, in some embodiments, the sense strand and the antisense strand, independently comprises a length of 16, 17, 18, 19, 20, 21, 22, 23, 24, 25, 26, 27, 28, 29, 30, 31, 32, 33, 34 or 35 nucleotides.

In certain embodiments, one strand of the saRNA has at least 75% (e.g., at least about 79%, about 80%, about 85%, about 90%, about 95% or about 99%) sequence homology or complementarity to a nucleotide sequence selected from SEQ ID NOs: 1-398. Specifically, the sense strand of the saRNA disclosed herein has at least 75% (e.g., at least about 79%, about 80%, about 85%, about 90%, about 95% or about 99%) sequence homology to any nucleotide sequence selected from SEQ ID NOs: 400-797, and the antisense strand of the saRNA disclosed herein has at least 75% (e.g., at least about 79%, about 80%, about 85%, about 90%, about 95% or about 99%) sequence homology to any nucleotide sequence selected from SEQ ID NOs: 800-1197. More specifically, the sense strand of the saRNA disclosed herein comprises or consists of any nucleotide sequence selected from SEQ ID NOs: 400-797; and the antisense strand of the saRNA disclosed herein comprises or consists of or is any nucleotide sequence selected from SEQ ID NOs: 800-1197.

In certain embodiments, one strand of the saRNA can have five or less, i.e., 5, 4, 3, 2, 1, or 0 nucleotide differences or mismatches relative to the nucleotide sequence selected from SEQ ID Nos: 1-398. Specifically, the sense strand of the saRNA disclosed herein can have five or less, i.e., 5, 4, 3, 2, 1, or 0 nucleotide differences relative to the nucleotide sequence selected from SEQ ID Nos: 400-797, and the antisense strand of the saRNA disclosed herein can have five or less, i.e., 5, 4, 3, 2, 1, or 0 nucleotide differences relative to the nucleotide sequence selected from SEQ ID NOs: 800-1197. In some embodiments, the differences or mismatches locate in the middle or 3′ terminus of the sense or antisense strand of the saRNA.

In certain embodiments, the antisense strand disclosed herein is capable of interacting with a target nucleic acid sequence of a promoter of a gene in a sequence specific manner, meaning that the antisense strand is capable of undergoing hybridization to a target nucleic acid through hydrogen bonding. In certain embodiments, an antisense strand has a nucleotide sequence that, when written in the 5′ to 3′ direction, comprises the reverse complement of the target portion of a target nucleic acid to which it is targeted. In certain such embodiments, an antisense strand has a nucleotide sequence that, when written in the 5′ to 3′ direction, comprises the reverse complement of the target portion in SEQ ID NO: 1200; specifically, the target portion is a nucleic acid sequence selected from SEQ ID NOs:1-398.

In the saRNAs disclosed herein, all nucleotides may be natural or non-chemically modified nucleotides, or at least one nucleotide is a chemically modified nucleotide. Non-limiting examples of the chemical modification include one or more of a combination of the following:
(1) modification of a phosphodiester bond of nucleotides in the nucleotide sequence of the saRNA;
(2) modification of 2′-OH of the ribose in the nucleotide sequence of the saRNA;
(3) modification of a base in the nucleotide of the saRNA; and
(4) at least one nucleotide in the nucleotide sequence of a small activating nucleic acid molecule being a locked nucleic acid.

The chemical modification described herein is well-known to those skilled in the art, and the modification of the phosphodiester bond refers to the modification of oxygen in the phosphodiester bond, including phosphorothioate modification and boranophosphate modification. The modifications disclosed herein stabilize an saRNA structure, maintaining high specificity and high affinity for base pairing.

In some embodiments, the saRNA of the present disclosure includes at least one chemically modified nucleotide which is modified at 2′-OH in pentose of a nucleotide, i.e., the introduction of certain substituents at the hydroxyl position of the ribose, such as 2′-fluoro modification, 2′-oxymethyl modification, 2′-oxyethylidene methoxy modification, 2,4′-dinitrophenol modification, locked nucleic acid (LNA), 2′-amino modification or 2′-deoxy modification, e.g., a 2′-deoxy-2′-fluoro modified nucleotide, a 2′-deoxy-modified nucleotide.

In some embodiments, the saRNA of the present disclosure includes at least one chemically modified nucleotide which is modified at the base of the nucleotide, e.g., 5′-bromouracil modification, 5′-iodouracil modification, N-methyluracil modification, or 2,6-diaminopurine modification.

In some embodiments, the chemical modification of the saRNA is an addition of a (E)-vinylphosphonate moiety at the 5′ end of the sense or antisense sequence. In some embodiments, the chemical modification of the at least one chemically modified nucleotide is an addition of a 5′-methyl cytosine moiety at the 5′ end of the sense or antisense sequence.

In some embodiments, the saRNA of the present disclosure includes at least one nucleotide in the nucleotide sequence of the small activating nucleic acid molecule being a chemically modified nucleic acid, e.g., a locked nucleotide, an abasic nucleotide, a 2′-amino-modified nucleotide, a 2′-alkyl-modified nucleotide, a morpholino nucleotide, a phosphoramidate, and a non-natural base comprising nucleotide. In some embodiments, the saRNA disclosed herein includes an “endo-light” modification with 2′-O-methyl modified nucleotides and nucleotides comprising a 5′-phosphorothioate group.

In some embodiments, the saRNA of the present disclosure is chemically modified to enhance stability or other beneficial characteristics. The nucleic acids featured in the present disclosure may be synthesized and/or modified by conventional methods, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of saRNA molecules that can be used in this present disclosure include, but are not limited to RNAs containing modified backbones or no natural internucleoside linkages. In some embodiments, RNAs having modified backbones include, among others, those that do not have a phosphorus atom in the backbone. In some embodiments, modified RNAs that do not have a phosphorus atom in their internucleoside backbone can also be considered to be oligonucleosides. In some embodiments, the modified oligonucleotide will have a phosphorus atom in its internucleoside backbone.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Non-limiting examples of preparation of the phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39464, which are hereby incorporated by reference in their entireties.

In certain embodiments, the small activating nucleic acid molecule is an RNA, a DNA, a BNA, an LNA or a peptide nucleic acid (PNA).

In addition, to facilitate entry of the saRNA into a cell, chemical conjugation moieties may be introduced at the ends of the sense or antisense strands of the saRNA on the basis of the above modifications to facilitate action through a cell membrane composed of lipid bilayers and gene promoter regions within the nuclear membrane and nucleus.

In certain embodiments, saRNAs disclosed in the present disclosure are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In some embodiments, the saRNA of the present disclosure relates to the sense strand or the antisense strand of the saRNA that is conjugated to one or more conjugation moieties selected from: intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In some embodiments, a conjugation moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In some embodiments, the saRNA of the present disclosure is conjugated to one or more conjugation moieties selected from: a lipid, a fatty acid, a fluorophore, a ligand, a saccharide, a peptide, and an antibody.

In some embodiments, the saRNA of the present disclosure relates to the sense strand or the antisense strand of the saRNA that is conjugated to one or more conjugation moieties selected from a cell-penetrating peptide, polyethylene glycol, an alkaloid, a tryptamine, a benzimidazole, a quinolone, an amino acid, a cholesterol, glucose and N-acetylgalactosamine.

In some embodiments, the saRNA conjugated to one or more conjugation moieties disclosed in the embodiments is directly contacted, transferred, delivered or administrated to a cell or a subject. “Patient”, “individual” or “subject” as used interchangeably herein can refer to a non-human (e.g., a mammal) subject or a human subject.

In some embodiments, the sense strand and the antisense strand of the saRNA independently have at least about 10%, at least about 20%, at least about 30%, at least about 40%, at least about 50%, at least about 60%, at least about 70%, at least about 80%, at least about 90%, at least about 95%, or about 100% nucleotides which are chemically modified nucleotides.

In some embodiments, at least about 50%, at least about 60%, at least about 70%, at least about 80%, at least about 90%, at least about 95%, or about 100% nucleotides of the saRNA are chemically modified nucleotides.

These modifications can increase the bioavailability of the saRNA, improve affinity to a target sequence, and enhance resistance to nuclease hydrolysis in a cell.

In some embodiments, the saRNA of the present disclosure which, upon contact with a cell, are effective in activating or up-regulating the expression of one or more genes in the cell, for example by at least 10% (e.g., at least 20%, at least 30%, at least 40%, at least 50%, at least 60%, at least 70%, at least 80%, at least 90%, at least 100%, at least 150%, at least 200%, at least 300%, at least 500%, at least 800%, at least 1000%, at least 2000%, or at least 5000%).

In a non-limiting example, an saRNA is designed based at least in part on the following criteria: (1) having a GC content between 35% and 70%; (2) with less than 5 consecutive identical nucleotides; (3) with 3 or less dinucleotide repeats; and (4) with 3 or less trinucleotide repeats. In some embodiments, an saRNA is designed/selected based, at least in part, on criteria that enables production of functional saRNA. For example, in some cases, a sequence located upstream of a TSS may include a sequence that does not favor synthesis of an saRNA despite being located in a hotspot region.

In some embodiments, an saRNA is designed/selected based, at least in part, on criteria that includes a sequence having a particular GC content (e.g., a GC content between 25% and 75%) and lacking consecutive identical nucleotides, consecutive dinucleotides, or consecutive trinucleotides. In some embodiments, an saRNA sequence comprises a sequence (1) having a GC content between 35% and 70%; (2) with less than 5 consecutive identical nucleotides; (3) with 3 or less dinucleotide repeats; and (4) with 3 or less trinucleotide repeats.

In some embodiments, an saRNA sequence comprises a sequence having a GC content between 25% and 75%, between 30% and 70%, between 35% and 70%, between 40% and 60%, or between 45% and 55%. In some embodiments, the saRNA comprises a sequence having a GC context between 35% and 70%.

In some embodiments, an saRNA sequence comprises a sequence having less than 7 consecutive identical nucleotides, less than 6 consecutive identical nucleotides, less than 5 consecutive identical nucleotides, less than 4 consecutive identical nucleotides, or less than 3 consecutive identical nucleotides. In some embodiments, the saRNA comprises a sequence having less than 5 consecutive identical nucleotides.

In some embodiments, an saRNA sequence comprises a sequence having 5 or less dinucleotide repeats, 4 or less dinucleotide repeats, 3 or less dinucleotide repeats, or 2 or less dinucleotide repeats. In some embodiments, the saRNA comprises a sequence having 3 or less dinucleotide repeats.

In some embodiments, an saRNA sequence comprises a sequence having 5 or less trinucleotide repeats, 4 or less trinucleotide repeats, 3 or less trinucleotide repeats, or 2 or less trinucleotide repeats. In some embodiments, the saRNA comprises a sequence having 3 or less trinucleotide repeats.

### Target Sequence

In certain embodiments, the present disclosure relates to an isolated target site of the saRNA of the present disclosure, specifically, the isolated target site is a nucleotide sequence having a length ranging from 16 to 35 nucleotides in the nucleotide sequence of SEQ ID NO: 1200. In certain embodiments, the isolated target site is a nucleic acid sequence selected from SEQ ID NOs:1-398. The isolated target site is capable of interacting with an antisense strand of the saRNA disclosed in the present disclosure, and thus capable of activating the expression of UTRN gene (e.g., mRNA expression, protein expression, UTRN expression). In some embodiments, the target site is selected based at least in part on a gene sequence. In some embodiments, the target site is selected based at least in part on a sequence close to a transcription start site (TSS) of the gene. In some embodiments, the target site is selected based at least in part on a promoter sequence upstream of the TSS. In some embodiments, the target site is selected based at least in part on a sequence from −5000 bp, −4000 bp, −3000 bp, −2000 bp, −1000 bp or −500 bp upstream of the TSS. In some embodiments, the target site is selected at least in part by moving toward the TSS by 1 bp each time, and resulting in a target sequence, followed by repeating this step and increasing towards the TSS by an additional base pair (e.g., n+1). In some embodiments, the target site has a length of about 8 to about 35 nucleotides. In some embodiments, the target site has a length of about 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides.

In certain embodiments, the present disclosure relates to an isolated oligonucleotide complex comprising the saRNA disclosed herein and the isolated target site disclosed in the present disclosure. In certain embodiments, the isolated oligonucleotide complex activates the expression of UTRN gene by at least 10% (e.g., activates/upregulates expression of the UTRN gene as compared to baseline UTRN gene expression levels).

### Hotspot

In certain embodiments, the present disclosure relates to an isolated nucleic acid sequence, or namely “hotspot region”, located upstream of the transcription start site of UTRN gene. In certain embodiments, isolated nucleic acid sequence disclosed herein is an oligonucleotide sequence having least 37 consecutive nucleotides in length and has at least 75%, or at least 80%, or at least 85%, or at least 90% sequence homology to an equal length region within the nucleotide sequence of SEQ ID NO:1200. In some embodiments, at least 25% (e.g., 28%, 35%, 40%, 45%, 50%, 55%, 60%, 65%, 70%, 75%, 80%, 85%, 90%, 95% or 100%) of the saRNAs designed to target a sequence within the hotspot is functional, i.e., can induce an at least 1.1-fold change in the mRNA expression of the target gene. In a non-limiting example, at least 50% of the saRNAs designed to the targeted hotspots is functional, i.e., can induce an at least 1.1-fold change in the mRNA expression of the target gene. In a non-limiting example, an saRNA is designed based at least in part on the following criteria: (1) having a GC content between 35% and 70%; (2) with less than 5 consecutive identical nucleotides; (3) with 3 or less dinucleotide repeats; and (4) with 3 or less trinucleotide repeats. In some embodiments, the same or similar criteria is used to select an isolated nucleic acid sequence and/or a target sequence. In a non-limiting example, an isolated nucleic acid sequence upstream of the UTRN gene's TSS is selected based at least in part on the following criteria: (1) having a GC content between 35% and 70%; (2) with less than 5 consecutive identical nucleotides; (3) with 3 or less dinucleotide repeats; and (4) with 3 or less trinucleotide repeats. In some embodiments, the isolated nucleic acid has about 19 to about 250 (e.g., about 27 to about 200, about 30 to about 200, about 33 to about 200, about 36 to about 150, about 39 to about 100, about 42 to about 75, about 45 to about 70, or about 48 to about 55) nucleotides in length. In some embodiments, a hotspot region is a polynucleotide sequence selected from the group consisting of SEQ ID NOs:1207-1210. In some embodiments, a hotspot region is a nucleic acid sequence selected from the group consisting of region −636 to −496 (SEQ ID NO:1207), region −351 to −294 (SEQ ID NO:1208), region −236 to −187 (SEQ ID NO:1209), or region −101 to −65 (SEQ ID NO:1210) upstream of the transcription start site of UTRN gene. The present disclosure also provides a method of designing saRNA, said method provide saRNA targeting said isolated nucleic acid sequence of the present disclosure.

In some embodiments, a target sequence is design/selected based, at least in part, on criteria that enables production of functional saRNA. For example, in some cases, a sequence located upstream of a TSS may include a sequence that does not favor synthesis of a target sequence despite being located in a hotspot region.

In some embodiments, a target sequence within a hotspot region is selected based, at least in part, on criteria that includes a sequence having a particular GC content (e.g., a GC content between 25% and 75%) and lacking consecutive identical nucleotides, consecutive dinucleotides, or consecutive trinucleotides. In some embodiments, a target sequence within a hotspot region comprises a sequence having a GC content between 35% and 70%; (2) with less than 5 consecutive identical nucleotides; (3) with 3 or less dinucleotide repeats; and (4) with 3 or less trinucleotide repeats.

In some embodiments, a target sequence comprises a sequence having a GC content between 25% and 75%, between 30% and 70%, between 35% and 70%, between 40% and 60%, or between 45% and 55%. In some embodiments, the saRNA comprises a sequence having a GC context between 35% and 70%.

In some embodiments, a target sequence comprises a sequence having less than 7 consecutive identical nucleotides, less than 6 consecutive identical nucleotides, less than 5 consecutive identical nucleotides, less than 4 consecutive identical nucleotides, or less than 3 consecutive identical nucleotides. In some embodiments, the saRNA comprises a sequence having less than 5 consecutive identical nucleotides.

In some embodiments, a target sequence comprises a sequence having 5 or less dinucleotide repeats, 4 or less dinucleotide repeats, 3 or less dinucleotide repeats, or 2 or less dinucleotide repeats. In some embodiments, the target sequence comprises a sequence having 3 or less dinucleotide repeats.

In some embodiments, a target sequence comprises a sequence having 5 or less trinucleotide repeats, 4 or less trinucleotide repeats, 3 or less trinucleotide repeats, or 2 or less trinucleotide repeats. In some embodiments, the target sequence comprises a sequence having 3 or less trinucleotide repeats.

RNAa activity of each designed saRNA is depended on a complex myriad of factors, such as chromatin environments, sequence features of the target per se and nearby regions, transcriptional factor binding etc. The core underlying determinant may be accessibility of the DNA target. In the regions with higher accessibility, dsRNAs may show a higher activity of RNAa. While dsRNAs designed targeting other regions of the promotor may exhibit non-functional or even transcriptional silencing effect. This may explain the existing of hotspot regions where functional saRNAs are clustered together. For example, a target sequence designed based at least in part on the following criteria: (1) having a GC content between 35% and 70%; (2) with less than 5 consecutive identical nucleotides; (3) with 3 or less dinucleotide repeats; and (4) with 3 or less trinucleotide repeats may not activate/upregulate the expression of UTRN gene by at least 10% as compared to baseline expression of the UTRN gene because the target sequence that the saRNA binds to is not within a hotspot region (e.g., any of hotspot regions described herein).

In certain embodiments, the present disclosure relates to an isolated nucleic acid complex comprising the saRNA disclosed in the present disclosure and the isolated nucleic acid sequence disclosed herein. In certain embodiments, the isolated nucleic acid complex activates the expression of UTRN gene by at least 10% as compared to baseline expression of the UTRN gene.

In some aspects, methods of using the isolated nucleic acid upstream of the transcription target site of UTRN gene is also provided.

DNA Encoding saRNA

In certain embodiments, the present disclosure relates to a nucleic acid or polynucleotide encoding the saRNA which can activate or upregulate the expression of UTRN gene in a cell by at least 10% (e.g., as compared to baseline expression of the UTRN gene). In certain embodiments, the nucleic acid is a DNA encoding an saRNA. In certain embodiments, the nucleic acid is a recombinant vector, specifically, a recombinant AAV vector. The vectors disclosed herein comprise a fragment of DNA that encodes an saRNA of the present disclosure.

Cell Comprising saRNA

After contacting a cell, the saRNA disclosed herein can effectively activate or upregulate the expression of UTRN gene in a cell, for example upregulate the expression by at least 10% (e.g., as compared to baseline expression of the UTRN gene).

In certain embodiments, the present disclosure relates to a cell comprising the saRNA disclosed herein. In some embodiments, the cell is a mammalian cell. In some embodiments, the cell is a human cell, such as a human malignant embryonic rhabdomyoma cells (e.g., a RD cell). The cell disclosed herein may be in vitro, or ex vivo, such as a cell line or a cell strain, or may exist in a mammalian body, such as a human body. The human body disclosed herein is a subject suffering from a disease or symptom caused by a UTRN gene mutation, low utrophin level, and/or insufficient levels of functional utrophin in muscle. In some embodiments, the cell is from a subject suffering from DMD.

Composition Comprising saRNA

In certain embodiments, the present disclosure relates to a composition or pharmaceutical composition comprising the saRNA or the nucleic acid of the present disclosure. In some embodiments, the composition comprises at least one pharmaceutically acceptable carrier. In some embodiments, the composition comprising at least one pharmaceutically acceptable carrier selected from an aqueous carrier, liposome or LNP, polymer, micelle, colloid, metal nanoparticle, non-metallic nanoparticle, bioconjugate (e.g., GalNAc), polypeptide, antibody and any combination thereof. In one embodiment, the aqueous carrier may be, for example, RNase-free water, or RNase-free buffer. In some embodiments, the composition may contain 0.001-200 nM (e.g., 0.01-100 nM, 0.1-50 nM, 1-150 nM, 1-200 nM, 1-20 nM, 0.001-1 nM, 1-10 nM, 10-100 nM, 10-50 nM, 20-50 nM, 20-100 nM) of the saRNA or isolated polynucleotide as described herein. In some embodiments, the composition includes 25 nM of the saRNA or isolated polynucleotide as described herein.

Methods of Using saRNA

Another aspect of the present disclosure relates to an saRNA for activating/upregulating the UTRN gene expression in a cell. The saRNA comprises an oligonucleotide sequence having a length of 16 to 35 consecutive nucleotides. In some embodiments, the oligonucleotide sequence has at least 75%, or at least 80%, or at least 85%, or at least 90% homology or complementary to an equal length region of SEQ ID NO:1200, specifically, the saRNA activates/up-regulates the expression of the UTRN gene by at least 10% (e.g., at least 20%, at least 30%, at least 40%, at least 50%, at least 60%, at least 70%, at least 80%, at least 90%, at least 100%, at least 150%, at least 200%, at least 300%, at least 500%, at least 800%, at least 1000%, at least 2000%, or at least 5000% as compared to baseline expression of the UTRN gene). In certain embodiments, upon administering the saRNA disclosed in the embodiments, e.g., to a cell or a subject, the expression of the UTRN gene is activated/up-regulated by at least 1.1 fold (e.g., at least 1.2 fold, at least 1.5 fold, at least 1.8 fold, at least 2.0 fold, or at least 2.2 fold compared to baseline expression of the UTRN gene). In certain embodiments, an saRNA activates or upregulates the expression of the UTRN gene by about 2.2-fold. In certain embodiments, the expression of UTRN gene is activated/up-regulated by administering the saRNA disclosed in the embodiments to a cell at a concentration of at least 0.01 nM, e.g., 0.02 nM, 0.05 nM, 0.08 nM, 0.1 nM, 0.2 nM, 0.3 nM, 0.4 nM, 0.5 nM, 0.6 nM, 0.8 nM, 1 nM, 5 nM, 10 nM, 25 nM, 50 nM, 75 nM, 100 nM, 150 nM or 200 nM. In certain embodiments, the induction of UTRN gene coding protein (utrophin) is activated/up-regulated by administering the saRNA disclosed in the embodiments, e.g., to a cell or a subject, the expression of the UTRN gene coding protein (utrophin) by at least 1.1 fold (e.g., at least 2 fold, at least 3 fold, at least 4 fold, at least 5 fold, at least 6 fold, at least 7 fold, or at least 8 fold compared to baseline expression of the utrophin protein). In certain embodiments, an saRNA activates or upregulates the expression of the utrophin protein by about 8.0-fold. In certain embodiments, the induction of UTRN gene coding protein (utrophin) is activated/up-regulated by administering the saRNA disclosed in the embodiments to a cell at a concentration of at least 0.01 nM, e.g., 0.02 nM, 0.05 nM, 0.08 nM, 0.1 nM, 0.2 nM, 0.3 nM, 0.4 nM, 0.5 nM, 0.6 nM, 0.8 nM, 1 nM, 2 nM, 3 nM, 4 nM, 5 nM, 10 nM, 25 nM, 50 nM, 75 nM, 100 nM, 150 nM or 200 nM.

Another aspect of the present disclosure relates to a method for preventing or treating a disorder or condition induced by insufficient expression of dystrophin, a dystrophin gene mutation, and/or low functional dystrophin levels in an individual comprising: administering an effective amount of the saRNA, the nucleic acid or isolated polynucleotide encoding the saRNA, or the composition comprising the saRNA disclosed herein to the individual. in certain embodiments, the effective amount of the saRNA disclosed herein can be a concentration ranging from 0.01 nM to 50 nM, e.g., 0.01 nM, 0.02 nM, 0.05 nM, 0.08 nM, 0.1 nM, 0.2 nM, 0.3 nM, 0.4 nM, 0.5 nM, 0.6 nM, 0.8 nM, 1 nM, 5 nM, 10 nM, 25 nM, 50 nM, 75 nM, 100 nM, 150 nM or 200 nM. In some embodiments, the disorder or condition is DMD. In some embodiments, the individual is a mammal. In some embodiments, the individual is a human.

In any of the embodiments provided herein, such saRNA, nucleic acids encoding the saRNA of the present disclosure, or compositions comprising such saRNA of the present disclosure may be introduced directly into a cell, or may be produced intracellularly upon introduction of a nucleotide sequence encoding the saRNA into a cell, for example a mammalian cell including, but not limited to RD cells, or a human cell. Such cells may be ex vivo, such as cell lines, and the like, or may be present in mammalian bodies, such as humans. In some embodiments, the human is a subject or individual suffering from a dystrophin-deficiency-related condition or DMD or BMD. In certain embodiments, a nucleic acid or an isolated polynucleotide encoding an saRNA or a composition comprising the aforementioned saRNA as described herein, in respective amounts sufficient to treat DMD or BMD.

Another aspect of the present disclosure relates administering an effective mount of the saRNA or the composition to an individual using administration pathway as described herein. In some embodiments, the administration pathway is selected from one or more of: parenteral infusions, oral administration, intranasal administration, inhaled administration, vaginal administration, and rectal administration. In some embodiments, the administration pathway is selected from one or more of: intrathecal, intramuscular, intravenous, intra-arterial, intraperitoneal, intravesical, intracerebroventricular, intravitreal and subcutaneous administrations.

### Dose Regiments and Route of Administration

Aspects of the present disclosure relate to a pharmaceutical composition comprising the saRNA of the present disclosure. In some embodiments, the pharmaceutical composition comprises the saRNA of the present disclosure and a pharmaceutically acceptable carrier, a therapeutically inert carrier, diluent or pharmaceutically acceptable excipient. The pharmaceutical composition disclosed herein is to be developed into a medicament preventing or treating the dystrophin-deficiency-related condition or DMD or BMD.

Aspects of the present disclosure also relate to methods of using the saRNAs of the present disclosure to prepare such compositions.

Another aspect of the present disclosure relates to use of the saRNA of the present disclosure in manufacturing the pharmaceutical composition disclosed herein.

Another aspect of the present disclosure relates to use of the saRNA or an isolated polynucleotide, according to any one of the embodiments described herein, or a composition according to any one of the embodiments described herein, in the manufacture of a medicament for the prevention or treatment of gene or protein-related symptom induced by the insufficient expression of dystrophin, a dystrophin gene mutation, and/or low functional dystrophin levels in an individual. For the use according to certain embodiments, the condition can include a dystrophin-mutation-related disorder or condition that comprises a DMD. For the use according to certain embodiments, the symptom induced by insufficient expression of utrophin is BMD or DMD. Also related is the use according to certain embodiments wherein the individual is a mammal, for example a human.

The dosage at which the saRNAs or compositions of the present disclosure can be administered can vary within wide limits and will be fitted to the individual requirements in each case. In certain embodiments, a first dose of a pharmaceutical composition according to the present disclosure is administered when the subject is less than one week old, less than one month old, less than 3 months old, less than 6 months old, less than one-year-old, less than 2 years old, less than 15 years old, or older than 15 years old.

The single dose of the saRNA can be a single dose ranging from 0.01 mg/kg to 1000 mg/kg for example, about 0.01, 0.02, 0.05, 0.1, 0.2, 0.5, 1, 2, 2.5, 5, 7.5, 10, 12.5, 15, 17.5, 20, 25, 30, 40, 50, 75, 100, 120, 150, 200, 250, 300, 400, 500, 750, or 1000 mg/kg. The doses described herein may contain two or more of any of the saRNA sequences described herein.

In some embodiments, the proposed dose frequency is approximate. For example, in certain embodiments if the proposed dose frequency is a dose at day 1 and a second dose at day 29, a DMD patient may receive a second dose 25, 26, 27, 28, 29, 30, 31, 32, 33, or 34 days after receipt of the first dose. In certain embodiments, if the proposed dose frequency is a dose at day 1 and a second dose at day 15, a DMD patient may receive a second dose 10, 11, 12, 13, 14, 15, 16, 17, 18, 19, or 20 days after receipt of the first dose. In certain embodiments, if the proposed dose frequency is a dose at day 1 and a second dose at day 85, a DMD patient may receive a second dose 80, 81, 82, 83, 84, 85, 86, 87, 88, 89, or 90 days after receipt of the first dose.

In certain embodiments, the dose and/or the volume of the injection will be adjusted based on the subject's age, the subject's body weight, and/or other factors that may require adjustment of the parameters of the injection.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may vary considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Examples of other compositions or components associated with the saRNA, compositions, pharmaceutical compositions, and methods described herein include, but are not limited to: diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use. 11471 In some embodiments, lipid moieties used in nucleic acid therapies can be applied in the present disclosure for delivery of the saRNA molecules disclosed herein. In such methods, the nucleic acid (e.g., one or more saRNAs described herein) is introduced into preformed liposomes or lipoplexes made of mixtures of cationic lipids and neutral lipids. In certain methods, saRNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present disclosure to specific tissues or cell types. For example, in certain embodiments, pharmaceutical compositions include liposomes coated with a tissue-specific antibody.

In some embodiments, the saRNA can be delivered or administered via a vector. Any vectors that may be used for gene delivery may be used. In some embodiments, a viral vector may be used. Non-limiting examples of viral vectors that may be used in the present disclosure include, but are not limited to, human immunodeficiency virus; HSV, herpes simplex virus; MMSV, Moloney murine sarcoma virus; MSCV, murine stem cell virus; SFV, Semliki Forest virus; SIN, Sindbis virus; VEE, Venezuelan equine encephalitis virus; VSV, vesicular stomatitis virus; VV, vaccinia virus; AAV, adeno-associated virus; adenovirus; lentivirus; and retrovirus.

In some embodiments, the vector is a recombinant AAV vector (rAAV). AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs (rAAV) according to the disclosure can be prepared by co-transfecting a plasmid containing the nucleic acid sequence of interest flanked by two AAV inverted terminal repeat (ITR) regions, and a plasmid carrying the AAV encapsulation genes (rep and cap genes), into a cell line that is infected with a human helper virus (for example an adenovirus). The AAV recombinants that are produced are then purified by standard techniques.

In some embodiments, the vector(s) for use in the methods of the disclosure are encapsulated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the disclosure may include a recombinant virus particle (recombinant because it contains a recombinant polynucleotide) comprising any of the vectors described herein. Methods of producing such particles are known in the art and are described in U.S. Pat. No. 6,596,535.

Preparations, pharmaceutical compositions, or medicaments of the present disclosure are formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners.

For the preparations, pharmaceutical compositions, or medicaments of the present disclosure, the delivery can be optionally through parenteral infusions including intrathecal, intramuscular, intravenous, intra-arterial, intraperitoneal, intravesical, intracerebroventricular, intravitreal or subcutaneous administration; or through oral administration, intranasal administration, inhaled administration, vaginal administration, or rectal administration.

A typical formulation of the oligonucleotide modulator in the present disclosure is prepared by mixing an saRNA of the present disclosure and a carrier or excipient. Suitable carriers and excipients are well known to those skilled in the art and are described in detail in, e.g., Ansel H. C. et al., Ansel's Pharmaceutical Dosage Forms and Drug Delivery Systems (2004) Lippincott, Williams & Wilkins, Philadelphia; Gennaro A. R. et al., Remington: The Science and Practice of Pharmacy (2000) Lippincott, Williams & Wilkins, Philadelphia; and Rowe R. C, Handbook of Pharmaceutical Excipients (2005) Pharmaceutical Press, Chicago. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., an saRNA of the present disclosure or pharmaceutical composition thereof) or aid in the manufacturing of the pharmaceutical product (i.e., medicament).

### Method of Diagnosis

Another aspect of the present disclosure relates to a method for detecting dystrophin, utrophin or dystrophin related protein (e.g., dystroglycan) in a cell. In certain embodiments, the method includes detecting dystrophin, utrophin or dystrophin related protein (e.g., dystroglycan) in a cell transfected with the saRNA, the isolated polynucleotide, or the composition comprising the saRNA as disclosed herein in the present disclosure. In certain embodiments, the method disclosed herein can be applied in detecting a specific sub-group of subjects suffering from a disorder or condition induced by insufficient expression of dystrophin, a dystrophin gene mutation, and/or low functional dystrophin levels in an individual. As alternative embodiments of the method disclosed herein, the method can be used in efficacy or safety monitoring of the aforementioned subjects treated by the saRNA, nucleic acid or isolated polynucleotide encoding the saRNA, composition, or medicament of the present disclosure.

In certain embodiments, a baseline measurement is obtained from a biological sample, as defined herein, obtained from an individual prior to administering the therapy described herein. In certain embodiments, a baseline expression of the dystrophin or DMD gene is the expression of the dystrophin or DMD gene obtained from a biological sample prior to administering the saRNA described herein. In certain embodiments, a baseline expression of the utrophin or UTRN gene is the expression of the utrophin or UTRN gene obtained from a biological sample prior to administering the saRNA described herein. In certain embodiments, the biological sample is peripheral blood cells, plasma, muscle cells, serum, skin tissue, cerebrospinal fluid (CSF).

In some embodiments, the saRNA provided herein activates the amount of functional utrophin in a cell as compared to the baseline measurement aforementioned, by at least 10% (e.g., at least 20%, at least 30%, at least 40%, at least 50%, at least 60%, at least 70%, at least 80%, at least 90%, at least 100%, at least 150%, at least 200%, at least 300%, at least 500%, at least 800%, at least 1000%, at least 2000%, or at least 5000%).

In some embodiments, the saRNA shows a greater than additive effect or synergy in the treatment, prevention, delaying progression and/or amelioration of diseases caused by the dystrophin gene mutation. In some embodiments, the saRNA shows a greater than additive effect or synergy in the protection of cells implicated in the pathophysiology of the disease, particularly for the treatment, prevention, delaying progression and/or amelioration DDD (e.g., DMD or BMD).

Another aspect of the present disclosure relates to a method for activating/up-regulating expression of UTRN gene in a cell comprising: administering the saRNA, or the isolated polynucleotide, or the composition of the embodiments disclosed herein. In some embodiments, the saRNA, or the isolated polynucleotide, or the composition is introduced directly into the cell. In some embodiments, the saRNA of the embodiments disclosed herein is produced in the cell after a nucleotide sequence encoding the saRNA is introduced into the cell. In some embodiments, the cell disclosed herein is a mammalian cell, for example a human cell.

Another aspect of the present disclosure relates to a method for increasing a level of utrophin in a cell or a level of functional utrophin in muscle of a subject, comprising introducing an effective amount of the saRNA, the nucleic acid or polynucleotide encoding the saRNA, or the composition of the embodiments disclosed herein into the cell or subject.

### Kit

Another aspect of the present disclosure relates to a kit for performing the method for increasing a level of utrophin in a cell or a level of functional utrophin in muscle, comprising the saRNA disclosed herein. In certain embodiments, the kit further comprises means for administering said saRNA to an individual. In certain embodiments, the kit is in a labeled package and the label on said package indicates that the saRNA or the composition can be used in preventing or treating a disease or condition induced by insufficient expression of dystrophin, a dystrophin gene mutation, and/or low functional dystrophin levels in an individual, or against DDD, e.g., DMD or BMD.

A “kit” as used herein, typically defines a package, assembly, or container (such as an insulated container) including one or more of the components or embodiments of the disclosure, and/or other components associated with the disclosure, for example, as previously described. Any of the agents or components of the kit may be provided in liquid form (e.g., in solution), or in solid form (e.g., a dried powder, frozen, etc.).

In additional embodiments, a kit can include instructions or instructions to a website or other source in any form that are provided for using the kit in connection with the components and/or methods described herein. For instance, the instructions may include instructions for the use, modification, mixing, diluting, preserving, assembly, storage, packaging, and/or preparation of the components and/or other components associated with the kit. In some cases, the instructions may also include instructions for the delivery of the components, for example, for shipping or storage at room temperature, sub-zero temperatures, cryogenic temperatures, etc. The instructions may be provided in any form that is useful to the user of the kit, such as written or oral (e.g., telephonic), digital, optical, visual (e.g., videotape, DVD, etc.) and/or electronic communications (including Internet or web-based communications), provided in any manner.

Another aspect of the present disclosure relates to a kit for detecting dystrophin, utrophin or dystrophin related protein (e.g., dystroglycan) in a cell. In certain embodiments, the kit is for detecting dystrophin, utrophin or dystrophin related protein (e.g., dystroglycan) in a cell transfected with any one or more of the saRNA disclosed herein, or the isolated polynucleotide, or the composition disclosed herein. Also provided herein is a kit for increasing a level of utrophin in a cell.

### Particular Embodiments

The present disclosure provides the following particular embodiments:

Embodiment 1 is a small activating RNA (saRNA) comprising an oligonucleotide sequence having a length ranging from 16 to 35 consecutive nucleotides, wherein the oligonucleotide sequence comprises a continuous nucleotide sequence having at least 75%, at least 80%, at least 85%, or at least 90% homology or complementarity to an equal length portion of SEQ ID NO:1200, wherein the saRNA upregulates the expression of UTRN gene by at least 10% as compared to baseline expression of the UTRN gene.

Embodiment 2 is the saRNA of embodiment 1, wherein the equal length portion of SEQ ID NO:1200 is located in the region −636 to −496 (SEQ ID NO:1207), region −351 to −294 (SEQ ID NO:1208), region −236 to −187 (SEQ ID NO:1209), or region −101 to −65 (SEQ ID NO:1210) upstream of the transcription start site of UTRN gene.

Embodiment 3 is the saRNA of any one of embodiments 1-2, wherein the saRNA (1) has a GC content between 35% and 70%; (2) with less than 5 consecutive identical nucleotides; (3) with 3 or less dinucleotide repeats; and (4) with 3 or less trinucleotide repeats.

Embodiment 4 is the saRNA of any one of embodiments 1-3, wherein the saRNA comprises a sense strand and an antisense strand.

Embodiment 5 is the saRNA of any one of embodiments 1-4, wherein the oligonucleotide sequence is the sense strand or the antisense strand of the saRNA.

Embodiment 6 is the saRNA of any one of embodiments 1-5, wherein the sense strand and the antisense strand each comprise complementary regions, wherein the complementary regions of the sense strand and the antisense strand form a double-stranded nucleic acid structure.

Embodiment 7 is the saRNA of any one of embodiments 4-6, wherein the sense strand and the antisense strand have a complementarity of at least 90%.

Embodiment 8 is the saRNA of embodiment 4, wherein the sense strand and the antisense strand are located on two different nucleic acid strands.

Embodiment 9 is the saRNA of embodiment 4, wherein the sense strand and the antisense strand are located on a contiguous nucleic acid strand, optionally a hairpin single-stranded nucleic acid molecule, wherein the complementary regions of the sense strand and the antisense strand form a double-stranded nucleic acid structure.

Embodiment 10 is the saRNA of embodiment 4, wherein at least one of the sense strand and the antisense strand comprises a 3′ overhang ranging from 0 to 6 nucleotides in length.

Embodiment 11 is the saRNA of embodiment 10, wherein the sense strand and the antisense strand comprise a 3′ overhang of ranging from 2 to 3 nucleotides in length.

Embodiment 12 is the saRNA of embodiment 10, wherein at least one of the nucleotides of the overhang is nucleotides selected from or complementary to the corresponding nucleotides on the UTRN gene.

Embodiment 13 is the saRNA of any of embodiments 4-12, wherein the sense strand and the antisense strand independently comprise a length of about 16 to about 35, about 17 to about 30, about 18 to about 25, or about 19 to about 22 consecutive nucleotides.

Embodiment 14 is the saRNA of any one of embodiments 4-12, wherein the sense strand has at least 75% sequence homology to a nucleotide sequence selected from SEQ ID NOs: 400-797, and the antisense strand has at least 75% sequence homology to a nucleotide sequence selected from SEQ ID NOs: 800-1197.

Embodiment 15 is the saRNA of embodiment 14, wherein the sense strand comprises a nucleotide sequence selected from SEQ ID NOs: 400-797, and the antisense strand comprises a nucleotide sequence selected from SEQ ID NOs: 800-1197.

Embodiment 16 is the saRNA of embodiment 1, wherein the oligonucleotide sequence has at least 75% sequence homology or complementarity to a nucleotide sequence selected from SEQ ID NOs: 1-398.

Embodiment 17 is the saRNA of embodiment 4, wherein the sense strand has at least 75% sequence homology to a nucleotide sequence selected from SEQ ID NOs: 1-398.

Embodiment 18 is the saRNA of embodiment 4, wherein the antisense strand has at least 75% sequence complementarity to a nucleotide sequence selected from SEQ ID NOs: 1-398.

Embodiment 19 is the saRNA of any of embodiments 1-18, wherein at least one nucleotide of the saRNA is a chemically modified nucleotide.

Embodiment 20 is the saRNA of embodiment 19, wherein at least one nucleotide of the antisense and/or sense strand of the saRNA is chemically modified.

Embodiment 21 is the saRNA of embodiment 19, wherein the chemically modified nucleotide is a nucleotide with at least one the following modifications:
a) modification of a phosphodiester bond connecting nucleotides in the nucleotide sequence of the saRNA;
b) modification of 2′-OH of a ribose in the nucleotide sequence of the saRNA; and
c) modification of a base in the nucleotide sequence of the saRNA.

Embodiment 22 is the saRNA of embodiment 19, wherein at least one nucleotide of the saRNA is a locked nucleic acid, an abasic nucleotide, a 2′-amino-modified nucleotide, a 2′-alkyl-modified nucleotide, a morpholino nucleotide, a phosphoramidate, or a non-natural base comprising nucleotide.

Embodiment 23 is the saRNA of embodiment 19, wherein the chemical modification of the at least one chemically modified nucleotide is an addition of a (E)-vinylphosphonate moiety at the 5′ end of the sense strand or the antisense strand.

Embodiment 24 is the saRNA of any one of embodiments 1-23 wherein the sense strand or the antisense strand of the saRNA is conjugated to one or more conjugation moieties selected from a lipid, a fatty acid, a fluorophore, a ligand, a saccharide, a peptide, and an antibody.

Embodiment 25 is the saRNA of embodiment 24, wherein the sense strand or the antisense strand of the saRNA is conjugated to one or more conjugation moieties selected from a cell-penetrating peptide, polyethylene glycol, an alkaloid, a tryptamine, a benzimidazole, a quinolone, an amino acid, a cholesterol, glucose, and N-acetylgalactosamine, and any combinations thereof.

Embodiment 26 is an oligonucleotide modulator comprising one or more saRNA according to any one of embodiments 1-25.

Embodiment 27 is the oligonucleotide modulator of embodiment 26, further comprising one or more moieties or components conjugated, combined or bonded with said saRNA(s).

Embodiment 28 is the oligonucleotide modulator of embodiment 27, wherein the sense strand and/or the antisense strand of the saRNA is conjugated to one or more conjugation moieties selected from the group consisting of a lipid, a fatty acid, a fluorophore, a ligand, a saccharide, a peptide, and an antibody.

Embodiment 29 is the oligonucleotide modulator of embodiment 27, wherein the conjugation moiety is each independently selected from a lipid, a cell-penetrating peptide, a polyethylene glycol, an alkaloid, a tryptamine, a benzimidazole, a quinolone, an amino acid, a cholesterol, a glucose, a N-acetylgalactosamine, and any combinations thereof.

Embodiment 30 is the oligonucleotide modulator of embodiment 26, wherein the oligonucleotide modulator further comprises a saRNA conjugated to or combined with one or more of other active moieties for UTRN associated diseases or disorder treatment, wherein the one or more of other active moieties are each independently selected from a saRNA, a single-stranded oligonucleotide, a chemical moiety, a polypeptide and an antibody.

Embodiment 31 is an isolated polynucleotide, wherein the isolated polynucleotide comprises the continuous nucleotide sequence of embodiment 1.

Embodiment 32 is the isolated polynucleotide of embodiment 31, wherein the isolated polynucleotide is a nucleic acid sequence selected from SEQ ID NOs:1-398.

Embodiment 33 is an isolated oligonucleotide complex comprising the antisense strand of the saRNA of any of embodiments 1-25 and the isolated polynucleotide of any of embodiments 31-32.

Embodiment 34 is the isolated oligonucleotide complex of embodiment 33, wherein the isolated oligonucleotide complex activates the expression of UTRN gene by at least 10% as compared to baseline expression of the UTRN gene.

Embodiment 35 is an isolated nucleic acid sequence upstream of the transcription start site of UTRN gene, wherein the isolated nucleic acid sequence is selected from SEQ ID NOs:1207-1210.

Embodiment 36 is the isolated nucleic acid sequence of embodiment 35, wherein the isolated nucleic acid sequence comprises the isolated polynucleotide of any one of embodiments 31-32.

Embodiment 37 is the isolated nucleic acid sequence of embodiment 35, wherein at least 25% of designed saRNA targeting the isolated nucleic acid sequence can activate the expression of UTRN gene by at least 10%, wherein the designed saRNA (1) having a GC content between 35% and 70%; (2) with less than 5 consecutive identical nucleotides; (3) with 3 or less dinucleotide repeats; and (4) with 3 or less trinucleotide repeats.

Embodiment 38 is an isolated nucleic acid complex comprising the antisense strand of the saRNA of any of embodiments 1-25 and the sense strand of the isolated nucleic acid sequence of any of embodiments 35-37.

Embodiment 39 is the isolated nucleic acid complex of embodiment 38, wherein the isolated nucleic acid complex activates the expression of UTRN gene by at least 10% as compared to baseline expression of the UTRN gene.

Embodiment 40 is an isolated polynucleotide encoding the saRNA of any one of embodiments 1-25.

Embodiment 41 is the isolated polynucleotide of embodiment 40, wherein the isolated polynucleotide is a DNA.

Embodiment 42 is a vector comprising the isolated polynucleotide of any one of embodiments 40-41.

Embodiment 43 is a host cell comprising the saRNA of any one of embodiments 1-25, the isolated polynucleotide of any one of embodiments 40-41, or the vector of embodiment 42.

Embodiment 44 is a composition comprising the saRNA of any one of embodiments 1-25, or the isolated polynucleotide of embodiment 40 or embodiment 41 and optionally, a pharmaceutically acceptable carrier.

Embodiment 45 is the composition of embodiment 44, wherein the pharmaceutically acceptable carrier is selected from the group consisting of an aqueous carrier, a liposome, a high-molecular polymer, a polypeptide and an antibody.

Embodiment 46 is the composition of embodiment 44 or 45, wherein the composition comprises 0.001-200 nM of the saRNA.

Embodiment 47 is the composition of embodiment 46, wherein the composition comprises 1-200 nM of the saRNA.

Embodiment 48 is an saRNA comprising an oligonucleotide sequence with a length ranging from 16 to 35 continuous nucleotides for activating/upregulating UTRN gene expression in a cell, wherein the oligonucleotide sequence has at least 75%, or at least 80%, or at least 85%, or at least 90% sequence homology or complementary to an equal length portion of SEQ ID NO:1200, wherein the saRNA activates the expression of UTRN gene by at least 10% as compared to its baseline expression.

Embodiment 49 is the saRNA of embodiment 48, wherein the equal length region of SEQ ID NO:1200 is located in the region −636 to −496 (SEQ ID NO:1207), region −351 to −294 (SEQ ID NO:1208), region −236 to −187 (SEQ ID NO:1209), or region −101 to −65 (SEQ ID NO:1210) upstream of the transcription start site of UTRN gene.

Embodiment 50 is the saRNA of embodiment 49, wherein the saRNA comprises a sense strand and an antisense strand, wherein the sense strand comprises a nucleotide sequence selected from SEQ ID NOs: 400-797, and the antisense strand comprises or is a nucleotide sequence selected from SEQ ID NOs: 800-1197.

Embodiment 51 is a product for activating/up-regulating UTRN gene expression in a cell, wherein the product activates the expression of UTRN gene by at least 10% as compared to baseline expression of the UTRN gene, and wherein the product comprises an active substance selected from one or more of the saRNA of any one of embodiments 1-25, the isolated polynucleotide of any one of embodiments 40-41, the vector of embodiment 42, or the composition of any one of embodiments 44-47.

Embodiment 52 is the product for activating/up-regulating UTRN gene expression in a cell, wherein the active substance is introduced directly into the cell; and/or
wherein the cell is in vitro, ex vivo or in vivo; and/or
wherein the cell is a mammalian cell.

Embodiment 53 is the product of embodiment 52, wherein the active substance is introduced directly into the cell by:
1) composing the active substance with a physiologically acceptable or pharmaceutically acceptable carrier, such as one or more selected from the group consisting of an aqueous carrier, a liposome, a high-molecular polymer, a polypeptide and an antibody, and/or
2) conjugating the active substance to one or more conjugation moieties, such as one or more selected from a lipid, a cell-penetrating peptide, a polyethylene glycol, an alkaloid, a tryptamine, a benzimidazole, a quinolone, an amino acid, a cholesterol, a glucose, and a N-acetylgalactosamine, and any combinations thereof (for example two conjugation moieties wherein one is a lipid and the other is a N-acetylgalactosamine).

Embodiment 54 is the product of embodiment 53, wherein the conjugation moiety is independently derived from a fluorophore, a ligand, a saccharide, a peptide, and an antibody.

Embodiment 55 is the product for activating/up-regulating UTRN gene expression in a cell, wherein the cell is from a patient suffering from or in risk of having a disease or condition induced by insufficient expression of the UTRN protein, a UTRN gene mutation, and/or low functional UTRN levels, wherein the active substance is administered in a sufficient amount to prevent or treat the disease or condition, such as Duchenne muscular dystrophy (DMD) or Becker muscular dystrophy (BMD).

Embodiment 56 is a method for preventing or treating a disease or condition induced by insufficient expression of dystrophin, a dystrophin gene mutation, and/or low functional dystrophin levels in an individual comprising: administering an effective amount of the saRNA of any one of embodiments 1-25, the isolated polynucleotide of any one of embodiments 40-41, the vector of embodiment 42, or the composition of any one of embodiments 44-46 to the individual.

Embodiment 57 is the method of embodiment 56, wherein the disease or condition is a dystrophin deficiency disorder (DDD).

Embodiment 58 is the method of embodiment 56, wherein the disease or condition is a Duchenne muscular dystrophy (DMD) or Becker muscular dystrophy (BMD).

Embodiment 59 is the method of embodiment 56, wherein the individual is a mammal, optionally wherein the individual is a human.

Embodiment 60 is the method of embodiment 56, wherein the individual suffers from a symptom induced by insufficient expression of dystrophin, a dystrophin gene mutation, and/or low functional dystrophin levels in an individual.

Embodiment 61 is the method of embodiment 56, wherein the saRNA of any one of embodiments 1-25, the isolated polynucleotide of any one of embodiments 40-41, the vector of embodiment 42, or the composition of any one of embodiments 44-47 is administrated to an individual by an administration pathway selected from one or more of: parenteral infusions, oral administration, intranasal administration, inhaled administration, vaginal administration, and rectal administration.

Embodiment 62 is the method of embodiment 61, wherein the administration pathway is selected from one or more of intrathecal, intramuscular, intravenous, intraarterial, intraperitoneal, intravesical, intracerebroventricular, intravitreal and subcutaneous administrations.

Embodiment 63 is the method of embodiment 56, wherein the method activates/up-regulates expression of the UTRN gene mRNA in the individual by at least 10% as compared to baseline expression of the UTRN gene.

Embodiment 64 is the method of embodiment 56, wherein the method increases a level of utrophin in the individual by at least 10% as compared to baseline expression of the UTRN gene.

Embodiment 65 is a method for detecting dystrophin, utrophin or dystrophin related protein (e.g., dystroglycan) in the host cell of embodiment 43.

Embodiment 66 is a kit for performing the method of embodiment 56, comprising a) saRNA of any one of embodiments 1-25.

Embodiment 67 is the kit of embodiment 66, wherein the kit comprises b) instructions for use, and c) optionally, means for administering the saRNA of any one of embodiments 1-25 to an individual.

Embodiment 68 is a kit comprising the saRNA of any one of embodiments 1-25, the isolated polynucleotide of any one of embodiments 40-41, the vector of embodiment 42, or the composition of any one of embodiments 44-47 in a labeled package and the label on package indicates that the saRNA, the isolated polynucleotide, the vector or the composition can be used in preventing or treating a disease or condition induced by insufficient expression of dystrophin, or against Duchenne muscular dystrophy (DMD) or Becker muscular dystrophy (BMD).

Embodiment 69 is a kit for detecting dystrophin, utrophin or dystrophin related protein (e.g., dystroglycan) in the host cell of embodiment 43.

Embodiment 70 is use of the saRNA of any one of embodiments 1-25, the isolated polynucleotide of any one of embodiments 40-41, the vector of embodiment 42, or the composition of any one of embodiments 44-47 in preparing a medicament for preventing or treating a disease or condition induced by insufficient expression of dystrophin, a dystrophin gene mutation, and/or low functional dystrophin levels in an individual.

Embodiment 71 is the use of embodiment 70, wherein the disease or condition is a Duchenne muscular dystrophy (DMD) or Becker muscular dystrophy (BMD).

Embodiment 72 is the use of embodiment 70, wherein the individual is a mammal, optionally wherein the mammal is a human.

Embodiment 73 is use of the saRNA of any one of embodiments 1-25, the isolated polynucleotide of any one of embodiments 40-41, the vector of embodiment 42, or the composition of any one of embodiments 44-47 in preparing a preparation for activating/up-regulating expression of UTRN gene in a cell.

Embodiment 74 is the use of embodiment 73, wherein the saRNA of any one of embodiments 1-25, or the isolated polynucleotide of any one of embodiments 40-41, the vector of embodiment 42, or the composition of any one of embodiments 44-47 is directly introduced into the cell.

Embodiment 75 is the use of embodiment 74, wherein the saRNA is produced in the cell after a nucleotide sequence encoding the saRNA is introduced into the cell.

Embodiment 76 is the use of any of embodiments 73-75, wherein the cell is a mammalian cell, optionally wherein the mammalian cell is a human cell.

Embodiment 77 is the use of embodiment 76, wherein the cell is in a human body.

Embodiment 78 is the use of embodiment 77, wherein the human body is a subject suffering from a symptom induced by the insufficient expression of dystrophin, a dystrophin gene mutation, and/or low functional dystrophin levels in an individual, wherein the saRNA, the isolated polynucleotide or the composition is administered in a sufficient amount to treat the symptom.

Embodiment 79 is the use of embodiment 78, wherein the symptom induced by insufficient expression of dystrophin is a Duchenne muscular dystrophy (DMD) or Becker muscular dystrophy (BMD).

Embodiment 80 is a method for activating/up-regulating expression of UTRN gene in a cell comprising: administering an effective amount of the saRNA of any one of embodiments 1-25, the isolated polynucleotide of any one of embodiments 40-41, the vector of embodiment 42, or the composition of any one of embodiments 44-47 to the cell.

Embodiment 81 is the method of embodiment 80, wherein the saRNA of any one of embodiments 1-25, the isolated polynucleotide of any one of embodiments 40-41, the vector of embodiment 42, or the composition of any one of embodiments 44-47 is introduced directly into the cell.

Embodiment 82 is the method of embodiment 81, wherein the method, for introducing directly into the cell, comprises:
1) composing the saRNA of any one of embodiments 1-25, the isolated polynucleotide of any one of embodiments 40-41, the vector of embodiment 42, or the saRNA in the composition of any one of embodiments 44-47 with a pharmaceutically acceptable carrier selected from the group consisting of an aqueous carrier, a liposome, a high-molecular polymer, a polypeptide and an antibody, and
2) conjugating the saRNA of any one of embodiments 1-25, the isolated polynucleotide of any one of embodiments 40-41, the vector of embodiment 42, or the saRNA in the composition of any one of embodiments 44-47 to one or more conjugation moieties selected from a cell-penetrating peptide, polyethylene glycol, an alkaloid, a tryptamine, a benzimidazole, a quinolone, an amino acid, a cholesterol, glucose, and N-acetylgalactosamine.

Embodiment 83 is the method of any of embodiments 80-82, wherein the cell is a mammalian cell, for example a cell from a human body.

Embodiment 84 is the method of embodiment 83, wherein the human body is a subject suffering from a symptom induced by insufficient expression of dystrophin, a dystrophin gene mutation, and/or low functional dystrophin levels in an individual, wherein the saRNA, the isolated polynucleotide or the composition is administered in a sufficient amount to treat the symptom.

Embodiment 85 is the method of embodiment 84, wherein the symptom caused by insufficient expression of dystrophin is Duchenne muscular dystrophy (DMD) or Becker muscular dystrophy (BMD).

Embodiment 86 is a method for increasing a level of utrophin in a cell, comprising introducing an effective amount of the saRNA of any one of embodiments 1-25, the isolated polynucleotide of any one of embodiments 40-41, the vector of embodiment 42, or the saRNA in the composition of any one of embodiments 44-47 into the cell, wherein the saRNA, the isolated polynucleotide or the composition activates expression of UTRN gene by at least 10% as compared to baseline expression of the UTRN gene.

### EXAMPLES

The present disclosure will be further illustrated with reference to specific examples and drawings below. It should be understood that these examples are merely intended to illustrate the present disclosure rather than limit the scope of the present disclosure. In the following examples, study methods without specific conditions were generally in accordance with conventional conditions, such as conditions described in Sambrook, et al., Molecular Cloning: Laboratory Manual (New York: Cold Spring Harbor Laboratory Press, 1989), or conditions recommended by the manufacturer.

### Materials and Methods

dsRNA Synthesis

The present disclosure provides a method for preparing the oligonucleotide modulator (dsRNA), which comprises sequence design and synthesis.

dsRNAs can be chemically synthesized or can be obtained from a biotechnology company specialized in nucleic acid synthesis. Generally speaking, chemical synthesis of nucleic acids comprises the following four steps: a) synthesis of oligomeric ribonucleotides; b) deprotection; c) purification and isolation; d) desalination and annealing. For example, the specific steps for chemically synthesizing dsRNAs described are as follows:

### a) Synthesis of Oligomeric Ribonucleotides

Synthesis of 1 μM RNA was set in an automatic DNA/RNA synthesizer (e.g., Applied Biosystems EXPEDITE8909), and the coupling time of each cycle was set as 10 to 15 min. With a solid phase-bonded 5′-O-p-dimethoxytriphenylmethyl-thymidine substrate as an initiator, one base was bonded to the solid phase substrate in the first cycle, and then, in the n^{th} (19≥n≥2) cycle, one base was bonded to the base bonded in the n−1^{th} cycle. This process was repeated until the synthesis of the whole nucleic acid sequence was completed.

### b) Deprotection

The solid phase substrate bonded with the dsRNA was put into a test tube, and 1 mL of a solution of the mixture of ethanol and ammonium hydroxide (volume ratio: 1:3) was added to the test tube. The test tube was then sealed and placed in an incubator, and the mixture was incubated at 25-70° C. for 2 to 30 h. The solution containing the solid phase substrate bonded with the dsRNA was filtered, and the filtrate was collected. The solid phase substrate was rinsed with double distilled water twice (1 mL each time), and the filtrate was collected. The collected eluents were combined and dried under vacuum for 1 to 12 h. Then the solution was added with 1 mL of a solution of tetrabutylammonium fluoride in tetrahydrofuran (1 M), let stand at room temperature for 4 to 12 h, followed by addition of 2 mL of n-butanol. Precipitate was collected to give a single stranded crude product of dsRNA by high-speed centrifugation.

### c) Purification and Isolation

The resulting crude product of dsRNA was dissolved in 2 mL of aqueous ammonium acetate solution with a concentration of 1 mol/mL, and the solution was separated by a reversed phase C18 column of high-pressure liquid chromatography to give a purified single-stranded product of dsRNA.

### d) Desalination and Annealing

Salts were removed by gel filtration (size exclusion chromatography). A single sense oligomeric ribonucleic acid strand and a single antisense oligomeric ribonucleic acid strand were mixed into 1 to 2 mL of buffer (10 mM Tris, pH 7.5-8.0, 50 mM NaCl) at a molar ratio of 1:1. The solution was heated to 95° C., and was then slowly cooled to room temperature to give a solution containing dsRNA.

### Cell Culture and Treatment

Human malignant embryonic rhabdomyoma cells (RD) (TCHu 45, Center for Excellence in Molecular Cell Science, Chinese Academy of Science, China) were cultured at 37° C. with 5% CO₂ in modified DMEM medium (Gibco, Thermo Fisher Scientific, Carlsbad, CA) supplemented with 10% bovine calf serum (Sigma-Aldrich) and 1% penicillin/streptomycin (Gibco). The RD cells were seeded into 96-well plates at 4000 cells/well. saRNAs were individually transfected into the RD cells in each well at 25 nM, or any other concentrations with 0.3 μL of RNAiMAX (Invitrogen, Carlsbad, CA) by following the reverse transfection protocol respectively, and the transfection duration was 3 or 5 days Mock (blank control) was transfected in the absence of an oligonucleotide. dsCon2 (SEQ ID NOs:799 and 1199) was transfected as a non-specific duplex control. DS18-si8 (SEQ ID NOs: 399, 798 and 1198) was a duplex siRNA targeting UTRN gene and transfected as a silencing dsRNA control.

### RNA Isolation and Reverse Transcription-Quantitative Polymerase Chain Reaction (RT-qPCR)

### (1) RNA Isolation and One-Step RT-qPCR

At the end of transfection, medium was discarded and cells were washed with 150 L of PBS once per well. After discarding the PBS, 100 μL of cell lysis buffer (Power SYBR® Green Cells-to-Ct™ Kit, Life Technologies) was added into each well and incubated at room temperature for 5 min. 0.5 μL of the cell lysis was taken from each well and analyzed by RT-qPCR using One Step TB Green® PrimeScrip™ RT-PCR kit II (Takara, RR086A, Shlga, Japan) in a Roche Lightcycler 480 real-time PCR machine. PCR reactions was prepared using Bravo Automated Liquid Handling Platform (Agilent, US). Each transfection sample was amplified in 3 repeat wells. PCR reaction conditions are shown in Table 2.

TABLE 2
PCR reaction

| | Volume |
|---|---|
| Reagent | (μL) |
| 2 × One Step TB Green RT-PCR buffer 4 | 2.5 |
| PrimeScript ™ 1 step enzyme Mix 2 | 0.2 |
| Forward and Reverse Primers Mix (5 μM) | 0.4 |
| dH₂O without RNase | 1.6 |
| Crude lysate (RNA) | 0.4 |
| Total volume | 5.1 |

The reaction conditions were as follows: reverse transcription reaction (stage 1): 42° C. for 5 min, 95° C. for 10 sec; PCR reaction (stage 2): 95° C. for 5 sec, 59° C. for 20 see, 72° C. for 10 see; 40 cycles of amplification; and melting curve (stage 3). Human UTRN gene was amplified as target genes. Geometric means of the mRNA levels of TBP and B2M were used as an internal reference for RNA loading. Primer sequences are listed in Table 3.

TABLE 3
Primer sequences for RT-qPCR assay

| Primer | Gene | SEQ ID NO | Sequence (5′-3′) | Product size (bp) |
|---|---|---|---|---|
| UTRN F | Human UTRN | 1201 | GCCGTGGCAAAGATCCATTT | 126 |
| UTRN R | | 1202 | ACATTATTCAGGTCAGCAAGGG | |
| TBP F | Human TBP | 1203 | TGCTCACCCACCAACAATTTAG | 139 |
| TBP R | | 1204 | TCTGCTCTGACTTTAGCACCTG | |
| B2M F | Human B2M | 1205 | GATAGTTAAGTGGGATCGAGACAT | 95 |
| B2M R | | 1206 | AGCAAGCAAGCAGAATTTGGA | |
| HPRT1 F | Human HPRT1 | 1211 | AAAGATGGTCAAGGTCGCAAG | 120 |
| HPRT1 R | | 1212 | TAGTCAAGGGCATATCCTACAAC | |

### (2) RNA Isolation and Two-Step RT-qPCR

For quantifying mRNA expression in cells, total cellular RNA was isolated from treated cells using an RNeasy Plus Mini kit (Qiagen, Hilden, Germany) according to its manual. The resultant RNA (˜1 μg) was reverse transcribed into cDNA by using a PrimeScript™ RT reagent kit with gDNA Eraser (Takara, RR047A, Shlga, Japan). The resultant cDNA was amplified in a Roche LightCycler 480 Multiwell Plate 384 (Roche, ref: 4729749001, US) using TB Green® Premix Ex Taq™ II (Takara, RR820A, Shlga, Japan) reagents and primers which specifically amplified target genes of interest. Reaction conditions were as follows: reverse transcription reaction (stage 1): 42° C. for 5 min, 95° C. for 10 sec; PCR reaction (stage 2): 95° C. for 5 see, 60° C. for 30 see, 72° C. for 10 sec; 40 cycles of amplification; and melting curve (stage 3). Primer sequences are listed in Table 3. PCR reaction conditions are shown in Table 4 and Table 5.

TABLE 4
RT reaction

| | Volume |
|---|---|
| | (μL) |
| Reaction-1 (Takara, RR047A) | |
| 5 × gDNA Eraser Buffer | 2 |
| gDNA Eraser | 1 |
| Total RNA (1 μg) + RNase Free dH2O | 7 |
| Total Volume | 10 |
| 42° C. 5 min, store at 4° C. | |
| Reaction-2 (Takara, RR047A) | |
| 5 × PrimeScript Buffer2 | 4 |
| PrimeScript RT Enzyme Mix I | 1 |
| RT Prime Mix | 1 |
| RNase free dH2O | 4 |
| Reaction-1 | 10 |
| Total Volume | 20 |

37° C. 15 min, 85° C. 5 sec, store at 4° C.

TABLE 5
RT-qPCR reaction

| | Volume |
|---|---|
| Reagent (Takara, RR820A) | (μL) |
| SYBR Premix Ex Taq II (2×) | 5 |
| PCR Primer (forward + reverse) 5 μM | 1 |
| cDNA (RT product) | 4 |
| Total | 10 |

To calculate the relative expression level (Eᵣₑₗ) of UTRN gene in an saRNA-transfected sample relative to control treatment (Mock), the Ct values of the target gene and the two internal reference genes were substituted into Formula I,

$${E}_{rel} = {2}^{( {CtT}_{m} - {CtT}_{s} )} / ( {( {2}^{( CtR {1}_{m} - CtR {1}_{s} )} * {2}^{( CtR {2}_{m} - CtR {2}_{s} )} )}^{( 1 / 2 )} ) \qquad ( Formula I )$$

wherein CtTₘ was the Ct value of the target gene from the mock-treated sample; CtTₛ was the Ct value of the target gene from the saRNA-treated sample; CtR1ₘ was the Ct value of the internal reference gene 1 from the mock-treated sample; CtR1ₛ was the Ct value of the internal reference gene 1 from the saRNA-treated sample; CtR2ₘ was the Ct value of the internal reference gene 2 from the mock-treated sample; and CtR2ₛ was the Ct value of the internal reference gene 2 from the saRNA-treated sample.

### Western Blotting

Proteins were harvested from transfected cells using 1×RIPA Buffer including protease inhibitors. The protein concentration was determined by BCA protein assay kits (Beyotime, P0010, Shanghai, China). Protein electrophoresis was performed (10 ug protein/well) with the use of a sodium dodecyl sulfate polyacrylamide gel electrophoresis (SDS-PAGE) gel, which was then transferred to a polyvinylidene difluoride (0.45 μm PVDF) membrane. The membranes were blotted with primary anti-UTRN (Santa Cruz, sc-33700, USA) or anti-α/β-Tubulin (CST, 2148s, USA) antibodies at 4° C. overnight. The membranes were incubated with horseradish peroxidase (HRP)-conjugated secondary antibodies (CST, 7076s, USA) for 1 h at room temperature (RT) after washing with TBST buffer 3 times. Then, the membranes were then washed with TBST buffer three times for 10 min each and analyzed by Image Lab (BIO-RAD, Chemistry Doctm MP Imaging System). Band densities of UTRN protein and α/β-Tubulin were quantified using ImageJ software.

### Digital Western Blotting

Proteins were harvested from transfected cells using denaturing cell lysis buffer (Invent, SD-001, USA) including protease inhibitors. The protein concentration was determined by BCA protein assay kits. Proteins were detected and analyzed using Simple Western Automated Western Blot Systems (ProteinSimple, 004-600, USA). Protein electrophoresis was performed (0.1 μg/μl per well) with the use of separation module (ProteinSimple, SW004&SW008, USA) in JESS. The capillaries were blotted with primary anti-Utrophin (Full length) (Leica biosystems, NCL-DRP2, Germany) or anti-α/β-Tubulin (CST, 2148S, USA) antibodies. After that, the capillaries were blotted with HRP conjugated secondary antibodies and signal was detected by detection kit (ProteinSimple, DM001&DM002, USA). Quantitative relative expression levels were calculated based on peak area.

It has been discovered in this disclosure that after being introduced into a cell, the aforementioned saRNA can effectively increase the expression level of UTRN mRNA and utrophin protein.

### Example 1. Design and Synthesis of dsRNAs Targeting the Human UTRN Promoter

A 1000-nt coding strand of the promoter sequence (SEQ ID NO: 1200) of human UTRN gene was retrieved from the ENSEMBL genome database (www.ensembl.org). This sequence is located immediately upstream of the first nucleotide of UTRN's first exon (ENST00000433557.1) as annotated by ENSEMBL. However, the 3′ part of this sequence also contains the first exon of a NCBI annotated UTRN RefSeq mRNA sequence (NM_007124.3). Therefore, the first nucleotide of NM_007124.3 was regarded as the true TSS (+1 position) and the downstream sequence was regarded as 5′ untranslated region (UTR) (Table 6).

TABLE 6
Putative human UTRN promoter sequence (5′-3′) (SEQ ID NO: 1200)

| | |
|---|---|
| -666 | atttatctct taaaaaaata tcaccctaac tagagacctg ttttgcctaa |
| -616 | ggggacgtga ctcacatttt cggataatct gaataagggg aattgtgtct |
| -566 | gctcgaggca tccattctgg ttoggtctcc ggactcccgg ctcccggcac |
| -516 | gcacggttca ctctggagcg cgcgccccag gccagccaag cgccgagccg |
| -466 | ggctgctgcg ggctgggagg gcgcgcaggg ccggcgctga ttgacggggc |
| -416 | gcgcagtcag gtgacttggg gcgccaagtt cccgacgcgg tggccgcggt |
| -366 | gaccgccgag gcccggcaga cgctgacccg ggaacgtagt ggggctgatc |
| -316 | ttccggaaca aagttgctgg gccggcggcg gcggggcgag agcgccgagg |
| -266 | gggagccgga gcgctgcaga ggcgcgggcc ggagggctgg cgctgatctg |
| -216 | cacccttctc atctggagag cggaccectg gctgcccgga ggcgagcccc |
| -166 | ttcccggggg gtgggggcgg caacgcgcga cccagcggtc ctgcgcccca |
| -116 | ccctccctcc tccgcctcca gcgcteggct ccaacaaagg ggcaggcccg |
| -66 | cagcggggag gaggaggagg agccgccgaa ggagcgagcc tctctcgcgc |
| -16 | 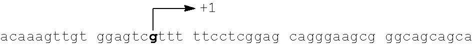 |
| 35 | gccggccgcg ggctttctcc cgccgagggg cgaggaggag cctctggctc |
| 85 | cagaagccga ttggggaatc acggggagcg gegcccccct tcttttgggt |
| 135 | catttctgca aacggaaaac tctgtagcgt ttggcaaagt tggtgcctgc |
| 185 | gcgccccttc caggtttgcg ctttgactgt tttgtttttg geggaactac |
| 235 | caggcaggaa gattgcacaa gtaaggggog ttttcagtcg ggtgtcaatt |
| 285 | tctctttctt tctttctttt tttaaaattt cggttcgtgt ctgcttctcc |

Note: The bent arrow and the letter in bold face indicate transcription start (TSS) of RefSeq NM 007124.3.

To identify functional dsRNAs capable of activating the expression of UTRN gene, a series of 21 nt dsRNA targets were selected on the 1000 bp UTRN promoter sequence, starting from −666 bp upstream of the TSS moving toward the TSS by 1 bp each time, and resulting in 985 target sequences. The target sequences were then filtered to keep those which met the following criteria: (1) having a GC content between 35% and 70%; (2) with less than 5 consecutive identical nucleotides; (3) with 3 or less dinucleotide repeats; and (4) with 3 or less trinucleotide repeats. After the filtration, 398 target sequences remained and were used to determine the sense strand sequence of candidate saRNAs, of which 212 targets were on the promoter and 186 on the 5′UTR. Strand composition and sequence of each dsRNA duplex including cognate target site in the UTRN promoter are listed in Table 1.

### Example 2. High Throughput Screening of dsRNAs Targeting the Human UTRN Promoter

To identify dsRNAs capable of activating UTRN transcription, RD cells were transfected with each of the aforementioned 398 dsRNAs with a transfection concentration of 25 nM for 72 hours followed by UTRN gene expression analysis via one-step RT-qPCR.

Of the 398 dsRNAs screened, 108 (27.1%) induced UTRN expression, in which 23 (5.8%), 56 (14.1%) and 29 (7.3%) dsRNAs showed high activation (≥1.5 fold), moderate activation (1.2-1.5 fold) and mild activation (1.1-1.2 fold) of UTRN mRNA expression, respectively (Table 7).

Of the 212 dsRNAs located on the true promoter, 99 (46.7%) induced UTRN expression, in which 20 (9.5%), 52 (24.5%) and 27 (12.7%) showed high activation (≥1.5 fold), moderate activation (1.2˜1.5 fold) and mild activation (1.1˜1.2 fold) of UTRN mRNA expression, respectively (Table 8).

Of the 186 dsRNAs targeting the 5′ UTR region, a majority inhibited UTRN mRNA expression (FIG. 2).

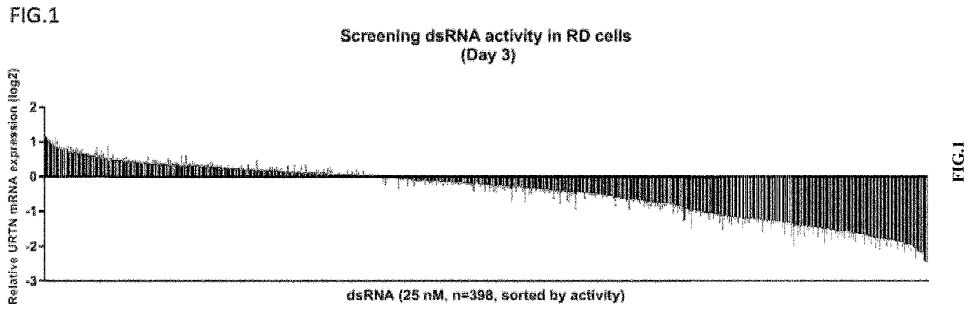

The dsRNAs with activating activity (≥1.1 fold) are exemplified in the present disclosure as “functional saRNAs”. Relative changes in UTRN mRNA expression caused by saRNA treatment are also summarized in Table 1, while expression data organized by gene induction is plotted in FIG. 1.

TABLE 7
Summary of activity of all screened dsRNAs

| | | Number | |
|---|---|---|---|
| | log₂ value of change in | of | Percentage |
| dsRNA activity | UTRN mRNA (fold) | dsRNAs | (%) |
| High activation | ≥0.49 (1.50)~≤1.13 (2.19) | 23 | 5.8 |
| Moderate activation | ≥0.26 (1.20)~<0.49 (1.50) | 56 | 14.1 |
| Mild activation | ≥0.13 (1.10)~<0.26 (1.20) | 29 | 7.3 |
| Down regulation/No | <0.13 (1.10) | 290 | 72.8 |
| activation effect | | | |
| Total | | 398 | 100 |

TABLE 8
Summary of activity of screened dsRNAs located on promoter only

| | | Number | |
|---|---|---|---|
| | log₂ value of change in | of | Percentage |
| dsRNA activity | UTRN mRNA( fold) | dsRNAs | (%) |
| High activation | ≥0.49 (1.50)~≤1.13 (2.19) | 20 | 9.5 |
| Moderate activation | ≥0.26 (1.20)~<0.49 (1.50) | 52 | 24.5 |
| Mild activation | ≥0.13 (1.10)~<0.26 (1.20) | 27 | 12.7 |
| Down regulation/No | <0.13 (1.10) | 113 | 53.3 |
| activation effect | | | |
| Total | | 212 | 100 |

When the dsRNAs were sorted by their location on the human UTRN promoter and 5′UTR (FIG. 2), it can be clearly seen that almost half of the dsRNAs targeting the true UTRN promoter (−666˜−1) induced UTRN mRNA expression.

Sorting expression data by target site location within the human UTRN promoter revealed four “hotspot regions” that were enriched for dsRNA activity including regions −636 to −496 (H1), −351 to −294 (H2), −236 to −187 (H3) and −101 to −65 (H4) relative to the TSS (FIG. 2). Nearly 55% of the targeted sequences of the functional dsRNAs located in the indicated “hotspot regions”. Each “hotspot region” corresponding to the promoter sequence is listed in Table 9.

By following the design criteria: (i) GC content between 35-65%; (ii) less than 5 consecutive identical nucleotides; (iii) 3 or less total dinucleotide repeats; and (iv) 3 or less total trinucleotide repeats, it appeared in the present example that at least 25% of designed saRNA targeting the provided hotspot region sequences can activate the expression of UTRN gene by at least 10%.

dsRNA duplexes capable of upregulating human UTRN expression by 1.1-fold or higher in “hotspot region” H1 (−636 to −496) were as followings: DS18-0384, DS18-0383, DS18-0382, DS18-0380, DS18-0379, DS18-0378, DS18-0377, DS18-0376, DS18-0375, DS18-0374, DS18-0373, DS18-0372, DS18-0371, DS18-0370, DS18-0368, DS18-0363, DS18-0362, DS18-0358, DS18-0357, DS18-0355, DS18-0354, DS18-0352, DS18-0350, DS18-0349, DS18-0348, DS18-0347, DS18-0345, DS18-0344, DS18-0343, DS18-0335, DS18-0334, DS18-0333, DS18-0332, DS18-0329, DS18-0328, DS18-0327, DS18-0325, DS18-0324, DS18-0323, DS18-0321, DS18-0320, DS18-0315, DS18-0314, DS18-0313, DS18-0312, DS18-0311, DS18-0310, DS18-0308, DS18-0305, DS18-0304, DS18-0301, DS18-0298.

dsRNA duplexes capable of upregulating human UTRN expression by 1.1-fold or higher in “hotspot region” H2 (−351 to −294) were as followings: DS18-0277, DS18-0272, DS18-0271, DS18-0264, DS18-0255, DS18-0253, DS18-0252, DS18-0251, DS18-0248, DS18-0243.

dsRNA duplexes capable of upregulating human UTRN expression by 1.1-fold or higher in “hotspot region” H3 (−236 to −187) were as followings: DS18-0237, DS18-0236, DS18-0234, DS18-0233, DS18-0232, DS18-0231, DS18-0230, DS18-0229, DS18-0228, DS18-0225, DS18-0223, DS18-0222, DS18-0221, DS18-0216, DS18-0211, DS18-0209, DS18-0208, DS18-0207, DS18-0206.

dsRNA duplexes capable of upregulating human UTRN expression by 1.1-fold or higher in “hotspot region” H4 (−101 to −65) were as followings: DS18-0205, DS18-0204, DS18-0202, DS18-0200, DS18-0199, DS18-0198, DS18-0197, DS18-0194, DS18-0193, DS18-0192, DS18-0191, DS18-0189, DS18-0188, DS18-0187.

These results indicated that functional dsRNAs were not randomly distributed on the promoter but were clustered in the specific hotspot regions. Their corresponding DNA sequences are listed in Table 9.

TABLE 9
Human UTRN dsRNA hotspot regions and their sequences

| | | SEQ | Size |
|---|---|---|---|
| Hotspot region | Hotspot region sequences on UTRN promoter (5′-3′) | ID NO | (nt) |
| H1 (−636 to −496) | tagagacctgttttgcctaaggggacgtgactcacattttcggataatctgaataagg | 1207 | 141 |
| | ggaattgtgtctgctcgaggcatccattctggttcggtctccggactcccggctcccg | | |
| | gcacgcacggttcactctggagcgc | | |
| H2 (−351 to −294) | gcagacgctgacccgggaacgtagtggggctgatcttccggaacaaagttgctgg | 1208 | 58 |
| | gcc | | |
| H3 (−236 to −187) | ggagggctggcgctgatctgcacccttctcatctggagagcggacccctg | 1209 | 50 |
| H4 (−101 to −65) | ctccagcgctoggctccaacaaaggggcaggcccgca | 1210 | 37 |

### Example 3. saRNA Treatment Increases the UTRN mRNA Levels in RD Cells

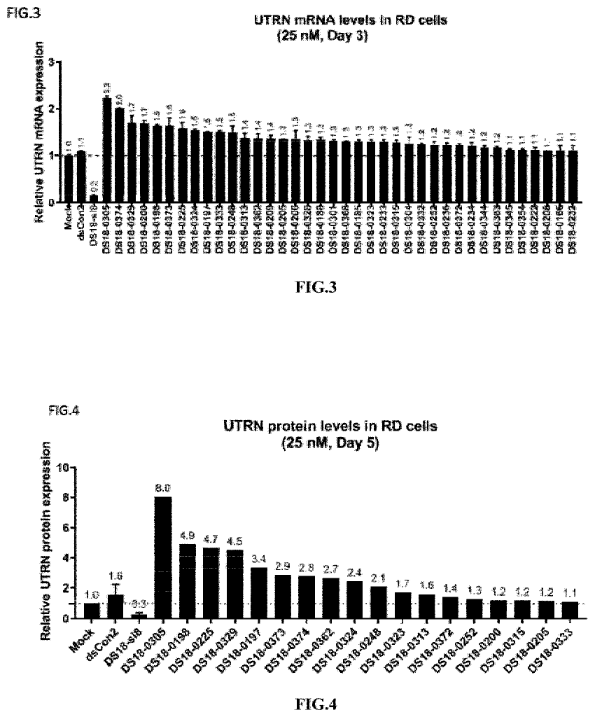

Based on the screening result for UTRN induction, the top 38 performing saRNAs (see Table 1) were transfected into RD cells at 25 nM of individual saRNA for 3 days. Then, the transfected RD cells were analyzed for UTRN mRNA expression by RT-qPCR. Both dsCon2 and DS18-si8 served as a non-specific duplex control for gene activation and a silencing dsRNA control, respectively. The UTRN mRNA expression of individual saRNAs are shown in FIG. 3.

### Example 4. saRNA Treatment Increases the Utrophin Protein Levels in RD Cells

A subset of the top performers (18 saRNAs) was transfected into RD cells at 25 nM concentrations to quantify the utrophin protein after 5 days by Western blotting. Both dsCon2 and DS18-si8 served as a non-specific duplex control for gene activation and a silencing dsRNA control, respectively. FIG. 4 summarizes relative fold changes of utrophin levels derived from quantifying the band intensity.

### Example 5. Impact of saRNA Structure and Sequence on saRNA Activity In Vitro

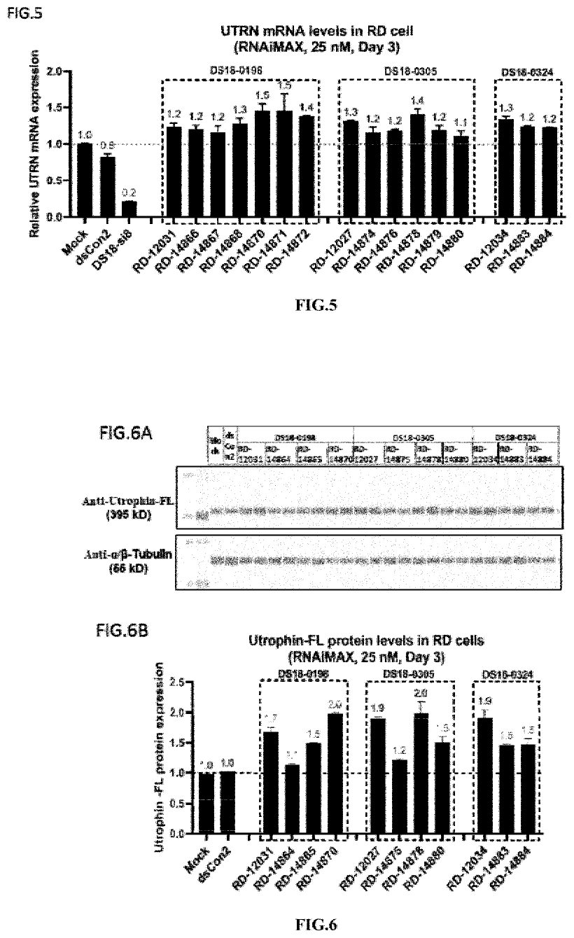

To assess impact of duplex structure and sequence specificity on saRNA activity, a series of saRNA variants were synthesized based on three of best performers (i.e., DS18-0198, DS18-0305 and DS18-0324) for activating human UTRN mRNA (FIG. 5) and utrophin protein (FIG. 6A-B). Table 10 lists the sequence composition and design for each saRNA variant. Each duplex was transfected into RD cells for 3 days at 25 nM concentrations. UTRN mRNA levels were analyzed via two-step RT-qPCR and utrophin protein levels were detected by JESS. Treatment with dsCon2 and DS18-si8 served as a non-specific duplex control for gene activation and a silencing dsRNA control, respectively. α/β-Tubulin protein served as a control for protein loading. UTRN mRNA levels are shown in FIG. 5. Utrophin protein bands are shown in FIG. 6A and utrophin protein levels are shown in FIG. 6B.

TABLE 10
Oligonucleotide sequences and duplex compositions of UTRN saRNA variant designs

| | | | SEQ | | |
|---|---|---|---|---|---|
| | | | ID | | Size |
| saRNA | Design | Strand | NO | Sequence (5′-3′) | (nt) |
| RD-12031 | Original hit from screen | sense | 597 | GCUCGGCUCCAACAAAGGGgc | 21 |
| (DS18-0198) | | antisense | 997 | CCCUUUGUUGGAGCCGAGCgc | 21 |
| RD-14864 | 16-bp with TT overhang | sense | 1213 | GCUCGGCUCCAACAAATT | 18 |
| | | antisense | 1214 | UUUGUUGGAGCCGAGCTT | 18 |
| RD-14865 | 18-bp with blunt end | sense | 1215 | GCUCGGCUCCAACAAAGG | 18 |
| | | antisense | 1216 | CCUUUGUUGGAGCCGAGC | 18 |
| RD-14866 | 22-nt of sense strand and 20-nt of antisense strand | sense | 1217 | GCUCGGCUCCAACAAAGGGGCA | 22 |
| | | antisense | 1218 | CCCCUUUGUUGGAGCCGAGC | 20 |
| RD-14867 | 30-nt of sense strand and 28-nt of antisense strand | sense | 1219 | GCUCGGCUCCAACAAAGGGGCAGGCCCGCA | 30 |
| | | antisense | 1220 | CGGGCCUGCCCCUUUGUUGGAGCCGAGC | 28 |
| RD-14868 | 5 nt ″seed″ sequence mutation to sense strand | sense | 1221 | GCUCGGCUCCAACAAAGGGGC | 21 |
| | | antisense | 1222 | CCCUUUGUUGGACCGGUGGGG | 21 |
| RD-14870 | 1 nt overhang | sense | 1223 | GCUCGGCUCCAACAAAGGGG | 20 |
| | | antisense | 1224 | CCCUUUGUUGGAGCCGAGC | 19 |
| RD-14871 | 3 nt overhang | sense | 1225 | GCUCGGCUCCAACAAAGGGGCA | 22 |
| | | antisense | 1224 | CCCUUUGUUGGAGCCGAGC | 19 |
| RD-14872 | 5 nt overhang | sense | 1226 | GCUCGGCUCCAACAAAGGGGCAGG | 24 |
| | | antisense | 1224 | CCCUUUGUUGGAGCCGAGC | 19 |
| RD-12027 | Original hit from screen | sense | 704 | GGCACGCACGGUUCACUCUgg | 21 |
| (DS18-0305) | | antisense | 1104 | AGAGUGAACCGUGCGUGCCgg | 21 |
| RD-14874 | 19-bp with blunt end | sense | 1227 | GGCACGCACGGUUCACUCU | 19 |
| | | antisense | 1228 | AGAGUGAACCGUGCGUGCC | 19 |
| RD-14875 | 23-nt of sense strand and 25-nt of antisense strand | sense | 1229 | UCCCGGCACGCACGGUUCACUCU | 23 |
| | | antisense | 1230 | AGAGUGAACCGUGCGUGCCGGGAGC | 25 |
| RD-14876 | 33-nt of sense strand and 35-nt of antisense strand | sense | 1231 | GACUCCCGGCUCCCGGCACGCACGGUUCACUCU | 33 |
| | | antisense | 1232 | AGAGUGAACCGUGCGUGCCGGGAGCCGGGAGUCCG | 35 |
| RD-14878 | 5 nt ″seed″ sequence mutation to antisense strand | sense | 1233 | GGCACGCACGGUACUCACAGC | 21 |
| | | antisense | 1234 | AGAGUGAACCGUGCGUGCCGG | 21 |
| RD-14879 | 4 nt overhang | sense | 1235 | GGCACGCACGGUUCACUCU | 19 |
| | | antisense | 1236 | AGAGUGAACCGUGCGUGCCGGGA | 23 |
| RD-14880 | 6 nt overhang | sense | 1235 | GGCACGCACGGUUCACUCU | 19 |
| | | antisense | 1237 | AGAGUGAACCGUGCGUGCCGGGAGC | 25 |
| RD-12034 | Original hit from screen | sense | 723 | CUCGAGGCAUCCAUUCUGGuu | 21 |
| (DS18-0324) | | antisense | 1123 | CCAGAAUGGAUGCCUCGAGca | 21 |
| RD-14883 | 16-bp with TT overhang | sense | 1238 | GAGGCAUCCAUUCUGGTT | 18 |
| | | antisense | 1239 | CCAGAAUGGAUGCCUCTT | 18 |
| RD-14884 | 29-nt of sense strand and 35-nt of antisense strand | sense | 1240 | AUUGUGUCUGCUCGAGGCAUCCAUUCUGG | 29 |
| | | antisense | 1241 | CCAGAAUGGAUGCCUCGAGCAGACACAAUUCCCCU | 35 |

Note:
upper case, RNA. The nucleotides in bold are overhang. The nucleotides in bold and italic are mismatch.

In summary, the high throughput screening data revealed a plurality of “hotspot regions” for saRNA activity in the promoter of human UTRN gene. Exemplary saRNAs increased expression of both UTRN mRNA and utrophin protein levels. These results provide evidence that targeted activation of UTRN expression is a promising strategy to treat DDD, e.g., DMD and BMD.

TABLE 1
dsRNA targets, sequences and activity of human UTRN saRNAs

| | | | | | | | Relative | Relative |
|---|---|---|---|---|---|---|---|---|
| | SEQ | | SEQ | | SEQ | | UTRN | UTRN |
| | ID | | ID | | ID | | mRNA | mRNA (log2 |
| dsRNA | NO | Target sequence (5′-3′) | NO | Sense (5′-3′) | NO | Antisense (5′-3′) | (fold change) | fold change) |
| DS18-0001 | 1 | ggttcgtgtctgcttctcc | 400 | GGUUCGUGUCUGCUUCUCCaa | 800 | GGAGAAGCAGACACGAACCga | 0.38 | −1.391 |
| DS18-0002 | 2 | cggttcgtgtctgcttctc | 401 | CGGUUCGUGUCUGCUUCUCca | 801 | GAGAAGCAGACACGAACCGaa | 0.30 | 1.754 |
| DS18-0003 | 3 | tcggttcgtgtctgcttct | 402 | UCGGUUCGUGUCUGCUUCUcc | 802 | AGAAGCAGACACGAACCGAaa | 0.27 | −1.915 |
| DS18-0004 | 4 | ttcggttcgtgtctgcttc | 403 | UUCGGUUCGUGUCUGCUUCuc | 803 | GAAGCAGACACGAACCGAAau | 0.42 | −1.248 |
| DS18-0005 | 5 | tttcggttcgtgtctgctt | 404 | UUUCGGUUCGUGUCUGCUUcu | 804 | AAGCAGACACGAACCGAAAuu | 0.39 | 1.370 |
| DS18-0006 | 6 | atttcggttcgtgtctgct | 405 | AUUUCGGUUCGUGUCUGCUuc | 805 | AGCAGACACGAACCGAAAUuu | 0.72 | 0.468 |
| DS18-0007 | 7 | aatttcggttcgtgtctgc | 406 | AAUUUCGGUUCGUGUCUGCuu | 806 | GCAGACACGAACCGAAAUUuu | 0.83 | 0.262 |
| DS18-0008 | 8 | aaatttcggttcgtgtctg | 407 | AAAUUUCGGUUCGUGUCUGcu | 807 | CAGACACGAACCGAAAUUUua | 0.39 | 1.342 |
| DS18-0009 | 9 | aaaatttcggttcgtgtct | 408 | AAAAUUUCGGUUCGUGUCUgc | 808 | AGACACGAACCGAAAUUUUaa | 0.86 | −0.221 |
| DS18-0010 | 10 | taaaatttcggttcgtgtc | 409 | UAAAAUUUCGGUUCGUGUCug | 809 | GACACGAACCGAAAUUUUAaa | 1.07 | 0.095 |
| DS18-0011 | 11 | ggtgtcaatttctctttct | 410 | GGUGUCAAUUUCUCUUUCUuu | 810 | AGAAAGAGAAAUUGACACCcg | 0.38 | 1.406 |
| DS18-0012 | 12 | gggtgtcaatttctctttc | 411 | GGGUGUCAAUUUCUCUUUCuu | 811 | GAAAGAGAAAUUGACACCCga | 0.34 | −1.553 |
| DS18-0013 | 13 | cgggtgtcaatttctcttt | 412 | CGGGUGUCAAUUUCUCUUUcu | 812 | AAAGAGAAAUUGACACCCGac | 0.56 | −0.829 |
| DS18-0014 | 14 | tcgggtgtcaatttctctt | 413 | UCGGGUGUCAAUUUCUCUUuc | 813 | AAGAGAAAUUGACACCCGAcu | 0.41 | −1.287 |
| DS18-0015 | 15 | gtcgggtgtcaatttctct | 414 | GUCGGGUGUCAAUUUCUCUuu | 814 | AGAGAAAUUGACACCCGACug | 0.49 | −1.036 |
| DS18-0016 | 16 | agtcgggtgtcaatttctc | 415 | AGUCGGGUGUCAAUUUCUCuu | 815 | GAGAAAUUGACACCCGACUga | 0.24 | −2.034 |
| DS18-0017 | 17 | cagtcgggtgtcaatttct | 416 | CAGUCGGGUGUCAAUUUCUcu | 816 | AGAAAUUGACACCCGACUGaa | 0.28 | −1.854 |
| DS18-0018 | 18 | tcagtcgggtgtcaatttc | 417 | UCAGUCGGGUGUCAAUUUCuc | 817 | GAAAUUGACACCCGACUGAaa | 0.65 | 0.625 |
| DS18-0019 | 19 | ttcagtcgggtgtcaattt | 418 | UUCAGUCGGGUGUCAAUUUcu | 818 | AAAUUGACACCCGACUGAAaa | 0.60 | 0.727 |
| DS18-0020 | 20 | tttcagtcgggtgtcaatt | 419 | UUUCAGUCGGGUGUCAAUUuc | 819 | AAUUGACACCCGACUGAAAac | 0.48 | −1.057 |
| DS18-0021 | 21 | ttttcagtcgggtgtcaat | 420 | UUUUCAGUCGGGUGUCAAUuu | 820 | AUUGACACCCGACUGAAAAcg | 0.30 | 1.735 |
| DS18-0022 | 22 | gttttcagtcgggtgtcaa | 421 | GUUUUCAGUCGGGUGUCAAuu | 821 | UUGACACCCGACUGAAAACgc | 0.35 | −1.530 |
| DS18-0023 | 23 | cgttttcagtcgggtgtca | 422 | CGUUUUCAGUCGGGUGUCAau | 822 | UGACACCCGACUGAAAACGcc | 0.35 | −1.499 |
| DS18-0024 | 24 | gcgttttcagtcgggtgtc | 423 | GCGUUUUCAGUCGGGUGUCaa | 823 | GACACCCGACUGAAAACGCcc | 0.25 | −1.994 |
| DS18-0025 | 25 | ggcgttttcagtcgggtgt | 424 | GGCGUUUUCAGUCGGGUGUca | 824 | ACACCCGACUGAAAACGCCcc | 0.23 | −2.114 |
| DS18-0026 | 26 | gggcgttttcagtcgggtg | 425 | GGGCGUUUUCAGUCGGGUGuc | 825 | CACCCGACUGAAAACGCCCcu | 0.22 | −2.159 |
| DS18-0027 | 27 | ggggcgttttcagtcgggt | 426 | GGGGCGUUUUCAGUCGGGUgu | 826 | ACCCGACUGAAAACGCCCCuu | 0.30 | −1.754 |
| DS18-0028 | 28 | aggggcgttttcagtcggg | 427 | AGGGGCGUUUUCAGUCGGGug | 827 | CCCGACUGAAAACGCCCCUua | 0.41 | −1.287 |
| DS18-0029 | 29 | aaggggcgttttcagtcgg | 428 | AAGGGGCGUUUUCAGUCGGgu | 828 | CCGACUGAAAACGCCCCUUac | 0.61 | −0.713 |
| DS18-0030 | 30 | taaggggcgttttcagtcg | 429 | UAAGGGGCGUUUUCAGUCGgg | 829 | CGACUGAAAACGCCCCUUAcu | 0.64 | 0.644 |
| DS18-0031 | 31 | gtaaggggcgttttcagtc | 430 | GUAAGGGGCGUUUUCAGUCgg | 830 | GACUGAAAACGCCCCUUACuu | 0.61 | −0.703 |
| DS18-0032 | 32 | agtaaggggcgttttcagt | 431 | AGUAAGGGGCGUUUUCAGUcg | 831 | ACUGAAAACGCCCCUUACUug | 0.60 | −0.738 |
| DS18-0033 | 33 | aagtaaggggcgttttcag | 432 | AAGUAAGGGGCGUUUUCAGuc | 832 | CUGAAAACGCCCCUUACUUgu | 0.52 | −0.957 |
| DS18-0034 | 34 | caagtaaggggcgttttca | 433 | CAAGUAAGGGGCGUUUUCAgu | 833 | UGAAAACGCCCCUUACUUGug | 0.33 | −1.596 |
| DS18-0035 | 35 | acaagtaaggggcgttttc | 434 | ACAAGUAAGGGGCGUUUUCag | 834 | GAAAACGCCCCUUACUUGUgc | 0.53 | −0.909 |
| DS18-0036 | 36 | cacaagtaaggggcgtttt | 435 | CACAAGUAAGGGGCGUUUUca | 835 | AAAACGCCCCUUACUUGUGca | 0.47 | −1.092 |
| DS18-0037 | 37 | gcacaagtaaggggcgttt | 436 | GCACAAGUAAGGGGCGUUUuc | 836 | AAACGCCCCUUACUUGUGCaa | 0.33 | −1.597 |
| DS18-0038 | 38 | tgcacaagtaaggggcgtt | 437 | UGCACAAGUAAGGGGCGUUuu | 837 | AACGCCCCUUACUUGUGCAau | 0.43 | −1.203 |
| DS18-0039 | 39 | ttgcacaagtaaggggcgt | 438 | UUGCACAAGUAAGGGGCGUuu | 838 | ACGCCCCUUACUUGUGCAAuc | 0.69 | −0.527 |
| DS18-0040 | 40 | attgcacaagtaaggggcg | 439 | AUUGCACAAGUAAGGGGCGuu | 839 | CGCCCCUUACUUGUGCAAUcu | 1.26 | 0.334 |
| DS18-0041 | 41 | gattgcacaagtaaggggc | 440 | GAUUGCACAAGUAAGGGGCgu | 840 | GCCCCUUACUUGUGCAAUCuu | 0.49 | −1.028 |
| DS18-0042 | 42 | agattgcacaagtaagggg | 441 | AGAUUGCACAAGUAAGGGGcg | 841 | CCCCUUACUUGUGCAAUCUuc | 0.86 | −0.214 |
| DS18-0043 | 43 | aagattgcacaagtaaggg | 442 | AAGAUUGCACAAGUAAGGGgc | 842 | CCCUUACUUGUGCAAUCUUcc | 0.81 | −0.310 |
| DS18-0044 | 44 | gaagattgcacaagtaagg | 443 | GAAGAUUGCACAAGUAAGGgg | 843 | CCUUACUUGUGCAAUCUUCcu | 0.63 | −0.666 |
| DS18-0045 | 45 | ggaagattgcacaagtaag | 444 | GGAAGAUUGCACAAGUAAGgg | 844 | CUUACUUGUGCAAUCUUCCug | 0.36 | −1.459 |
| DS18-0046 | 46 | aggaagattgcacaagtaa | 445 | AGGAAGAUUGCACAAGUAAgg | 845 | UUACUUGUGCAAUCUUCCUgc | 0.63 | −0.670 |
| DS18-0047 | 47 | caggaagattgcacaagta | 446 | CAGGAAGAUUGCACAAGUAag | 846 | UACUUGUGCAAUCUUCCUGcc | 0.37 | −1.435 |
| DS18-0048 | 48 | gcaggaagattgcacaagt | 447 | GCAGGAAGAUUGCACAAGUaa | 847 | ACUUGUGCAAUCUUCCUGCcu | 0.30 | 1.742 |
| DS18-0049 | 49 | ggcaggaagattgcacaag | 448 | GGCAGGAAGAUUGCACAAGua | 848 | CUUGUGCAAUCUUCCUGCCug | 0.34 | −1.553 |
| DS18-0050 | 50 | aggcaggaagattgcacaa | 449 | AGGCAGGAAGAUUGCACAAgu | 849 | UUGUGCAAUCUUCCUGCCUgg | 0.38 | −1.396 |
| DS18-0051 | 51 | caggcaggaagattgcaca | 450 | CAGGCAGGAAGAUUGCACAag | 850 | UGUGCAAUCUUCCUGCCUGgu | 0.28 | −1.849 |
| DS18-0052 | 52 | ccaggcaggaagattgcac | 451 | CCAGGCAGGAAGAUUGCACaa | 851 | GUGCAAUCUUCCUGCCUGGua | 0.43 | −1.201 |
| DS18-0053 | 53 | accaggcaggaagattgca | 452 | ACCAGGCAGGAAGAUUGCAca | 852 | UGCAAUCUUCCUGCCUGGUag | 0.29 | −1.788 |
| DS18-0054 | 54 | taccaggcaggaagattgc | 453 | UACCAGGCAGGAAGAUUGCac | 853 | GCAAUCUUCCUGCCUGGUAgu | 0.75 | −0.408 |
| DS18-0055 | 55 | ctaccaggcaggaagattg | 454 | CUACCAGGCAGGAAGAUUGca | 854 | CAAUCUUCCUGCCUGGUAGuu | 0.54 | −0.882 |
| DS18-0056 | 56 | actaccaggcaggaagatt | 455 | ACUACCAGGCAGGAAGAUUgc | 855 | AAUCUUCCUGCCUGGUAGUuc | 0.45 | −1.158 |
| DS18-0057 | 57 | aactaccaggcaggaagat | 456 | AACUACCAGGCAGGAAGAUug | 856 | AUCUUCCUGCCUGGUAGUUcc | 0.52 | −0.957 |
| DS18-0058 | 58 | gaactaccaggcaggaaga | 457 | GAACUACCAGGCAGGAAGAuu | 857 | UCUUCCUGCCUGGUAGUUCcg | 0.32 | −1.632 |
| DS18-0059 | 59 | ggaactaccaggcaggaag | 458 | GGAACUACCAGGCAGGAAGau | 858 | CUUCCUGCCUGGUAGUUCCgc | 0.38 | −1.410 |
| DS18-0060 | 60 | cggaactaccaggcaggaa | 459 | CGGAACUACCAGGCAGGAAga | 859 | UUCCUGCCUGGUAGUUCCGcc | 0.49 | −1.018 |
| DS18-0061 | 61 | gcggaactaccaggcagga | 460 | GCGGAACUACCAGGCAGGAag | 860 | UCCUGCCUGGUAGUUCCGCca | 1.05 | 0.077 |
| DS18-0062 | 62 | ggcggaactaccaggcagg | 461 | GGCGGAACUACCAGGCAGGaa | 861 | CCUGCCUGGUAGUUCCGCCaa | 0.46 | −1.131 |
| DS18-0063 | 63 | tggcggaactaccaggcag | 462 | UGGCGGAACUACCAGGCAGga | 862 | CUGCCUGGUAGUUCCGCCAaa | 0.44 | −1.178 |
| DS18-0064 | 64 | ttggcggaactaccaggca | 463 | UUGGCGGAACUACCAGGCAgg | 863 | UGCCUGGUAGUUCCGCCAAaa | 0.45 | −1.142 |
| DS18-0065 | 65 | tttggcggaactaccaggc | 464 | UUUGGCGGAACUACCAGGCag | 864 | GCCUGGUAGUUCCGCCAAAaa | 1.02 | 0.025 |
| DS18-0066 | 66 | ttttggcggaactaccagg | 465 | UUUUGGCGGAACUACCAGGca | 865 | CCUGGUAGUUCCGCCAAAAac | 1.16 | 0.216 |
| DS18-0067 | 67 | cgctttgactgttttgttt | 466 | CGCUUUGACUGUUUUGUUUuu | 866 | AAACAAAACAGUCAAAGCGca | 0.34 | −1.553 |
| DS18-0068 | 68 | gcgctttgactgttttgtt | 467 | GCGCUUUGACUGUUUUGUUuu | 867 | AACAAAACAGUCAAAGCGCaa | 0.29 | −1.777 |
| DS18-0069 | 69 | tgcgctttgactgttttgt | 468 | UGCGCUUUGACUGUUUUGUuu | 868 | ACAAAACAGUCAAAGCGCAaa | 0.37 | −1.442 |
| DS18-0070 | 70 | ttgcgctttgactgttttg | 469 | UUGCGCUUUGACUGUUUUGuu | 869 | CAAAACAGUCAAAGCGCAAac | 0.40 | −1.317 |
| DS18-0071 | 71 | tttgcgctttgactgtttt | 470 | UUUGCGCUUUGACUGUUUUgu | 870 | AAAACAGUCAAAGCGCAAAcc | 0.33 | −1.594 |
| DS18-0072 | 72 | gtttgcgctttgactgttt | 471 | GUUUGCGCUUUGACUGUUUug | 871 | AAACAGUCAAAGCGCAAACcu | 0.26 | −1.934 |
| DS18-0073 | 73 | ggtttgcgctttgactgtt | 472 | GGUUUGCGCUUUGACUGUUuu | 872 | AACAGUCAAAGCGCAAACCug | 0.34 | −1.548 |
| DS18-0074 | 74 | aggtttgcgctttgactgt | 473 | AGGUUUGCGCUUUGACUGUuu | 873 | ACAGUCAAAGCGCAAACCUgg | 0.44 | −1.190 |
| DS18-0075 | 75 | caggtttgcgctttgactg | 474 | CAGGUUUGCGCUUUGACUGuu | 874 | CAGUCAAAGCGCAAACCUGga | 0.27 | −1.864 |
| DS18-0076 | 76 | ccaggtttgcgctttgact | 475 | CCAGGUUUGCGCUUUGACUgu | 875 | AGUCAAAGCGCAAACCUGGaa | 0.26 | −1.952 |
| DS18-0077 | 77 | tccaggtttgcgctttgac | 476 | UCCAGGUUUGCGCUUUGACug | 876 | GUCAAAGCGCAAACCUGGAag | 0.62 | −0.695 |
| DS18-0078 | 78 | ttccaggtttgcgctttga | 477 | UUCCAGGUUUGCGCUUUGAcu | 877 | UCAAAGCGCAAACCUGGAAgg | 0.44 | −1.182 |
| DS18-0079 | 79 | cttccaggtttgcgctttg | 478 | CUUCCAGGUUUGCGCUUUGac | 878 | CAAAGCGCAAACCUGGAAGgg | 0.31 | −1.673 |
| DS18-0080 | 80 | ccttccaggtttgcgcttt | 479 | CCUUCCAGGUUUGCGCUUUga | 879 | AAAGCGCAAACCUGGAAGGgg | 0.32 | −1.651 |
| DS18-0081 | 81 | cccttccaggtttgcgctt | 480 | CCCUUCCAGGUUUGCGCUUug | 880 | AAGCGCAAACCUGGAAGGGgc | 0.38 | −1.403 |
| DS18-0082 | 82 | ccccttccaggtttgcgct | 481 | CCCCUUCCAGGUUUGCGCUuu | 881 | AGCGCAAACCUGGAAGGGGcg | 0.19 | −2.392 |
| DS18-0083 | 83 | gccccttccaggtttgcgc | 482 | GCCCCUUCCAGGUUUGCGCuu | 882 | GCGCAAACCUGGAAGGGGCgc | 0.34 | −1.550 |
| DS18-0084 | 84 | cgccccttccaggtttgcg | 483 | CGCCCCUUCCAGGUUUGCGcu | 883 | CGCAAACCUGGAAGGGGCGcg | 0.6 | −0.719 |
| DS18-0085 | 85 | gcgccccttccaggtttgc | 484 | GCGCCCCUUCCAGGUUUGCgc | 884 | GCAAACCUGGAAGGGGCGCgc | 0.54 | −0.898 |
| DS18-0086 | 86 | cgcgccccttccaggtttg | 485 | CGCGCCCCUUCCAGGUUUGcg | 885 | CAAACCUGGAAGGGGCGCGca | 0.40 | −1.306 |
| DS18-0087 | 87 | caaagttggtgcctgcgcg | 486 | CAAAGUUGGUGCCUGCGCGcc | 886 | CGCGCAGGCACCAACUUUGcc | 0.7 | −0.493 |
| DS18-0088 | 88 | gcaaagttggtgcctgcgc | 487 | GCAAAGUUGGUGCCUGCGCgc | 887 | GCGCAGGCACCAACUUUGCca | 0.62 | −0.689 |
| DS18-0089 | 89 | ggcaaagttggtgcctgcg | 488 | GGCAAAGUUGGUGCCUGCGcg | 888 | CGCAGGCACCAACUUUGCCaa | 0.52 | −0.933 |
| DS18-0090 | 90 | tggcaaagttggtgcctgc | 489 | UGGCAAAGUUGGUGCCUGCgc | 889 | GCAGGCACCAACUUUGCCAaa | 0.51 | −0.985 |
| DS18-0091 | 91 | ttggcaaagttggtgcctg | 490 | UUGGCAAAGUUGGUGCCUGcg | 890 | CAGGCACCAACUUUGCCAAac | 0.88 | −0.181 |
| DS18-0092 | 92 | tttggcaaagttggtgcct | 491 | UUUGGCAAAGUUGGUGCCUgc | 891 | AGGCACCAACUUUGCCAAAcg | 0.98 | −0.027 |
| DS18-0093 | 93 | gtttggcaaagttggtgcc | 492 | GUUUGGCAAAGUUGGUGCCug | 892 | GGCACCAACUUUGCCAAACgc | 0.44 | −1.200 |
| DS18-0094 | 94 | cgtttggcaaagttggtgc | 493 | CGUUUGGCAAAGUUGGUGCcu | 893 | GCACCAACUUUGCCAAACGcu | 0.60 | −0.747 |
| DS18-0095 | 95 | gcgtttggcaaagttggtg | 494 | GCGUUUGGCAAAGUUGGUGcc | 894 | CACCAACUUUGCCAAACGCua | 0.43 | −1.215 |
| DS18-0096 | 96 | agcgtttggcaaagttggt | 495 | AGCGUUUGGCAAAGUUGGUgc | 895 | ACCAACUUUGCCAAACGCUac | 0.30 | −1.755 |
| DS18-0097 | 97 | tagcgtttggcaaagttgg | 496 | UAGCGUUUGGCAAAGUUGGug | 896 | CCAACUUUGCCAAACGCUAca | 0.8 | −0.308 |
| DS18-0098 | 98 | gtagcgtttggcaaagttg | 497 | GUAGCGUUUGGCAAAGUUGgu | 897 | CAACUUUGCCAAACGCUACag | 0.61 | −0.720 |
| DS18-0099 | 99 | tgtagcgtttggcaaagtt | 498 | UGUAGCGUUUGGCAAAGUUgg | 898 | AACUUUGCCAAACGCUACAga | 0.35 | −1.507 |
| DS18-0100 | 100 | ctgtagcgtttggcaaagt | 499 | CUGUAGCGUUUGGCAAAGUug | 899 | ACUUUGCCAAACGCUACAGag | 0.36 | −1.477 |
| DS18-0101 | 101 | tctgtagcgtttggcaaag | 500 | UCUGUAGCGUUUGGCAAAGuu | 900 | CUUUGCCAAACGCUACAGAgu | 0.50 | −1.012 |
| DS18-0102 | 102 | ctctgtagcgtttggcaaa | 501 | CUCUGUAGCGUUUGGCAAAgu | 901 | UUUGCCAAACGCUACAGAGuu | 0.53 | −0.908 |
| DS18-0103 | 103 | actctgtagcgtttggcaa | 502 | ACUCUGUAGCGUUUGGCAAag | 902 | UUGCCAAACGCUACAGAGUuu | 0.76 | −0.404 |
| DS18-0104 | 104 | aactctgtagcgtttggca | 503 | AACUCUGUAGCGUUUGGCAaa | 903 | UGCCAAACGCUACAGAGUUuu | 0.98 | −0.025 |
| DS18-0105 | 105 | aaactctgtagcgtttggc | 504 | AAACUCUGUAGCGUUUGGCaa | 904 | GCCAAACGCUACAGAGUUUuc | 0.89 | −0.175 |
| DS18-0106 | 106 | aaaactctgtagcgtttgg | 505 | AAAACUCUGUAGCGUUUGGca | 905 | CCAAACGCUACAGAGUUUUcc | 0.44 | −1.184 |
| DS18-0107 | 107 | gaaaactctgtagcgtttg | 506 | GAAAACUCUGUAGCGUUUGgc | 906 | CAAACGCUACAGAGUUUUCcg | 0.42 | −1.242 |
| DS18-0108 | 108 | ggaaaactctgtagcgttt | 507 | GGAAAACUCUGUAGCGUUUgg | 907 | AAACGCUACAGAGUUUUCCgu | 0.45 | −1.165 |
| DS18-0109 | 109 | cggaaaactctgtagcgtt | 508 | CGGAAAACUCUGUAGCGUUug | 908 | AACGCUACAGAGUUUUCCGuu | 0.41 | −1.277 |
| DS18-0110 | 110 | acggaaaactctgtagcgt | 509 | ACGGAAAACUCUGUAGCGUuu | 909 | ACGCUACAGAGUUUUCCGUuu | 0.36 | −1.476 |
| DS18-0111 | 111 | aacggaaaactctgtagcg | 510 | AACGGAAAACUCUGUAGCGuu | 910 | CGCUACAGAGUUUUCCGUUug | 0.57 | −0.815 |
| DS18-0112 | 112 | aaacggaaaactctgtagc | 511 | AAACGGAAAACUCUGUAGCgu | 911 | GCUACAGAGUUUUCCGUUUgc | 0.92 | −0.125 |
| DS18-0113 | 113 | caaacggaaaactctgtag | 512 | CAAACGGAAAACUCUGUAGcg | 912 | CUACAGAGUUUUCCGUUUGca | 0.74 | −0.440 |
| DS18-0114 | 114 | gcaaacggaaaactctgta | 513 | GCAAACGGAAAACUCUGUAgc | 913 | UACAGAGUUUUCCGUUUGCag | 0.81 | −0.311 |
| DS18-0115 | 115 | tgcaaacggaaaactctgt | 514 | UGCAAACGGAAAACUCUGUag | 914 | ACAGAGUUUUCCGUUUGCAga | 0.47 | −1.094 |
| DS18-0116 | 116 | ctgcaaacggaaaactctg | 515 | CUGCAAACGGAAAACUCUGua | 915 | CAGAGUUUUCCGUUUGCAGaa | 0.69 | −0.538 |
| DS18-0117 | 117 | tctgcaaacggaaaactct | 516 | UCUGCAAACGGAAAACUCUgu | 916 | AGAGUUUUCCGUUUGCAGAaa | 0.51 | −0.972 |
| DS18-0118 | 118 | ttctgcaaacggaaaactc | 517 | UUCUGCAAACGGAAAACUCug | 917 | GAGUUUUCCGUUUGCAGAAau | 1.63 | 0.702 |
| DS18-0119 | 119 | tttctgcaaacggaaaact | 518 | UUUCUGCAAACGGAAAACUcu | 918 | AGUUUUCCGUUUGCAGAAAug | 1.62 | 0.698 |
| DS18-0120 | 120 | atttctgcaaacggaaaac | 519 | AUUUCUGCAAACGGAAAACuc | 919 | GUUUUCCGUUUGCAGAAAUga | 0.96 | −0.060 |
| DS18-0121 | 121 | catttctgcaaacggaaaa | 520 | CAUUUCUGCAAACGGAAAAcu | 920 | UUUUCCGUUUGCAGAAAUGac | 0.32 | −1.638 |
| DS18-0122 | 122 | tcatttctgcaaacggaaa | 521 | UCAUUUCUGCAAACGGAAAac | 921 | UUUCCGUUUGCAGAAAUGAcc | 0.23 | −2.150 |
| DS18-0123 | 123 | gtcatttctgcaaacggaa | 522 | GUCAUUUCUGCAAACGGAAaa | 922 | UUCCGUUUGCAGAAAUGACcc | 0.28 | −1.811 |
| DS18-0124 | 124 | ggtcatttctgcaaacgga | 523 | GGUCAUUUCUGCAAACGGAaa | 923 | UCCGUUUGCAGAAAUGACCca | 0.29 | −1.783 |
| DS18-0125 | 125 | gggtcatttctgcaaacgg | 524 | GGGUCAUUUCUGCAAACGGaa | 924 | CCGUUUGCAGAAAUGACCCaa | 0.56 | −0.846 |
| DS18-0126 | 126 | tgggtcatttctgcaaacg | 525 | UGGGUCAUUUCUGCAAACGga | 925 | CGUUUGCAGAAAUGACCCAaa | 0.97 | −0.051 |
| DS18-0127 | 127 | ttgggtcatttctgcaaac | 526 | UUGGGUCAUUUCUGCAAACgg | 926 | GUUUGCAGAAAUGACCCAAaa | 0.75 | −0.408 |
| DS18-0128 | 128 | tttgggtcatttctgcaaa | 527 | UUUGGGUCAUUUCUGCAAAcg | 927 | UUUGCAGAAAUGACCCAAAag | 0.49 | −1.023 |
| DS18-0129 | 129 | ttttgggtcatttctgcaa | 528 | UUUUGGGUCAUUUCUGCAAac | 928 | UUGCAGAAAUGACCCAAAAga | 0.74 | −0.428 |
| DS18-0130 | 130 | cttttgggtcatttctgca | 529 | CUUUUGGGUCAUUUCUGCAaa | 929 | UGCAGAAAUGACCCAAAAGaa | 0.48 | −1.056 |
| DS18-0131 | 131 | tcttttgggtcatttctgc | 530 | UCUUUUGGGUCAUUUCUGCaa | 930 | GCAGAAAUGACCCAAAAGAag | 0.60 | −0.748 |
| DS18-0132 | 132 | ttcttttgggtcatttctg | 531 | UUCUUUUGGGUCAUUUCUGca | 931 | CAGAAAUGACCCAAAAGAAgg | 0.47 | −1.103 |
| DS18-0133 | 133 | cttcttttgggtcatttct | 532 | CUUCUUUUGGGUCAUUUCUgc | 932 | AGAAAUGACCCAAAAGAAGgg | 0.38 | −1.392 |
| DS18-0134 | 134 | ccttcttttgggtcatttc | 533 | CCUUCUUUUGGGUCAUUUCug | 933 | GAAAUGACCCAAAAGAAGGgg | 0.32 | −1.624 |
| DS18-0135 | 135 | cccttcttttgggtcattt | 534 | CCCUUCUUUUGGGUCAUUUcu | 934 | AAAUGACCCAAAAGAAGGGgg | 0.39 | −1.371 |
| DS18-0136 | 136 | ccccttcttttgggtcatt | 535 | CCCCUUCUUUUGGGUCAUUuc | 935 | AAUGACCCAAAAGAAGGGGgg | 0.32 | −1.641 |
| DS18-0137 | 137 | tggggaatcacggggagcg | 536 | UGGGGAAUCACGGGGAGCGgc | 936 | CGCUCCCCGUGAUUCCCCAau | 0.82 | −0.283 |
| DS18-0138 | 138 | ttggggaatcacggggagc | 537 | UUGGGGAAUCACGGGGAGCgg | 937 | GCUCCCCGUGAUUCCCCAAuc | 0.74 | −0.436 |
| DS18-0139 | 139 | attggggaatcacggggag | 538 | AUUGGGGAAUCACGGGGAGcg | 938 | CUCCCCGUGAUUCCCCAAUcg | 0.67 | −0.588 |
| DS18-0140 | 140 | gattggggaatcacgggga | 539 | GAUUGGGGAAUCACGGGGAgc | 939 | UCCCCGUGAUUCCCCAAUCgg | 0.44 | −1.185 |
| DS18-0141 | 141 | cgattggggaatcacgggg | 540 | CGAUUGGGGAAUCACGGGGag | 940 | CCCCGUGAUUCCCCAAUCGgc | 0.41 | −1.276 |
| DS18-0142 | 142 | ccgattggggaatcacggg | 541 | CCGAUUGGGGAAUCACGGGga | 941 | CCCGUGAUUCCCCAAUCGGcu | 0.36 | −1.491 |
| DS18-0143 | 143 | gccgattggggaatcacgg | 542 | GCCGAUUGGGGAAUCACGGgg | 942 | CCGUGAUUCCCCAAUCGGCuu | 0.42 | −1.248 |
| DS18-0144 | 144 | agccgattggggaatcacg | 543 | AGCCGAUUGGGGAAUCACGgg | 943 | CGUGAUUCCCCAAUCGGCUuc | 0.37 | −1.443 |
| DS18-0145 | 145 | aagccgattggggaatcac | 544 | AAGCCGAUUGGGGAAUCACgg | 944 | GUGAUUCCCCAAUCGGCUUcu | 0.34 | −1.544 |
| DS18-0146 | 146 | gaagccgattggggaatca | 545 | GAAGCCGAUUGGGGAAUCAcg | 945 | UGAUUCCCCAAUCGGCUUCug | 0.26 | −1.920 |
| DS18-0147 | 147 | agaagccgattggggaatc | 546 | AGAAGCCGAUUGGGGAAUCac | 946 | GAUUCCCCAAUCGGCUUCUgg | 0.30 | −1.739 |
| DS18-0148 | 148 | cagaagccgattggggaat | 547 | CAGAAGCCGAUUGGGGAAUca | 947 | AUUCCCCAAUCGGCUUCUGga | 0.48 | −1.047 |
| DS18-0149 | 149 | ccagaagccgattggggaa | 548 | CCAGAAGCCGAUUGGGGAAuc | 948 | UUCCCCAAUCGGCUUCUGGag | 0.29 | −1.771 |
| DS18-0150 | 150 | tccagaagccgattgggg | 549 | UCCAGAAGCCGAUUGGGGAau | 949 | UCCCCAAUCGGCUUCUGGAgc | 0.45 | −1.155 |
| DS18-0151 | 151 | ctccagaagccgattgggg | 550 | CUCCAGAAGCCGAUUGGGGaa | 950 | CCCCAAUCGGCUUCUGGAGcc | 0.44 | −1.183 |
| DS18-0152 | 152 | gctccagaagccgattggg | 551 | GCUCCAGAAGCCGAUUGGGga | 951 | CCCAAUCGGCUUCUGGAGCca | 0.37 | −1.442 |
| DS18-0153 | 153 | ggctccagaagccgattgg | 552 | GGCUCCAGAAGCCGAUUGGgg | 952 | CCAAUCGGCUUCUGGAGCCag | 0.41 | −1.272 |
| DS18-0154 | 154 | tggctccagaagccgattg | 553 | UGGCUCCAGAAGCCGAUUGgg | 953 | CAAUCGGCUUCUGGAGCCAga | 0.41 | −1.275 |
| DS18-0155 | 155 | ctggctccagaagccgatt | 554 | CUGGCUCCAGAAGCCGAUUgg | 954 | AAUCGGCUUCUGGAGCCAGag | 0.43 | −1.226 |
| DS18-0156 | 156 | tctggctccagaagccgat | 555 | UCUGGCUCCAGAAGCCGAUug | 955 | AUCGGCUUCUGGAGCCAGAgg | 0.66 | −0.608 |
| DS18-0157 | 157 | ctctggctccagaagccga | 556 | CUCUGGCUCCAGAAGCCGAuu | 956 | UCGGCUUCUGGAGCCAGAGgc | 0.43 | −1.203 |
| DS18-0158 | 158 | cctctggctccagaagccg | 557 | CCUCUGGCUCCAGAAGCCGau | 957 | CGGCUUCUGGAGCCAGAGGcu | 0.44 | −1.175 |
| DS18-0159 | 159 | gcctctggctccagaagcc | 558 | GCCUCUGGCUCCAGAAGCCga | 958 | GGCUUCUGGAGCCAGAGGCuc | 0.63 | −0.666 |
| DS18-0160 | 160 | agcctctggctccagaagc | 559 | AGCCUCUGGCUCCAGAAGCcg | 959 | GCUUCUGGAGCCAGAGGCUcc | 1.04 | 0.054 |
| DS18-0161 | 161 | gagcctctggctccagaag | 560 | GAGCCUCUGGCUCCAGAAGcc | 960 | CUUCUGGAGCCAGAGGCUCcu | 0.94 | −0.096 |
| DS18-0162 | 162 | ggagcctctggctccagaa | 561 | GGAGCCUCUGGCUCCAGAAgc | 961 | UUCUGGAGCCAGAGGCUCCuc | 0.81 | −0.310 |
| DS18-0163 | 163 | aggagcctctggctccaga | 562 | AGGAGCCUCUGGCUCCAGAag | 962 | UCUGGAGCCAGAGGCUCCUcc | 0.89 | −0.175 |
| DS18-0164 | 164 | gaggagcctctggctccag | 563 | GAGGAGCCUCUGGCUCCAGaa | 963 | CUGGAGCCAGAGGCUCCUCcu | 1.03 | 0.040 |
| DS18-0165 | 165 | ggaggagcctctggctcca | 564 | GGAGGAGCCUCUGGCUCCAga | 964 | UGGAGCCAGAGGCUCCUCCuc | 1.01 | 0.007 |
| DS18-0166 | 166 | aggaggagcctctggctcc | 565 | AGGAGGAGCCUCUGGCUCCag | 965 | GGAGCCAGAGGCUCCUCCUcg | 1.39 | 0.479 |
| DS18-0167 | 167 | agcagggaagcgggcagca | 566 | AGCAGGGAAGCGGGCAGCAgc | 966 | UGCUGCCCGCUUCCCUGCUcc | 0.68 | −0.556 |
| DS18-0168 | 168 | tcctcggagcagggaagcg | 567 | UCCUCGGAGCAGGGAAGCGgg | 967 | CGCUUCCCUGCUCCGAGGAaa | 1.28 | 0.361 |
| DS18-0169 | 169 | ttcctcggagcagggaagc | 568 | UUCCUCGGAGCAGGGAAGCgg | 968 | GCUUCCCUGCUCCGAGGAAaa | 0.85 | −0.227 |
| DS18-0170 | 170 | tttcctcggagcagggaag | 569 | UUUCCUCGGAGCAGGGAAGcg | 969 | CUUCCCUGCUCCGAGGAAAaa | 1.08 | 0.111 |
| DS18-0171 | 171 | ttttcctcggagcagggaa | 570 | UUUUCCUCGGAGCAGGGAAgc | 970 | UUCCCUGCUCCGAGGAAAAac | 0.81 | −0.302 |
| DS18-0172 | 172 | aaagttgtggagtcgtttt | 571 | AAAGUUGUGGAGUCGUUUUuc | 971 | AAAACGACUCCACAACUUUgu | 0.83 | −0.265 |
| DS18-0173 | 173 | caaagttgtggagtcgttt | 572 | CAAAGUUGUGGAGUCGUUUuu | 972 | AAACGACUCCACAACUUUGug | 0.69 | −0.534 |
| DS18-0174 | 174 | acaaagttgtggagtcgtt | 573 | ACAAAGUUGUGGAGUCGUUuu | 973 | AACGACUCCACAACUUUGUgc | 0.67 | −0.570 |
| DS18-0175 | 175 | cacaaagttgtggagtcgt | 574 | CACAAAGUUGUGGAGUCGUuu | 974 | ACGACUCCACAACUUUGUGcg | 0.71 | −0.484 |
| DS18-0176 | 176 | gcacaaagttgtggagtcg | 575 | GCACAAAGUUGUGGAGUCGuu | 975 | CGACUCCACAACUUUGUGCgc | 0.64 | −0.634 |
| DS18-0177 | 177 | cgcacaaagttgtggagtc | 576 | CGCACAAAGUUGUGGAGUCgu | 976 | GACUCCACAACUUUGUGCGcg | 0.29 | −1.802 |
| DS18-0178 | 178 | gcgcacaaagttgtggagt | 577 | GCGCACAAAGUUGUGGAGUcg | 977 | ACUCCACAACUUUGUGCGCga | 0.75 | −0.413 |
| DS18-0179 | 179 | cgcgcacaaagttgtggag | 578 | CGCGCACAAAGUUGUGGAGuc | 978 | CUCCACAACUUUGUGCGCGag | 0.87 | −0.208 |
| DS18-0180 | 180 | tcgcgcacaaagttgtgga | 579 | UCGCGCACAAAGUUGUGGAgu | 979 | UCCACAACUUUGUGCGCGAga | 0.82 | −0.293 |
| DS18-0181 | 181 | ctcgcgcacaaagttgtgg | 580 | CUCGCGCACAAAGUUGUGGag | 980 | CCACAACUUUGUGCGCGAGag | 1.14 | 0.184 |
| DS18-0182 | 182 | tctcgcgcacaaagttgtg | 581 | UCUCGCGCACAAAGUUGUGga | 981 | CACAACUUUGUGCGCGAGAga | 0.96 | −0.065 |
| DS18-0183 | 183 | ctctcgcgcacaaagttgt | 582 | CUCUCGCGCACAAAGUUGUgg | 982 | ACAACUUUGUGCGCGAGAGag | 1.01 | 0.017 |
| DS18-0184 | 184 | ccgaaggagcgagcctctc | 583 | CCGAAGGAGCGAGCCUCUCuc | 983 | GAGAGGCUCGCUCCUUCGGcg | 1.27 | 0.341 |
| DS18-0185 | 185 | gccgaaggagcgagcctct | 584 | GCCGAAGGAGCGAGCCUCUcu | 984 | AGAGGCUCGCUCCUUCGGCgg | 1.51 | 0.598 |
| DS18-0186 | 186 | aggaggagccgccgaagga | 585 | AGGAGGAGCCGCCGAAGGAgc | 985 | UCCUUCGGCGGCUCCUCCUcc | 0.85 | −0.232 |
| DS18-0187 | 187 | acaaaggggcaggcccgca | 586 | ACAAAGGGGCAGGCCCGCAgc | 986 | UGCGGGCCUGCCCCUUUGUug | 1.18 | 0.242 |
| DS18-0188 | 188 | aacaaaggggcaggcccgc | 587 | AACAAAGGGGCAGGCCCGCag | 987 | GCGGGCCUGCCCCUUUGUUgg | 1.29 | 0.363 |
| DS18-0189 | 189 | caacaaaggggcaggcccg | 588 | CAACAAAGGGGCAGGCCCGca | 988 | CGGGCCUGCCCCUUUGUUGga | 1.17 | 0.228 |
| DS18-0190 | 190 | ccaacaaaggggcaggccc | 589 | CCAACAAAGGGGCAGGCCCgc | 989 | GGGCCUGCCCCUUUGUUGGag | 1.04 | 0.054 |
| DS18-0191 | 191 | tccaacaaaggggcaggcc | 590 | UCCAACAAAGGGGCAGGCCcg | 990 | GGCCUGCCCCUUUGUUGGAgc | 1.14 | 0.192 |
| DS18-0192 | 192 | ctccaacaaaggggcaggc | 591 | CUCCAACAAAGGGGCAGGCcc | 991 | GCCUGCCCCUUUGUUGGAGcc | 1.17 | 0.225 |
| DS18-0193 | 193 | gctccaacaaaggggcagg | 592 | GCUCCAACAAAGGGGCAGGcc | 992 | CCUGCCCCUUUGUUGGAGCcg | 1.42 | 0.502 |
| DS18-0194 | 194 | ggctccaacaaaggggcag | 593 | GGCUCCAACAAAGGGGCAGgc | 993 | CUGCCCCUUUGUUGGAGCCga | 1.25 | 0.327 |
| DS18-0195 | 195 | cggctccaacaaaggggca | 594 | CGGCUCCAACAAAGGGGCAgg | 994 | UGCCCCUUUGUUGGAGCCGag | 1.05 | 0.064 |
| DS18-0196 | 196 | tcggctccaacaaaggggc | 595 | UCGGCUCCAACAAAGGGGCag | 995 | GCCCCUUUGUUGGAGCCGAgc | 0.88 | −0.181 |
| DS18-0197 | 197 | ctcggctccaacaaagggg | 596 | CUCGGCUCCAACAAAGGGGca | 996 | CCCCUUUGUUGGAGCCGAGcg | 1.93 | 0.946 |
| DS18-0198 | 198 | gctcggctccaacaaaggg | 597 | GCUCGGCUCCAACAAAGGGgc | 997 | CCCUUUGUUGGAGCCGAGCgc | 1.77 | 0.821 |
| DS18-0199 | 199 | cgctcggctccaacaaagg | 598 | CGCUCGGCUCCAACAAAGGgg | 998 | CCUUUGUUGGAGCCGAGCGcu | 1.33 | 0.410 |
| DS18-0200 | 200 | gcgctcggctccaacaaag | 599 | GCGCUCGGCUCCAACAAAGgg | 999 | CUUUGUUGGAGCCGAGCGCug | 1.59 | 0.668 |
| DS18-0201 | 201 | agcgctcggctccaacaaa | 600 | AGCGCUCGGCUCCAACAAAgg | 1000 | UUUGUUGGAGCCGAGCGCUgg | 0.79 | −0.347 |
| DS18-0202 | 202 | cagcgctcggctccaacaa | 601 | CAGCGCUCGGCUCCAACAAag | 1001 | UUGUUGGAGCCGAGCGCUGga | 1.10 | 0.140 |
| DS18-0203 | 203 | ccagcgctcggctccaaca | 602 | CCAGCGCUCGGCUCCAACAaa | 1002 | UGUUGGAGCCGAGCGCUGGag | 0.96 | −0.052 |
| DS18-0204 | 204 | tccagcgctcggctccaac | 603 | UCCAGCGCUCGGCUCCAACaa | 1003 | GUUGGAGCCGAGCGCUGGAgg | 1.23 | 0.297 |
| DS18-0205 | 205 | ctccagcgctcggctccaa | 604 | CUCCAGCGCUCGGCUCCAAca | 1004 | UUGGAGCCGAGCGCUGGAGgc | 1.73 | 0.789 |
| DS18-0206 | 206 | tctggagagcggacccctg | 605 | UCUGGAGAGCGGACCCCUGgc | 1005 | CAGGGGUCCGCUCUCCAGAug | 1.38 | 0.460 |
| DS18-0207 | 207 | atctggagagcggacccct | 606 | AUCUGGAGAGCGGACCCCUgg | 1006 | AGGGGUCCGCUCUCCAGAUga | 1.48 | 0.567 |
| DS18-0208 | 208 | catctggagagcggacccc | 607 | CAUCUGGAGAGCGGACCCCug | 1007 | GGGGUCCGCUCUCCAGAUGag | 1.30 | 0.378 |
| DS18-0209 | 209 | tcatctggagagcggaccc | 608 | UCAUCUGGAGAGCGGACCCcu | 1008 | GGGUCCGCUCUCCAGAUGAga | 1.45 | 0.540 |
| DS18-0210 | 210 | ctcatctggagagcggacc | 609 | CUCAUCUGGAGAGCGGACCcc | 1009 | GGUCCGCUCUCCAGAUGAGaa | 0.99 | −0.019 |
| DS18-0211 | 211 | tctcatctggagagcggac | 610 | UCUCAUCUGGAGAGCGGACcc | 1010 | GUCCGCUCUCCAGAUGAGAag | 1.15 | 0.197 |
| DS18-0212 | 212 | ttctcatctggagagcgga | 611 | UUCUCAUCUGGAGAGCGGAcc | 1011 | UCCGCUCUCCAGAUGAGAAgg | 1.09 | 0.124 |
| DS18-0213 | 213 | cttctcatctggagagcgg | 612 | CUUCUCAUCUGGAGAGCGGac | 1012 | CCGCUCUCCAGAUGAGAAGgg | 0.94 | −0.096 |
| DS18-0214 | 214 | ccttctcatctggagagcg | 613 | CCUUCUCAUCUGGAGAGCGga | 1013 | CGCUCUCCAGAUGAGAAGGgu | 0.86 | −0.221 |
| DS18-0215 | 215 | cccttctcatctggagagc | 614 | CCCUUCUCAUCUGGAGAGCgg | 1014 | GCUCUCCAGAUGAGAAGGGug | 0.90 | −0.153 |
| DS18-0216 | 216 | acccttctcatctggagag | 615 | ACCCUUCUCAUCUGGAGAGcg | 1015 | CUCUCCAGAUGAGAAGGGUgc | 1.12 | 0.157 |
| DS18-0217 | 217 | cacccttctcatctggaga | 616 | CACCCUUCUCAUCUGGAGAgc | 1016 | UCUCCAGAUGAGAAGGGUGca | 0.84 | −0.253 |
| DS18-0218 | 218 | gcacccttctcatctggag | 617 | GCACCCUUCUCAUCUGGAGag | 1017 | CUCCAGAUGAGAAGGGUGCag | 0.87 | −0.199 |
| DS18-0219 | 219 | tgcacccttctcatctgga | 618 | UGCACCCUUCUCAUCUGGAga | 1018 | UCCAGAUGAGAAGGGUGCAga | 0.99 | −0.019 |
| DS18-0220 | 220 | ctgcacccttctcatctgg | 619 | CUGCACCCUUCUCAUCUGGag | 1019 | CCAGAUGAGAAGGGUGCAGau | 0.95 | −0.078 |
| DS18-0221 | 221 | tctgcacccttctcatctg | 620 | UCUGCACCCUUCUCAUCUGga | 1020 | CAGAUGAGAAGGGUGCAGAuc | 1.31 | 0.386 |
| DS18-0222 | 222 | atctgcacccttctcatct | 621 | AUCUGCACCCUUCUCAUCUgg | 1021 | AGAUGAGAAGGGUGCAGAUca | 1.57 | 0.655 |
| DS18-0223 | 223 | gatctgcacccttctcatc | 622 | GAUCUGCACCCUUCUCAUCug | 1022 | GAUGAGAAGGGUGCAGAUCag | 1.25 | 0.322 |
| DS18-0224 | 224 | tgatctgcacccttctcat | 623 | UGAUCUGCACCCUUCUCAUcu | 1023 | AUGAGAAGGGUGCAGAUCAgc | 1.00 | −0.004 |
| DS18-0225 | 225 | ctgatctgcacccttctca | 624 | CUGAUCUGCACCCUUCUCAuc | 1024 | UGAGAAGGGUGCAGAUCAGcg | 1.59 | 0.674 |
| DS18-0226 | 226 | gctgatctgcacccttctc | 625 | GCUGAUCUGCACCCUUCUCau | 1025 | GAGAAGGGUGCAGAUCAGCgc | 0.65 | −0.624 |
| DS18-0227 | 227 | cgctgatctgcacccttct | 626 | CGCUGAUCUGCACCCUUCUca | 1026 | AGAAGGGUGCAGAUCAGCGcc | 0.60 | −0.747 |
| DS18-0228 | 228 | gcgctgatctgcacccttc | 627 | GCGCUGAUCUGCACCCUUCuc | 1027 | GAAGGGUGCAGAUCAGCGCca | 1.12 | 0.158 |
| DS18-0229 | 229 | ggcgctgatctgcaccctt | 628 | GGCGCUGAUCUGCACCCUUcu | 1028 | AAGGGUGCAGAUCAGCGCCag | 1.21 | 0.276 |
| DS18-0230 | 230 | tggcgctgatctgcaccct | 629 | UGGCGCUGAUCUGCACCCUuc | 1029 | AGGGUGCAGAUCAGCGCCAgc | 1.40 | 0.483 |
| DS18-0231 | 231 | ctggcgctgatctgcaccc | 630 | CUGGCGCUGAUCUGCACCCuu | 1030 | GGGUGCAGAUCAGCGCCAGcc | 1.26 | 0.338 |
| DS18-0232 | 232 | gctggcgctgatctgcacc | 631 | GCUGGCGCUGAUCUGCACCcu | 1031 | GGUGCAGAUCAGCGCCAGCcc | 1.29 | 0.368 |
| DS18-0233 | 233 | ggctggcgctgatctgcac | 632 | GGCUGGCGCUGAUCUGCACcc | 1032 | GUGCAGAUCAGCGCCAGCCcu | 1.33 | 0.410 |
| DS18-0234 | 234 | gggctggcgctgatctgca | 633 | GGGCUGGCGCUGAUCUGCAcc | 1033 | UGCAGAUCAGCGCCAGCCCuc | 1.29 | 0.367 |
| DS18-0235 | 235 | agggctggcgctgatctgc | 634 | AGGGCUGGCGCUGAUCUGCac | 1034 | GCAGAUCAGCGCCAGCCCUcc | 0.76 | −0.399 |
| DS18-0236 | 236 | gagggctggcgctgatctg | 635 | GAGGGCUGGCGCUGAUCUGca | 1035 | CAGAUCAGCGCCAGCCCUCcg | 1.39 | 0.470 |
| DS18-0237 | 237 | ggagggctggcgctgatct | 636 | GGAGGGCUGGCGCUGAUCUgc | 1036 | AGAUCAGCGCCAGCCCUCCgg | 1.13 | 0.173 |
| DS18-0238 | 238 | aaagttgctgggccggcgg | 637 | AAAGUUGCUGGGCCGGCGGcg | 1037 | CCGCCGGCCCAGCAACUUUgu | 1.05 | 0.069 |
| DS18-0239 | 239 | caaagttgctgggccggcg | 638 | CAAAGUUGCUGGGCCGGCGgc | 1038 | CGCCGGCCCAGCAACUUUGuu | 1.09 | 0.126 |
| DS18-0240 | 240 | acaaagttgctgggccggc | 639 | ACAAAGUUGCUGGGCCGGCgg | 1039 | GCCGGCCCAGCAACUUUGUuc | 1.04 | 0.052 |
| DS18-0241 | 241 | aacaaagttgctgggccgg | 640 | AACAAAGUUGCUGGGCCGGcg | 1040 | CCGGCCCAGCAACUUUGUUcc | 1.06 | 0.078 |
| DS18-0242 | 242 | gaacaaagttgctgggccg | 641 | GAACAAAGUUGCUGGGCCGgc | 1041 | CGGCCCAGCAACUUUGUUCcg | 1.06 | 0.090 |
| DS18-0243 | 243 | ggaacaaagttgctgggcc | 642 | GGAACAAAGUUGCUGGGCCgg | 1042 | GGCCCAGCAACUUUGUUCCgg | 1.23 | 0.294 |
| DS18-0244 | 244 | cggaacaaagttgctgggc | 643 | CGGAACAAAGUUGCUGGGCcg | 1043 | GCCCAGCAACUUUGUUCCGga | 1.08 | 0.113 |
| DS18-0245 | 245 | ccggaacaaagttgctggg | 644 | CCGGAACAAAGUUGCUGGGcc | 1044 | CCCAGCAACUUUGUUCCGGaa | 0.72 | −0.468 |
| DS18-0246 | 246 | tccggaacaaagttgctgg | 645 | UCCGGAACAAAGUUGCUGGgc | 1045 | CCAGCAACUUUGUUCCGGAag | 0.75 | −0.418 |
| DS18-0247 | 247 | ttccggaacaaagttgctg | 646 | UUCCGGAACAAAGUUGCUGgg | 1046 | CAGCAACUUUGUUCCGGAAga | 0.81 | −0.295 |
| DS18-0248 | 248 | cttccggaacaaagttgct | 647 | CUUCCGGAACAAAGUUGCUgg | 1047 | AGCAACUUUGUUCCGGAAGau | 1.30 | 0.381 |
| DS18-0249 | 249 | tcttccggaacaaagttgc | 648 | UCUUCCGGAACAAAGUUGCug | 1048 | GCAACUUUGUUCCGGAAGAuc | 0.99 | −0.017 |
| DS18-0250 | 250 | atcttccggaacaaagttg | 649 | AUCUUCCGGAACAAAGUUGcu | 1049 | CAACUUUGUUCCGGAAGAUca | 0.63 | −0.657 |
| DS18-0251 | 251 | gatcttccggaacaaagtt | 650 | GAUCUUCCGGAACAAAGUUgc | 1050 | AACUUUGUUCCGGAAGAUCag | 1.24 | 0.307 |
| DS18-0252 | 252 | tgatcttccggaacaaagt | 651 | UGAUCUUCCGGAACAAAGUug | 1051 | ACUUUGUUCCGGAAGAUCAgc | 1.27 | 0.340 |
| DS18-0253 | 253 | ctgatcttccggaacaaag | 652 | CUGAUCUUCCGGAACAAAGuu | 1052 | CUUUGUUCCGGAAGAUCAGcc | 1.50 | 0.585 |
| DS18-0254 | 254 | gctgatcttccggaacaaa | 653 | GCUGAUCUUCCGGAACAAAgu | 1053 | UUUGUUCCGGAAGAUCAGCcc | 1.09 | 0.120 |
| DS18-0255 | 255 | ggctgatcttccggaacaa | 654 | GGCUGAUCUUCCGGAACAAag | 1054 | UUGUUCCGGAAGAUCAGCCcc | 1.15 | 0.203 |
| DS18-0256 | 256 | gggctgatcttccggaaca | 655 | GGGCUGAUCUUCCGGAACAaa | 1055 | UGUUCCGGAAGAUCAGCCCca | 0.78 | −0.365 |
| DS18-0257 | 257 | ggggctgatcttccggaac | 656 | GGGGCUGAUCUUCCGGAACaa | 1056 | GUUCCGGAAGAUCAGCCCCac | 0.77 | −0.374 |
| DS18-0258 | 258 | tggggctgatcttccggaa | 657 | UGGGGCUGAUCUUCCGGAAca | 1057 | UUCCGGAAGAUCAGCCCCAcu | 0.65 | −0.617 |
| DS18-0259 | 259 | gtggggctgatcttccgga | 658 | GUGGGGCUGAUCUUCCGGAac | 1058 | UCCGGAAGAUCAGCCCCACua | 0.70 | −0.509 |
| DS18-0260 | 260 | agtggggctgatcttccgg | 659 | AGUGGGGCUGAUCUUCCGGaa | 1059 | CCGGAAGAUCAGCCCCACUac | 0.76 | −0.393 |
| DS18-0261 | 261 | tagtggggctgatcttccg | 660 | UAGUGGGGCUGAUCUUCCGga | 1060 | CGGAAGAUCAGCCCCACUAcg | 0.70 | −0.513 |
| DS18-0262 | 262 | gtagtggggctgatcttcc | 661 | GUAGUGGGGCUGAUCUUCCgg | 1061 | GGAAGAUCAGCCCCACUACgu | 0.64 | −0.641 |
| DS18-0263 | 263 | cgtagtggggctgatcttc | 662 | CGUAGUGGGGCUGAUCUUCcg | 1062 | GAAGAUCAGCCCCACUACGuu | 0.94 | −0.092 |
| DS18-0264 | 264 | acgtagtggggctgatctt | 663 | ACGUAGUGGGGCUGAUCUUcc | 1063 | AAGAUCAGCCCCACUACGUuc | 1.25 | 0.325 |
| DS18-0265 | 265 | aacgtagtggggctgatct | 664 | AACGUAGUGGGGCUGAUCUuc | 1064 | AGAUCAGCCCCACUACGUUcc | 0.76 | −0.405 |
| DS18-0266 | 266 | gaacgtagtggggctgatc | 665 | GAACGUAGUGGGGCUGAUCuu | 1065 | GAUCAGCCCCACUACGUUCcc | 0.99 | −0.014 |
| DS18-0267 | 267 | ggaacgtagtggggctgat | 666 | GGAACGUAGUGGGGCUGAUcu | 1066 | AUCAGCCCCACUACGUUCCeg | 0.93 | −0.099 |
| DS18-0268 | 268 | gggaacgtagtggggctga | 667 | GGGAACGUAGUGGGGCUGAuc | 1067 | UCAGCCCCACUACGUUCCCgg | 1.01 | 0.020 |
| DS18-0269 | 269 | cgggaacgtagtggggctg | 668 | CGGGAACGUAGUGGGGCUGau | 1068 | CAGCCCCACUACGUUCCCGgg | 0.95 | −0.081 |
| DS18-0270 | 270 | ccgggaacgtagtggggct | 669 | CCGGGAACGUAGUGGGGCUga | 1069 | AGCCCCACUACGUUCCCGGgu | 0.89 | −0.169 |
| DS18-0271 | 271 | acccgggaacgtagtgggg | 670 | ACCCGGGAACGUAGUGGGGcu | 1070 | CCCCACUACGUUCCCGGGUca | 1.15 | 0.207 |
| DS18-0272 | 272 | gacccgggaacgtagtggg | 671 | GACCCGGGAACGUAGUGGGgc | 1071 | CCCACUACGUUCCCGGGUCag | 1.26 | 0.336 |
| DS18-0273 | 273 | tgacccgggaacgtagtgg | 672 | UGACCCGGGAACGUAGUGGgg | 1072 | CCACUACGUUCCCGGGUCAgc | 0.86 | −0.220 |
| DS18-0274 | 274 | ctgacccgggaacgtagtg | 673 | CUGACCCGGGAACGUAGUGgg | 1073 | CACUACGUUCCCGGGUCAGcg | 0.88 | −0.185 |
| DS18-0275 | 275 | gctgacccgggaacgtagt | 674 | GCUGACCCGGGAACGUAGUgg | 1074 | ACUACGUUCCCGGGUCAGCgu | 0.84 | −0.251 |
| DS18-0276 | 276 | cagacgctgacccgggaac | 675 | CAGACGCUGACCCGGGAACgu | 1075 | GUUCCCGGGUCAGCGUCUGcc | 1.05 | 0.065 |
| DS18-0277 | 277 | gcagacgctgacccgggaa | 676 | GCAGACGCUGACCCGGGAAcg | 1076 | UUCCCGGGUCAGCGUCUGCcg | 1.21 | 0.281 |
| DS18-0278 | 278 | tggggcgccaagttcccga | 677 | UGGGGCGCCAAGUUCCCGAcg | 1077 | UCGGGAACUUGGCGCCCCAag | 0.91 | −0.135 |
| DS18-0279 | 279 | ttggggcgccaagttcccg | 678 | UUGGGGCGCCAAGUUCCCGac | 1078 | CGGGAACUUGGCGCCCCAAgu | 1.04 | 0.051 |
| DS18-0280 | 280 | cttggggcgccaagttccc | 679 | CUUGGGGCGCCAAGUUCCCga | 1079 | GGGAACUUGGCGCCCCAAGuc | 1.14 | 0.189 |
| DS18-0281 | 281 | acttggggcgccaagttcc | 680 | ACUUGGGGCGCCAAGUUCCcg | 1080 | GGAACUUGGCGCCCCAAGUca | 0.89 | −0.165 |
| DS18-0282 | 282 | gacttggggcgccaagttc | 681 | GACUUGGGGCGCCAAGUUCcc | 1081 | GAACUUGGCGCCCCAAGUCac | 0.97 | −0.049 |
| DS18-0283 | 283 | tgacttggggcgccaagtt | 682 | UGACUUGGGGCGCCAAGUUcc | 1082 | AACUUGGCGCCCCAAGUCAcc | 1.00 | 0.004 |
| DS18-0284 | 284 | gtgacttggggcgccaagt | 683 | GUGACUUGGGGCGCCAAGUuc | 1083 | ACUUGGCGCCCCAAGUCACcu | 1.16 | 0.218 |
| DS18-0285 | 285 | ggtgacttggggcgccaag | 684 | GGUGACUUGGGGCGCCAAGuu | 1084 | CUUGGCGCCCCAAGUCACCug | 1.15 | 0.202 |
| DS18-0286 | 286 | aggtgacttggggcgccaa | 685 | AGGUGACUUGGGGCGCCAAgu | 1085 | UUGGCGCCCCAAGUCACCUga | 0.91 | −0.138 |
| DS18-0287 | 287 | caggtgacttggggcgcca | 686 | CAGGUGACUUGGGGCGCCAag | 1086 | UGGCGCCCCAAGUCACCUGac | 1.09 | 0.123 |
| DS18-0288 | 288 | tcaggtgacttggggcgcc | 687 | UCAGGUGACUUGGGGCGCCaa | 1087 | GGCGCCCCAAGUCACCUGAcu | 1.05 | 0.064 |
| DS18-0289 | 289 | gtcaggtgacttggggcgc | 688 | GUCAGGUGACUUGGGGCGCca | 1088 | GCGCCCCAAGUCACCUGACug | 1.03 | 0.049 |
| DS18-0290 | 290 | agtcaggtgacttggggcg | 689 | AGUCAGGUGACUUGGGGCGcc | 1089 | CGCCCCAAGUCACCUGACUgc | 0.58 | −0.777 |
| DS18-0291 | 291 | cagtcaggtgacttggggc | 690 | CAGUCAGGUGACUUGGGGCgc | 1090 | GCCCCAAGUCACCUGACUGog | 0.96 | −0.066 |
| DS18-0292 | 292 | gcagtcaggtgacttgggg | 691 | GCAGUCAGGUGACUUGGGGcg | 1091 | CCCCAAGUCACCUGACUGCgc | 0.92 | −0.118 |
| DS18-0293 | 293 | cgcagtcaggtgacttggg | 692 | CGCAGUCAGGUGACUUGGGgc | 1092 | CCCAAGUCACCUGACUGCGcg | 0.93 | −0.104 |
| DS18-0294 | 294 | gcgcagtcaggtgacttgg | 693 | GCGCAGUCAGGUGACUUGGgg | 1093 | CCAAGUCACCUGACUGCGCgc | 0.93 | −0.100 |
| DS18-0295 | 295 | cgcgcagtcaggtgacttg | 694 | CGCGCAGUCAGGUGACUUGgg | 1094 | CAAGUCACCUGACUGCGCGcc | 1.01 | 0.007 |
| DS18-0296 | 296 | agggccggcgctgattgac | 695 | AGGGCCGGCGCUGAUUGACgg | 1095 | GUCAAUCAGCGCCGGCCCUgc | 1.01 | 0.021 |
| DS18-0297 | 297 | cagggccggcgctgattga | 696 | CAGGGCCGGCGCUGAUUGAcg | 1096 | UCAAUCAGCGCCGGCCCUGcg | 1.30 | 0.379 |
| DS18-0298 | 298 | acggttcactctggagcgc | 697 | ACGGUUCACUCUGGAGCGCgc | 1097 | GCGCUCCAGAGUGAACCGUgc | 1.25 | 0.322 |
| DS18-0299 | 299 | cacggttcactctggagcg | 698 | CACGGUUCACUCUGGAGCGcg | 1098 | CGCUCCAGAGUGAACCGUGcg | 0.74 | −0.441 |
| DS18-0300 | 300 | gcacggttcactctggagc | 699 | GCACGGUUCACUCUGGAGCgc | 1099 | GCUCCAGAGUGAACCGUGCgu | 1.06 | 0.084 |
| DS18-0301 | 301 | cgcacggttcactctggag | 700 | CGCACGGUUCACUCUGGAGcg | 1100 | CUCCAGAGUGAACCGUGCGug | 1.58 | 0.658 |
| DS18-0302 | 302 | acgcacggttcactctgga | 701 | ACGCACGGUUCACUCUGGAgc | 1101 | UCCAGAGUGAACCGUGCGUgc | 1.07 | 0.092 |
| DS18-0303 | 303 | cacgcacggttcactctgg | 702 | CACGCACGGUUCACUCUGGag | 1102 | CCAGAGUGAACCGUGCGUGcc | 1.08 | 0.111 |
| DS18-0304 | 304 | gcacgcacggttcactctg | 703 | GCACGCACGGUUCACUCUGga | 1103 | CAGAGUGAACCGUGCGUGCog | 1.42 | 0.510 |
| DS18-0305 | 305 | ggcacgcacggttcactct | 704 | GGCACGCACGGUUCACUCUgg | 1104 | AGAGUGAACCGUGCGUGCCgg | 2.08 | 1.059 |
| DS18-0306 | 306 | gttcggtctccggactccc | 705 | GUUCGGUCUCCGGACUCCCgg | 1105 | GGGAGUCCGGAGACCGAACca | 1.02 | 0.024 |
| DS18-0307 | 307 | ggttcggtctccggactcc | 706 | GGUUCGGUCUCCGGACUCCcg | 1106 | GGAGUCCGGAGACCGAACCag | 1.03 | 0.037 |
| DS18-0308 | 308 | tggttcggtctccggactc | 707 | UGGUUCGGUCUCCGGACUCcc | 1107 | GAGUCCGGAGACCGAACCAga | 1.23 | 0.299 |
| DS18-0309 | 309 | ctggttcggtctccggact | 708 | CUGGUUCGGUCUCCGGACUcc | 1108 | AGUCCGGAGACCGAACCAGaa | 0.90 | −0.145 |
| DS18-0310 | 310 | tctggttcggtctccggac | 709 | UCUGGUUCGGUCUCCGGACuc | 1109 | GUCCGGAGACCGAACCAGAau | 1.14 | 0.190 |
| DS18-0311 | 311 | ttctggttcggtctccgga | 710 | UUCUGGUUCGGUCUCCGGAcu | 1110 | UCCGGAGACCGAACCAGAAug | 1.12 | 0.162 |
| DS18-0312 | 312 | attctggttcggtctccgg | 711 | AUUCUGGUUCGGUCUCCGGac | 1111 | CCGGAGACCGAACCAGAAUgg | 1.33 | 0.408 |
| DS18-0313 | 313 | cattctggttcggtctccg | 712 | CAUUCUGGUUCGGUCUCCGga | 1112 | CGGAGACCGAACCAGAAUGga | 1.75 | 0.808 |
| DS18-0314 | 314 | ccattctggttcggtctcc | 713 | CCAUUCUGGUUCGGUCUCCgg | 1113 | GGAGACCGAACCAGAAUGGau | 1.28 | 0.355 |
| DS18-0315 | 315 | tccattctggttcggtctc | 714 | UCCAUUCUGGUUCGGUCUCcg | 1114 | GAGACCGAACCAGAAUGGAug | 1.33 | 0.414 |
| DS18-0316 | 316 | atccattctggttcggtct | 715 | AUCCAUUCUGGUUCGGUCUcc | 1115 | AGACCGAACCAGAAUGGAUgc | 0.97 | −0.038 |
| DS18-0317 | 317 | catccattctggttcggtc | 716 | CAUCCAUUCUGGUUCGGUCuc | 1116 | GACCGAACCAGAAUGGAUGcc | 1.04 | 0.051 |
| DS18-0318 | 318 | gcatccattctggttcggt | 717 | GCAUCCAUUCUGGUUCGGUcu | 1117 | ACCGAACCAGAAUGGAUGCcu | 1.04 | 0.055 |
| DS18-0319 | 319 | ggcatccattctggttcgg | 718 | GGCAUCCAUUCUGGUUCGGuc | 1118 | CCGAACCAGAAUGGAUGCCuc | 0.95 | −0.079 |
| DS18-0320 | 320 | aggcatccattctggttcg | 719 | AGGCAUCCAUUCUGGUUCGgu | 1119 | CGAACCAGAAUGGAUGCCUcg | 1.14 | 0.187 |
| DS18-0321 | 321 | gaggcatccattctggttc | 720 | GAGGCAUCCAUUCUGGUUCgg | 1120 | GAACCAGAAUGGAUGCCUCga | 1.42 | 0.510 |
| DS18-0322 | 322 | cgaggcatccattctggtt | 721 | CGAGGCAUCCAUUCUGGUUcg | 1121 | AACCAGAAUGGAUGCCUCGag | 1.08 | 0.108 |
| DS18-0323 | 323 | tcgaggcatccattctggt | 722 | UCGAGGCAUCCAUUCUGGUuc | 1122 | ACCAGAAUGGAUGCCUCGAgc | 1.64 | 0.710 |
| DS18-0324 | 324 | ctcgaggcatccattctgg | 723 | CUCGAGGCAUCCAUUCUGGuu | 1123 | CCAGAAUGGAUGCCUCGAGca | 1.72 | 0.783 |
| DS18-0325 | 325 | gctcgaggcatccattctg | 724 | GCUCGAGGCAUCCAUUCUGgu | 1124 | CAGAAUGGAUGCCUCGAGCag | 1.25 | 0.325 |
| DS18-0326 | 326 | tgctcgaggcatccattct | 725 | UGCUCGAGGCAUCCAUUCUgg | 1125 | AGAAUGGAUGCCUCGAGCAga | 1.00 | −0.004 |
| DS18-0327 | 327 | ctgctcgaggcatccattc | 726 | CUGCUCGAGGCAUCCAUUCug | 1126 | GAAUGGAUGCCUCGAGCAGac | 1.14 | 0.191 |
| DS18-0328 | 328 | tctgctcgaggcatccatt | 727 | UCUGCUCGAGGCAUCCAUUcu | 1127 | AAUGGAUGCCUCGAGCAGAca | 1.29 | 0.363 |
| DS18-0329 | 329 | gtctgctcgaggcatccat | 728 | GUCUGCUCGAGGCAUCCAUuc | 1128 | AUGGAUGCCUCGAGCAGACac | 1.51 | 0.591 |
| DS18-0330 | 330 | tgtctgctcgaggcatcca | 729 | UGUCUGCUCGAGGCAUCCAuu | 1129 | UGGAUGCCUCGAGCAGACAca | 0.79 | −0.347 |
| DS18-0331 | 331 | gtgtctgctcgaggcatcc | 730 | GUGUCUGCUCGAGGCAUCCau | 1130 | GGAUGCCUCGAGCAGACACaa | 1.08 | 0.116 |
| DS18-0332 | 332 | tgtgtctgctcgaggcatc | 731 | UGUGUCUGCUCGAGGCAUCca | 1131 | GAUGCCUCGAGCAGACACAau | 1.59 | 0.670 |
| DS18-0333 | 333 | ttgtgtctgctcgaggcat | 732 | UUGUGUCUGCUCGAGGCAUcc | 1132 | AUGCCUCGAGCAGACACAAuu | 1.94 | 0.955 |
| DS18-0334 | 334 | attgtgtctgctcgaggca | 733 | AUUGUGUCUGCUCGAGGCAuc | 1133 | UGCCUCGAGCAGACACAAUuc | 1.31 | 0.392 |
| DS18-0335 | 335 | aattgtgtctgctcgaggc | 734 | AAUUGUGUCUGCUCGAGGCau | 1134 | GCCUCGAGCAGACACAAUUcc | 1.24 | 0.306 |
| DS18-0336 | 336 | gaattgtgtctgctcgagg | 735 | GAAUUGUGUCUGCUCGAGGca | 1135 | CCUCGAGCAGACACAAUUCcc | 0.73 | −0.454 |
| DS18-0337 | 337 | ggaattgtgtctgctcgag | 736 | GGAAUUGUGUCUGCUCGAGgc | 1136 | CUCGAGCAGACACAAUUCCcc | 0.58 | −0.775 |
| DS18-0338 | 338 | gggaattgtgtctgctcga | 737 | GGGAAUUGUGUCUGCUCGAgg | 1137 | UCGAGCAGACACAAUUCCCcu | 1.01 | 0.010 |
| DS18-0339 | 339 | ggggaattgtgtctgctcg | 738 | GGGGAAUUGUGUCUGCUCGag | 1138 | CGAGCAGACACAAUUCCCCuu | 1.09 | 0.122 |
| DS18-0340 | 340 | aggggaattgtgtctgctc | 739 | AGGGGAAUUGUGUCUGCUCga | 1139 | GAGCAGACACAAUUCCCCUua | 0.83 | −0.271 |
| DS18-0341 | 341 | aaggggaattgtgtctgct | 740 | AAGGGGAAUUGUGUCUGCUcg | 1140 | AGCAGACACAAUUCCCCUUau | 0.77 | −0.386 |
| DS18-0342 | 342 | taaggggaattgtgtctgc | 741 | UAAGGGGAAUUGUGUCUGCuc | 1141 | GCAGACACAAUUCCCCUUAuu | 0.91 | −0.139 |
| DS18-0343 | 343 | ataaggggaattgtgtctg | 742 | AUAAGGGGAAUUGUGUCUGcu | 1142 | CAGACACAAUUCCCCUUAUuc | 1.48 | 0.567 |
| DS18-0344 | 344 | aataaggggaattgtgtct | 743 | AAUAAGGGGAAUUGUGUCUgc | 1143 | AGACACAAUUCCCCUUAUUca | 1.62 | 0.699 |
| DS18-0345 | 345 | gaataaggggaattgtgtc | 744 | GAAUAAGGGGAAUUGUGUCug | 1144 | GACACAAUUCCCCUUAUUCag | 1.34 | 0.419 |
| DS18-0346 | 346 | tgaataaggggaattgtgt | 745 | UGAAUAAGGGGAAUUGUGUcu | 1145 | ACACAAUUCCCCUUAUUCAga | 1.07 | 0.094 |
| DS18-0347 | 347 | ctgaataaggggaattgtg | 746 | CUGAAUAAGGGGAAUUGUGuc | 1146 | CACAAUUCCCCUUAUUCAGau | 1.44 | 0.526 |
| DS18-0348 | 348 | tctgaataaggggaattgt | 747 | UCUGAAUAAGGGGAAUUGUgu | 1147 | ACAAUUCCCCUUAUUCAGAuu | 1.16 | 0.211 |
| DS18-0349 | 349 | atctgaataaggggaattg | 748 | AUCUGAAUAAGGGGAAUUGug | 1148 | CAAUUCCCCUUAUUCAGAUua | 1.27 | 0.340 |
| DS18-0350 | 350 | gataatctgaataagggga | 749 | GAUAAUCUGAAUAAGGGGAau | 1149 | UCCCCUUAUUCAGAUUAUCcg | 1.18 | 0.234 |
| DS18-0351 | 351 | ggataatctgaataagggg | 750 | GGAUAAUCUGAAUAAGGGGaa | 1150 | CCCCUUAUUCAGAUUAUCCga | 1.08 | 0.104 |
| DS18-0352 | 352 | cggataatctgaataaggg | 751 | CGGAUAAUCUGAAUAAGGGga | 1151 | CCCUUAUUCAGAUUAUCCGaa | 1.13 | 0.180 |
| DS18-0353 | 353 | tcggataatctgaataagg | 752 | UCGGAUAAUCUGAAUAAGGgg | 1152 | CCUUAUUCAGAUUAUCCGAaa | 1.09 | 0.124 |
| DS18-0354 | 354 | cacattttcggataatctg | 753 | CACAUUUUCGGAUAAUCUGaa | 1153 | CAGAUUAUCCGAAAAUGUGag | 1.52 | 0.604 |
| DS18-0355 | 355 | ctcacattttcggataatc | 754 | CUCACAUUUUCGGAUAAUCug | 1154 | GAUUAUCCGAAAAUGUGAGuc | 1.46 | 0.544 |
| DS18-0356 | 356 | gactcacattttcggataa | 755 | GACUCACAUUUUCGGAUAAuc | 1155 | UUAUCCGAAAAUGUGAGUCac | 0.83 | −0.275 |
| DS18-0357 | 357 | tgactcacattttcggata | 756 | UGACUCACAUUUUCGGAUAau | 1156 | UAUCCGAAAAUGUGAGUCAcg | 1.17 | 0.233 |
| DS18-0358 | 358 | gtgactcacattttcggat | 757 | GUGACUCACAUUUUCGGAUaa | 1157 | AUCCGAAAAUGUGAGUCACgu | 1.13 | 0.177 |
| DS18-0359 | 359 | cgtgactcacattttcgga | 758 | CGUGACUCACAUUUUCGGAua | 1158 | UCCGAAAAUGUGAGUCACGuc | 0.87 | −0.203 |
| DS18-0360 | 360 | acgtgactcacattttcgg | 759 | ACGUGACUCACAUUUUCGGau | 1159 | CCGAAAAUGUGAGUCACGUcc | 0.63 | −0.664 |
| DS18-0361 | 361 | gacgtgactcacattttcg | 760 | GACGUGACUCACAUUUUCGga | 1160 | CGAAAAUGUGAGUCACGUCcc | 0.90 | −0.148 |
| DS18-0362 | 362 | ggacgtgactcacattttc | 761 | GGACGUGACUCACAUUUUCgg | 1161 | GAAAAUGUGAGUCACGUCCcc | 1.36 | 0.448 |
| DS18-0363 | 363 | gggacgtgactcacatttt | 762 | GGGACGUGACUCACAUUUUcg | 1162 | AAAAUGUGAGUCACGUCCCcu | 1.36 | 0.439 |
| DS18-0364 | 364 | ggggacgtgactcacattt | 763 | GGGGACGUGACUCACAUUUuc | 1163 | AAAUGUGAGUCACGUCCCCuu | 0.91 | −0.132 |
| DS18-0365 | 365 | aggggacgtgactcacatt | 764 | AGGGGACGUGACUCACAUUuu | 1164 | AAUGUGAGUCACGUCCCCUua | 0.73 | −0.463 |
| DS18-0366 | 366 | aaggggacgtgactcacat | 765 | AAGGGGACGUGACUCACAUuu | 1165 | AUGUGAGUCACGUCCCCUUag | 0.18 | −2.438 |
| DS18-0367 | 367 | taaggggacgtgactcaca | 766 | UAAGGGGACGUGACUCACAuu | 1166 | UGUGAGUCACGUCCCCUUAgg | 0.80 | −0.326 |
| DS18-0368 | 368 | ctaaggggacgtgactcac | 767 | CUAAGGGGACGUGACUCACau | 1167 | GUGAGUCACGUCCCCUUAGgc | 1.2 | 0.339 |
| DS18-0369 | 369 | cctaaggggacgtgactca | 768 | CCUAAGGGGACGUGACUCAca | 1168 | UGAGUCACGUCCCCUUAGGca | 0.85 | −0.226 |
| DS18-0370 | 370 | gcctaaggggacgtgactc | 769 | GCCUAAGGGGACGUGACUCac | 1169 | GAGUCACGUCCCCUUAGGCaa | 1.19 | 0.251 |
| DS18-0371 | 371 | tgcctaaggggacgtgact | 770 | UGCCUAAGGGGACGUGACUca | 1170 | AGUCACGUCCCCUUAGGCAaa | 1.18 | 0.234 |
| DS18-0372 | 372 | ttgcctaaggggacgtgac | 771 | UUGCCUAAGGGGACGUGACuc | 1171 | GUCACGUCCCCUUAGGCAAaa | 1.27 | 0.350 |
| DS18-0373 | 373 | tttgcctaaggggacgtga | 772 | UUUGCCUAAGGGGACGUGAcu | 1172 | UCACGUCCCCUUAGGCAAAac | 1.72 | 0.784 |
| DS18-0374 | 374 | ttttgcctaaggggacgtg | 773 | UUUUGCCUAAGGGGACGUGac | 1173 | CACGUCCCCUUAGGCAAAAca | 2.19 | 1.130 |
| DS18-0375 | 375 | gttttgcctaaggggacgt | 774 | GUUUUGCCUAAGGGGACGUga | 1174 | ACGUCCCCUUAGGCAAAACag | 1.39 | 0.473 |
| DS18-0376 | 376 | tgttttgcctaaggggacg | 775 | UGUUUUGCCUAAGGGGACGug | 1175 | CGUCCCCUUAGGCAAAACAgg | 1.86 | 0.892 |
| DS18-0377 | 377 | ctgttttgcctaaggggac | 776 | CUGUUUUGCCUAAGGGGACgu | 1176 | GUCCCCUUAGGCAAAACAGgu | 1.21 | 0.277 |
| DS18-0378 | 378 | cctgttttgcctaagggga | 777 | CCUGUUUUGCCUAAGGGGAcg | 1177 | UCCCCUUAGGCAAAACAGGuc | 1.13 | 0.177 |
| DS18-0379 | 379 | acctgttttgcctaagggg | 778 | ACCUGUUUUGCCUAAGGGGac | 1178 | CCCCUUAGGCAAAACAGGUcu | 1.32 | 0.398 |
| DS18-0380 | 380 | gacctgttttgcctaaggg | 779 | GACCUGUUUUGCCUAAGGGga | 1179 | CCCUUAGGCAAAACAGGUCuc | 1.21 | 0.274 |
| DS18-0381 | 381 | agacctgttttgcctaagg | 780 | AGACCUGUUUUGCCUAAGGgg | 1180 | CCUUAGGCAAAACAGGUCUcu | 0.90 | −0.151 |
| DS18-0382 | 382 | gagacctgttttgcctaag | 781 | GAGACCUGUUUUGCCUAAGgg | 1181 | CUUAGGCAAAACAGGUCUCua | 1.27 | 0.341 |
| DS18-0383 | 383 | agagacctgttttgcctaa | 782 | AGAGACCUGUUUUGCCUAAgg | 1182 | UUAGGCAAAACAGGUCUCUag | 1.22 | 0.292 |
| DS18-0384 | 384 | tagagacctgttttgccta | 783 | UAGAGACCUGUUUUGCCUAag | 1183 | UAGGCAAAACAGGUCUCUAgu | 1.19 | 0.248 |
| DS18-0385 | 385 | ctagagacctgttttgcct | 784 | CUAGAGACCUGUUUUGCCUaa | 1184 | AGGCAAAACAGGUCUCUAGuu | 0.79 | −0.331 |
| DS18-0386 | 386 | actagagacctgttttgcc | 785 | ACUAGAGACCUGUUUUGCCua | 1185 | GGCAAAACAGGUCUCUAGUua | 0.79 | −0.338 |
| DS18-0387 | 387 | aactagagacctgttttgc | 786 | AACUAGAGACCUGUUUUGCcu | 1186 | GCAAAACAGGUCUCUAGUUag | 0.75 | −0.411 |
| DS18-0388 | 388 | taactagagacctgttttg | 787 | UAACUAGAGACCUGUUUUGcc | 1187 | CAAAACAGGUCUCUAGUUAgg | 0.88 | −0.187 |
| DS18-0389 | 389 | ctaactagagacctgtttt | 788 | CUAACUAGAGACCUGUUUUgc | 1188 | AAAACAGGUCUCUAGUUAGgg | 1.10 | 0.133 |
| DS18-0390 | 390 | cctaactagagacctgttt | 789 | CCUAACUAGAGACCUGUUUug | 1189 | AAACAGGUCUCUAGUUAGGgu | 0.94 | −0.094 |
| DS18-0391 | 391 | ccctaactagagacctgtt | 790 | CCCUAACUAGAGACCUGUUuu | 1190 | AACAGGUCUCUAGUUAGGGug | 0.68 | −0.555 |
| DS18-0392 | 392 | accctaactagagacctgt | 791 | ACCCUAACUAGAGACCUGUuu | 1191 | ACAGGUCUCUAGUUAGGGUga | 0.84 | −0.247 |
| DS18-0393 | 393 | caccctaactagagacctg | 792 | CACCCUAACUAGAGACCUGuu | 1192 | CAGGUCUCUAGUUAGGGUGau | 0.87 | −0.201 |
| DS18-0394 | 394 | tcaccctaactagagacct | 793 | UCACCCUAACUAGAGACCUgu | 1193 | AGGUCUCUAGUUAGGGUGAua | 1.01 | 0.008 |
| DS18-0395 | 395 | atcaccctaactagagacc | 794 | AUCACCCUAACUAGAGACCug | 1194 | GGUCUCUAGUUAGGGUGAUau | 0.76 | −0.404 |
| DS18-0396 | 396 | tatcaccctaactagagac | 795 | UAUCACCCUAACUAGAGACcu | 1195 | GUCUCUAGUUAGGGUGAUAuu | 0.74 | −0.433 |
| DS18-0397 | 397 | atatcaccctaactagaga | 796 | AUAUCACCCUAACUAGAGAcc | 1196 | UCUCUAGUUAGGGUGAUAUuu | 0.99 | −0.018 |
| DS18-0398 | 398 | aatatcaccctaactagag | 797 | AAUAUCACCCUAACUAGAGac | 1197 | CUCUAGUUAGGGUGAUAUUuu | 1.09 | 0.125 |
| RAG18-si8 | 399 | actactgagtgacagtaga | 798 | GGUUGCUGAUGUCCUUAGATT | 1198 | UCUAAGGACAUCAGCAACCTT | 0.09 | −3.511 |

| | | | | | | | |
|---|---|---|---|---|---|---|---|
| dsCon2 | Non-specific duplex control | 799 | ACUACUGAGUGACAGUAGATT | 1199 | UCUACUGUCACUCAGUAGUTT | 1.04 | 0.057 |

Note:
Target sequence is identical to the identified sense sequence but the nucleotide ″U″ is converted to ″T″ excluding 2-nt nucleotides natural overhang selected from or complementary to the corresponding nucleotides on the DNA target. DS18-si8 is a silencing dsRNA control.

What is claimed is:

1. A small activating RNA (saRNA) comprising an oligonucleotide sequence having a length ranging from 16 to 35 consecutive nucleotides, wherein the oligonucleotide sequence comprises a continuous nucleotide sequence having at least 75%, at least 80%, at least 85%, or at least 90% homology or complementarity to an equal length portion of SEQ ID NO:1200, wherein the saRNA upregulates the expression of UTRN gene by at least 10% as compared to baseline expression of the UTRN gene.

2. The saRNA of claim 1, wherein the equal length portion of SEQ ID NO:1200 is located in the region −636 to −496 (SEQ ID NO:1207), region −351 to −294 (SEQ ID NO:1208), region −236 to −187 (SEQ ID NO:1209), or region −101 to −65 (SEQ ID NO:1210) upstream of the transcription start site of UTRN gene.

3. The saRNA of any one of claims 1-2, wherein the saRNA (1) has a GC content between 35% and 70%; (2) with less than 5 consecutive identical nucleotides; (3) with 3 or less dinucleotide repeats; and (4) with 3 or less trinucleotide repeats.

4. The saRNA of any one of claims 1-3, wherein the saRNA comprises a sense strand and an antisense strand.

5. The saRNA of any one of claims 1-4, wherein the oligonucleotide sequence is the sense strand or the antisense strand of the saRNA.

6. The saRNA of any one of claims 1-5, wherein the sense strand and the antisense strand each comprise complementary regions, wherein the complementary regions of the sense strand and the antisense strand form a double-stranded nucleic acid structure.

7. The saRNA of any one of claims 4-6, wherein the sense strand and the antisense strand have a complementarity of at least 90%.

8. The saRNA of claim 4, wherein the sense strand and the antisense strand are located on two different nucleic acid strands.

9. The saRNA of claim 4, wherein the sense strand and the antisense strand are located on a contiguous nucleic acid strand, optionally a hairpin single-stranded nucleic acid molecule, wherein the complementary regions of the sense strand and the antisense strand form a double-stranded nucleic acid structure.

10. The saRNA of claim 4, wherein at least one of the sense strand and the antisense strand comprises a 3′ overhang ranging from 0 to 6 nucleotides in length.

11. The saRNA of claim 10, wherein the sense strand and the antisense strand comprise a 3′ overhang of ranging from 2 to 3 nucleotides in length.

12. The saRNA of claim 10, wherein at least one of the nucleotides of the overhang is nucleotides selected from or complementary to the corresponding nucleotides on the UTRN gene.

13. The saRNA of any of claims 4-12, wherein the sense strand and the antisense strand independently comprise a length of about 16 to about 35, about 17 to about 30, about 18 to about 25, or about 19 to about 22 consecutive nucleotides.

14. The saRNA of any one of claims 4-12, wherein the sense strand has at least 75% sequence homology to a nucleotide sequence selected from SEQ ID NOs: 400-797, and the antisense strand has at least 75% sequence homology to a nucleotide sequence selected from SEQ ID NOs: 800-1197.

15. The saRNA of claim 14, wherein the sense strand comprises a nucleotide sequence selected from SEQ ID NOs: 400-797, and the antisense strand comprises a nucleotide sequence selected from SEQ ID NOs: 800-1197.

16. The saRNA of claim 1, wherein the oligonucleotide sequence has at least 75% sequence homology or complementarity to a nucleotide sequence selected from SEQ ID NOs: 1-398.

17. The saRNA of claim 4, wherein the sense strand has at least 75% sequence homology to a nucleotide sequence selected from SEQ ID NOs: 1-398.

18. The saRNA of claim 4, wherein the antisense strand has at least 75% sequence complementarity to a nucleotide sequence selected from SEQ ID NOs: 1-398.

19. The saRNA of any of claims 1-18, wherein at least one nucleotide of the saRNA is a chemically modified nucleotide.

20. The saRNA of claim 19, wherein at least one nucleotide of the antisense and/or sense strand of the saRNA is chemically modified.

21. The saRNA of claim 19, wherein the chemically modified nucleotide is a nucleotide with at least one the following modifications:
a) modification of a phosphodiester bond connecting nucleotides in the nucleotide sequence of the saRNA;
b) modification of 2′-OH of a ribose in the nucleotide sequence of the saRNA; and
c) modification of a base in the nucleotide sequence of the saRNA.

22. The saRNA of claim 19, wherein at least one nucleotide of the saRNA is a locked nucleic acid, an abasic nucleotide, a 2′-amino-modified nucleotide, a 2′-alkyl-modified nucleotide, a morpholino nucleotide, a phosphoramidate, or a non-natural base comprising nucleotide.

23. The saRNA of claim 19, wherein the chemical modification of the at least one chemically modified nucleotide is an addition of a (E)-vinylphosphonate moiety at the 5′ end of the sense strand or the antisense strand.

24. The saRNA of any one of claims 1-23 wherein the sense strand or the antisense strand of the saRNA is conjugated to one or more conjugation moieties selected from a lipid, a fatty acid, a fluorophore, a ligand, a saccharide, a peptide, and an antibody.

25. The saRNA of claim 24, wherein the sense strand or the antisense strand of the saRNA is conjugated to one or more conjugation moieties selected from a cell-penetrating peptide, polyethylene glycol, an alkaloid, a tryptamine, a benzimidazole, a quinolone, an amino acid, a cholesterol, glucose, and N-acetylgalactosamine, and any combinations thereof.

26. An oligonucleotide modulator comprising one or more saRNA according to any one of claims 1-25.

27. The oligonucleotide modulator of claim 26, further comprising one or more moieties or components conjugated, combined or bonded with said saRNA(s).

28. The oligonucleotide modulator of claim 27, wherein the sense strand and/or the antisense strand of the saRNA is conjugated to one or more conjugation moieties selected from the group consisting of a lipid, a fatty acid, a fluorophore, a ligand, a saccharide, a peptide, and an antibody.

29. The oligonucleotide modulator of claim 27, wherein the conjugation moiety is each independently selected from a lipid, a cell-penetrating peptide, a polyethylene glycol, an alkaloid, a tryptamine, a benzimidazole, a quinolone, an amino acid, a cholesterol, a glucose, a N-acetylgalactosamine, and any combinations thereof.

30. The oligonucleotide modulator of claim 26, wherein the oligonucleotide modulator further comprises a saRNA conjugated to or combined with one or more of other active moieties for UTRN associated diseases or disorder treatment, wherein the one or more of other active moieties are each independently selected from a saRNA, a single-stranded oligonucleotide, a chemical moiety, a polypeptide and an antibody.

31. An isolated polynucleotide, wherein the isolated polynucleotide comprises the continuous nucleotide sequence of claim 1.

32. The isolated polynucleotide of claim 31, wherein the isolated polynucleotide is a nucleic acid sequence selected from SEQ ID NOs:1-398.

33. An isolated oligonucleotide complex comprising the antisense strand of the saRNA of any of claims 1-25 and the isolated polynucleotide of any of claims 31-32.

34. The isolated oligonucleotide complex of claim 33, wherein the isolated oligonucleotide complex activates the expression of UTRN gene by at least 10% as compared to baseline expression of the UTRN gene.

35. An isolated nucleic acid sequence upstream of the transcription start site of UTRN gene, wherein the isolated nucleic acid sequence is selected from SEQ ID NOs:1207-1210.

36. The isolated nucleic acid sequence of claim 35, wherein the isolated nucleic acid sequence comprises the isolated polynucleotide of any one of claims 31-32.

37. The isolated nucleic acid sequence of claim 35, wherein at least 25% of designed saRNA targeting the isolated nucleic acid sequence can activate the expression of UTRN gene by at least 10%, wherein the designed saRNA (1) having a GC content between 35% and 70%; (2) with less than 5 consecutive identical nucleotides; (3) with 3 or less dinucleotide repeats; and (4) with 3 or less trinucleotide repeats.

38. An isolated nucleic acid complex comprising the antisense strand of the saRNA of any of claims 1-25 and the sense strand of the isolated nucleic acid sequence of any of claims 35-37.

39. The isolated nucleic acid complex of claim 38, wherein the isolated nucleic acid complex activates the expression of UTRN gene by at least 10% as compared to baseline expression of the UTRN gene.

40. An isolated polynucleotide encoding the saRNA of any one of claims 1-25.

41. The isolated polynucleotide of claim 40, wherein the isolated polynucleotide is a DNA.

42. A vector comprising the isolated polynucleotide of any one of claims 40-41.

43. A host cell comprising the saRNA of any one of claims 1-25, the isolated polynucleotide of any one of claims 40-41, or the vector of claim 42.

44. A composition comprising the saRNA of any one of claims 1-25, or the isolated polynucleotide of claim 40 or claim 41 and optionally, a pharmaceutically acceptable carrier.

45. The composition of claim 44, wherein the pharmaceutically acceptable carrier is selected from the group consisting of an aqueous carrier, a liposome, a high-molecular polymer, a polypeptide and an antibody.

46. The composition of claim 44 or 45, wherein the composition comprises 0.001-200 nM of the saRNA.

47. The composition of claim 46, wherein the composition comprises 1-200 nM of the saRNA.

48. An saRNA comprising an oligonucleotide sequence with a length ranging from 16 to 35 continuous nucleotides for activating/upregulating UTRN gene expression in a cell, wherein the oligonucleotide sequence has at least 75%, or at least 80%, or at least 85%, or at least 90% sequence homology or complementary to an equal length portion of SEQ ID NO:1200, wherein the saRNA activates the expression of UTRN gene by at least 10% as compared to its baseline expression.

49. The saRNA of claim 48, wherein the equal length region of SEQ ID NO:1200 is located in the region −636 to −496 (SEQ ID NO:1207), region −351 to −294 (SEQ ID NO:1208), region −236 to −187 (SEQ ID NO:1209), or region −101 to −65 (SEQ ID NO:1210) upstream of the transcription start site of UTRN gene.

50. The saRNA of claim 49, wherein the saRNA comprises a sense strand and an antisense strand, wherein the sense strand comprises a nucleotide sequence selected from SEQ ID NOs: 400-797, and the antisense strand comprises or is a nucleotide sequence selected from SEQ ID NOs: 800-1197.

51. A product for activating/up-regulating UTRN gene expression in a cell, wherein the product activates the expression of UTRN gene by at least 10% as compared to baseline expression of the UTRN gene, and wherein the product comprises an active substance selected from one or more of the saRNA of any one of claims 1-25, the isolated polynucleotide of any one of claims 40-41, the vector of claim 42, or the composition of any one of claims 44-47.

52. The product for activating/up-regulating UTRN gene expression in a cell, wherein the active substance is introduced directly into the cell; and/or
wherein the cell is in vitro, ex vivo or in vivo; and/or
wherein the cell is a mammalian cell.

53. The product of claim 52, wherein the active substance is introduced directly into the cell by:
1) composing the active substance with a physiologically acceptable or pharmaceutically acceptable carrier, such as one or more selected from the group consisting of an aqueous carrier, a liposome, a high-molecular polymer, a polypeptide and an antibody, and/or
2) conjugating the active substance to one or more conjugation moieties, such as one or more selected from a lipid, a cell-penetrating peptide, a polyethylene glycol, an alkaloid, a tryptamine, a benzimidazole, a quinolone, an amino acid, a cholesterol, a glucose, and a N-acetylgalactosamine, and any combinations thereof (for example two conjugation moieties wherein one is a lipid and the other is a N-acetylgalactosamine).

54. The product of claim 53, wherein the conjugation moiety is independently derived from a fluorophore, a ligand, a saccharide, a peptide, and an antibody.

55. The product for activating/up-regulating UTRN gene expression in a cell, wherein the cell is from a patient suffering from or in risk of having a disease or condition induced by insufficient expression of the UTRN protein, a UTRN gene mutation, and/or low functional UTRN levels, wherein the active substance is administered in a sufficient amount to prevent or treat the disease or condition, such as Duchenne muscular dystrophy (DMD) or Becker muscular dystrophy (BMD).

56. A method for preventing or treating a disease or condition induced by insufficient expression of dystrophin, a dystrophin gene mutation, and/or low functional dystrophin levels in an individual comprising: administering an effective amount of the saRNA of any one of claims 1-25, the isolated polynucleotide of any one of claims 40-41, the vector of claim 42, or the composition of any one of claims 44-46 to the individual.

57. The method of claim 56, wherein the disease or condition is a dystrophin deficiency disorder (DDD).

58. The method of claim 56, wherein the disease or condition is a Duchenne muscular dystrophy (DMD) or Becker muscular dystrophy (BMD).

59. The method of claim 56, wherein the individual is a mammal, optionally wherein the individual is a human.

60. The method of claim 56, wherein the individual suffers from a symptom induced by insufficient expression of dystrophin, a dystrophin gene mutation, and/or low functional dystrophin levels in an individual.

61. The method of claim 56, wherein the saRNA of any one of claims 1-25, the isolated polynucleotide of any one of claims 40-41, the vector of claim 42, or the composition of any one of claims 44-47 is administrated to an individual by an administration pathway selected from one or more of: parenteral infusions, oral administration, intranasal administration, inhaled administration, vaginal administration, and rectal administration.

62. The method of claim 61, wherein the administration pathway is selected from one or more of intrathecal, intramuscular, intravenous, intraarterial, intraperitoneal, intravesical, intracerebroventricular, intravitreal and subcutaneous administrations.

63. The method of claim 56, wherein the method activates/up-regulates expression of the UTRN gene mRNA in the individual by at least 10% as compared to baseline expression of the UTRN gene.

64. The method of claim 56, wherein the method increases a level of utrophin in the individual by at least 10% as compared to baseline expression of the UTRN gene.

65. A method for detecting dystrophin, utrophin or dystrophin related protein (e.g., dystroglycan) in the host cell of claim 43.

66. A kit for performing the method of claim 56, comprising a) saRNA of claims 1-25.

67. The kit of claim 66, wherein the kit comprises b) instructions for use, and c) optionally, means for administering the saRNA of claims 1-25 to an individual.

68. A kit comprising the saRNA of any one of claims 1-25, the isolated polynucleotide of any one of claims 40-41, the vector of claim 42, or the composition of any one of claims 44-47 in a labeled package and the label on package indicates that the saRNA, the isolated polynucleotide, the vector or the composition can be used in preventing or treating a disease or condition induced by insufficient expression of dystrophin, or against Duchenne muscular dystrophy (DMD) or Becker muscular dystrophy (BMD).

69. A kit for detecting dystrophin, utrophin or dystrophin related protein (e.g., dystroglycan) in the host cell of claim 43.

70. Use of the saRNA of any one of claims 1-25, the isolated polynucleotide of any one of claims 40-41, the vector of claim 42, or the composition of any one of claims 44-47 in preparing a medicament for preventing or treating a disease or condition induced by insufficient expression of dystrophin, a dystrophin gene mutation, and/or low functional dystrophin levels in an individual.

71. The use of claim 70, wherein the disease or condition is a Duchenne muscular dystrophy (DMD) or Becker muscular dystrophy (BMD).

72. The use of claim 70, wherein the individual is a mammal, optionally wherein the mammal is a human.

73. Use of the saRNA of any one of claims 1-25, the isolated polynucleotide of any one of claims 40-41, the vector of claim 42, or the composition of any one of claims 44-47 in preparing a preparation for activating/up-regulating expression of UTRN gene in a cell.

74. The use of claim 73, wherein the saRNA of any one of claims 1-25, or the isolated polynucleotide of any one of claims 40-41, the vector of claim 42, or the composition of any one of claims 44-47 is directly introduced into the cell.

75. The use of claim 74, wherein the saRNA is produced in the cell after a nucleotide sequence encoding the saRNA is introduced into the cell.

76. The use of any of claims 73-75, wherein the cell is a mammalian cell, optionally wherein the mammalian cell is a human cell.

77. The use of claim 76, wherein the cell is in a human body.

78. The use of claim 77, wherein the human body is a subject suffering from a symptom induced by the insufficient expression of dystrophin, a dystrophin gene mutation, and/or low functional dystrophin levels in an individual, wherein the saRNA, the isolated polynucleotide or the composition is administered in a sufficient amount to treat the symptom.

79. The use of claim 78, wherein the symptom induced by insufficient expression of dystrophin is a Duchenne muscular dystrophy (DMD) or Becker muscular dystrophy (BMD).

80. A method for activating/up-regulating expression of UTRN gene in a cell comprising: administering an effective amount of the saRNA of any one of claims 1-25, the isolated polynucleotide of any one of claims 40-41, the vector of claim 42, or the composition of any one of claims 44-47 to the cell.

81. The method of claim 80, wherein the saRNA of any one of claims 1-25, the isolated polynucleotide of any one of claims 40-41, the vector of claim 42, or the composition of any one of claims 44-47 is introduced directly into the cell.

82. The method of claim 81, wherein the method, for introducing directly into the cell, comprises:
1) composing the saRNA of any one of claims 1-25, the isolated polynucleotide of any one of claims 40-41, the vector of claim 42, or the saRNA in the composition of any one of claims 44-47 with a pharmaceutically acceptable carrier selected from the group consisting of an aqueous carrier, a liposome, a high-molecular polymer, a polypeptide and an antibody, and
2) conjugating the saRNA of any one of claims 1-25, the isolated polynucleotide of any one of claims 40-41, the vector of claim 42, or the saRNA in the composition of any one of claims 44-47 to one or more conjugation moieties selected from a cell-penetrating peptide, polyethylene glycol, an alkaloid, a tryptamine, a benzimidazole, a quinolone, an amino acid, a cholesterol, glucose, and N-acetylgalactosamine.

83. The method of any of claims 80-82, wherein the cell is a mammalian cell, for example a cell from a human body.

84. The method of claim 83, wherein the human body is a subject suffering from a symptom induced by insufficient expression of dystrophin, a dystrophin gene mutation, and/or low functional dystrophin levels in an individual, wherein the saRNA, the isolated polynucleotide or the composition is administered in a sufficient amount to treat the symptom.

85. The method of claim 84, wherein the symptom caused by insufficient expression of dystrophin is Duchenne muscular dystrophy (DMD) or Becker muscular dystrophy (BMD).

86. A method for increasing a level of utrophin in a cell, comprising introducing an effective amount of the saRNA of any one of claims 1-25, the isolated polynucleotide of any one of claims 40-41, the vector of claim 42, or the saRNA in the composition of any one of claims 44-47 into the cell, wherein the saRNA, the isolated polynucleotide or the composition activates expression of UTRN gene by at least 10% as compared to baseline expression of the UTRN gene.


SEQUENCE LISTING (1241 sequences)

SEQ ID NO: 1 - DNA, 19 bp, synthetic construct
  misc_feature 1..19: note = Synthetic oligonucleotide
  source 1..19: mol_type = other DNA
ggttcgtgtctgcttctcc

SEQ ID NO: 2 - DNA, 19 bp, synthetic construct
  misc_feature 1..19: note = Synthetic oligonucleotide
  source 1..19: mol_type = other DNA
cggttcgtgtctgcttctc

SEQ ID NO: 3 - DNA, 19 bp, synthetic construct
  misc_feature 1..19: note = Synthetic oligonucleotide
  source 1..19: mol_type = other DNA
tcggttcgtgtctgcttct

SEQ ID NO: 4 - DNA, 19 bp, synthetic construct
  misc_feature 1..19: note = Synthetic oligonucleotide
  source 1..19: mol_type = other DNA
ttcggttcgtgtctgcttc

SEQ ID NO: 5 - DNA, 19 bp, synthetic construct
  misc_feature 1..19: note = Synthetic oligonucleotide
  source 1..19: mol_type = other DNA
tttcggttcgtgtctgctt

SEQ ID NO: 6 - DNA, 19 bp, synthetic construct
  misc_feature 1..19: note = Synthetic oligonucleotide
  source 1..19: mol_type = other DNA
atttcggttcgtgtctgct

SEQ ID NO: 7 - DNA, 19 bp, synthetic construct
  misc_feature 1..19: note = Synthetic oligonucleotide
  source 1..19: mol_type = other DNA
aatttcggttcgtgtctgc

SEQ ID NO: 8 - DNA, 19 bp, synthetic construct
  misc_feature 1..19: note = Synthetic oligonucleotide
  source 1..19: mol_type = other DNA
aaatttcggttcgtgtctg

SEQ ID NO: 9 - DNA, 19 bp, synthetic construct
  misc_feature 1..19: note = Synthetic oligonucleotide
  source 1..19: mol_type = other DNA
aaaatttcggttcgtgtct

SEQ ID NO: 10 - DNA, 19 bp, synthetic construct
  misc_feature 1..19: note = Synthetic oligonucleotide
  source 1..19: mol_type = other DNA
taaaatttcggttcgtgtc

SEQ ID NO: 11 - DNA, 19 bp, synthetic construct
  misc_feature 1..19: note = Synthetic oligonucleotide
  source 1..19: mol_type = other DNA
ggtgtcaatttctctttct

SEQ ID NO: 12 - DNA, 19 bp, synthetic construct
  misc_feature 1..19: note = Synthetic oligonucleotide
  source 1..19: mol_type = other DNA
gggtgtcaatttctctttc

SEQ ID NO: 13 - DNA, 19 bp, synthetic construct
  misc_feature 1..19: note = Synthetic oligonucleotide
  source 1..19: mol_type = other DNA
cgggtgtcaatttctcttt

SEQ ID NO: 14 - DNA, 19 bp, synthetic construct
  misc_feature 1..19: note = Synthetic oligonucleotide
  source 1..19: mol_type = other DNA
tcgggtgtcaatttctctt

SEQ ID NO: 15 - DNA, 19 bp, synthetic construct
  misc_feature 1..19: note = Synthetic oligonucleotide
  source 1..19: mol_type = other DNA
gtcgggtgtcaatttctct

SEQ ID NO: 16 - DNA, 19 bp, synthetic construct
  misc_feature 1..19: note = Synthetic oligonucleotide
  source 1..19: mol_type = other DNA
agtcgggtgtcaatttctc

SEQ ID NO: 17 - DNA, 19 bp, synthetic construct
  misc_feature 1..19: note = Synthetic oligonucleotide
  source 1..19: mol_type = other DNA
cagtcgggtgtcaatttct

SEQ ID NO: 18 - DNA, 19 bp, synthetic construct
  misc_feature 1..19: note = Synthetic oligonucleotide
  source 1..19: mol_type = other DNA
tcagtcgggtgtcaatttc

SEQ ID NO: 19 - DNA, 19 bp, synthetic construct
  misc_feature 1..19: note = Synthetic oligonucleotide
  source 1..19: mol_type = other DNA
ttcagtcgggtgtcaattt

SEQ ID NO: 20 - DNA, 19 bp, synthetic construct
  misc_feature 1..19: note = Synthetic oligonucleotide
  source 1..19: mol_type = other DNA
tttcagtcgggtgtcaatt

SEQ ID NO: 21 - DNA, 19 bp, synthetic construct
  misc_feature 1..19: note = Synthetic oligonucleotide
  source 1..19: mol_type = other DNA
ttttcagtcgggtgtcaat

SEQ ID NO: 22 - DNA, 19 bp, synthetic construct
  misc_feature 1..19: note = Synthetic oligonucleotide
  source 1..19: mol_type = other DNA
gttttcagtcgggtgtcaa

SEQ ID NO: 23 - DNA, 19 bp, synthetic construct
  misc_feature 1..19: note = Synthetic oligonucleotide
  source 1..19: mol_type = other DNA
cgttttcagtcgggtgtca

SEQ ID NO: 24 - DNA, 19 bp, synthetic construct
  misc_feature 1..19: note = Synthetic oligonucleotide
  source 1..19: mol_type = other DNA
gcgttttcagtcgggtgtc

SEQ ID NO: 25 - DNA, 19 bp, synthetic construct
  misc_feature 1..19: note = Synthetic oligonucleotide
  source 1..19: mol_type = other DNA
ggcgttttcagtcgggtgt

SEQ ID NO: 26 - DNA, 19 bp, synthetic construct
  misc_feature 1..19: note = Synthetic oligonucleotide
  source 1..19: mol_type = other DNA
gggcgttttcagtcgggtg

SEQ ID NO: 27 - DNA, 19 bp, synthetic construct
  misc_feature 1..19: note = Synthetic oligonucleotide
  source 1..19: mol_type = other DNA
ggggcgttttcagtcgggt

SEQ ID NO: 28 - DNA, 19 bp, synthetic construct
  misc_feature 1..19: note = Synthetic oligonucleotide
  source 1..19: mol_type = other DNA
aggggcgttttcagtcggg

SEQ ID NO: 29 - DNA, 19 bp, synthetic construct
  misc_feature 1..19: note = Synthetic oligonucleotide
  source 1..19: mol_type = other DNA
aaggggcgttttcagtcgg

SEQ ID NO: 30 - DNA, 19 bp, synthetic construct
  misc_feature 1..19: note = Synthetic oligonucleotide
  source 1..19: mol_type = other DNA
taaggggcgttttcagtcg

SEQ ID NO: 31 - DNA, 19 bp, synthetic construct
  misc_feature 1..19: note = Synthetic oligonucleotide
  source 1..19: mol_type = other DNA
gtaaggggcgttttcagtc

SEQ ID NO: 32 - DNA, 19 bp, synthetic construct
  misc_feature 1..19: note = Synthetic oligonucleotide
  source 1..19: mol_type = other DNA
agtaaggggcgttttcagt

SEQ ID NO: 33 - DNA, 19 bp, synthetic construct
  misc_feature 1..19: note = Synthetic oligonucleotide
  source 1..19: mol_type = other DNA
aagtaaggggcgttttcag

SEQ ID NO: 34 - DNA, 19 bp, synthetic construct
  misc_feature 1..19: note = Synthetic oligonucleotide
  source 1..19: mol_type = other DNA
caagtaaggggcgttttca

SEQ ID NO: 35 - DNA, 19 bp, synthetic construct
  misc_feature 1..19: note = Synthetic oligonucleotide
  source 1..19: mol_type = other DNA
acaagtaaggggcgttttc

SEQ ID NO: 36 - DNA, 19 bp, synthetic construct
  misc_feature 1..19: note = Synthetic oligonucleotide
  source 1..19: mol_type = other DNA
cacaagtaaggggcgtttt

SEQ ID NO: 37 - DNA, 19 bp, synthetic construct
  misc_feature 1..19: note = Synthetic oligonucleotide
  source 1..19: mol_type = other DNA
gcacaagtaaggggcgttt

SEQ ID NO: 38 - DNA, 19 bp, synthetic construct
  misc_feature 1..19: note = Synthetic oligonucleotide
  source 1..19: mol_type = other DNA
tgcacaagtaaggggcgtt

SEQ ID NO: 39 - DNA, 19 bp, synthetic construct
  misc_feature 1..19: note = Synthetic oligonucleotide
  source 1..19: mol_type = other DNA
ttgcacaagtaaggggcgt

SEQ ID NO: 40 - DNA, 19 bp, synthetic construct
  misc_feature 1..19: note = Synthetic oligonucleotide
  source 1..19: mol_type = other DNA
attgcacaagtaaggggcg

SEQ ID NO: 41 - DNA, 19 bp, synthetic construct
  misc_feature 1..19: note = Synthetic oligonucleotide
  source 1..19: mol_type = other DNA
gattgcacaagtaaggggc

SEQ ID NO: 42 - DNA, 19 bp, synthetic construct
  misc_feature 1..19: note = Synthetic oligonucleotide
  source 1..19: mol_type = other DNA
agattgcacaagtaagggg

SEQ ID NO: 43 - DNA, 19 bp, synthetic construct
  misc_feature 1..19: note = Synthetic oligonucleotide
  source 1..19: mol_type = other DNA
aagattgcacaagtaaggg

SEQ ID NO: 44 - DNA, 19 bp, synthetic construct
  misc_feature 1..19: note = Synthetic oligonucleotide
  source 1..19: mol_type = other DNA
gaagattgcacaagtaagg

SEQ ID NO: 45 - DNA, 19 bp, synthetic construct
  misc_feature 1..19: note = Synthetic oligonucleotide
  source 1..19: mol_type = other DNA
ggaagattgcacaagtaag

SEQ ID NO: 46 - DNA, 19 bp, synthetic construct
  misc_feature 1..19: note = Synthetic oligonucleotide
  source 1..19: mol_type = other DNA
aggaagattgcacaagtaa

SEQ ID NO: 47 - DNA, 19 bp, synthetic construct
  misc_feature 1..19: note = Synthetic oligonucleotide
  source 1..19: mol_type = other DNA
caggaagattgcacaagta

SEQ ID NO: 48 - DNA, 19 bp, synthetic construct
  misc_feature 1..19: note = Synthetic oligonucleotide
  source 1..19: mol_type = other DNA
gcaggaagattgcacaagt

SEQ ID NO: 49 - DNA, 19 bp, synthetic construct
  misc_feature 1..19: note = Synthetic oligonucleotide
  source 1..19: mol_type = other DNA
ggcaggaagattgcacaag

SEQ ID NO: 50 - DNA, 19 bp, synthetic construct
  misc_feature 1..19: note = Synthetic oligonucleotide
  source 1..19: mol_type = other DNA
aggcaggaagattgcacaa

SEQ ID NO: 51 - DNA, 19 bp, synthetic construct
  misc_feature 1..19: note = Synthetic oligonucleotide
  source 1..19: mol_type = other DNA
caggcaggaagattgcaca

SEQ ID NO: 52 - DNA, 19 bp, synthetic construct
  misc_feature 1..19: note = Synthetic oligonucleotide
  source 1..19: mol_type = other DNA
ccaggcaggaagattgcac

SEQ ID NO: 53 - DNA, 19 bp, synthetic construct
  misc_feature 1..19: note = Synthetic oligonucleotide
  source 1..19: mol_type = other DNA
accaggcaggaagattgca

SEQ ID NO: 54 - DNA, 19 bp, synthetic construct
  misc_feature 1..19: note = Synthetic oligonucleotide
  source 1..19: mol_type = other DNA
taccaggcaggaagattgc

SEQ ID NO: 55 - DNA, 19 bp, synthetic construct
  misc_feature 1..19: note = Synthetic oligonucleotide
  source 1..19: mol_type = other DNA
ctaccaggcaggaagattg

SEQ ID NO: 56 - DNA, 19 bp, synthetic construct
  misc_feature 1..19: note = Synthetic oligonucleotide
  source 1..19: mol_type = other DNA
actaccaggcaggaagatt

SEQ ID NO: 57 - DNA, 19 bp, synthetic construct
  misc_feature 1..19: note = Synthetic oligonucleotide
  source 1..19: mol_type = other DNA
aactaccaggcaggaagat

SEQ ID NO: 58 - DNA, 19 bp, synthetic construct
  misc_feature 1..19: note = Synthetic oligonucleotide
  source 1..19: mol_type = other DNA
gaactaccaggcaggaaga

SEQ ID NO: 59 - DNA, 19 bp, synthetic construct
  misc_feature 1..19: note = Synthetic oligonucleotide
  source 1..19: mol_type = other DNA
ggaactaccaggcaggaag

SEQ ID NO: 60 - DNA, 19 bp, synthetic construct
  misc_feature 1..19: note = Synthetic oligonucleotide
  source 1..19: mol_type = other DNA
cggaactaccaggcaggaa

SEQ ID NO: 61 - DNA, 19 bp, synthetic construct
  misc_feature 1..19: note = Synthetic oligonucleotide
  source 1..19: mol_type = other DNA
gcggaactaccaggcagga

SEQ ID NO: 62 - DNA, 19 bp, synthetic construct
  misc_feature 1..19: note = Synthetic oligonucleotide
  source 1..19: mol_type = other DNA
ggcggaactaccaggcagg

SEQ ID NO: 63 - DNA, 19 bp, synthetic construct
  misc_feature 1..19: note = Synthetic oligonucleotide
  source 1..19: mol_type = other DNA
tggcggaactaccaggcag

SEQ ID NO: 64 - DNA, 19 bp, synthetic construct
  misc_feature 1..19: note = Synthetic oligonucleotide
  source 1..19: mol_type = other DNA
ttggcggaactaccaggca

SEQ ID NO: 65 - DNA, 19 bp, synthetic construct
  misc_feature 1..19: note = Synthetic oligonucleotide
  source 1..19: mol_type = other DNA
tttggcggaactaccaggc

SEQ ID NO: 66 - DNA, 19 bp, synthetic construct
  misc_feature 1..19: note = Synthetic oligonucleotide
  source 1..19: mol_type = other DNA
ttttggcggaactaccagg

SEQ ID NO: 67 - DNA, 19 bp, synthetic construct
  misc_feature 1..19: note = Synthetic oligonucleotide
  source 1..19: mol_type = other DNA
cgctttgactgttttgttt

SEQ ID NO: 68 - DNA, 19 bp, synthetic construct
  misc_feature 1..19: note = Synthetic oligonucleotide
  source 1..19: mol_type = other DNA
gcgctttgactgttttgtt

SEQ ID NO: 69 - DNA, 19 bp, synthetic construct
  misc_feature 1..19: note = Synthetic oligonucleotide
  source 1..19: mol_type = other DNA
tgcgctttgactgttttgt

SEQ ID NO: 70 - DNA, 19 bp, synthetic construct
  misc_feature 1..19: note = Synthetic oligonucleotide
  source 1..19: mol_type = other DNA
ttgcgctttgactgttttg

SEQ ID NO: 71 - DNA, 19 bp, synthetic construct
  misc_feature 1..19: note = Synthetic oligonucleotide
  source 1..19: mol_type = other DNA
tttgcgctttgactgtttt

SEQ ID NO: 72 - DNA, 19 bp, synthetic construct
  misc_feature 1..19: note = Synthetic oligonucleotide
  source 1..19: mol_type = other DNA
gtttgcgctttgactgttt

SEQ ID NO: 73 - DNA, 19 bp, synthetic construct
  misc_feature 1..19: note = Synthetic oligonucleotide
  source 1..19: mol_type = other DNA
ggtttgcgctttgactgtt

SEQ ID NO: 74 - DNA, 19 bp, synthetic construct
  misc_feature 1..19: note = Synthetic oligonucleotide
  source 1..19: mol_type = other DNA
aggtttgcgctttgactgt

SEQ ID NO: 75 - DNA, 19 bp, synthetic construct
  misc_feature 1..19: note = Synthetic oligonucleotide
  source 1..19: mol_type = other DNA
caggtttgcgctttgactg

SEQ ID NO: 76 - DNA, 19 bp, synthetic construct
  misc_feature 1..19: note = Synthetic oligonucleotide
  source 1..19: mol_type = other DNA
ccaggtttgcgctttgact

SEQ ID NO: 77 - DNA, 19 bp, synthetic construct
  misc_feature 1..19: note = Synthetic oligonucleotide
  source 1..19: mol_type = other DNA
tccaggtttgcgctttgac

SEQ ID NO: 78 - DNA, 19 bp, synthetic construct
  misc_feature 1..19: note = Synthetic oligonucleotide
  source 1..19: mol_type = other DNA
ttccaggtttgcgctttga

SEQ ID NO: 79 - DNA, 19 bp, synthetic construct
  misc_feature 1..19: note = Synthetic oligonucleotide
  source 1..19: mol_type = other DNA
cttccaggtttgcgctttg

SEQ ID NO: 80 - DNA, 19 bp, synthetic construct
  misc_feature 1..19: note = Synthetic oligonucleotide
  source 1..19: mol_type = other DNA
ccttccaggtttgcgcttt

SEQ ID NO: 81 - DNA, 19 bp, synthetic construct
  misc_feature 1..19: note = Synthetic oligonucleotide
  source 1..19: mol_type = other DNA
cccttccaggtttgcgctt

SEQ ID NO: 82 - DNA, 19 bp, synthetic construct
  misc_feature 1..19: note = Synthetic oligonucleotide
  source 1..19: mol_type = other DNA
ccccttccaggtttgcgct

SEQ ID NO: 83 - DNA, 19 bp, synthetic construct
  misc_feature 1..19: note = Synthetic oligonucleotide
  source 1..19: mol_type = other DNA
gccccttccaggtttgcgc

SEQ ID NO: 84 - DNA, 19 bp, synthetic construct
  misc_feature 1..19: note = Synthetic oligonucleotide
  source 1..19: mol_type = other DNA
cgccccttccaggtttgcg

SEQ ID NO: 85 - DNA, 19 bp, synthetic construct
  misc_feature 1..19: note = Synthetic oligonucleotide
  source 1..19: mol_type = other DNA
gcgccccttccaggtttgc

SEQ ID NO: 86 - DNA, 19 bp, synthetic construct
  misc_feature 1..19: note = Synthetic oligonucleotide
  source 1..19: mol_type = other DNA
cgcgccccttccaggtttg

SEQ ID NO: 87 - DNA, 19 bp, synthetic construct
  misc_feature 1..19: note = Synthetic oligonucleotide
  source 1..19: mol_type = other DNA
caaagttggtgcctgcgcg

SEQ ID NO: 88 - DNA, 19 bp, synthetic construct
  misc_feature 1..19: note = Synthetic oligonucleotide
  source 1..19: mol_type = other DNA
gcaaagttggtgcctgcgc

SEQ ID NO: 89 - DNA, 19 bp, synthetic construct
  misc_feature 1..19: note = Synthetic oligonucleotide
  source 1..19: mol_type = other DNA
ggcaaagttggtgcctgcg

SEQ ID NO: 90 - DNA, 19 bp, synthetic construct
  misc_feature 1..19: note = Synthetic oligonucleotide
  source 1..19: mol_type = other DNA
tggcaaagttggtgcctgc

SEQ ID NO: 91 - DNA, 19 bp, synthetic construct
  misc_feature 1..19: note = Synthetic oligonucleotide
  source 1..19: mol_type = other DNA
ttggcaaagttggtgcctg

SEQ ID NO: 92 - DNA, 19 bp, synthetic construct
  misc_feature 1..19: note = Synthetic oligonucleotide
  source 1..19: mol_type = other DNA
tttggcaaagttggtgcct

SEQ ID NO: 93 - DNA, 19 bp, synthetic construct
  misc_feature 1..19: note = Synthetic oligonucleotide
  source 1..19: mol_type = other DNA
gtttggcaaagttggtgcc

SEQ ID NO: 94 - DNA, 19 bp, synthetic construct
  misc_feature 1..19: note = Synthetic oligonucleotide
  source 1..19: mol_type = other DNA
cgtttggcaaagttggtgc

SEQ ID NO: 95 - DNA, 19 bp, synthetic construct
  misc_feature 1..19: note = Synthetic oligonucleotide
  source 1..19: mol_type = other DNA
gcgtttggcaaagttggtg

SEQ ID NO: 96 - DNA, 19 bp, synthetic construct
  misc_feature 1..19: note = Synthetic oligonucleotide
  source 1..19: mol_type = other DNA
agcgtttggcaaagttggt

SEQ ID NO: 97 - DNA, 19 bp, synthetic construct
  misc_feature 1..19: note = Synthetic oligonucleotide
  source 1..19: mol_type = other DNA
tagcgtttggcaaagttgg

SEQ ID NO: 98 - DNA, 19 bp, synthetic construct
  misc_feature 1..19: note = Synthetic oligonucleotide
  source 1..19: mol_type = other DNA
gtagcgtttggcaaagttg

SEQ ID NO: 99 - DNA, 19 bp, synthetic construct
  misc_feature 1..19: note = Synthetic oligonucleotide
  source 1..19: mol_type = other DNA
tgtagcgtttggcaaagtt

SEQ ID NO: 100 - DNA, 19 bp, synthetic construct
  misc_feature 1..19: note = Synthetic oligonucleotide
  source 1..19: mol_type = other DNA
ctgtagcgtttggcaaagt

SEQ ID NO: 101 - DNA, 19 bp, synthetic construct
  misc_feature 1..19: note = Synthetic oligonucleotide
  source 1..19: mol_type = other DNA
tctgtagcgtttggcaaag

SEQ ID NO: 102 - DNA, 19 bp, synthetic construct
  misc_feature 1..19: note = Synthetic oligonucleotide
  source 1..19: mol_type = other DNA
ctctgtagcgtttggcaaa

SEQ ID NO: 103 - DNA, 19 bp, synthetic construct
  misc_feature 1..19: note = Synthetic oligonucleotide
  source 1..19: mol_type = other DNA
actctgtagcgtttggcaa

SEQ ID NO: 104 - DNA, 19 bp, synthetic construct
  misc_feature 1..19: note = Synthetic oligonucleotide
  source 1..19: mol_type = other DNA
aactctgtagcgtttggca

SEQ ID NO: 105 - DNA, 19 bp, synthetic construct
  misc_feature 1..19: note = Synthetic oligonucleotide
  source 1..19: mol_type = other DNA
aaactctgtagcgtttggc

SEQ ID NO: 106 - DNA, 19 bp, synthetic construct
  misc_feature 1..19: note = Synthetic oligonucleotide
  source 1..19: mol_type = other DNA
aaaactctgtagcgtttgg

SEQ ID NO: 107 - DNA, 19 bp, synthetic construct
  misc_feature 1..19: note = Synthetic oligonucleotide
  source 1..19: mol_type = other DNA
gaaaactctgtagcgtttg

SEQ ID NO: 108 - DNA, 19 bp, synthetic construct
  misc_feature 1..19: note = Synthetic oligonucleotide
  source 1..19: mol_type = other DNA
ggaaaactctgtagcgttt

SEQ ID NO: 109 - DNA, 19 bp, synthetic construct
  misc_feature 1..19: note = Synthetic oligonucleotide
  source 1..19: mol_type = other DNA
cggaaaactctgtagcgtt

SEQ ID NO: 110 - DNA, 19 bp, synthetic construct
  misc_feature 1..19: note = Synthetic oligonucleotide
  source 1..19: mol_type = other DNA
acggaaaactctgtagcgt

SEQ ID NO: 111 - DNA, 19 bp, synthetic construct
  misc_feature 1..19: note = Synthetic oligonucleotide
  source 1..19: mol_type = other DNA
aacggaaaactctgtagcg

SEQ ID NO: 112 - DNA, 19 bp, synthetic construct
  misc_feature 1..19: note = Synthetic oligonucleotide
  source 1..19: mol_type = other DNA
aaacggaaaactctgtagc

SEQ ID NO: 113 - DNA, 19 bp, synthetic construct
  misc_feature 1..19: note = Synthetic oligonucleotide
  source 1..19: mol_type = other DNA
caaacggaaaactctgtag

SEQ ID NO: 114 - DNA, 19 bp, synthetic construct
  misc_feature 1..19: note = Synthetic oligonucleotide
  source 1..19: mol_type = other DNA
gcaaacggaaaactctgta

SEQ ID NO: 115 - DNA, 19 bp, synthetic construct
  misc_feature 1..19: note = Synthetic oligonucleotide
  source 1..19: mol_type = other DNA
tgcaaacggaaaactctgt

SEQ ID NO: 116 - DNA, 19 bp, synthetic construct
  misc_feature 1..19: note = Synthetic oligonucleotide
  source 1..19: mol_type = other DNA
ctgcaaacggaaaactctg

SEQ ID NO: 117 - DNA, 19 bp, synthetic construct
  misc_feature 1..19: note = Synthetic oligonucleotide
  source 1..19: mol_type = other DNA
tctgcaaacggaaaactct

SEQ ID NO: 118 - DNA, 19 bp, synthetic construct
  misc_feature 1..19: note = Synthetic oligonucleotide
  source 1..19: mol_type = other DNA
ttctgcaaacggaaaactc

SEQ ID NO: 119 - DNA, 19 bp, synthetic construct
  misc_feature 1..19: note = Synthetic oligonucleotide
  source 1..19: mol_type = other DNA
tttctgcaaacggaaaact

SEQ ID NO: 120 - DNA, 19 bp, synthetic construct
  misc_feature 1..19: note = Synthetic oligonucleotide
  source 1..19: mol_type = other DNA
atttctgcaaacggaaaac

SEQ ID NO: 121 - DNA, 19 bp, synthetic construct
  misc_feature 1..19: note = Synthetic oligonucleotide
  source 1..19: mol_type = other DNA
catttctgcaaacggaaaa

SEQ ID NO: 122 - DNA, 19 bp, synthetic construct
  misc_feature 1..19: note = Synthetic oligonucleotide
  source 1..19: mol_type = other DNA
tcatttctgcaaacggaaa

SEQ ID NO: 123 - DNA, 19 bp, synthetic construct
  misc_feature 1..19: note = Synthetic oligonucleotide
  source 1..19: mol_type = other DNA
gtcatttctgcaaacggaa

SEQ ID NO: 124 - DNA, 19 bp, synthetic construct
  misc_feature 1..19: note = Synthetic oligonucleotide
  source 1..19: mol_type = other DNA
ggtcatttctgcaaacgga

SEQ ID NO: 125 - DNA, 19 bp, synthetic construct
  misc_feature 1..19: note = Synthetic oligonucleotide
  source 1..19: mol_type = other DNA
gggtcatttctgcaaacgg

SEQ ID NO: 126 - DNA, 19 bp, synthetic construct
  misc_feature 1..19: note = Synthetic oligonucleotide
  source 1..19: mol_type = other DNA
tgggtcatttctgcaaacg

SEQ ID NO: 127 - DNA, 19 bp, synthetic construct
  misc_feature 1..19: note = Synthetic oligonucleotide
  source 1..19: mol_type = other DNA
ttgggtcatttctgcaaac

SEQ ID NO: 128 - DNA, 19 bp, synthetic construct
  misc_feature 1..19: note = Synthetic oligonucleotide
  source 1..19: mol_type = other DNA
tttgggtcatttctgcaaa

SEQ ID NO: 129 - DNA, 19 bp, synthetic construct
  misc_feature 1..19: note = Synthetic oligonucleotide
  source 1..19: mol_type = other DNA
ttttgggtcatttctgcaa

SEQ ID NO: 130 - DNA, 19 bp, synthetic construct
  misc_feature 1..19: note = Synthetic oligonucleotide
  source 1..19: mol_type = other DNA
cttttgggtcatttctgca

SEQ ID NO: 131 - DNA, 19 bp, synthetic construct
  misc_feature 1..19: note = Synthetic oligonucleotide
  source 1..19: mol_type = other DNA
tcttttgggtcatttctgc

SEQ ID NO: 132 - DNA, 19 bp, synthetic construct
  misc_feature 1..19: note = Synthetic oligonucleotide
  source 1..19: mol_type = other DNA
ttcttttgggtcatttctg

SEQ ID NO: 133 - DNA, 19 bp, synthetic construct
  misc_feature 1..19: note = Synthetic oligonucleotide
  source 1..19: mol_type = other DNA
cttcttttgggtcatttct

SEQ ID NO: 134 - DNA, 19 bp, synthetic construct
  misc_feature 1..19: note = Synthetic oligonucleotide
  source 1..19: mol_type = other DNA
ccttcttttgggtcatttc

SEQ ID NO: 135 - DNA, 19 bp, synthetic construct
  misc_feature 1..19: note = Synthetic oligonucleotide
  source 1..19: mol_type = other DNA
cccttcttttgggtcattt

SEQ ID NO: 136 - DNA, 19 bp, synthetic construct
  misc_feature 1..19: note = Synthetic oligonucleotide
  source 1..19: mol_type = other DNA
ccccttcttttgggtcatt

SEQ ID NO: 137 - DNA, 19 bp, synthetic construct
  misc_feature 1..19: note = Synthetic oligonucleotide
  source 1..19: mol_type = other DNA
tggggaatcacggggagcg

SEQ ID NO: 138 - DNA, 19 bp, synthetic construct
  misc_feature 1..19: note = Synthetic oligonucleotide
  source 1..19: mol_type = other DNA
ttggggaatcacggggagc

SEQ ID NO: 139 - DNA, 19 bp, synthetic construct
  misc_feature 1..19: note = Synthetic oligonucleotide
  source 1..19: mol_type = other DNA
attggggaatcacggggag

SEQ ID NO: 140 - DNA, 19 bp, synthetic construct
  misc_feature 1..19: note = Synthetic oligonucleotide
  source 1..19: mol_type = other DNA
gattggggaatcacgggga

SEQ ID NO: 141 - DNA, 19 bp, synthetic construct
  misc_feature 1..19: note = Synthetic oligonucleotide
  source 1..19: mol_type = other DNA
cgattggggaatcacgggg

SEQ ID NO: 142 - DNA, 19 bp, synthetic construct
  misc_feature 1..19: note = Synthetic oligonucleotide
  source 1..19: mol_type = other DNA
ccgattggggaatcacggg

SEQ ID NO: 143 - DNA, 19 bp, synthetic construct
  misc_feature 1..19: note = Synthetic oligonucleotide
  source 1..19: mol_type = other DNA
gccgattggggaatcacgg

SEQ ID NO: 144 - DNA, 19 bp, synthetic construct
  misc_feature 1..19: note = Synthetic oligonucleotide
  source 1..19: mol_type = other DNA
agccgattggggaatcacg

SEQ ID NO: 145 - DNA, 19 bp, synthetic construct
  misc_feature 1..19: note = Synthetic oligonucleotide
  source 1..19: mol_type = other DNA
aagccgattggggaatcac

SEQ ID NO: 146 - DNA, 19 bp, synthetic construct
  misc_feature 1..19: note = Synthetic oligonucleotide
  source 1..19: mol_type = other DNA
gaagccgattggggaatca

SEQ ID NO: 147 - DNA, 19 bp, synthetic construct
  misc_feature 1..19: note = Synthetic oligonucleotide
  source 1..19: mol_type = other DNA
agaagccgattggggaatc

SEQ ID NO: 148 - DNA, 19 bp, synthetic construct
  misc_feature 1..19: note = Synthetic oligonucleotide
  source 1..19: mol_type = other DNA
cagaagccgattggggaat

SEQ ID NO: 149 - DNA, 19 bp, synthetic construct
  misc_feature 1..19: note = Synthetic oligonucleotide
  source 1..19: mol_type = other DNA
ccagaagccgattggggaa

SEQ ID NO: 150 - DNA, 19 bp, synthetic construct
  misc_feature 1..19: note = Synthetic oligonucleotide
  source 1..19: mol_type = other DNA
tccagaagccgattgggga

SEQ ID NO: 151 - DNA, 19 bp, synthetic construct
  misc_feature 1..19: note = Synthetic oligonucleotide
  source 1..19: mol_type = other DNA
ctccagaagccgattgggg

SEQ ID NO: 152 - DNA, 19 bp, synthetic construct
  misc_feature 1..19: note = Synthetic oligonucleotide
  source 1..19: mol_type = other DNA
gctccagaagccgattggg

SEQ ID NO: 153 - DNA, 19 bp, synthetic construct
  misc_feature 1..19: note = Synthetic oligonucleotide
  source 1..19: mol_type = other DNA
ggctccagaagccgattgg

SEQ ID NO: 154 - DNA, 19 bp, synthetic construct
  misc_feature 1..19: note = Synthetic oligonucleotide
  source 1..19: mol_type = other DNA
tggctccagaagccgattg

SEQ ID NO: 155 - DNA, 19 bp, synthetic construct
  misc_feature 1..19: note = Synthetic oligonucleotide
  source 1..19: mol_type = other DNA
ctggctccagaagccgatt

SEQ ID NO: 156 - DNA, 19 bp, synthetic construct
  misc_feature 1..19: note = Synthetic oligonucleotide
  source 1..19: mol_type = other DNA
tctggctccagaagccgat

SEQ ID NO: 157 - DNA, 19 bp, synthetic construct
  misc_feature 1..19: note = Synthetic oligonucleotide
  source 1..19: mol_type = other DNA
ctctggctccagaagccga

SEQ ID NO: 158 - DNA, 19 bp, synthetic construct
  misc_feature 1..19: note = Synthetic oligonucleotide
  source 1..19: mol_type = other DNA
cctctggctccagaagccg

SEQ ID NO: 159 - DNA, 19 bp, synthetic construct
  misc_feature 1..19: note = Synthetic oligonucleotide
  source 1..19: mol_type = other DNA
gcctctggctccagaagcc

SEQ ID NO: 160 - DNA, 19 bp, synthetic construct
  misc_feature 1..19: note = Synthetic oligonucleotide
  source 1..19: mol_type = other DNA
agcctctggctccagaagc

SEQ ID NO: 161 - DNA, 19 bp, synthetic construct
  misc_feature 1..19: note = Synthetic oligonucleotide
  source 1..19: mol_type = other DNA
gagcctctggctccagaag

SEQ ID NO: 162 - DNA, 19 bp, synthetic construct
  misc_feature 1..19: note = Synthetic oligonucleotide
  source 1..19: mol_type = other DNA
ggagcctctggctccagaa

SEQ ID NO: 163 - DNA, 19 bp, synthetic construct
  misc_feature 1..19: note = Synthetic oligonucleotide
  source 1..19: mol_type = other DNA
aggagcctctggctccaga

SEQ ID NO: 164 - DNA, 19 bp, synthetic construct
  misc_feature 1..19: note = Synthetic oligonucleotide
  source 1..19: mol_type = other DNA
gaggagcctctggctccag

SEQ ID NO: 165 - DNA, 19 bp, synthetic construct
  misc_feature 1..19: note = Synthetic oligonucleotide
  source 1..19: mol_type = other DNA
ggaggagcctctggctcca

SEQ ID NO: 166 - DNA, 19 bp, synthetic construct
  misc_feature 1..19: note = Synthetic oligonucleotide
  source 1..19: mol_type = other DNA
aggaggagcctctggctcc

SEQ ID NO: 167 - DNA, 19 bp, synthetic construct
  misc_feature 1..19: note = Synthetic oligonucleotide
  source 1..19: mol_type = other DNA
agcagggaagcgggcagca

SEQ ID NO: 168 - DNA, 19 bp, synthetic construct
  misc_feature 1..19: note = Synthetic oligonucleotide
  source 1..19: mol_type = other DNA
tcctcggagcagggaagcg

SEQ ID NO: 169 - DNA, 19 bp, synthetic construct
  misc_feature 1..19: note = Synthetic oligonucleotide
  source 1..19: mol_type = other DNA
ttcctcggagcagggaagc

SEQ ID NO: 170 - DNA, 19 bp, synthetic construct
  misc_feature 1..19: note = Synthetic oligonucleotide
  source 1..19: mol_type = other DNA
tttcctcggagcagggaag

SEQ ID NO: 171 - DNA, 19 bp, synthetic construct
  misc_feature 1..19: note = Synthetic oligonucleotide
  source 1..19: mol_type = other DNA
ttttcctcggagcagggaa

SEQ ID NO: 172 - DNA, 19 bp, synthetic construct
  misc_feature 1..19: note = Synthetic oligonucleotide
  source 1..19: mol_type = other DNA
aaagttgtggagtcgtttt

SEQ ID NO: 173 - DNA, 19 bp, synthetic construct
  misc_feature 1..19: note = Synthetic oligonucleotide
  source 1..19: mol_type = other DNA
caaagttgtggagtcgttt

SEQ ID NO: 174 - DNA, 19 bp, synthetic construct
  misc_feature 1..19: note = Synthetic oligonucleotide
  source 1..19: mol_type = other DNA
acaaagttgtggagtcgtt

SEQ ID NO: 175 - DNA, 19 bp, synthetic construct
  misc_feature 1..19: note = Synthetic oligonucleotide
  source 1..19: mol_type = other DNA
cacaaagttgtggagtcgt

SEQ ID NO: 176 - DNA, 19 bp, synthetic construct
  misc_feature 1..19: note = Synthetic oligonucleotide
  source 1..19: mol_type = other DNA
gcacaaagttgtggagtcg

SEQ ID NO: 177 - DNA, 19 bp, synthetic construct
  misc_feature 1..19: note = Synthetic oligonucleotide
  source 1..19: mol_type = other DNA
cgcacaaagttgtggagtc

SEQ ID NO: 178 - DNA, 19 bp, synthetic construct
  misc_feature 1..19: note = Synthetic oligonucleotide
  source 1..19: mol_type = other DNA
gcgcacaaagttgtggagt

SEQ ID NO: 179 - DNA, 19 bp, synthetic construct
  misc_feature 1..19: note = Synthetic oligonucleotide
  source 1..19: mol_type = other DNA
cgcgcacaaagttgtggag

SEQ ID NO: 180 - DNA, 19 bp, synthetic construct
  misc_feature 1..19: note = Synthetic oligonucleotide
  source 1..19: mol_type = other DNA
tcgcgcacaaagttgtgga

SEQ ID NO: 181 - DNA, 19 bp, synthetic construct
  misc_feature 1..19: note = Synthetic oligonucleotide
  source 1..19: mol_type = other DNA
ctcgcgcacaaagttgtgg

SEQ ID NO: 182 - DNA, 19 bp, synthetic construct
  misc_feature 1..19: note = Synthetic oligonucleotide
  source 1..19: mol_type = other DNA
tctcgcgcacaaagttgtg

SEQ ID NO: 183 - DNA, 19 bp, synthetic construct
  misc_feature 1..19: note = Synthetic oligonucleotide
  source 1..19: mol_type = other DNA
ctctcgcgcacaaagttgt

SEQ ID NO: 184 - DNA, 19 bp, synthetic construct
  misc_feature 1..19: note = Synthetic oligonucleotide
  source 1..19: mol_type = other DNA
ccgaaggagcgagcctctc

SEQ ID NO: 185 - DNA, 19 bp, synthetic construct
  misc_feature 1..19: note = Synthetic oligonucleotide
  source 1..19: mol_type = other DNA
gccgaaggagcgagcctct

SEQ ID NO: 186 - DNA, 19 bp, synthetic construct
  misc_feature 1..19: note = Synthetic oligonucleotide
  source 1..19: mol_type = other DNA
aggaggagccgccgaagga

SEQ ID NO: 187 - DNA, 19 bp, synthetic construct
  misc_feature 1..19: note = Synthetic oligonucleotide
  source 1..19: mol_type = other DNA
acaaaggggcaggcccgca

SEQ ID NO: 188 - DNA, 19 bp, synthetic construct
  misc_feature 1..19: note = Synthetic oligonucleotide
  source 1..19: mol_type = other DNA
aacaaaggggcaggcccgc

SEQ ID NO: 189 - DNA, 19 bp, synthetic construct
  misc_feature 1..19: note = Synthetic oligonucleotide
  source 1..19: mol_type = other DNA
caacaaaggggcaggcccg

SEQ ID NO: 190 - DNA, 19 bp, synthetic construct
  misc_feature 1..19: note = Synthetic oligonucleotide
  source 1..19: mol_type = other DNA
ccaacaaaggggcaggccc

SEQ ID NO: 191 - DNA, 19 bp, synthetic construct
  misc_feature 1..19: note = Synthetic oligonucleotide
  source 1..19: mol_type = other DNA
tccaacaaaggggcaggcc

SEQ ID NO: 192 - DNA, 19 bp, synthetic construct
  misc_feature 1..19: note = Synthetic oligonucleotide
  source 1..19: mol_type = other DNA
ctccaacaaaggggcaggc

SEQ ID NO: 193 - DNA, 19 bp, synthetic construct
  misc_feature 1..19: note = Synthetic oligonucleotide
  source 1..19: mol_type = other DNA
gctccaacaaaggggcagg

SEQ ID NO: 194 - DNA, 19 bp, synthetic construct
  misc_feature 1..19: note = Synthetic oligonucleotide
  source 1..19: mol_type = other DNA
ggctccaacaaaggggcag

SEQ ID NO: 195 - DNA, 19 bp, synthetic construct
  misc_feature 1..19: note = Synthetic oligonucleotide
  source 1..19: mol_type = other DNA
cggctccaacaaaggggca

SEQ ID NO: 196 - DNA, 19 bp, synthetic construct
  misc_feature 1..19: note = Synthetic oligonucleotide
  source 1..19: mol_type = other DNA
tcggctccaacaaaggggc

SEQ ID NO: 197 - DNA, 19 bp, synthetic construct
  misc_feature 1..19: note = Synthetic oligonucleotide
  source 1..19: mol_type = other DNA
ctcggctccaacaaagggg

SEQ ID NO: 198 - DNA, 19 bp, synthetic construct
  misc_feature 1..19: note = Synthetic oligonucleotide
  source 1..19: mol_type = other DNA
gctcggctccaacaaaggg

SEQ ID NO: 199 - DNA, 19 bp, synthetic construct
  misc_feature 1..19: note = Synthetic oligonucleotide
  source 1..19: mol_type = other DNA
cgctcggctccaacaaagg

SEQ ID NO: 200 - DNA, 19 bp, synthetic construct
  misc_feature 1..19: note = Synthetic oligonucleotide
  source 1..19: mol_type = other DNA
gcgctcggctccaacaaag

SEQ ID NO: 201 - DNA, 19 bp, synthetic construct
  misc_feature 1..19: note = Synthetic oligonucleotide
  source 1..19: mol_type = other DNA
agcgctcggctccaacaaa

SEQ ID NO: 202 - DNA, 19 bp, synthetic construct
  misc_feature 1..19: note = Synthetic oligonucleotide
  source 1..19: mol_type = other DNA
cagcgctcggctccaacaa

SEQ ID NO: 203 - DNA, 19 bp, synthetic construct
  misc_feature 1..19: note = Synthetic oligonucleotide
  source 1..19: mol_type = other DNA
ccagcgctcggctccaaca

SEQ ID NO: 204 - DNA, 19 bp, synthetic construct
  misc_feature 1..19: note = Synthetic oligonucleotide
  source 1..19: mol_type = other DNA
tccagcgctcggctccaac

SEQ ID NO: 205 - DNA, 19 bp, synthetic construct
  misc_feature 1..19: note = Synthetic oligonucleotide
  source 1..19: mol_type = other DNA
ctccagcgctcggctccaa

SEQ ID NO: 206 - DNA, 19 bp, synthetic construct
  misc_feature 1..19: note = Synthetic oligonucleotide
  source 1..19: mol_type = other DNA
tctggagagcggacccctg

SEQ ID NO: 207 - DNA, 19 bp, synthetic construct
  misc_feature 1..19: note = Synthetic oligonucleotide
  source 1..19: mol_type = other DNA
atctggagagcggacccct

SEQ ID NO: 208 - DNA, 19 bp, synthetic construct
  misc_feature 1..19: note = Synthetic oligonucleotide
  source 1..19: mol_type = other DNA
catctggagagcggacccc

SEQ ID NO: 209 - DNA, 19 bp, synthetic construct
  misc_feature 1..19: note = Synthetic oligonucleotide
  source 1..19: mol_type = other DNA
tcatctggagagcggaccc

SEQ ID NO: 210 - DNA, 19 bp, synthetic construct
  misc_feature 1..19: note = Synthetic oligonucleotide
  source 1..19: mol_type = other DNA
ctcatctggagagcggacc

SEQ ID NO: 211 - DNA, 19 bp, synthetic construct
  misc_feature 1..19: note = Synthetic oligonucleotide
  source 1..19: mol_type = other DNA
tctcatctggagagcggac

SEQ ID NO: 212 - DNA, 19 bp, synthetic construct
  misc_feature 1..19: note = Synthetic oligonucleotide
  source 1..19: mol_type = other DNA
ttctcatctggagagcgga

SEQ ID NO: 213 - DNA, 19 bp, synthetic construct
  misc_feature 1..19: note = Synthetic oligonucleotide
  source 1..19: mol_type = other DNA
cttctcatctggagagcgg

SEQ ID NO: 214 - DNA, 19 bp, synthetic construct
  misc_feature 1..19: note = Synthetic oligonucleotide
  source 1..19: mol_type = other DNA
ccttctcatctggagagcg

SEQ ID NO: 215 - DNA, 19 bp, synthetic construct
  misc_feature 1..19: note = Synthetic oligonucleotide
  source 1..19: mol_type = other DNA
cccttctcatctggagagc

SEQ ID NO: 216 - DNA, 19 bp, synthetic construct
  misc_feature 1..19: note = Synthetic oligonucleotide
  source 1..19: mol_type = other DNA
acccttctcatctggagag

SEQ ID NO: 217 - DNA, 19 bp, synthetic construct
  misc_feature 1..19: note = Synthetic oligonucleotide
  source 1..19: mol_type = other DNA
cacccttctcatctggaga

SEQ ID NO: 218 - DNA, 19 bp, synthetic construct
  misc_feature 1..19: note = Synthetic oligonucleotide
  source 1..19: mol_type = other DNA
gcacccttctcatctggag

SEQ ID NO: 219 - DNA, 19 bp, synthetic construct
  misc_feature 1..19: note = Synthetic oligonucleotide
  source 1..19: mol_type = other DNA
tgcacccttctcatctgga

SEQ ID NO: 220 - DNA, 19 bp, synthetic construct
  misc_feature 1..19: note = Synthetic oligonucleotide
  source 1..19: mol_type = other DNA
ctgcacccttctcatctgg

SEQ ID NO: 221 - DNA, 19 bp, synthetic construct
  misc_feature 1..19: note = Synthetic oligonucleotide
  source 1..19: mol_type = other DNA
tctgcacccttctcatctg

SEQ ID NO: 222 - DNA, 19 bp, synthetic construct
  misc_feature 1..19: note = Synthetic oligonucleotide
  source 1..19: mol_type = other DNA
atctgcacccttctcatct

SEQ ID NO: 223 - DNA, 19 bp, synthetic construct
  misc_feature 1..19: note = Synthetic oligonucleotide
  source 1..19: mol_type = other DNA
gatctgcacccttctcatc

SEQ ID NO: 224 - DNA, 19 bp, synthetic construct
  misc_feature 1..19: note = Synthetic oligonucleotide
  source 1..19: mol_type = other DNA
tgatctgcacccttctcat

SEQ ID NO: 225 - DNA, 19 bp, synthetic construct
  misc_feature 1..19: note = Synthetic oligonucleotide
  source 1..19: mol_type = other DNA
ctgatctgcacccttctca

SEQ ID NO: 226 - DNA, 19 bp, synthetic construct
  misc_feature 1..19: note = Synthetic oligonucleotide
  source 1..19: mol_type = other DNA
gctgatctgcacccttctc

SEQ ID NO: 227 - DNA, 19 bp, synthetic construct
  misc_feature 1..19: note = Synthetic oligonucleotide
  source 1..19: mol_type = other DNA
cgctgatctgcacccttct

SEQ ID NO: 228 - DNA, 19 bp, synthetic construct
  misc_feature 1..19: note = Synthetic oligonucleotide
  source 1..19: mol_type = other DNA
gcgctgatctgcacccttc

SEQ ID NO: 229 - DNA, 19 bp, synthetic construct
  misc_feature 1..19: note = Synthetic oligonucleotide
  source 1..19: mol_type = other DNA
ggcgctgatctgcaccctt

SEQ ID NO: 230 - DNA, 19 bp, synthetic construct
  misc_feature 1..19: note = Synthetic oligonucleotide
  source 1..19: mol_type = other DNA
tggcgctgatctgcaccct

SEQ ID NO: 231 - DNA, 19 bp, synthetic construct
  misc_feature 1..19: note = Synthetic oligonucleotide
  source 1..19: mol_type = other DNA
ctggcgctgatctgcaccc

SEQ ID NO: 232 - DNA, 19 bp, synthetic construct
  misc_feature 1..19: note = Synthetic oligonucleotide
  source 1..19: mol_type = other DNA
gctggcgctgatctgcacc

SEQ ID NO: 233 - DNA, 19 bp, synthetic construct
  misc_feature 1..19: note = Synthetic oligonucleotide
  source 1..19: mol_type = other DNA
ggctggcgctgatctgcac

SEQ ID NO: 234 - DNA, 19 bp, synthetic construct
  misc_feature 1..19: note = Synthetic oligonucleotide
  source 1..19: mol_type = other DNA
gggctggcgctgatctgca

SEQ ID NO: 235 - DNA, 19 bp, synthetic construct
  misc_feature 1..19: note = Synthetic oligonucleotide
  source 1..19: mol_type = other DNA
agggctggcgctgatctgc

SEQ ID NO: 236 - DNA, 19 bp, synthetic construct
  misc_feature 1..19: note = Synthetic oligonucleotide
  source 1..19: mol_type = other DNA
gagggctggcgctgatctg

SEQ ID NO: 237 - DNA, 19 bp, synthetic construct
  misc_feature 1..19: note = Synthetic oligonucleotide
  source 1..19: mol_type = other DNA
ggagggctggcgctgatct

SEQ ID NO: 238 - DNA, 19 bp, synthetic construct
  misc_feature 1..19: note = Synthetic oligonucleotide
  source 1..19: mol_type = other DNA
aaagttgctgggccggcgg

SEQ ID NO: 239 - DNA, 19 bp, synthetic construct
  misc_feature 1..19: note = Synthetic oligonucleotide
  source 1..19: mol_type = other DNA
caaagttgctgggccggcg

SEQ ID NO: 240 - DNA, 19 bp, synthetic construct
  misc_feature 1..19: note = Synthetic oligonucleotide
  source 1..19: mol_type = other DNA
acaaagttgctgggccggc

SEQ ID NO: 241 - DNA, 19 bp, synthetic construct
  misc_feature 1..19: note = Synthetic oligonucleotide
  source 1..19: mol_type = other DNA
aacaaagttgctgggccgg

SEQ ID NO: 242 - DNA, 19 bp, synthetic construct
  misc_feature 1..19: note = Synthetic oligonucleotide
  source 1..19: mol_type = other DNA
gaacaaagttgctgggccg

SEQ ID NO: 243 - DNA, 19 bp, synthetic construct
  misc_feature 1..19: note = Synthetic oligonucleotide
  source 1..19: mol_type = other DNA
ggaacaaagttgctgggcc

SEQ ID NO: 244 - DNA, 19 bp, synthetic construct
  misc_feature 1..19: note = Synthetic oligonucleotide
  source 1..19: mol_type = other DNA
cggaacaaagttgctgggc

SEQ ID NO: 245 - DNA, 19 bp, synthetic construct
  misc_feature 1..19: note = Synthetic oligonucleotide
  source 1..19: mol_type = other DNA
ccggaacaaagttgctggg

SEQ ID NO: 246 - DNA, 19 bp, synthetic construct
  misc_feature 1..19: note = Synthetic oligonucleotide
  source 1..19: mol_type = other DNA
tccggaacaaagttgctgg

SEQ ID NO: 247 - DNA, 19 bp, synthetic construct
  misc_feature 1..19: note = Synthetic oligonucleotide
  source 1..19: mol_type = other DNA
ttccggaacaaagttgctg

SEQ ID NO: 248 - DNA, 19 bp, synthetic construct
  misc_feature 1..19: note = Synthetic oligonucleotide
  source 1..19: mol_type = other DNA
cttccggaacaaagttgct

SEQ ID NO: 249 - DNA, 19 bp, synthetic construct
  misc_feature 1..19: note = Synthetic oligonucleotide
  source 1..19: mol_type = other DNA
tcttccggaacaaagttgc

SEQ ID NO: 250 - DNA, 19 bp, synthetic construct
  misc_feature 1..19: note = Synthetic oligonucleotide
  source 1..19: mol_type = other DNA
atcttccggaacaaagttg

SEQ ID NO: 251 - DNA, 19 bp, synthetic construct
  misc_feature 1..19: note = Synthetic oligonucleotide
  source 1..19: mol_type = other DNA
gatcttccggaacaaagtt

SEQ ID NO: 252 - DNA, 19 bp, synthetic construct
  misc_feature 1..19: note = Synthetic oligonucleotide
  source 1..19: mol_type = other DNA
tgatcttccggaacaaagt

SEQ ID NO: 253 - DNA, 19 bp, synthetic construct
  misc_feature 1..19: note = Synthetic oligonucleotide
  source 1..19: mol_type = other DNA
ctgatcttccggaacaaag

SEQ ID NO: 254 - DNA, 19 bp, synthetic construct
  misc_feature 1..19: note = Synthetic oligonucleotide
  source 1..19: mol_type = other DNA
gctgatcttccggaacaaa

SEQ ID NO: 255 - DNA, 19 bp, synthetic construct
  misc_feature 1..19: note = Synthetic oligonucleotide
  source 1..19: mol_type = other DNA
ggctgatcttccggaacaa

SEQ ID NO: 256 - DNA, 19 bp, synthetic construct
  misc_feature 1..19: note = Synthetic oligonucleotide
  source 1..19: mol_type = other DNA
gggctgatcttccggaaca

SEQ ID NO: 257 - DNA, 19 bp, synthetic construct
  misc_feature 1..19: note = Synthetic oligonucleotide
  source 1..19: mol_type = other DNA
ggggctgatcttccggaac

SEQ ID NO: 258 - DNA, 19 bp, synthetic construct
  misc_feature 1..19: note = Synthetic oligonucleotide
  source 1..19: mol_type = other DNA
tggggctgatcttccggaa

SEQ ID NO: 259 - DNA, 19 bp, synthetic construct
  misc_feature 1..19: note = Synthetic oligonucleotide
  source 1..19: mol_type = other DNA
gtggggctgatcttccgga

SEQ ID NO: 260 - DNA, 19 bp, synthetic construct
  misc_feature 1..19: note = Synthetic oligonucleotide
  source 1..19: mol_type = other DNA
agtggggctgatcttccgg

SEQ ID NO: 261 - DNA, 19 bp, synthetic construct
  misc_feature 1..19: note = Synthetic oligonucleotide
  source 1..19: mol_type = other DNA
tagtggggctgatcttccg

SEQ ID NO: 262 - DNA, 19 bp, synthetic construct
  misc_feature 1..19: note = Synthetic oligonucleotide
  source 1..19: mol_type = other DNA
gtagtggggctgatcttcc

SEQ ID NO: 263 - DNA, 19 bp, synthetic construct
  misc_feature 1..19: note = Synthetic oligonucleotide
  source 1..19: mol_type = other DNA
cgtagtggggctgatcttc

SEQ ID NO: 264 - DNA, 19 bp, synthetic construct
  misc_feature 1..19: note = Synthetic oligonucleotide
  source 1..19: mol_type = other DNA
acgtagtggggctgatctt

SEQ ID NO: 265 - DNA, 19 bp, synthetic construct
  misc_feature 1..19: note = Synthetic oligonucleotide
  source 1..19: mol_type = other DNA
aacgtagtggggctgatct

SEQ ID NO: 266 - DNA, 19 bp, synthetic construct
  misc_feature 1..19: note = Synthetic oligonucleotide
  source 1..19: mol_type = other DNA
gaacgtagtggggctgatc

SEQ ID NO: 267 - DNA, 19 bp, synthetic construct
  misc_feature 1..19: note = Synthetic oligonucleotide
  source 1..19: mol_type = other DNA
ggaacgtagtggggctgat

SEQ ID NO: 268 - DNA, 19 bp, synthetic construct
  misc_feature 1..19: note = Synthetic oligonucleotide
  source 1..19: mol_type = other DNA
gggaacgtagtggggctga

SEQ ID NO: 269 - DNA, 19 bp, synthetic construct
  misc_feature 1..19: note = Synthetic oligonucleotide
  source 1..19: mol_type = other DNA
cgggaacgtagtggggctg

SEQ ID NO: 270 - DNA, 19 bp, synthetic construct
  misc_feature 1..19: note = Synthetic oligonucleotide
  source 1..19: mol_type = other DNA
ccgggaacgtagtggggct

SEQ ID NO: 271 - DNA, 19 bp, synthetic construct
  misc_feature 1..19: note = Synthetic oligonucleotide
  source 1..19: mol_type = other DNA
acccgggaacgtagtgggg

SEQ ID NO: 272 - DNA, 19 bp, synthetic construct
  misc_feature 1..19: note = Synthetic oligonucleotide
  source 1..19: mol_type = other DNA
gacccgggaacgtagtggg

SEQ ID NO: 273 - DNA, 19 bp, synthetic construct
  misc_feature 1..19: note = Synthetic oligonucleotide
  source 1..19: mol_type = other DNA
tgacccgggaacgtagtgg

SEQ ID NO: 274 - DNA, 19 bp, synthetic construct
  misc_feature 1..19: note = Synthetic oligonucleotide
  source 1..19: mol_type = other DNA
ctgacccgggaacgtagtg

SEQ ID NO: 275 - DNA, 19 bp, synthetic construct
  misc_feature 1..19: note = Synthetic oligonucleotide
  source 1..19: mol_type = other DNA
gctgacccgggaacgtagt

SEQ ID NO: 276 - DNA, 19 bp, synthetic construct
  misc_feature 1..19: note = Synthetic oligonucleotide
  source 1..19: mol_type = other DNA
cagacgctgacccgggaac

SEQ ID NO: 277 - DNA, 19 bp, synthetic construct
  misc_feature 1..19: note = Synthetic oligonucleotide
  source 1..19: mol_type = other DNA
gcagacgctgacccgggaa

SEQ ID NO: 278 - DNA, 19 bp, synthetic construct
  misc_feature 1..19: note = Synthetic oligonucleotide
  source 1..19: mol_type = other DNA
tggggcgccaagttcccga

SEQ ID NO: 279 - DNA, 19 bp, synthetic construct
  misc_feature 1..19: note = Synthetic oligonucleotide
  source 1..19: mol_type = other DNA
ttggggcgccaagttcccg

SEQ ID NO: 280 - DNA, 19 bp, synthetic construct
  misc_feature 1..19: note = Synthetic oligonucleotide
  source 1..19: mol_type = other DNA
cttggggcgccaagttccc

SEQ ID NO: 281 - DNA, 19 bp, synthetic construct
  misc_feature 1..19: note = Synthetic oligonucleotide
  source 1..19: mol_type = other DNA
acttggggcgccaagttcc

SEQ ID NO: 282 - DNA, 19 bp, synthetic construct
  misc_feature 1..19: note = Synthetic oligonucleotide
  source 1..19: mol_type = other DNA
gacttggggcgccaagttc

SEQ ID NO: 283 - DNA, 19 bp, synthetic construct
  misc_feature 1..19: note = Synthetic oligonucleotide
  source 1..19: mol_type = other DNA
tgacttggggcgccaagtt

SEQ ID NO: 284 - DNA, 19 bp, synthetic construct
  misc_feature 1..19: note = Synthetic oligonucleotide
  source 1..19: mol_type = other DNA
gtgacttggggcgccaagt

SEQ ID NO: 285 - DNA, 19 bp, synthetic construct
  misc_feature 1..19: note = Synthetic oligonucleotide
  source 1..19: mol_type = other DNA
ggtgacttggggcgccaag

SEQ ID NO: 286 - DNA, 19 bp, synthetic construct
  misc_feature 1..19: note = Synthetic oligonucleotide
  source 1..19: mol_type = other DNA
aggtgacttggggcgccaa

SEQ ID NO: 287 - DNA, 19 bp, synthetic construct
  misc_feature 1..19: note = Synthetic oligonucleotide
  source 1..19: mol_type = other DNA
caggtgacttggggcgcca

SEQ ID NO: 288 - DNA, 19 bp, synthetic construct
  misc_feature 1..19: note = Synthetic oligonucleotide
  source 1..19: mol_type = other DNA
tcaggtgacttggggcgcc

SEQ ID NO: 289 - DNA, 19 bp, synthetic construct
  misc_feature 1..19: note = Synthetic oligonucleotide
  source 1..19: mol_type = other DNA
gtcaggtgacttggggcgc

SEQ ID NO: 290 - DNA, 19 bp, synthetic construct
  misc_feature 1..19: note = Synthetic oligonucleotide
  source 1..19: mol_type = other DNA
agtcaggtgacttggggcg

SEQ ID NO: 291 - DNA, 19 bp, synthetic construct
  misc_feature 1..19: note = Synthetic oligonucleotide
  source 1..19: mol_type = other DNA
cagtcaggtgacttggggc

SEQ ID NO: 292 - DNA, 19 bp, synthetic construct
  misc_feature 1..19: note = Synthetic oligonucleotide
  source 1..19: mol_type = other DNA
gcagtcaggtgacttgggg

SEQ ID NO: 293 - DNA, 19 bp, synthetic construct
  misc_feature 1..19: note = Synthetic oligonucleotide
  source 1..19: mol_type = other DNA
cgcagtcaggtgacttggg

SEQ ID NO: 294 - DNA, 19 bp, synthetic construct
  misc_feature 1..19: note = Synthetic oligonucleotide
  source 1..19: mol_type = other DNA
gcgcagtcaggtgacttgg

SEQ ID NO: 295 - DNA, 19 bp, synthetic construct
  misc_feature 1..19: note = Synthetic oligonucleotide
  source 1..19: mol_type = other DNA
cgcgcagtcaggtgacttg

SEQ ID NO: 296 - DNA, 19 bp, synthetic construct
  misc_feature 1..19: note = Synthetic oligonucleotide
  source 1..19: mol_type = other DNA
agggccggcgctgattgac

SEQ ID NO: 297 - DNA, 19 bp, synthetic construct
  misc_feature 1..19: note = Synthetic oligonucleotide
  source 1..19: mol_type = other DNA
cagggccggcgctgattga

SEQ ID NO: 298 - DNA, 19 bp, synthetic construct
  misc_feature 1..19: note = Synthetic oligonucleotide
  source 1..19: mol_type = other DNA
acggttcactctggagcgc

SEQ ID NO: 299 - DNA, 19 bp, synthetic construct
  misc_feature 1..19: note = Synthetic oligonucleotide
  source 1..19: mol_type = other DNA
cacggttcactctggagcg

SEQ ID NO: 300 - DNA, 19 bp, synthetic construct
  misc_feature 1..19: note = Synthetic oligonucleotide
  source 1..19: mol_type = other DNA
gcacggttcactctggagc

SEQ ID NO: 301 - DNA, 19 bp, synthetic construct
  misc_feature 1..19: note = Synthetic oligonucleotide
  source 1..19: mol_type = other DNA
cgcacggttcactctggag

SEQ ID NO: 302 - DNA, 19 bp, synthetic construct
  misc_feature 1..19: note = Synthetic oligonucleotide
  source 1..19: mol_type = other DNA
acgcacggttcactctgga

SEQ ID NO: 303 - DNA, 19 bp, synthetic construct
  misc_feature 1..19: note = Synthetic oligonucleotide
  source 1..19: mol_type = other DNA
cacgcacggttcactctgg

SEQ ID NO: 304 - DNA, 19 bp, synthetic construct
  misc_feature 1..19: note = Synthetic oligonucleotide
  source 1..19: mol_type = other DNA
gcacgcacggttcactctg

SEQ ID NO: 305 - DNA, 19 bp, synthetic construct
  misc_feature 1..19: note = Synthetic oligonucleotide
  source 1..19: mol_type = other DNA
ggcacgcacggttcactct

SEQ ID NO: 306 - DNA, 19 bp, synthetic construct
  misc_feature 1..19: note = Synthetic oligonucleotide
  source 1..19: mol_type = other DNA
gttcggtctccggactccc

SEQ ID NO: 307 - DNA, 19 bp, synthetic construct
  misc_feature 1..19: note = Synthetic oligonucleotide
  source 1..19: mol_type = other DNA
ggttcggtctccggactcc

SEQ ID NO: 308 - DNA, 19 bp, synthetic construct
  misc_feature 1..19: note = Synthetic oligonucleotide
  source 1..19: mol_type = other DNA
tggttcggtctccggactc

SEQ ID NO: 309 - DNA, 19 bp, synthetic construct
  misc_feature 1..19: note = Synthetic oligonucleotide
  source 1..19: mol_type = other DNA
ctggttcggtctccggact

SEQ ID NO: 310 - DNA, 19 bp, synthetic construct
  misc_feature 1..19: note = Synthetic oligonucleotide
  source 1..19: mol_type = other DNA
tctggttcggtctccggac

SEQ ID NO: 311 - DNA, 19 bp, synthetic construct
  misc_feature 1..19: note = Synthetic oligonucleotide
  source 1..19: mol_type = other DNA
ttctggttcggtctccgga

SEQ ID NO: 312 - DNA, 19 bp, synthetic construct
  misc_feature 1..19: note = Synthetic oligonucleotide
  source 1..19: mol_type = other DNA
attctggttcggtctccgg

SEQ ID NO: 313 - DNA, 19 bp, synthetic construct
  misc_feature 1..19: note = Synthetic oligonucleotide
  source 1..19: mol_type = other DNA
cattctggttcggtctccg

SEQ ID NO: 314 - DNA, 19 bp, synthetic construct
  misc_feature 1..19: note = Synthetic oligonucleotide
  source 1..19: mol_type = other DNA
ccattctggttcggtctcc

SEQ ID NO: 315 - DNA, 19 bp, synthetic construct
  misc_feature 1..19: note = Synthetic oligonucleotide
  source 1..19: mol_type = other DNA
tccattctggttcggtctc

SEQ ID NO: 316 - DNA, 19 bp, synthetic construct
  misc_feature 1..19: note = Synthetic oligonucleotide
  source 1..19: mol_type = other DNA
atccattctggttcggtct

SEQ ID NO: 317 - DNA, 19 bp, synthetic construct
  misc_feature 1..19: note = Synthetic oligonucleotide
  source 1..19: mol_type = other DNA
catccattctggttcggtc

SEQ ID NO: 318 - DNA, 19 bp, synthetic construct
  misc_feature 1..19: note = Synthetic oligonucleotide
  source 1..19: mol_type = other DNA
gcatccattctggttcggt

SEQ ID NO: 319 - DNA, 19 bp, synthetic construct
  misc_feature 1..19: note = Synthetic oligonucleotide
  source 1..19: mol_type = other DNA
ggcatccattctggttcgg

SEQ ID NO: 320 - DNA, 19 bp, synthetic construct
  misc_feature 1..19: note = Synthetic oligonucleotide
  source 1..19: mol_type = other DNA
aggcatccattctggttcg

SEQ ID NO: 321 - DNA, 19 bp, synthetic construct
  misc_feature 1..19: note = Synthetic oligonucleotide
  source 1..19: mol_type = other DNA
gaggcatccattctggttc

SEQ ID NO: 322 - DNA, 19 bp, synthetic construct
  misc_feature 1..19: note = Synthetic oligonucleotide
  source 1..19: mol_type = other DNA
cgaggcatccattctggtt

SEQ ID NO: 323 - DNA, 19 bp, synthetic construct
  misc_feature 1..19: note = Synthetic oligonucleotide
  source 1..19: mol_type = other DNA
tcgaggcatccattctggt

SEQ ID NO: 324 - DNA, 19 bp, synthetic construct
  misc_feature 1..19: note = Synthetic oligonucleotide
  source 1..19: mol_type = other DNA
ctcgaggcatccattctgg

SEQ ID NO: 325 - DNA, 19 bp, synthetic construct
  misc_feature 1..19: note = Synthetic oligonucleotide
  source 1..19: mol_type = other DNA
gctcgaggcatccattctg

SEQ ID NO: 326 - DNA, 19 bp, synthetic construct
  misc_feature 1..19: note = Synthetic oligonucleotide
  source 1..19: mol_type = other DNA
tgctcgaggcatccattct

SEQ ID NO: 327 - DNA, 19 bp, synthetic construct
  misc_feature 1..19: note = Synthetic oligonucleotide
  source 1..19: mol_type = other DNA
ctgctcgaggcatccattc

SEQ ID NO: 328 - DNA, 19 bp, synthetic construct
  misc_feature 1..19: note = Synthetic oligonucleotide
  source 1..19: mol_type = other DNA
tctgctcgaggcatccatt

SEQ ID NO: 329 - DNA, 19 bp, synthetic construct
  misc_feature 1..19: note = Synthetic oligonucleotide
  source 1..19: mol_type = other DNA
gtctgctcgaggcatccat

SEQ ID NO: 330 - DNA, 19 bp, synthetic construct
  misc_feature 1..19: note = Synthetic oligonucleotide
  source 1..19: mol_type = other DNA
tgtctgctcgaggcatcca

SEQ ID NO: 331 - DNA, 19 bp, synthetic construct
  misc_feature 1..19: note = Synthetic oligonucleotide
  source 1..19: mol_type = other DNA
gtgtctgctcgaggcatcc

SEQ ID NO: 332 - DNA, 19 bp, synthetic construct
  misc_feature 1..19: note = Synthetic oligonucleotide
  source 1..19: mol_type = other DNA
tgtgtctgctcgaggcatc

SEQ ID NO: 333 - DNA, 19 bp, synthetic construct
  misc_feature 1..19: note = Synthetic oligonucleotide
  source 1..19: mol_type = other DNA
ttgtgtctgctcgaggcat

SEQ ID NO: 334 - DNA, 19 bp, synthetic construct
  misc_feature 1..19: note = Synthetic oligonucleotide
  source 1..19: mol_type = other DNA
attgtgtctgctcgaggca

SEQ ID NO: 335 - DNA, 19 bp, synthetic construct
  misc_feature 1..19: note = Synthetic oligonucleotide
  source 1..19: mol_type = other DNA
aattgtgtctgctcgaggc

SEQ ID NO: 336 - DNA, 19 bp, synthetic construct
  misc_feature 1..19: note = Synthetic oligonucleotide
  source 1..19: mol_type = other DNA
gaattgtgtctgctcgagg

SEQ ID NO: 337 - DNA, 19 bp, synthetic construct
  misc_feature 1..19: note = Synthetic oligonucleotide
  source 1..19: mol_type = other DNA
ggaattgtgtctgctcgag

SEQ ID NO: 338 - DNA, 19 bp, synthetic construct
  misc_feature 1..19: note = Synthetic oligonucleotide
  source 1..19: mol_type = other DNA
gggaattgtgtctgctcga

SEQ ID NO: 339 - DNA, 19 bp, synthetic construct
  misc_feature 1..19: note = Synthetic oligonucleotide
  source 1..19: mol_type = other DNA
ggggaattgtgtctgctcg

SEQ ID NO: 340 - DNA, 19 bp, synthetic construct
  misc_feature 1..19: note = Synthetic oligonucleotide
  source 1..19: mol_type = other DNA
aggggaattgtgtctgctc

SEQ ID NO: 341 - DNA, 19 bp, synthetic construct
  misc_feature 1..19: note = Synthetic oligonucleotide
  source 1..19: mol_type = other DNA
aaggggaattgtgtctgct

SEQ ID NO: 342 - DNA, 19 bp, synthetic construct
  misc_feature 1..19: note = Synthetic oligonucleotide
  source 1..19: mol_type = other DNA
taaggggaattgtgtctgc

SEQ ID NO: 343 - DNA, 19 bp, synthetic construct
  misc_feature 1..19: note = Synthetic oligonucleotide
  source 1..19: mol_type = other DNA
ataaggggaattgtgtctg

SEQ ID NO: 344 - DNA, 19 bp, synthetic construct
  misc_feature 1..19: note = Synthetic oligonucleotide
  source 1..19: mol_type = other DNA
aataaggggaattgtgtct

SEQ ID NO: 345 - DNA, 19 bp, synthetic construct
  misc_feature 1..19: note = Synthetic oligonucleotide
  source 1..19: mol_type = other DNA
gaataaggggaattgtgtc

SEQ ID NO: 346 - DNA, 19 bp, synthetic construct
  misc_feature 1..19: note = Synthetic oligonucleotide
  source 1..19: mol_type = other DNA
tgaataaggggaattgtgt

SEQ ID NO: 347 - DNA, 19 bp, synthetic construct
  misc_feature 1..19: note = Synthetic oligonucleotide
  source 1..19: mol_type = other DNA
ctgaataaggggaattgtg

SEQ ID NO: 348 - DNA, 19 bp, synthetic construct
  misc_feature 1..19: note = Synthetic oligonucleotide
  source 1..19: mol_type = other DNA
tctgaataaggggaattgt

SEQ ID NO: 349 - DNA, 19 bp, synthetic construct
  misc_feature 1..19: note = Synthetic oligonucleotide
  source 1..19: mol_type = other DNA
atctgaataaggggaattg

SEQ ID NO: 350 - DNA, 19 bp, synthetic construct
  misc_feature 1..19: note = Synthetic oligonucleotide
  source 1..19: mol_type = other DNA
gataatctgaataagggga

SEQ ID NO: 351 - DNA, 19 bp, synthetic construct
  misc_feature 1..19: note = Synthetic oligonucleotide
  source 1..19: mol_type = other DNA
ggataatctgaataagggg

SEQ ID NO: 352 - DNA, 19 bp, synthetic construct
  misc_feature 1..19: note = Synthetic oligonucleotide
  source 1..19: mol_type = other DNA
cggataatctgaataaggg

SEQ ID NO: 353 - DNA, 19 bp, synthetic construct
  misc_feature 1..19: note = Synthetic oligonucleotide
  source 1..19: mol_type = other DNA
tcggataatctgaataagg

SEQ ID NO: 354 - DNA, 19 bp, synthetic construct
  misc_feature 1..19: note = Synthetic oligonucleotide
  source 1..19: mol_type = other DNA
cacattttcggataatctg

SEQ ID NO: 355 - DNA, 19 bp, synthetic construct
  misc_feature 1..19: note = Synthetic oligonucleotide
  source 1..19: mol_type = other DNA
ctcacattttcggataatc

SEQ ID NO: 356 - DNA, 19 bp, synthetic construct
  misc_feature 1..19: note = Synthetic oligonucleotide
  source 1..19: mol_type = other DNA
gactcacattttcggataa

SEQ ID NO: 357 - DNA, 19 bp, synthetic construct
  misc_feature 1..19: note = Synthetic oligonucleotide
  source 1..19: mol_type = other DNA
tgactcacattttcggata

SEQ ID NO: 358 - DNA, 19 bp, synthetic construct
  misc_feature 1..19: note = Synthetic oligonucleotide
  source 1..19: mol_type = other DNA
gtgactcacattttcggat

SEQ ID NO: 359 - DNA, 19 bp, synthetic construct
  misc_feature 1..19: note = Synthetic oligonucleotide
  source 1..19: mol_type = other DNA
cgtgactcacattttcgga

SEQ ID NO: 360 - DNA, 19 bp, synthetic construct
  misc_feature 1..19: note = Synthetic oligonucleotide
  source 1..19: mol_type = other DNA
acgtgactcacattttcgg

SEQ ID NO: 361 - DNA, 19 bp, synthetic construct
  misc_feature 1..19: note = Synthetic oligonucleotide
  source 1..19: mol_type = other DNA
gacgtgactcacattttcg

SEQ ID NO: 362 - DNA, 19 bp, synthetic construct
  misc_feature 1..19: note = Synthetic oligonucleotide
  source 1..19: mol_type = other DNA
ggacgtgactcacattttc

SEQ ID NO: 363 - DNA, 19 bp, synthetic construct
  misc_feature 1..19: note = Synthetic oligonucleotide
  source 1..19: mol_type = other DNA
gggacgtgactcacatttt

SEQ ID NO: 364 - DNA, 19 bp, synthetic construct
  misc_feature 1..19: note = Synthetic oligonucleotide
  source 1..19: mol_type = other DNA
ggggacgtgactcacattt

SEQ ID NO: 365 - DNA, 19 bp, synthetic construct
  misc_feature 1..19: note = Synthetic oligonucleotide
  source 1..19: mol_type = other DNA
aggggacgtgactcacatt

SEQ ID NO: 366 - DNA, 19 bp, synthetic construct
  misc_feature 1..19: note = Synthetic oligonucleotide
  source 1..19: mol_type = other DNA
aaggggacgtgactcacat

SEQ ID NO: 367 - DNA, 19 bp, synthetic construct
  misc_feature 1..19: note = Synthetic oligonucleotide
  source 1..19: mol_type = other DNA
taaggggacgtgactcaca

SEQ ID NO: 368 - DNA, 19 bp, synthetic construct
  misc_feature 1..19: note = Synthetic oligonucleotide
  source 1..19: mol_type = other DNA
ctaaggggacgtgactcac

SEQ ID NO: 369 - DNA, 19 bp, synthetic construct
  misc_feature 1..19: note = Synthetic oligonucleotide
  source 1..19: mol_type = other DNA
cctaaggggacgtgactca

SEQ ID NO: 370 - DNA, 19 bp, synthetic construct
  misc_feature 1..19: note = Synthetic oligonucleotide
  source 1..19: mol_type = other DNA
gcctaaggggacgtgactc

SEQ ID NO: 371 - DNA, 19 bp, synthetic construct
  misc_feature 1..19: note = Synthetic oligonucleotide
  source 1..19: mol_type = other DNA
tgcctaaggggacgtgact

SEQ ID NO: 372 - DNA, 19 bp, synthetic construct
  misc_feature 1..19: note = Synthetic oligonucleotide
  source 1..19: mol_type = other DNA
ttgcctaaggggacgtgac

SEQ ID NO: 373 - DNA, 19 bp, synthetic construct
  misc_feature 1..19: note = Synthetic oligonucleotide
  source 1..19: mol_type = other DNA
tttgcctaaggggacgtga

SEQ ID NO: 374 - DNA, 19 bp, synthetic construct
  misc_feature 1..19: note = Synthetic oligonucleotide
  source 1..19: mol_type = other DNA
ttttgcctaaggggacgtg

SEQ ID NO: 375 - DNA, 19 bp, synthetic construct
  misc_feature 1..19: note = Synthetic oligonucleotide
  source 1..19: mol_type = other DNA
gttttgcctaaggggacgt

SEQ ID NO: 376 - DNA, 19 bp, synthetic construct
  misc_feature 1..19: note = Synthetic oligonucleotide
  source 1..19: mol_type = other DNA
tgttttgcctaaggggacg

SEQ ID NO: 377 - DNA, 19 bp, synthetic construct
  misc_feature 1..19: note = Synthetic oligonucleotide
  source 1..19: mol_type = other DNA
ctgttttgcctaaggggac

SEQ ID NO: 378 - DNA, 19 bp, synthetic construct
  misc_feature 1..19: note = Synthetic oligonucleotide
  source 1..19: mol_type = other DNA
cctgttttgcctaagggga

SEQ ID NO: 379 - DNA, 19 bp, synthetic construct
  misc_feature 1..19: note = Synthetic oligonucleotide
  source 1..19: mol_type = other DNA
acctgttttgcctaagggg

SEQ ID NO: 380 - DNA, 19 bp, synthetic construct
  misc_feature 1..19: note = Synthetic oligonucleotide
  source 1..19: mol_type = other DNA
gacctgttttgcctaaggg

SEQ ID NO: 381 - DNA, 19 bp, synthetic construct
  misc_feature 1..19: note = Synthetic oligonucleotide
  source 1..19: mol_type = other DNA
agacctgttttgcctaagg

SEQ ID NO: 382 - DNA, 19 bp, synthetic construct
  misc_feature 1..19: note = Synthetic oligonucleotide
  source 1..19: mol_type = other DNA
gagacctgttttgcctaag

SEQ ID NO: 383 - DNA, 19 bp, synthetic construct
  misc_feature 1..19: note = Synthetic oligonucleotide
  source 1..19: mol_type = other DNA
agagacctgttttgcctaa

SEQ ID NO: 384 - DNA, 19 bp, synthetic construct
  misc_feature 1..19: note = Synthetic oligonucleotide
  source 1..19: mol_type = other DNA
tagagacctgttttgccta

SEQ ID NO: 385 - DNA, 19 bp, synthetic construct
  misc_feature 1..19: note = Synthetic oligonucleotide
  source 1..19: mol_type = other DNA
ctagagacctgttttgcct

SEQ ID NO: 386 - DNA, 19 bp, synthetic construct
  misc_feature 1..19: note = Synthetic oligonucleotide
  source 1..19: mol_type = other DNA
actagagacctgttttgcc

SEQ ID NO: 387 - DNA, 19 bp, synthetic construct
  misc_feature 1..19: note = Synthetic oligonucleotide
  source 1..19: mol_type = other DNA
aactagagacctgttttgc

SEQ ID NO: 388 - DNA, 19 bp, synthetic construct
  misc_feature 1..19: note = Synthetic oligonucleotide
  source 1..19: mol_type = other DNA
taactagagacctgttttg

SEQ ID NO: 389 - DNA, 19 bp, synthetic construct
  misc_feature 1..19: note = Synthetic oligonucleotide
  source 1..19: mol_type = other DNA
ctaactagagacctgtttt

SEQ ID NO: 390 - DNA, 19 bp, synthetic construct
  misc_feature 1..19: note = Synthetic oligonucleotide
  source 1..19: mol_type = other DNA
cctaactagagacctgttt

SEQ ID NO: 391 - DNA, 19 bp, synthetic construct
  misc_feature 1..19: note = Synthetic oligonucleotide
  source 1..19: mol_type = other DNA
ccctaactagagacctgtt

SEQ ID NO: 392 - DNA, 19 bp, synthetic construct
  misc_feature 1..19: note = Synthetic oligonucleotide
  source 1..19: mol_type = other DNA
accctaactagagacctgt

SEQ ID NO: 393 - DNA, 19 bp, synthetic construct
  misc_feature 1..19: note = Synthetic oligonucleotide
  source 1..19: mol_type = other DNA
caccctaactagagacctg

SEQ ID NO: 394 - DNA, 19 bp, synthetic construct
  misc_feature 1..19: note = Synthetic oligonucleotide
  source 1..19: mol_type = other DNA
tcaccctaactagagacct

SEQ ID NO: 395 - DNA, 19 bp, synthetic construct
  misc_feature 1..19: note = Synthetic oligonucleotide
  source 1..19: mol_type = other DNA
atcaccctaactagagacc

SEQ ID NO: 396 - DNA, 19 bp, synthetic construct
  misc_feature 1..19: note = Synthetic oligonucleotide
  source 1..19: mol_type = other DNA
tatcaccctaactagagac

SEQ ID NO: 397 - DNA, 19 bp, synthetic construct
  misc_feature 1..19: note = Synthetic oligonucleotide
  source 1..19: mol_type = other DNA
atatcaccctaactagaga

SEQ ID NO: 398 - DNA, 19 bp, synthetic construct
  misc_feature 1..19: note = Synthetic oligonucleotide
  source 1..19: mol_type = other DNA
aatatcaccctaactagag

SEQ ID NO: 399 - DNA, 19 bp, synthetic construct
  misc_feature 1..19: note = Synthetic oligonucleotide
  source 1..19: mol_type = other DNA
actactgagtgacagtaga

SEQ ID NO: 400 - RNA, 21 nt, synthetic construct
  misc_feature 1..21: note = Synthetic oligonucleotide
  source 1..21: mol_type = other RNA
ggttcgtgtctgcttctccaa

SEQ ID NO: 401 - RNA, 21 nt, synthetic construct
  misc_feature 1..21: note = Synthetic oligonucleotide
  source 1..21: mol_type = other RNA
cggttcgtgtctgcttctcca

SEQ ID NO: 402 - RNA, 21 nt, synthetic construct
  misc_feature 1..21: note = Synthetic oligonucleotide
  source 1..21: mol_type = other RNA
tcggttcgtgtctgcttctcc

SEQ ID NO: 403 - RNA, 21 nt, synthetic construct
  misc_feature 1..21: note = Synthetic oligonucleotide
  source 1..21: mol_type = other RNA
ttcggttcgtgtctgcttctc

SEQ ID NO: 404 - RNA, 21 nt, synthetic construct
  misc_feature 1..21: note = Synthetic oligonucleotide
  source 1..21: mol_type = other RNA
tttcggttcgtgtctgcttct

SEQ ID NO: 405 - RNA, 21 nt, synthetic construct
  misc_feature 1..21: note = Synthetic oligonucleotide
  source 1..21: mol_type = other RNA
atttcggttcgtgtctgcttc

SEQ ID NO: 406 - RNA, 21 nt, synthetic construct
  misc_feature 1..21: note = Synthetic oligonucleotide
  source 1..21: mol_type = other RNA
aatttcggttcgtgtctgctt

SEQ ID NO: 407 - RNA, 21 nt, synthetic construct
  misc_feature 1..21: note = Synthetic oligonucleotide
  source 1..21: mol_type = other RNA
aaatttcggttcgtgtctgct

SEQ ID NO: 408 - RNA, 21 nt, synthetic construct
  misc_feature 1..21: note = Synthetic oligonucleotide
  source 1..21: mol_type = other RNA
aaaatttcggttcgtgtctgc

SEQ ID NO: 409 - RNA, 21 nt, synthetic construct
  misc_feature 1..21: note = Synthetic oligonucleotide
  source 1..21: mol_type = other RNA
taaaatttcggttcgtgtctg

SEQ ID NO: 410 - RNA, 21 nt, synthetic construct
  misc_feature 1..21: note = Synthetic oligonucleotide
  source 1..21: mol_type = other RNA
ggtgtcaatttctctttcttt

SEQ ID NO: 411 - RNA, 21 nt, synthetic construct
  misc_feature 1..21: note = Synthetic oligonucleotide
  source 1..21: mol_type = other RNA
gggtgtcaatttctctttctt

SEQ ID NO: 412 - RNA, 21 nt, synthetic construct
  misc_feature 1..21: note = Synthetic oligonucleotide
  source 1..21: mol_type = other RNA
cgggtgtcaatttctctttct

SEQ ID NO: 413 - RNA, 21 nt, synthetic construct
  misc_feature 1..21: note = Synthetic oligonucleotide
  source 1..21: mol_type = other RNA
tcgggtgtcaatttctctttc

SEQ ID NO: 414 - RNA, 21 nt, synthetic construct
  misc_feature 1..21: note = Synthetic oligonucleotide
  source 1..21: mol_type = other RNA
gtcgggtgtcaatttctcttt

SEQ ID NO: 415 - RNA, 21 nt, synthetic construct
  misc_feature 1..21: note = Synthetic oligonucleotide
  source 1..21: mol_type = other RNA
agtcgggtgtcaatttctctt

SEQ ID NO: 416 - RNA, 21 nt, synthetic construct
  misc_feature 1..21: note = Synthetic oligonucleotide
  source 1..21: mol_type = other RNA
cagtcgggtgtcaatttctct

SEQ ID NO: 417 - RNA, 21 nt, synthetic construct
  misc_feature 1..21: note = Synthetic oligonucleotide
  source 1..21: mol_type = other RNA
tcagtcgggtgtcaatttctc

SEQ ID NO: 418 - RNA, 21 nt, synthetic construct
  misc_feature 1..21: note = Synthetic oligonucleotide
  source 1..21: mol_type = other RNA
ttcagtcgggtgtcaatttct

SEQ ID NO: 419 - RNA, 21 nt, synthetic construct
  misc_feature 1..21: note = Synthetic oligonucleotide
  source 1..21: mol_type = other RNA
tttcagtcgggtgtcaatttc

SEQ ID NO: 420 - RNA, 21 nt, synthetic construct
  misc_feature 1..21: note = Synthetic oligonucleotide
  source 1..21: mol_type = other RNA
ttttcagtcgggtgtcaattt

SEQ ID NO: 421 - RNA, 21 nt, synthetic construct
  misc_feature 1..21: note = Synthetic oligonucleotide
  source 1..21: mol_type = other RNA
gttttcagtcgggtgtcaatt

SEQ ID NO: 422 - RNA, 21 nt, synthetic construct
  misc_feature 1..21: note = Synthetic oligonucleotide
  source 1..21: mol_type = other RNA
cgttttcagtcgggtgtcaat

SEQ ID NO: 423 - RNA, 21 nt, synthetic construct
  misc_feature 1..21: note = Synthetic oligonucleotide
  source 1..21: mol_type = other RNA
gcgttttcagtcgggtgtcaa

SEQ ID NO: 424 - RNA, 21 nt, synthetic construct
  misc_feature 1..21: note = Synthetic oligonucleotide
  source 1..21: mol_type = other RNA
ggcgttttcagtcgggtgtca

SEQ ID NO: 425 - RNA, 21 nt, synthetic construct
  misc_feature 1..21: note = Synthetic oligonucleotide
  source 1..21: mol_type = other RNA
gggcgttttcagtcgggtgtc

SEQ ID NO: 426 - RNA, 21 nt, synthetic construct
  misc_feature 1..21: note = Synthetic oligonucleotide
  source 1..21: mol_type = other RNA
ggggcgttttcagtcgggtgt

SEQ ID NO: 427 - RNA, 21 nt, synthetic construct
  misc_feature 1..21: note = Synthetic oligonucleotide
  source 1..21: mol_type = other RNA
aggggcgttttcagtcgggtg

SEQ ID NO: 428 - RNA, 21 nt, synthetic construct
  misc_feature 1..21: note = Synthetic oligonucleotide
  source 1..21: mol_type = other RNA
aaggggcgttttcagtcgggt

SEQ ID NO: 429 - RNA, 21 nt, synthetic construct
  misc_feature 1..21: note = Synthetic oligonucleotide
  source 1..21: mol_type = other RNA
taaggggcgttttcagtcggg

SEQ ID NO: 430 - RNA, 21 nt, synthetic construct
  misc_feature 1..21: note = Synthetic oligonucleotide
  source 1..21: mol_type = other RNA
gtaaggggcgttttcagtcgg

SEQ ID NO: 431 - RNA, 21 nt, synthetic construct
  misc_feature 1..21: note = Synthetic oligonucleotide
  source 1..21: mol_type = other RNA
agtaaggggcgttttcagtcg

SEQ ID NO: 432 - RNA, 21 nt, synthetic construct
  misc_feature 1..21: note = Synthetic oligonucleotide
  source 1..21: mol_type = other RNA
aagtaaggggcgttttcagtc

SEQ ID NO: 433 - RNA, 21 nt, synthetic construct
  misc_feature 1..21: note = Synthetic oligonucleotide
  source 1..21: mol_type = other RNA
caagtaaggggcgttttcagt

SEQ ID NO: 434 - RNA, 21 nt, synthetic construct
  misc_feature 1..21: note = Synthetic oligonucleotide
  source 1..21: mol_type = other RNA
acaagtaaggggcgttttcag

SEQ ID NO: 435 - RNA, 21 nt, synthetic construct
  misc_feature 1..21: note = Synthetic oligonucleotide
  source 1..21: mol_type = other RNA
cacaagtaaggggcgttttca

SEQ ID NO: 436 - RNA, 21 nt, synthetic construct
  misc_feature 1..21: note = Synthetic oligonucleotide
  source 1..21: mol_type = other RNA
gcacaagtaaggggcgttttc

SEQ ID NO: 437 - RNA, 21 nt, synthetic construct
  misc_feature 1..21: note = Synthetic oligonucleotide
  source 1..21: mol_type = other RNA
tgcacaagtaaggggcgtttt

SEQ ID NO: 438 - RNA, 21 nt, synthetic construct
  misc_feature 1..21: note = Synthetic oligonucleotide
  source 1..21: mol_type = other RNA
ttgcacaagtaaggggcgttt

SEQ ID NO: 439 - RNA, 21 nt, synthetic construct
  misc_feature 1..21: note = Synthetic oligonucleotide
  source 1..21: mol_type = other RNA
attgcacaagtaaggggcgtt

SEQ ID NO: 440 - RNA, 21 nt, synthetic construct
  misc_feature 1..21: note = Synthetic oligonucleotide
  source 1..21: mol_type = other RNA
gattgcacaagtaaggggcgt

SEQ ID NO: 441 - RNA, 21 nt, synthetic construct
  misc_feature 1..21: note = Synthetic oligonucleotide
  source 1..21: mol_type = other RNA
agattgcacaagtaaggggcg

SEQ ID NO: 442 - RNA, 21 nt, synthetic construct
  misc_feature 1..21: note = Synthetic oligonucleotide
  source 1..21: mol_type = other RNA
aagattgcacaagtaaggggc

SEQ ID NO: 443 - RNA, 21 nt, synthetic construct
  misc_feature 1..21: note = Synthetic oligonucleotide
  source 1..21: mol_type = other RNA
gaagattgcacaagtaagggg

SEQ ID NO: 444 - RNA, 21 nt, synthetic construct
  misc_feature 1..21: note = Synthetic oligonucleotide
  source 1..21: mol_type = other RNA
ggaagattgcacaagtaaggg

SEQ ID NO: 445 - RNA, 21 nt, synthetic construct
  misc_feature 1..21: note = Synthetic oligonucleotide
  source 1..21: mol_type = other RNA
aggaagattgcacaagtaagg

SEQ ID NO: 446 - RNA, 21 nt, synthetic construct
  misc_feature 1..21: note = Synthetic oligonucleotide
  source 1..21: mol_type = other RNA
caggaagattgcacaagtaag

SEQ ID NO: 447 - RNA, 21 nt, synthetic construct
  misc_feature 1..21: note = Synthetic oligonucleotide
  source 1..21: mol_type = other RNA
gcaggaagattgcacaagtaa

SEQ ID NO: 448 - RNA, 21 nt, synthetic construct
  misc_feature 1..21: note = Synthetic oligonucleotide
  source 1..21: mol_type = other RNA
ggcaggaagattgcacaagta

SEQ ID NO: 449 - RNA, 21 nt, synthetic construct
  misc_feature 1..21: note = Synthetic oligonucleotide
  source 1..21: mol_type = other RNA
aggcaggaagattgcacaagt

SEQ ID NO: 450 - RNA, 21 nt, synthetic construct
  misc_feature 1..21: note = Synthetic oligonucleotide
  source 1..21: mol_type = other RNA
caggcaggaagattgcacaag

SEQ ID NO: 451 - RNA, 21 nt, synthetic construct
  misc_feature 1..21: note = Synthetic oligonucleotide
  source 1..21: mol_type = other RNA
ccaggcaggaagattgcacaa

SEQ ID NO: 452 - RNA, 21 nt, synthetic construct
  misc_feature 1..21: note = Synthetic oligonucleotide
  source 1..21: mol_type = other RNA
accaggcaggaagattgcaca

SEQ ID NO: 453 - RNA, 21 nt, synthetic construct
  misc_feature 1..21: note = Synthetic oligonucleotide
  source 1..21: mol_type = other RNA
taccaggcaggaagattgcac

SEQ ID NO: 454 - RNA, 21 nt, synthetic construct
  misc_feature 1..21: note = Synthetic oligonucleotide
  source 1..21: mol_type = other RNA
ctaccaggcaggaagattgca

SEQ ID NO: 455 - RNA, 21 nt, synthetic construct
  misc_feature 1..21: note = Synthetic oligonucleotide
  source 1..21: mol_type = other RNA
actaccaggcaggaagattgc

SEQ ID NO: 456 - RNA, 21 nt, synthetic construct
  misc_feature 1..21: note = Synthetic oligonucleotide
  source 1..21: mol_type = other RNA
aactaccaggcaggaagattg

SEQ ID NO: 457 - RNA, 21 nt, synthetic construct
  misc_feature 1..21: note = Synthetic oligonucleotide
  source 1..21: mol_type = other RNA
gaactaccaggcaggaagatt

SEQ ID NO: 458 - RNA, 21 nt, synthetic construct
  misc_feature 1..21: note = Synthetic oligonucleotide
  source 1..21: mol_type = other RNA
ggaactaccaggcaggaagat

SEQ ID NO: 459 - RNA, 21 nt, synthetic construct
  misc_feature 1..21: note = Synthetic oligonucleotide
  source 1..21: mol_type = other RNA
cggaactaccaggcaggaaga

SEQ ID NO: 460 - RNA, 21 nt, synthetic construct
  misc_feature 1..21: note = Synthetic oligonucleotide
  source 1..21: mol_type = other RNA
gcggaactaccaggcaggaag

SEQ ID NO: 461 - RNA, 21 nt, synthetic construct
  misc_feature 1..21: note = Synthetic oligonucleotide
  source 1..21: mol_type = other RNA
ggcggaactaccaggcaggaa

SEQ ID NO: 462 - RNA, 21 nt, synthetic construct
  misc_feature 1..21: note = Synthetic oligonucleotide
  source 1..21: mol_type = other RNA
tggcggaactaccaggcagga

SEQ ID NO: 463 - RNA, 21 nt, synthetic construct
  misc_feature 1..21: note = Synthetic oligonucleotide
  source 1..21: mol_type = other RNA
ttggcggaactaccaggcagg

SEQ ID NO: 464 - RNA, 21 nt, synthetic construct
  misc_feature 1..21: note = Synthetic oligonucleotide
  source 1..21: mol_type = other RNA
tttggcggaactaccaggcag

SEQ ID NO: 465 - RNA, 21 nt, synthetic construct
  misc_feature 1..21: note = Synthetic oligonucleotide
  source 1..21: mol_type = other RNA
ttttggcggaactaccaggca

SEQ ID NO: 466 - RNA, 21 nt, synthetic construct
  misc_feature 1..21: note = Synthetic oligonucleotide
  source 1..21: mol_type = other RNA
cgctttgactgttttgttttt

SEQ ID NO: 467 - RNA, 21 nt, synthetic construct
  misc_feature 1..21: note = Synthetic oligonucleotide
  source 1..21: mol_type = other RNA
gcgctttgactgttttgtttt

SEQ ID NO: 468 - RNA, 21 nt, synthetic construct
  misc_feature 1..21: note = Synthetic oligonucleotide
  source 1..21: mol_type = other RNA
tgcgctttgactgttttgttt

SEQ ID NO: 469 - RNA, 21 nt, synthetic construct
  misc_feature 1..21: note = Synthetic oligonucleotide
  source 1..21: mol_type = other RNA
ttgcgctttgactgttttgtt

SEQ ID NO: 470 - RNA, 21 nt, synthetic construct
  misc_feature 1..21: note = Synthetic oligonucleotide
  source 1..21: mol_type = other RNA
tttgcgctttgactgttttgt

SEQ ID NO: 471 - RNA, 21 nt, synthetic construct
  misc_feature 1..21: note = Synthetic oligonucleotide
  source 1..21: mol_type = other RNA
gtttgcgctttgactgttttg

SEQ ID NO: 472 - RNA, 21 nt, synthetic construct
  misc_feature 1..21: note = Synthetic oligonucleotide
  source 1..21: mol_type = other RNA
ggtttgcgctttgactgtttt

SEQ ID NO: 473 - RNA, 21 nt, synthetic construct
  misc_feature 1..21: note = Synthetic oligonucleotide
  source 1..21: mol_type = other RNA
aggtttgcgctttgactgttt

SEQ ID NO: 474 - RNA, 21 nt, synthetic construct
  misc_feature 1..21: note = Synthetic oligonucleotide
  source 1..21: mol_type = other RNA
caggtttgcgctttgactgtt

SEQ ID NO: 475 - RNA, 21 nt, synthetic construct
  misc_feature 1..21: note = Synthetic oligonucleotide
  source 1..21: mol_type = other RNA
ccaggtttgcgctttgactgt

SEQ ID NO: 476 - RNA, 21 nt, synthetic construct
  misc_feature 1..21: note = Synthetic oligonucleotide
  source 1..21: mol_type = other RNA
tccaggtttgcgctttgactg

SEQ ID NO: 477 - RNA, 21 nt, synthetic construct
  misc_feature 1..21: note = Synthetic oligonucleotide
  source 1..21: mol_type = other RNA
ttccaggtttgcgctttgact

SEQ ID NO: 478 - RNA, 21 nt, synthetic construct
  misc_feature 1..21: note = Synthetic oligonucleotide
  source 1..21: mol_type = other RNA
cttccaggtttgcgctttgac

SEQ ID NO: 479 - RNA, 21 nt, synthetic construct
  misc_feature 1..21: note = Synthetic oligonucleotide
  source 1..21: mol_type = other RNA
ccttccaggtttgcgctttga

SEQ ID NO: 480 - RNA, 21 nt, synthetic construct
  misc_feature 1..21: note = Synthetic oligonucleotide
  source 1..21: mol_type = other RNA
cccttccaggtttgcgctttg

SEQ ID NO: 481 - RNA, 21 nt, synthetic construct
  misc_feature 1..21: note = Synthetic oligonucleotide
  source 1..21: mol_type = other RNA
ccccttccaggtttgcgcttt

SEQ ID NO: 482 - RNA, 21 nt, synthetic construct
  misc_feature 1..21: note = Synthetic oligonucleotide
  source 1..21: mol_type = other RNA
gccccttccaggtttgcgctt

SEQ ID NO: 483 - RNA, 21 nt, synthetic construct
  misc_feature 1..21: note = Synthetic oligonucleotide
  source 1..21: mol_type = other RNA
cgccccttccaggtttgcgct

SEQ ID NO: 484 - RNA, 21 nt, synthetic construct
  misc_feature 1..21: note = Synthetic oligonucleotide
  source 1..21: mol_type = other RNA
gcgccccttccaggtttgcgc

SEQ ID NO: 485 - RNA, 21 nt, synthetic construct
  misc_feature 1..21: note = Synthetic oligonucleotide
  source 1..21: mol_type = other RNA
cgcgccccttccaggtttgcg

SEQ ID NO: 486 - RNA, 21 nt, synthetic construct
  misc_feature 1..21: note = Synthetic oligonucleotide
  source 1..21: mol_type = other RNA
caaagttggtgcctgcgcgcc

SEQ ID NO: 487 - RNA, 21 nt, synthetic construct
  misc_feature 1..21: note = Synthetic oligonucleotide
  source 1..21: mol_type = other RNA
gcaaagttggtgcctgcgcgc

SEQ ID NO: 488 - RNA, 21 nt, synthetic construct
  misc_feature 1..21: note = Synthetic oligonucleotide
  source 1..21: mol_type = other RNA
ggcaaagttggtgcctgcgcg

SEQ ID NO: 489 - RNA, 21 nt, synthetic construct
  misc_feature 1..21: note = Synthetic oligonucleotide
  source 1..21: mol_type = other RNA
tggcaaagttggtgcctgcgc

SEQ ID NO: 490 - RNA, 21 nt, synthetic construct
  misc_feature 1..21: note = Synthetic oligonucleotide
  source 1..21: mol_type = other RNA
ttggcaaagttggtgcctgcg

SEQ ID NO: 491 - RNA, 21 nt, synthetic construct
  misc_feature 1..21: note = Synthetic oligonucleotide
  source 1..21: mol_type = other RNA
tttggcaaagttggtgcctgc

SEQ ID NO: 492 - RNA, 21 nt, synthetic construct
  misc_feature 1..21: note = Synthetic oligonucleotide
  source 1..21: mol_type = other RNA
gtttggcaaagttggtgcctg

SEQ ID NO: 493 - RNA, 21 nt, synthetic construct
  misc_feature 1..21: note = Synthetic oligonucleotide
  source 1..21: mol_type = other RNA
cgtttggcaaagttggtgcct

SEQ ID NO: 494 - RNA, 21 nt, synthetic construct
  misc_feature 1..21: note = Synthetic oligonucleotide
  source 1..21: mol_type = other RNA
gcgtttggcaaagttggtgcc

SEQ ID NO: 495 - RNA, 21 nt, synthetic construct
  misc_feature 1..21: note = Synthetic oligonucleotide
  source 1..21: mol_type = other RNA
agcgtttggcaaagttggtgc

SEQ ID NO: 496 - RNA, 21 nt, synthetic construct
  misc_feature 1..21: note = Synthetic oligonucleotide
  source 1..21: mol_type = other RNA
tagcgtttggcaaagttggtg

SEQ ID NO: 497 - RNA, 21 nt, synthetic construct
  misc_feature 1..21: note = Synthetic oligonucleotide
  source 1..21: mol_type = other RNA
gtagcgtttggcaaagttggt

SEQ ID NO: 498 - RNA, 21 nt, synthetic construct
  misc_feature 1..21: note = Synthetic oligonucleotide
  source 1..21: mol_type = other RNA
tgtagcgtttggcaaagttgg

SEQ ID NO: 499 - RNA, 21 nt, synthetic construct
  misc_feature 1..21: note = Synthetic oligonucleotide
  source 1..21: mol_type = other RNA
ctgtagcgtttggcaaagttg

SEQ ID NO: 500 - RNA, 21 nt, synthetic construct
  misc_feature 1..21: note = Synthetic oligonucleotide
  source 1..21: mol_type = other RNA
tctgtagcgtttggcaaagtt

SEQ ID NO: 501 - RNA, 21 nt, synthetic construct
  misc_feature 1..21: note = Synthetic oligonucleotide
  source 1..21: mol_type = other RNA
ctctgtagcgtttggcaaagt

SEQ ID NO: 502 - RNA, 21 nt, synthetic construct
  misc_feature 1..21: note = Synthetic oligonucleotide
  source 1..21: mol_type = other RNA
actctgtagcgtttggcaaag

SEQ ID NO: 503 - RNA, 21 nt, synthetic construct
  misc_feature 1..21: note = Synthetic oligonucleotide
  source 1..21: mol_type = other RNA
aactctgtagcgtttggcaaa

SEQ ID NO: 504 - RNA, 21 nt, synthetic construct
  misc_feature 1..21: note = Synthetic oligonucleotide
  source 1..21: mol_type = other RNA
aaactctgtagcgtttggcaa

SEQ ID NO: 505 - RNA, 21 nt, synthetic construct
  misc_feature 1..21: note = Synthetic oligonucleotide
  source 1..21: mol_type = other RNA
aaaactctgtagcgtttggca

SEQ ID NO: 506 - RNA, 21 nt, synthetic construct
  misc_feature 1..21: note = Synthetic oligonucleotide
  source 1..21: mol_type = other RNA
gaaaactctgtagcgtttggc

SEQ ID NO: 507 - RNA, 21 nt, synthetic construct
  misc_feature 1..21: note = Synthetic oligonucleotide
  source 1..21: mol_type = other RNA
ggaaaactctgtagcgtttgg

SEQ ID NO: 508 - RNA, 21 nt, synthetic construct
  misc_feature 1..21: note = Synthetic oligonucleotide
  source 1..21: mol_type = other RNA
cggaaaactctgtagcgtttg

SEQ ID NO: 509 - RNA, 21 nt, synthetic construct
  misc_feature 1..21: note = Synthetic oligonucleotide
  source 1..21: mol_type = other RNA
acggaaaactctgtagcgttt

SEQ ID NO: 510 - RNA, 21 nt, synthetic construct
  misc_feature 1..21: note = Synthetic oligonucleotide
  source 1..21: mol_type = other RNA
aacggaaaactctgtagcgtt

SEQ ID NO: 511 - RNA, 21 nt, synthetic construct
  misc_feature 1..21: note = Synthetic oligonucleotide
  source 1..21: mol_type = other RNA
aaacggaaaactctgtagcgt

SEQ ID NO: 512 - RNA, 21 nt, synthetic construct
  misc_feature 1..21: note = Synthetic oligonucleotide
  source 1..21: mol_type = other RNA
caaacggaaaactctgtagcg

SEQ ID NO: 513 - RNA, 21 nt, synthetic construct
  misc_feature 1..21: note = Synthetic oligonucleotide
  source 1..21: mol_type = other RNA
gcaaacggaaaactctgtagc

SEQ ID NO: 514 - RNA, 21 nt, synthetic construct
  misc_feature 1..21: note = Synthetic oligonucleotide
  source 1..21: mol_type = other RNA
tgcaaacggaaaactctgtag

SEQ ID NO: 515 - RNA, 21 nt, synthetic construct
  misc_feature 1..21: note = Synthetic oligonucleotide
  source 1..21: mol_type = other RNA
ctgcaaacggaaaactctgta

SEQ ID NO: 516 - RNA, 21 nt, synthetic construct
  misc_feature 1..21: note = Synthetic oligonucleotide
  source 1..21: mol_type = other RNA
tctgcaaacggaaaactctgt

SEQ ID NO: 517 - RNA, 21 nt, synthetic construct
  misc_feature 1..21: note = Synthetic oligonucleotide
  source 1..21: mol_type = other RNA
ttctgcaaacggaaaactctg

SEQ ID NO: 518 - RNA, 21 nt, synthetic construct
  misc_feature 1..21: note = Synthetic oligonucleotide
  source 1..21: mol_type = other RNA
tttctgcaaacggaaaactct

SEQ ID NO: 519 - RNA, 21 nt, synthetic construct
  misc_feature 1..21: note = Synthetic oligonucleotide
  source 1..21: mol_type = other RNA
atttctgcaaacggaaaactc

SEQ ID NO: 520 - RNA, 21 nt, synthetic construct
  misc_feature 1..21: note = Synthetic oligonucleotide
  source 1..21: mol_type = other RNA
catttctgcaaacggaaaact

SEQ ID NO: 521 - RNA, 21 nt, synthetic construct
  misc_feature 1..21: note = Synthetic oligonucleotide
  source 1..21: mol_type = other RNA
tcatttctgcaaacggaaaac

SEQ ID NO: 522 - RNA, 21 nt, synthetic construct
  misc_feature 1..21: note = Synthetic oligonucleotide
  source 1..21: mol_type = other RNA
gtcatttctgcaaacggaaaa

SEQ ID NO: 523 - RNA, 21 nt, synthetic construct
  misc_feature 1..21: note = Synthetic oligonucleotide
  source 1..21: mol_type = other RNA
ggtcatttctgcaaacggaaa

SEQ ID NO: 524 - RNA, 21 nt, synthetic construct
  misc_feature 1..21: note = Synthetic oligonucleotide
  source 1..21: mol_type = other RNA
gggtcatttctgcaaacggaa

SEQ ID NO: 525 - RNA, 21 nt, synthetic construct
  misc_feature 1..21: note = Synthetic oligonucleotide
  source 1..21: mol_type = other RNA
tgggtcatttctgcaaacgga

SEQ ID NO: 526 - RNA, 21 nt, synthetic construct
  misc_feature 1..21: note = Synthetic oligonucleotide
  source 1..21: mol_type = other RNA
ttgggtcatttctgcaaacgg

SEQ ID NO: 527 - RNA, 21 nt, synthetic construct
  misc_feature 1..21: note = Synthetic oligonucleotide
  source 1..21: mol_type = other RNA
tttgggtcatttctgcaaacg

SEQ ID NO: 528 - RNA, 21 nt, synthetic construct
  misc_feature 1..21: note = Synthetic oligonucleotide
  source 1..21: mol_type = other RNA
ttttgggtcatttctgcaaac

SEQ ID NO: 529 - RNA, 21 nt, synthetic construct
  misc_feature 1..21: note = Synthetic oligonucleotide
  source 1..21: mol_type = other RNA
cttttgggtcatttctgcaaa

SEQ ID NO: 530 - RNA, 21 nt, synthetic construct
  misc_feature 1..21: note = Synthetic oligonucleotide
  source 1..21: mol_type = other RNA
tcttttgggtcatttctgcaa

SEQ ID NO: 531 - RNA, 21 nt, synthetic construct
  misc_feature 1..21: note = Synthetic oligonucleotide
  source 1..21: mol_type = other RNA
ttcttttgggtcatttctgca

SEQ ID NO: 532 - RNA, 21 nt, synthetic construct
  misc_feature 1..21: note = Synthetic oligonucleotide
  source 1..21: mol_type = other RNA
cttcttttgggtcatttctgc

SEQ ID NO: 533 - RNA, 21 nt, synthetic construct
  misc_feature 1..21: note = Synthetic oligonucleotide
  source 1..21: mol_type = other RNA
ccttcttttgggtcatttctg

SEQ ID NO: 534 - RNA, 21 nt, synthetic construct
  misc_feature 1..21: note = Synthetic oligonucleotide
  source 1..21: mol_type = other RNA
cccttcttttgggtcatttct

SEQ ID NO: 535 - RNA, 21 nt, synthetic construct
  misc_feature 1..21: note = Synthetic oligonucleotide
  source 1..21: mol_type = other RNA
ccccttcttttgggtcatttc

SEQ ID NO: 536 - RNA, 21 nt, synthetic construct
  misc_feature 1..21: note = Synthetic oligonucleotide
  source 1..21: mol_type = other RNA
tggggaatcacggggagcggc

SEQ ID NO: 537 - RNA, 21 nt, synthetic construct
  misc_feature 1..21: note = Synthetic oligonucleotide
  source 1..21: mol_type = other RNA
ttggggaatcacggggagcgg

SEQ ID NO: 538 - RNA, 21 nt, synthetic construct
  misc_feature 1..21: note = Synthetic oligonucleotide
  source 1..21: mol_type = other RNA
attggggaatcacggggagcg

SEQ ID NO: 539 - RNA, 21 nt, synthetic construct
  misc_feature 1..21: note = Synthetic oligonucleotide
  source 1..21: mol_type = other RNA
gattggggaatcacggggagc

SEQ ID NO: 540 - RNA, 21 nt, synthetic construct
  misc_feature 1..21: note = Synthetic oligonucleotide
  source 1..21: mol_type = other RNA
cgattggggaatcacggggag

SEQ ID NO: 541 - RNA, 21 nt, synthetic construct
  misc_feature 1..21: note = Synthetic oligonucleotide
  source 1..21: mol_type = other RNA
ccgattggggaatcacgggga

SEQ ID NO: 542 - RNA, 21 nt, synthetic construct
  misc_feature 1..21: note = Synthetic oligonucleotide
  source 1..21: mol_type = other RNA
gccgattggggaatcacgggg

SEQ ID NO: 543 - RNA, 21 nt, synthetic construct
  misc_feature 1..21: note = Synthetic oligonucleotide
  source 1..21: mol_type = other RNA
agccgattggggaatcacggg

SEQ ID NO: 544 - RNA, 21 nt, synthetic construct
  misc_feature 1..21: note = Synthetic oligonucleotide
  source 1..21: mol_type = other RNA
aagccgattggggaatcacgg

SEQ ID NO: 545 - RNA, 21 nt, synthetic construct
  misc_feature 1..21: note = Synthetic oligonucleotide
  source 1..21: mol_type = other RNA
gaagccgattggggaatcacg

SEQ ID NO: 546 - RNA, 21 nt, synthetic construct
  misc_feature 1..21: note = Synthetic oligonucleotide
  source 1..21: mol_type = other RNA
agaagccgattggggaatcac

SEQ ID NO: 547 - RNA, 21 nt, synthetic construct
  misc_feature 1..21: note = Synthetic oligonucleotide
  source 1..21: mol_type = other RNA
cagaagccgattggggaatca

SEQ ID NO: 548 - RNA, 21 nt, synthetic construct
  misc_feature 1..21: note = Synthetic oligonucleotide
  source 1..21: mol_type = other RNA
ccagaagccgattggggaatc

SEQ ID NO: 549 - RNA, 21 nt, synthetic construct
  misc_feature 1..21: note = Synthetic oligonucleotide
  source 1..21: mol_type = other RNA
tccagaagccgattggggaat

SEQ ID NO: 550 - RNA, 21 nt, synthetic construct
  misc_feature 1..21: note = Synthetic oligonucleotide
  source 1..21: mol_type = other RNA
ctccagaagccgattggggaa

SEQ ID NO: 551 - RNA, 21 nt, synthetic construct
  misc_feature 1..21: note = Synthetic oligonucleotide
  source 1..21: mol_type = other RNA
gctccagaagccgattgggga

SEQ ID NO: 552 - RNA, 21 nt, synthetic construct
  misc_feature 1..21: note = Synthetic oligonucleotide
  source 1..21: mol_type = other RNA
ggctccagaagccgattgggg

SEQ ID NO: 553 - RNA, 21 nt, synthetic construct
  misc_feature 1..21: note = Synthetic oligonucleotide
  source 1..21: mol_type = other RNA
tggctccagaagccgattggg

SEQ ID NO: 554 - RNA, 21 nt, synthetic construct
  misc_feature 1..21: note = Synthetic oligonucleotide
  source 1..21: mol_type = other RNA
ctggctccagaagccgattgg

SEQ ID NO: 555 - RNA, 21 nt, synthetic construct
  misc_feature 1..21: note = Synthetic oligonucleotide
  source 1..21: mol_type = other RNA
tctggctccagaagccgattg

SEQ ID NO: 556 - RNA, 21 nt, synthetic construct
  misc_feature 1..21: note = Synthetic oligonucleotide
  source 1..21: mol_type = other RNA
ctctggctccagaagccgatt

SEQ ID NO: 557 - RNA, 21 nt, synthetic construct
  misc_feature 1..21: note = Synthetic oligonucleotide
  source 1..21: mol_type = other RNA
cctctggctccagaagccgat

SEQ ID NO: 558 - RNA, 21 nt, synthetic construct
  misc_feature 1..21: note = Synthetic oligonucleotide
  source 1..21: mol_type = other RNA
gcctctggctccagaagccga

SEQ ID NO: 559 - RNA, 21 nt, synthetic construct
  misc_feature 1..21: note = Synthetic oligonucleotide
  source 1..21: mol_type = other RNA
agcctctggctccagaagccg

SEQ ID NO: 560 - RNA, 21 nt, synthetic construct
  misc_feature 1..21: note = Synthetic oligonucleotide
  source 1..21: mol_type = other RNA
gagcctctggctccagaagcc

SEQ ID NO: 561 - RNA, 21 nt, synthetic construct
  misc_feature 1..21: note = Synthetic oligonucleotide
  source 1..21: mol_type = other RNA
ggagcctctggctccagaagc

SEQ ID NO: 562 - RNA, 21 nt, synthetic construct
  misc_feature 1..21: note = Synthetic oligonucleotide
  source 1..21: mol_type = other RNA
aggagcctctggctccagaag

SEQ ID NO: 563 - RNA, 21 nt, synthetic construct
  misc_feature 1..21: note = Synthetic oligonucleotide
  source 1..21: mol_type = other RNA
gaggagcctctggctccagaa

SEQ ID NO: 564 - RNA, 21 nt, synthetic construct
  misc_feature 1..21: note = Synthetic oligonucleotide
  source 1..21: mol_type = other RNA
ggaggagcctctggctccaga

SEQ ID NO: 565 - RNA, 21 nt, synthetic construct
  misc_feature 1..21: note = Synthetic oligonucleotide
  source 1..21: mol_type = other RNA
aggaggagcctctggctccag

SEQ ID NO: 566 - RNA, 21 nt, synthetic construct
  misc_feature 1..21: note = Synthetic oligonucleotide
  source 1..21: mol_type = other RNA
agcagggaagcgggcagcagc

SEQ ID NO: 567 - RNA, 21 nt, synthetic construct
  misc_feature 1..21: note = Synthetic oligonucleotide
  source 1..21: mol_type = other RNA
tcctcggagcagggaagcggg

SEQ ID NO: 568 - RNA, 21 nt, synthetic construct
  misc_feature 1..21: note = Synthetic oligonucleotide
  source 1..21: mol_type = other RNA
ttcctcggagcagggaagcgg

SEQ ID NO: 569 - RNA, 21 nt, synthetic construct
  misc_feature 1..21: note = Synthetic oligonucleotide
  source 1..21: mol_type = other RNA
tttcctcggagcagggaagcg

SEQ ID NO: 570 - RNA, 21 nt, synthetic construct
  misc_feature 1..21: note = Synthetic oligonucleotide
  source 1..21: mol_type = other RNA
ttttcctcggagcagggaagc

SEQ ID NO: 571 - RNA, 21 nt, synthetic construct
  misc_feature 1..21: note = Synthetic oligonucleotide
  source 1..21: mol_type = other RNA
aaagttgtggagtcgtttttc

SEQ ID NO: 572 - RNA, 21 nt, synthetic construct
  misc_feature 1..21: note = Synthetic oligonucleotide
  source 1..21: mol_type = other RNA
caaagttgtggagtcgttttt

SEQ ID NO: 573 - RNA, 21 nt, synthetic construct
  misc_feature 1..21: note = Synthetic oligonucleotide
  source 1..21: mol_type = other RNA
acaaagttgtggagtcgtttt

SEQ ID NO: 574 - RNA, 21 nt, synthetic construct
  misc_feature 1..21: note = Synthetic oligonucleotide
  source 1..21: mol_type = other RNA
cacaaagttgtggagtcgttt

SEQ ID NO: 575 - RNA, 21 nt, synthetic construct
  misc_feature 1..21: note = Synthetic oligonucleotide
  source 1..21: mol_type = other RNA
gcacaaagttgtggagtcgtt

SEQ ID NO: 576 - RNA, 21 nt, synthetic construct
  misc_feature 1..21: note = Synthetic oligonucleotide
  source 1..21: mol_type = other RNA
cgcacaaagttgtggagtcgt

SEQ ID NO: 577 - RNA, 21 nt, synthetic construct
  misc_feature 1..21: note = Synthetic oligonucleotide
  source 1..21: mol_type = other RNA
gcgcacaaagttgtggagtcg

SEQ ID NO: 578 - RNA, 21 nt, synthetic construct
  misc_feature 1..21: note = Synthetic oligonucleotide
  source 1..21: mol_type = other RNA
cgcgcacaaagttgtggagtc

SEQ ID NO: 579 - RNA, 21 nt, synthetic construct
  misc_feature 1..21: note = Synthetic oligonucleotide
  source 1..21: mol_type = other RNA
tcgcgcacaaagttgtggagt

SEQ ID NO: 580 - RNA, 21 nt, synthetic construct
  misc_feature 1..21: note = Synthetic oligonucleotide
  source 1..21: mol_type = other RNA
ctcgcgcacaaagttgtggag

SEQ ID NO: 581 - RNA, 21 nt, synthetic construct
  misc_feature 1..21: note = Synthetic oligonucleotide
  source 1..21: mol_type = other RNA
tctcgcgcacaaagttgtgga

SEQ ID NO: 582 - RNA, 21 nt, synthetic construct
  misc_feature 1..21: note = Synthetic oligonucleotide
  source 1..21: mol_type = other RNA
ctctcgcgcacaaagttgtgg

SEQ ID NO: 583 - RNA, 21 nt, synthetic construct
  misc_feature 1..21: note = Synthetic oligonucleotide
  source 1..21: mol_type = other RNA
ccgaaggagcgagcctctctc

SEQ ID NO: 584 - RNA, 21 nt, synthetic construct
  misc_feature 1..21: note = Synthetic oligonucleotide
  source 1..21: mol_type = other RNA
gccgaaggagcgagcctctct

SEQ ID NO: 585 - RNA, 21 nt, synthetic construct
  misc_feature 1..21: note = Synthetic oligonucleotide
  source 1..21: mol_type = other RNA
aggaggagccgccgaaggagc

SEQ ID NO: 586 - RNA, 21 nt, synthetic construct
  misc_feature 1..21: note = Synthetic oligonucleotide
  source 1..21: mol_type = other RNA
acaaaggggcaggcccgcagc

SEQ ID NO: 587 - RNA, 21 nt, synthetic construct
  misc_feature 1..21: note = Synthetic oligonucleotide
  source 1..21: mol_type = other RNA
aacaaaggggcaggcccgcag

SEQ ID NO: 588 - RNA, 21 nt, synthetic construct
  misc_feature 1..21: note = Synthetic oligonucleotide
  source 1..21: mol_type = other RNA
caacaaaggggcaggcccgca

SEQ ID NO: 589 - RNA, 21 nt, synthetic construct
  misc_feature 1..21: note = Synthetic oligonucleotide
  source 1..21: mol_type = other RNA
ccaacaaaggggcaggcccgc

SEQ ID NO: 590 - RNA, 21 nt, synthetic construct
  misc_feature 1..21: note = Synthetic oligonucleotide
  source 1..21: mol_type = other RNA
tccaacaaaggggcaggcccg

SEQ ID NO: 591 - RNA, 21 nt, synthetic construct
  misc_feature 1..21: note = Synthetic oligonucleotide
  source 1..21: mol_type = other RNA
ctccaacaaaggggcaggccc

SEQ ID NO: 592 - RNA, 21 nt, synthetic construct
  misc_feature 1..21: note = Synthetic oligonucleotide
  source 1..21: mol_type = other RNA
gctccaacaaaggggcaggcc

SEQ ID NO: 593 - RNA, 21 nt, synthetic construct
  misc_feature 1..21: note = Synthetic oligonucleotide
  source 1..21: mol_type = other RNA
ggctccaacaaaggggcaggc

SEQ ID NO: 594 - RNA, 21 nt, synthetic construct
  misc_feature 1..21: note = Synthetic oligonucleotide
  source 1..21: mol_type = other RNA
cggctccaacaaaggggcagg

SEQ ID NO: 595 - RNA, 21 nt, synthetic construct
  misc_feature 1..21: note = Synthetic oligonucleotide
  source 1..21: mol_type = other RNA
tcggctccaacaaaggggcag

SEQ ID NO: 596 - RNA, 21 nt, synthetic construct
  misc_feature 1..21: note = Synthetic oligonucleotide
  source 1..21: mol_type = other RNA
ctcggctccaacaaaggggca

SEQ ID NO: 597 - RNA, 21 nt, synthetic construct
  misc_feature 1..21: note = Synthetic oligonucleotide
  source 1..21: mol_type = other RNA
gctcggctccaacaaaggggc

SEQ ID NO: 598 - RNA, 21 nt, synthetic construct
  misc_feature 1..21: note = Synthetic oligonucleotide
  source 1..21: mol_type = other RNA
cgctcggctccaacaaagggg

SEQ ID NO: 599 - RNA, 21 nt, synthetic construct
  misc_feature 1..21: note = Synthetic oligonucleotide
  source 1..21: mol_type = other RNA
gcgctcggctccaacaaaggg

SEQ ID NO: 600 - RNA, 21 nt, synthetic construct
  misc_feature 1..21: note = Synthetic oligonucleotide
  source 1..21: mol_type = other RNA
agcgctcggctccaacaaagg

SEQ ID NO: 601 - RNA, 21 nt, synthetic construct
  misc_feature 1..21: note = Synthetic oligonucleotide
  source 1..21: mol_type = other RNA
cagcgctcggctccaacaaag

SEQ ID NO: 602 - RNA, 21 nt, synthetic construct
  misc_feature 1..21: note = Synthetic oligonucleotide
  source 1..21: mol_type = other RNA
ccagcgctcggctccaacaaa

SEQ ID NO: 603 - RNA, 21 nt, synthetic construct
  misc_feature 1..21: note = Synthetic oligonucleotide
  source 1..21: mol_type = other RNA
tccagcgctcggctccaacaa

SEQ ID NO: 604 - RNA, 21 nt, synthetic construct
  misc_feature 1..21: note = Synthetic oligonucleotide
  source 1..21: mol_type = other RNA
ctccagcgctcggctccaaca

SEQ ID NO: 605 - RNA, 21 nt, synthetic construct
  misc_feature 1..21: note = Synthetic oligonucleotide
  source 1..21: mol_type = other RNA
tctggagagcggacccctggc

SEQ ID NO: 606 - RNA, 21 nt, synthetic construct
  misc_feature 1..21: note = Synthetic oligonucleotide
  source 1..21: mol_type = other RNA
atctggagagcggacccctgg

SEQ ID NO: 607 - RNA, 21 nt, synthetic construct
  misc_feature 1..21: note = Synthetic oligonucleotide
  source 1..21: mol_type = other RNA
catctggagagcggacccctg

SEQ ID NO: 608 - RNA, 21 nt, synthetic construct
  misc_feature 1..21: note = Synthetic oligonucleotide
  source 1..21: mol_type = other RNA
tcatctggagagcggacccct

SEQ ID NO: 609 - RNA, 21 nt, synthetic construct
  misc_feature 1..21: note = Synthetic oligonucleotide
  source 1..21: mol_type = other RNA
ctcatctggagagcggacccc

SEQ ID NO: 610 - RNA, 21 nt, synthetic construct
  misc_feature 1..21: note = Synthetic oligonucleotide
  source 1..21: mol_type = other RNA
tctcatctggagagcggaccc

SEQ ID NO: 611 - RNA, 21 nt, synthetic construct
  misc_feature 1..21: note = Synthetic oligonucleotide
  source 1..21: mol_type = other RNA
ttctcatctggagagcggacc

SEQ ID NO: 612 - RNA, 21 nt, synthetic construct
  misc_feature 1..21: note = Synthetic oligonucleotide
  source 1..21: mol_type = other RNA
cttctcatctggagagcggac

SEQ ID NO: 613 - RNA, 21 nt, synthetic construct
  misc_feature 1..21: note = Synthetic oligonucleotide
  source 1..21: mol_type = other RNA
ccttctcatctggagagcgga

SEQ ID NO: 614 - RNA, 21 nt, synthetic construct
  misc_feature 1..21: note = Synthetic oligonucleotide
  source 1..21: mol_type = other RNA
cccttctcatctggagagcgg

SEQ ID NO: 615 - RNA, 21 nt, synthetic construct
  misc_feature 1..21: note = Synthetic oligonucleotide
  source 1..21: mol_type = other RNA
acccttctcatctggagagcg

SEQ ID NO: 616 - RNA, 21 nt, synthetic construct
  misc_feature 1..21: note = Synthetic oligonucleotide
  source 1..21: mol_type = other RNA
cacccttctcatctggagagc

SEQ ID NO: 617 - RNA, 21 nt, synthetic construct
  misc_feature 1..21: note = Synthetic oligonucleotide
  source 1..21: mol_type = other RNA
gcacccttctcatctggagag

SEQ ID NO: 618 - RNA, 21 nt, synthetic construct
  misc_feature 1..21: note = Synthetic oligonucleotide
  source 1..21: mol_type = other RNA
tgcacccttctcatctggaga

SEQ ID NO: 619 - RNA, 21 nt, synthetic construct
  misc_feature 1..21: note = Synthetic oligonucleotide
  source 1..21: mol_type = other RNA
ctgcacccttctcatctggag

SEQ ID NO: 620 - RNA, 21 nt, synthetic construct
  misc_feature 1..21: note = Synthetic oligonucleotide
  source 1..21: mol_type = other RNA
tctgcacccttctcatctgga

SEQ ID NO: 621 - RNA, 21 nt, synthetic construct
  misc_feature 1..21: note = Synthetic oligonucleotide
  source 1..21: mol_type = other RNA
atctgcacccttctcatctgg

SEQ ID NO: 622 - RNA, 21 nt, synthetic construct
  misc_feature 1..21: note = Synthetic oligonucleotide
  source 1..21: mol_type = other RNA
gatctgcacccttctcatctg

SEQ ID NO: 623 - RNA, 21 nt, synthetic construct
  misc_feature 1..21: note = Synthetic oligonucleotide
  source 1..21: mol_type = other RNA
tgatctgcacccttctcatct

SEQ ID NO: 624 - RNA, 21 nt, synthetic construct
  misc_feature 1..21: note = Synthetic oligonucleotide
  source 1..21: mol_type = other RNA
ctgatctgcacccttctcatc

SEQ ID NO: 625 - RNA, 21 nt, synthetic construct
  misc_feature 1..21: note = Synthetic oligonucleotide
  source 1..21: mol_type = other RNA
gctgatctgcacccttctcat

SEQ ID NO: 626 - RNA, 21 nt, synthetic construct
  misc_feature 1..21: note = Synthetic oligonucleotide
  source 1..21: mol_type = other RNA
cgctgatctgcacccttctca

SEQ ID NO: 627 - RNA, 21 nt, synthetic construct
  misc_feature 1..21: note = Synthetic oligonucleotide
  source 1..21: mol_type = other RNA
gcgctgatctgcacccttctc

SEQ ID NO: 628 - RNA, 21 nt, synthetic construct
  misc_feature 1..21: note = Synthetic oligonucleotide
  source 1..21: mol_type = other RNA
ggcgctgatctgcacccttct

SEQ ID NO: 629 - RNA, 21 nt, synthetic construct
  misc_feature 1..21: note = Synthetic oligonucleotide
  source 1..21: mol_type = other RNA
tggcgctgatctgcacccttc

SEQ ID NO: 630 - RNA, 21 nt, synthetic construct
  misc_feature 1..21: note = Synthetic oligonucleotide
  source 1..21: mol_type = other RNA
ctggcgctgatctgcaccctt

SEQ ID NO: 631 - RNA, 21 nt, synthetic construct
  misc_feature 1..21: note = Synthetic oligonucleotide
  source 1..21: mol_type = other RNA
gctggcgctgatctgcaccct

SEQ ID NO: 632 - RNA, 21 nt, synthetic construct
  misc_feature 1..21: note = Synthetic oligonucleotide
  source 1..21: mol_type = other RNA
ggctggcgctgatctgcaccc

SEQ ID NO: 633 - RNA, 21 nt, synthetic construct
  misc_feature 1..21: note = Synthetic oligonucleotide
  source 1..21: mol_type = other RNA
gggctggcgctgatctgcacc

SEQ ID NO: 634 - RNA, 21 nt, synthetic construct
  misc_feature 1..21: note = Synthetic oligonucleotide
  source 1..21: mol_type = other RNA
agggctggcgctgatctgcac

SEQ ID NO: 635 - RNA, 21 nt, synthetic construct
  misc_feature 1..21: note = Synthetic oligonucleotide
  source 1..21: mol_type = other RNA
gagggctggcgctgatctgca

SEQ ID NO: 636 - RNA, 21 nt, synthetic construct
  misc_feature 1..21: note = Synthetic oligonucleotide
  source 1..21: mol_type = other RNA
ggagggctggcgctgatctgc

SEQ ID NO: 637 - RNA, 21 nt, synthetic construct
  misc_feature 1..21: note = Synthetic oligonucleotide
  source 1..21: mol_type = other RNA
aaagttgctgggccggcggcg

SEQ ID NO: 638 - RNA, 21 nt, synthetic construct
  misc_feature 1..21: note = Synthetic oligonucleotide
  source 1..21: mol_type = other RNA
caaagttgctgggccggcggc

SEQ ID NO: 639 - RNA, 21 nt, synthetic construct
  misc_feature 1..21: note = Synthetic oligonucleotide
  source 1..21: mol_type = other RNA
acaaagttgctgggccggcgg

SEQ ID NO: 640 - RNA, 21 nt, synthetic construct
  misc_feature 1..21: note = Synthetic oligonucleotide
  source 1..21: mol_type = other RNA
aacaaagttgctgggccggcg

SEQ ID NO: 641 - RNA, 21 nt, synthetic construct
  misc_feature 1..21: note = Synthetic oligonucleotide
  source 1..21: mol_type = other RNA
gaacaaagttgctgggccggc

SEQ ID NO: 642 - RNA, 21 nt, synthetic construct
  misc_feature 1..21: note = Synthetic oligonucleotide
  source 1..21: mol_type = other RNA
ggaacaaagttgctgggccgg

SEQ ID NO: 643 - RNA, 21 nt, synthetic construct
  misc_feature 1..21: note = Synthetic oligonucleotide
  source 1..21: mol_type = other RNA
cggaacaaagttgctgggccg

SEQ ID NO: 644 - RNA, 21 nt, synthetic construct
  misc_feature 1..21: note = Synthetic oligonucleotide
  source 1..21: mol_type = other RNA
ccggaacaaagttgctgggcc

SEQ ID NO: 645 - RNA, 21 nt, synthetic construct
  misc_feature 1..21: note = Synthetic oligonucleotide
  source 1..21: mol_type = other RNA
tccggaacaaagttgctgggc

SEQ ID NO: 646 - RNA, 21 nt, synthetic construct
  misc_feature 1..21: note = Synthetic oligonucleotide
  source 1..21: mol_type = other RNA
ttccggaacaaagttgctggg

SEQ ID NO: 647 - RNA, 21 nt, synthetic construct
  misc_feature 1..21: note = Synthetic oligonucleotide
  source 1..21: mol_type = other RNA
cttccggaacaaagttgctgg

SEQ ID NO: 648 - RNA, 21 nt, synthetic construct
  misc_feature 1..21: note = Synthetic oligonucleotide
  source 1..21: mol_type = other RNA
tcttccggaacaaagttgctg

SEQ ID NO: 649 - RNA, 21 nt, synthetic construct
  misc_feature 1..21: note = Synthetic oligonucleotide
  source 1..21: mol_type = other RNA
atcttccggaacaaagttgct

SEQ ID NO: 650 - RNA, 21 nt, synthetic construct
  misc_feature 1..21: note = Synthetic oligonucleotide
  source 1..21: mol_type = other RNA
gatcttccggaacaaagttgc

SEQ ID NO: 651 - RNA, 21 nt, synthetic construct
  misc_feature 1..21: note = Synthetic oligonucleotide
  source 1..21: mol_type = other RNA
tgatcttccggaacaaagttg

SEQ ID NO: 652 - RNA, 21 nt, synthetic construct
  misc_feature 1..21: note = Synthetic oligonucleotide
  source 1..21: mol_type = other RNA
ctgatcttccggaacaaagtt

SEQ ID NO: 653 - RNA, 21 nt, synthetic construct
  misc_feature 1..21: note = Synthetic oligonucleotide
  source 1..21: mol_type = other RNA
gctgatcttccggaacaaagt

SEQ ID NO: 654 - RNA, 21 nt, synthetic construct
  misc_feature 1..21: note = Synthetic oligonucleotide
  source 1..21: mol_type = other RNA
ggctgatcttccggaacaaag

SEQ ID NO: 655 - RNA, 21 nt, synthetic construct
  misc_feature 1..21: note = Synthetic oligonucleotide
  source 1..21: mol_type = other RNA
gggctgatcttccggaacaaa

SEQ ID NO: 656 - RNA, 21 nt, synthetic construct
  misc_feature 1..21: note = Synthetic oligonucleotide
  source 1..21: mol_type = other RNA
ggggctgatcttccggaacaa

SEQ ID NO: 657 - RNA, 21 nt, synthetic construct
  misc_feature 1..21: note = Synthetic oligonucleotide
  source 1..21: mol_type = other RNA
tggggctgatcttccggaaca

SEQ ID NO: 658 - RNA, 21 nt, synthetic construct
  misc_feature 1..21: note = Synthetic oligonucleotide
  source 1..21: mol_type = other RNA
gtggggctgatcttccggaac

SEQ ID NO: 659 - RNA, 21 nt, synthetic construct
  misc_feature 1..21: note = Synthetic oligonucleotide
  source 1..21: mol_type = other RNA
agtggggctgatcttccggaa

SEQ ID NO: 660 - RNA, 21 nt, synthetic construct
  misc_feature 1..21: note = Synthetic oligonucleotide
  source 1..21: mol_type = other RNA
tagtggggctgatcttccgga

SEQ ID NO: 661 - RNA, 21 nt, synthetic construct
  misc_feature 1..21: note = Synthetic oligonucleotide
  source 1..21: mol_type = other RNA
gtagtggggctgatcttccgg

SEQ ID NO: 662 - RNA, 21 nt, synthetic construct
  misc_feature 1..21: note = Synthetic oligonucleotide
  source 1..21: mol_type = other RNA
cgtagtggggctgatcttccg

SEQ ID NO: 663 - RNA, 21 nt, synthetic construct
  misc_feature 1..21: note = Synthetic oligonucleotide
  source 1..21: mol_type = other RNA
acgtagtggggctgatcttcc

SEQ ID NO: 664 - RNA, 21 nt, synthetic construct
  misc_feature 1..21: note = Synthetic oligonucleotide
  source 1..21: mol_type = other RNA
aacgtagtggggctgatcttc

SEQ ID NO: 665 - RNA, 21 nt, synthetic construct
  misc_feature 1..21: note = Synthetic oligonucleotide
  source 1..21: mol_type = other RNA
gaacgtagtggggctgatctt

SEQ ID NO: 666 - RNA, 21 nt, synthetic construct
  misc_feature 1..21: note = Synthetic oligonucleotide
  source 1..21: mol_type = other RNA
ggaacgtagtggggctgatct

SEQ ID NO: 667 - RNA, 21 nt, synthetic construct
  misc_feature 1..21: note = Synthetic oligonucleotide
  source 1..21: mol_type = other RNA
gggaacgtagtggggctgatc

SEQ ID NO: 668 - RNA, 21 nt, synthetic construct
  misc_feature 1..21: note = Synthetic oligonucleotide
  source 1..21: mol_type = other RNA
cgggaacgtagtggggctgat

SEQ ID NO: 669 - RNA, 21 nt, synthetic construct
  misc_feature 1..21: note = Synthetic oligonucleotide
  source 1..21: mol_type = other RNA
ccgggaacgtagtggggctga

SEQ ID NO: 670 - RNA, 21 nt, synthetic construct
  misc_feature 1..21: note = Synthetic oligonucleotide
  source 1..21: mol_type = other RNA
acccgggaacgtagtggggct

SEQ ID NO: 671 - RNA, 21 nt, synthetic construct
  misc_feature 1..21: note = Synthetic oligonucleotide
  source 1..21: mol_type = other RNA
gacccgggaacgtagtggggc

SEQ ID NO: 672 - RNA, 21 nt, synthetic construct
  misc_feature 1..21: note = Synthetic oligonucleotide
  source 1..21: mol_type = other RNA
tgacccgggaacgtagtgggg

SEQ ID NO: 673 - RNA, 21 nt, synthetic construct
  misc_feature 1..21: note = Synthetic oligonucleotide
  source 1..21: mol_type = other RNA
ctgacccgggaacgtagtggg

SEQ ID NO: 674 - RNA, 21 nt, synthetic construct
  misc_feature 1..21: note = Synthetic oligonucleotide
  source 1..21: mol_type = other RNA
gctgacccgggaacgtagtgg

SEQ ID NO: 675 - RNA, 21 nt, synthetic construct
  misc_feature 1..21: note = Synthetic oligonucleotide
  source 1..21: mol_type = other RNA
cagacgctgacccgggaacgt

SEQ ID NO: 676 - RNA, 21 nt, synthetic construct
  misc_feature 1..21: note = Synthetic oligonucleotide
  source 1..21: mol_type = other RNA
gcagacgctgacccgggaacg

SEQ ID NO: 677 - RNA, 21 nt, synthetic construct
  misc_feature 1..21: note = Synthetic oligonucleotide
  source 1..21: mol_type = other RNA
tggggcgccaagttcccgacg

SEQ ID NO: 678 - RNA, 21 nt, synthetic construct
  misc_feature 1..21: note = Synthetic oligonucleotide
  source 1..21: mol_type = other RNA
ttggggcgccaagttcccgac

SEQ ID NO: 679 - RNA, 21 nt, synthetic construct
  misc_feature 1..21: note = Synthetic oligonucleotide
  source 1..21: mol_type = other RNA
cttggggcgccaagttcccga

SEQ ID NO: 680 - RNA, 21 nt, synthetic construct
  misc_feature 1..21: note = Synthetic oligonucleotide
  source 1..21: mol_type = other RNA
acttggggcgccaagttcccg

SEQ ID NO: 681 - RNA, 21 nt, synthetic construct
  misc_feature 1..21: note = Synthetic oligonucleotide
  source 1..21: mol_type = other RNA
gacttggggcgccaagttccc

SEQ ID NO: 682 - RNA, 21 nt, synthetic construct
  misc_feature 1..21: note = Synthetic oligonucleotide
  source 1..21: mol_type = other RNA
tgacttggggcgccaagttcc

SEQ ID NO: 683 - RNA, 21 nt, synthetic construct
  misc_feature 1..21: note = Synthetic oligonucleotide
  source 1..21: mol_type = other RNA
gtgacttggggcgccaagttc

SEQ ID NO: 684 - RNA, 21 nt, synthetic construct
  misc_feature 1..21: note = Synthetic oligonucleotide
  source 1..21: mol_type = other RNA
ggtgacttggggcgccaagtt

SEQ ID NO: 685 - RNA, 21 nt, synthetic construct
  misc_feature 1..21: note = Synthetic oligonucleotide
  source 1..21: mol_type = other RNA
aggtgacttggggcgccaagt

SEQ ID NO: 686 - RNA, 21 nt, synthetic construct
  misc_feature 1..21: note = Synthetic oligonucleotide
  source 1..21: mol_type = other RNA
caggtgacttggggcgccaag

SEQ ID NO: 687 - RNA, 21 nt, synthetic construct
  misc_feature 1..21: note = Synthetic oligonucleotide
  source 1..21: mol_type = other RNA
tcaggtgacttggggcgccaa

SEQ ID NO: 688 - RNA, 21 nt, synthetic construct
  misc_feature 1..21: note = Synthetic oligonucleotide
  source 1..21: mol_type = other RNA
gtcaggtgacttggggcgcca

SEQ ID NO: 689 - RNA, 21 nt, synthetic construct
  misc_feature 1..21: note = Synthetic oligonucleotide
  source 1..21: mol_type = other RNA
agtcaggtgacttggggcgcc

SEQ ID NO: 690 - RNA, 21 nt, synthetic construct
  misc_feature 1..21: note = Synthetic oligonucleotide
  source 1..21: mol_type = other RNA
cagtcaggtgacttggggcgc

SEQ ID NO: 691 - RNA, 21 nt, synthetic construct
  misc_feature 1..21: note = Synthetic oligonucleotide
  source 1..21: mol_type = other RNA
gcagtcaggtgacttggggcg

SEQ ID NO: 692 - RNA, 21 nt, synthetic construct
  misc_feature 1..21: note = Synthetic oligonucleotide
  source 1..21: mol_type = other RNA
cgcagtcaggtgacttggggc

SEQ ID NO: 693 - RNA, 21 nt, synthetic construct
  misc_feature 1..21: note = Synthetic oligonucleotide
  source 1..21: mol_type = other RNA
gcgcagtcaggtgacttgggg

SEQ ID NO: 694 - RNA, 21 nt, synthetic construct
  misc_feature 1..21: note = Synthetic oligonucleotide
  source 1..21: mol_type = other RNA
cgcgcagtcaggtgacttggg

SEQ ID NO: 695 - RNA, 21 nt, synthetic construct
  misc_feature 1..21: note = Synthetic oligonucleotide
  source 1..21: mol_type = other RNA
agggccggcgctgattgacgg

SEQ ID NO: 696 - RNA, 21 nt, synthetic construct
  misc_feature 1..21: note = Synthetic oligonucleotide
  source 1..21: mol_type = other RNA
cagggccggcgctgattgacg

SEQ ID NO: 697 - RNA, 21 nt, synthetic construct
  misc_feature 1..21: note = Synthetic oligonucleotide
  source 1..21: mol_type = other RNA
acggttcactctggagcgcgc

SEQ ID NO: 698 - RNA, 21 nt, synthetic construct
  misc_feature 1..21: note = Synthetic oligonucleotide
  source 1..21: mol_type = other RNA
cacggttcactctggagcgcg

SEQ ID NO: 699 - RNA, 21 nt, synthetic construct
  misc_feature 1..21: note = Synthetic oligonucleotide
  source 1..21: mol_type = other RNA
gcacggttcactctggagcgc

SEQ ID NO: 700 - RNA, 21 nt, synthetic construct
  misc_feature 1..21: note = Synthetic oligonucleotide
  source 1..21: mol_type = other RNA
cgcacggttcactctggagcg

SEQ ID NO: 701 - RNA, 21 nt, synthetic construct
  misc_feature 1..21: note = Synthetic oligonucleotide
  source 1..21: mol_type = other RNA
acgcacggttcactctggagc

SEQ ID NO: 702 - RNA, 21 nt, synthetic construct
  misc_feature 1..21: note = Synthetic oligonucleotide
  source 1..21: mol_type = other RNA
cacgcacggttcactctggag

SEQ ID NO: 703 - RNA, 21 nt, synthetic construct
  misc_feature 1..21: note = Synthetic oligonucleotide
  source 1..21: mol_type = other RNA
gcacgcacggttcactctgga

SEQ ID NO: 704 - RNA, 21 nt, synthetic construct
  misc_feature 1..21: note = Synthetic oligonucleotide
  source 1..21: mol_type = other RNA
ggcacgcacggttcactctgg

SEQ ID NO: 705 - RNA, 21 nt, synthetic construct
  misc_feature 1..21: note = Synthetic oligonucleotide
  source 1..21: mol_type = other RNA
gttcggtctccggactcccgg

SEQ ID NO: 706 - RNA, 21 nt, synthetic construct
  misc_feature 1..21: note = Synthetic oligonucleotide
  source 1..21: mol_type = other RNA
ggttcggtctccggactcccg

SEQ ID NO: 707 - RNA, 21 nt, synthetic construct
  misc_feature 1..21: note = Synthetic oligonucleotide
  source 1..21: mol_type = other RNA
tggttcggtctccggactccc

SEQ ID NO: 708 - RNA, 21 nt, synthetic construct
  misc_feature 1..21: note = Synthetic oligonucleotide
  source 1..21: mol_type = other RNA
ctggttcggtctccggactcc

SEQ ID NO: 709 - RNA, 21 nt, synthetic construct
  misc_feature 1..21: note = Synthetic oligonucleotide
  source 1..21: mol_type = other RNA
tctggttcggtctccggactc

SEQ ID NO: 710 - RNA, 21 nt, synthetic construct
  misc_feature 1..21: note = Synthetic oligonucleotide
  source 1..21: mol_type = other RNA
ttctggttcggtctccggact

SEQ ID NO: 711 - RNA, 21 nt, synthetic construct
  misc_feature 1..21: note = Synthetic oligonucleotide
  source 1..21: mol_type = other RNA
attctggttcggtctccggac

SEQ ID NO: 712 - RNA, 21 nt, synthetic construct
  misc_feature 1..21: note = Synthetic oligonucleotide
  source 1..21: mol_type = other RNA
cattctggttcggtctccgga

SEQ ID NO: 713 - RNA, 21 nt, synthetic construct
  misc_feature 1..21: note = Synthetic oligonucleotide
  source 1..21: mol_type = other RNA
ccattctggttcggtctccgg

SEQ ID NO: 714 - RNA, 21 nt, synthetic construct
  misc_feature 1..21: note = Synthetic oligonucleotide
  source 1..21: mol_type = other RNA
tccattctggttcggtctccg

SEQ ID NO: 715 - RNA, 21 nt, synthetic construct
  misc_feature 1..21: note = Synthetic oligonucleotide
  source 1..21: mol_type = other RNA
atccattctggttcggtctcc

SEQ ID NO: 716 - RNA, 21 nt, synthetic construct
  misc_feature 1..21: note = Synthetic oligonucleotide
  source 1..21: mol_type = other RNA
catccattctggttcggtctc

SEQ ID NO: 717 - RNA, 21 nt, synthetic construct
  misc_feature 1..21: note = Synthetic oligonucleotide
  source 1..21: mol_type = other RNA
gcatccattctggttcggtct

SEQ ID NO: 718 - RNA, 21 nt, synthetic construct
  misc_feature 1..21: note = Synthetic oligonucleotide
  source 1..21: mol_type = other RNA
ggcatccattctggttcggtc

SEQ ID NO: 719 - RNA, 21 nt, synthetic construct
  misc_feature 1..21: note = Synthetic oligonucleotide
  source 1..21: mol_type = other RNA
aggcatccattctggttcggt

SEQ ID NO: 720 - RNA, 21 nt, synthetic construct
  misc_feature 1..21: note = Synthetic oligonucleotide
  source 1..21: mol_type = other RNA
gaggcatccattctggttcgg

SEQ ID NO: 721 - RNA, 21 nt, synthetic construct
  misc_feature 1..21: note = Synthetic oligonucleotide
  source 1..21: mol_type = other RNA
cgaggcatccattctggttcg

SEQ ID NO: 722 - RNA, 21 nt, synthetic construct
  misc_feature 1..21: note = Synthetic oligonucleotide
  source 1..21: mol_type = other RNA
tcgaggcatccattctggttc

SEQ ID NO: 723 - RNA, 21 nt, synthetic construct
  misc_feature 1..21: note = Synthetic oligonucleotide
  source 1..21: mol_type = other RNA
ctcgaggcatccattctggtt

SEQ ID NO: 724 - RNA, 21 nt, synthetic construct
  misc_feature 1..21: note = Synthetic oligonucleotide
  source 1..21: mol_type = other RNA
gctcgaggcatccattctggt

SEQ ID NO: 725 - RNA, 21 nt, synthetic construct
  misc_feature 1..21: note = Synthetic oligonucleotide
  source 1..21: mol_type = other RNA
tgctcgaggcatccattctgg

SEQ ID NO: 726 - RNA, 21 nt, synthetic construct
  misc_feature 1..21: note = Synthetic oligonucleotide
  source 1..21: mol_type = other RNA
ctgctcgaggcatccattctg

SEQ ID NO: 727 - RNA, 21 nt, synthetic construct
  misc_feature 1..21: note = Synthetic oligonucleotide
  source 1..21: mol_type = other RNA
tctgctcgaggcatccattct

SEQ ID NO: 728 - RNA, 21 nt, synthetic construct
  misc_feature 1..21: note = Synthetic oligonucleotide
  source 1..21: mol_type = other RNA
gtctgctcgaggcatccattc

SEQ ID NO: 729 - RNA, 21 nt, synthetic construct
  misc_feature 1..21: note = Synthetic oligonucleotide
  source 1..21: mol_type = other RNA
tgtctgctcgaggcatccatt

SEQ ID NO: 730 - RNA, 21 nt, synthetic construct
  misc_feature 1..21: note = Synthetic oligonucleotide
  source 1..21: mol_type = other RNA
gtgtctgctcgaggcatccat

SEQ ID NO: 731 - RNA, 21 nt, synthetic construct
  misc_feature 1..21: note = Synthetic oligonucleotide
  source 1..21: mol_type = other RNA
tgtgtctgctcgaggcatcca

SEQ ID NO: 732 - RNA, 21 nt, synthetic construct
  misc_feature 1..21: note = Synthetic oligonucleotide
  source 1..21: mol_type = other RNA
ttgtgtctgctcgaggcatcc

SEQ ID NO: 733 - RNA, 21 nt, synthetic construct
  misc_feature 1..21: note = Synthetic oligonucleotide
  source 1..21: mol_type = other RNA
attgtgtctgctcgaggcatc

SEQ ID NO: 734 - RNA, 21 nt, synthetic construct
  misc_feature 1..21: note = Synthetic oligonucleotide
  source 1..21: mol_type = other RNA
aattgtgtctgctcgaggcat

SEQ ID NO: 735 - RNA, 21 nt, synthetic construct
  misc_feature 1..21: note = Synthetic oligonucleotide
  source 1..21: mol_type = other RNA
gaattgtgtctgctcgaggca

SEQ ID NO: 736 - RNA, 21 nt, synthetic construct
  misc_feature 1..21: note = Synthetic oligonucleotide
  source 1..21: mol_type = other RNA
ggaattgtgtctgctcgaggc

SEQ ID NO: 737 - RNA, 21 nt, synthetic construct
  misc_feature 1..21: note = Synthetic oligonucleotide
  source 1..21: mol_type = other RNA
gggaattgtgtctgctcgagg

SEQ ID NO: 738 - RNA, 21 nt, synthetic construct
  misc_feature 1..21: note = Synthetic oligonucleotide
  source 1..21: mol_type = other RNA
ggggaattgtgtctgctcgag

SEQ ID NO: 739 - RNA, 21 nt, synthetic construct
  misc_feature 1..21: note = Synthetic oligonucleotide
  source 1..21: mol_type = other RNA
aggggaattgtgtctgctcga

SEQ ID NO: 740 - RNA, 21 nt, synthetic construct
  misc_feature 1..21: note = Synthetic oligonucleotide
  source 1..21: mol_type = other RNA
aaggggaattgtgtctgctcg

SEQ ID NO: 741 - RNA, 21 nt, synthetic construct
  misc_feature 1..21: note = Synthetic oligonucleotide
  source 1..21: mol_type = other RNA
taaggggaattgtgtctgctc

SEQ ID NO: 742 - RNA, 21 nt, synthetic construct
  misc_feature 1..21: note = Synthetic oligonucleotide
  source 1..21: mol_type = other RNA
ataaggggaattgtgtctgct

SEQ ID NO: 743 - RNA, 21 nt, synthetic construct
  misc_feature 1..21: note = Synthetic oligonucleotide
  source 1..21: mol_type = other RNA
aataaggggaattgtgtctgc

SEQ ID NO: 744 - RNA, 21 nt, synthetic construct
  misc_feature 1..21: note = Synthetic oligonucleotide
  source 1..21: mol_type = other RNA
gaataaggggaattgtgtctg

SEQ ID NO: 745 - RNA, 21 nt, synthetic construct
  misc_feature 1..21: note = Synthetic oligonucleotide
  source 1..21: mol_type = other RNA
tgaataaggggaattgtgtct

SEQ ID NO: 746 - RNA, 21 nt, synthetic construct
  misc_feature 1..21: note = Synthetic oligonucleotide
  source 1..21: mol_type = other RNA
ctgaataaggggaattgtgtc

SEQ ID NO: 747 - RNA, 21 nt, synthetic construct
  misc_feature 1..21: note = Synthetic oligonucleotide
  source 1..21: mol_type = other RNA
tctgaataaggggaattgtgt

SEQ ID NO: 748 - RNA, 21 nt, synthetic construct
  misc_feature 1..21: note = Synthetic oligonucleotide
  source 1..21: mol_type = other RNA
atctgaataaggggaattgtg

SEQ ID NO: 749 - RNA, 21 nt, synthetic construct
  misc_feature 1..21: note = Synthetic oligonucleotide
  source 1..21: mol_type = other RNA
gataatctgaataaggggaat

SEQ ID NO: 750 - RNA, 21 nt, synthetic construct
  misc_feature 1..21: note = Synthetic oligonucleotide
  source 1..21: mol_type = other RNA
ggataatctgaataaggggaa

SEQ ID NO: 751 - RNA, 21 nt, synthetic construct
  misc_feature 1..21: note = Synthetic oligonucleotide
  source 1..21: mol_type = other RNA
cggataatctgaataagggga

SEQ ID NO: 752 - RNA, 21 nt, synthetic construct
  misc_feature 1..21: note = Synthetic oligonucleotide
  source 1..21: mol_type = other RNA
tcggataatctgaataagggg

SEQ ID NO: 753 - RNA, 21 nt, synthetic construct
  misc_feature 1..21: note = Synthetic oligonucleotide
  source 1..21: mol_type = other RNA
cacattttcggataatctgaa

SEQ ID NO: 754 - RNA, 21 nt, synthetic construct
  misc_feature 1..21: note = Synthetic oligonucleotide
  source 1..21: mol_type = other RNA
ctcacattttcggataatctg

SEQ ID NO: 755 - RNA, 21 nt, synthetic construct
  misc_feature 1..21: note = Synthetic oligonucleotide
  source 1..21: mol_type = other RNA
gactcacattttcggataatc

SEQ ID NO: 756 - RNA, 21 nt, synthetic construct
  misc_feature 1..21: note = Synthetic oligonucleotide
  source 1..21: mol_type = other RNA
tgactcacattttcggataat

SEQ ID NO: 757 - RNA, 21 nt, synthetic construct
  misc_feature 1..21: note = Synthetic oligonucleotide
  source 1..21: mol_type = other RNA
gtgactcacattttcggataa

SEQ ID NO: 758 - RNA, 21 nt, synthetic construct
  misc_feature 1..21: note = Synthetic oligonucleotide
  source 1..21: mol_type = other RNA
cgtgactcacattttcggata

SEQ ID NO: 759 - RNA, 21 nt, synthetic construct
  misc_feature 1..21: note = Synthetic oligonucleotide
  source 1..21: mol_type = other RNA
acgtgactcacattttcggat

SEQ ID NO: 760 - RNA, 21 nt, synthetic construct
  misc_feature 1..21: note = Synthetic oligonucleotide
  source 1..21: mol_type = other RNA
gacgtgactcacattttcgga

SEQ ID NO: 761 - RNA, 21 nt, synthetic construct
  misc_feature 1..21: note = Synthetic oligonucleotide
  source 1..21: mol_type = other RNA
ggacgtgactcacattttcgg

SEQ ID NO: 762 - RNA, 21 nt, synthetic construct
  misc_feature 1..21: note = Synthetic oligonucleotide
  source 1..21: mol_type = other RNA
gggacgtgactcacattttcg

SEQ ID NO: 763 - RNA, 21 nt, synthetic construct
  misc_feature 1..21: note = Synthetic oligonucleotide
  source 1..21: mol_type = other RNA
ggggacgtgactcacattttc

SEQ ID NO: 764 - RNA, 21 nt, synthetic construct
  misc_feature 1..21: note = Synthetic oligonucleotide
  source 1..21: mol_type = other RNA
aggggacgtgactcacatttt

SEQ ID NO: 765 - RNA, 21 nt, synthetic construct
  misc_feature 1..21: note = Synthetic oligonucleotide
  source 1..21: mol_type = other RNA
aaggggacgtgactcacattt

SEQ ID NO: 766 - RNA, 21 nt, synthetic construct
  misc_feature 1..21: note = Synthetic oligonucleotide
  source 1..21: mol_type = other RNA
taaggggacgtgactcacatt

SEQ ID NO: 767 - RNA, 21 nt, synthetic construct
  misc_feature 1..21: note = Synthetic oligonucleotide
  source 1..21: mol_type = other RNA
ctaaggggacgtgactcacat

SEQ ID NO: 768 - RNA, 21 nt, synthetic construct
  misc_feature 1..21: note = Synthetic oligonucleotide
  source 1..21: mol_type = other RNA
cctaaggggacgtgactcaca

SEQ ID NO: 769 - RNA, 21 nt, synthetic construct
  misc_feature 1..21: note = Synthetic oligonucleotide
  source 1..21: mol_type = other RNA
gcctaaggggacgtgactcac

SEQ ID NO: 770 - RNA, 21 nt, synthetic construct
  misc_feature 1..21: note = Synthetic oligonucleotide
  source 1..21: mol_type = other RNA
tgcctaaggggacgtgactca

SEQ ID NO: 771 - RNA, 21 nt, synthetic construct
  misc_feature 1..21: note = Synthetic oligonucleotide
  source 1..21: mol_type = other RNA
ttgcctaaggggacgtgactc

SEQ ID NO: 772 - RNA, 21 nt, synthetic construct
  misc_feature 1..21: note = Synthetic oligonucleotide
  source 1..21: mol_type = other RNA
tttgcctaaggggacgtgact

SEQ ID NO: 773 - RNA, 21 nt, synthetic construct
  misc_feature 1..21: note = Synthetic oligonucleotide
  source 1..21: mol_type = other RNA
ttttgcctaaggggacgtgac

SEQ ID NO: 774 - RNA, 21 nt, synthetic construct
  misc_feature 1..21: note = Synthetic oligonucleotide
  source 1..21: mol_type = other RNA
gttttgcctaaggggacgtga

SEQ ID NO: 775 - RNA, 21 nt, synthetic construct
  misc_feature 1..21: note = Synthetic oligonucleotide
  source 1..21: mol_type = other RNA
tgttttgcctaaggggacgtg

SEQ ID NO: 776 - RNA, 21 nt, synthetic construct
  misc_feature 1..21: note = Synthetic oligonucleotide
  source 1..21: mol_type = other RNA
ctgttttgcctaaggggacgt

SEQ ID NO: 777 - RNA, 21 nt, synthetic construct
  misc_feature 1..21: note = Synthetic oligonucleotide
  source 1..21: mol_type = other RNA
cctgttttgcctaaggggacg

SEQ ID NO: 778 - RNA, 21 nt, synthetic construct
  misc_feature 1..21: note = Synthetic oligonucleotide
  source 1..21: mol_type = other RNA
acctgttttgcctaaggggac

SEQ ID NO: 779 - RNA, 21 nt, synthetic construct
  misc_feature 1..21: note = Synthetic oligonucleotide
  source 1..21: mol_type = other RNA
gacctgttttgcctaagggga

SEQ ID NO: 780 - RNA, 21 nt, synthetic construct
  misc_feature 1..21: note = Synthetic oligonucleotide
  source 1..21: mol_type = other RNA
agacctgttttgcctaagggg

SEQ ID NO: 781 - RNA, 21 nt, synthetic construct
  misc_feature 1..21: note = Synthetic oligonucleotide
  source 1..21: mol_type = other RNA
gagacctgttttgcctaaggg

SEQ ID NO: 782 - RNA, 21 nt, synthetic construct
  misc_feature 1..21: note = Synthetic oligonucleotide
  source 1..21: mol_type = other RNA
agagacctgttttgcctaagg

SEQ ID NO: 783 - RNA, 21 nt, synthetic construct
  misc_feature 1..21: note = Synthetic oligonucleotide
  source 1..21: mol_type = other RNA
tagagacctgttttgcctaag

SEQ ID NO: 784 - RNA, 21 nt, synthetic construct
  misc_feature 1..21: note = Synthetic oligonucleotide
  source 1..21: mol_type = other RNA
ctagagacctgttttgcctaa

SEQ ID NO: 785 - RNA, 21 nt, synthetic construct
  misc_feature 1..21: note = Synthetic oligonucleotide
  source 1..21: mol_type = other RNA
actagagacctgttttgccta

SEQ ID NO: 786 - RNA, 21 nt, synthetic construct
  misc_feature 1..21: note = Synthetic oligonucleotide
  source 1..21: mol_type = other RNA
aactagagacctgttttgcct

SEQ ID NO: 787 - RNA, 21 nt, synthetic construct
  misc_feature 1..21: note = Synthetic oligonucleotide
  source 1..21: mol_type = other RNA
taactagagacctgttttgcc

SEQ ID NO: 788 - RNA, 21 nt, synthetic construct
  misc_feature 1..21: note = Synthetic oligonucleotide
  source 1..21: mol_type = other RNA
ctaactagagacctgttttgc

SEQ ID NO: 789 - RNA, 21 nt, synthetic construct
  misc_feature 1..21: note = Synthetic oligonucleotide
  source 1..21: mol_type = other RNA
cctaactagagacctgttttg

SEQ ID NO: 790 - RNA, 21 nt, synthetic construct
  misc_feature 1..21: note = Synthetic oligonucleotide
  source 1..21: mol_type = other RNA
ccctaactagagacctgtttt

SEQ ID NO: 791 - RNA, 21 nt, synthetic construct
  misc_feature 1..21: note = Synthetic oligonucleotide
  source 1..21: mol_type = other RNA
accctaactagagacctgttt

SEQ ID NO: 792 - RNA, 21 nt, synthetic construct
  misc_feature 1..21: note = Synthetic oligonucleotide
  source 1..21: mol_type = other RNA
caccctaactagagacctgtt

SEQ ID NO: 793 - RNA, 21 nt, synthetic construct
  misc_feature 1..21: note = Synthetic oligonucleotide
  source 1..21: mol_type = other RNA
tcaccctaactagagacctgt

SEQ ID NO: 794 - RNA, 21 nt, synthetic construct
  misc_feature 1..21: note = Synthetic oligonucleotide
  source 1..21: mol_type = other RNA
atcaccctaactagagacctg

SEQ ID NO: 795 - RNA, 21 nt, synthetic construct
  misc_feature 1..21: note = Synthetic oligonucleotide
  source 1..21: mol_type = other RNA
tatcaccctaactagagacct

SEQ ID NO: 796 - RNA, 21 nt, synthetic construct
  misc_feature 1..21: note = Synthetic oligonucleotide
  source 1..21: mol_type = other RNA
atatcaccctaactagagacc

SEQ ID NO: 797 - RNA, 21 nt, synthetic construct
  misc_feature 1..21: note = Synthetic oligonucleotide
  source 1..21: mol_type = other RNA
aatatcaccctaactagagac

SEQ ID NO: 798 - DNA, 21 bp, synthetic construct
  misc_feature 1..19: note = RNA at (1,2,3,4,5,6,7,8,9,10,11,12,13,14,15,16,17,18,19)
  misc_feature 20..21: note = DNA at (20,21)
  misc_feature 1..21: note = Synthetic oligonucleotide
  source 1..21: mol_type = other DNA
ggttgctgatgtccttagatt

SEQ ID NO: 799 - DNA, 21 bp, synthetic construct
  misc_feature 1..19: note = RNA at (1,2,3,4,5,6,7,8,9,10,11,12,13,14,15,16,17,18,19)
  misc_feature 20..21: note = DNA at (20,21)
  misc_feature 1..21: note = Synthetic oligonucleotide
  source 1..21: mol_type = other DNA
actactgagtgacagtagatt

SEQ ID NO: 800 - RNA, 21 nt, synthetic construct
  misc_feature 1..21: note = Synthetic oligonucleotide
  source 1..21: mol_type = other RNA
ggagaagcagacacgaaccga

SEQ ID NO: 801 - RNA, 21 nt, synthetic construct
  misc_feature 1..21: note = Synthetic oligonucleotide
  source 1..21: mol_type = other RNA
gagaagcagacacgaaccgaa

SEQ ID NO: 802 - RNA, 21 nt, synthetic construct
  misc_feature 1..21: note = Synthetic oligonucleotide
  source 1..21: mol_type = other RNA
agaagcagacacgaaccgaaa

SEQ ID NO: 803 - RNA, 21 nt, synthetic construct
  misc_feature 1..21: note = Synthetic oligonucleotide
  source 1..21: mol_type = other RNA
gaagcagacacgaaccgaaat

SEQ ID NO: 804 - RNA, 21 nt, synthetic construct
  misc_feature 1..21: note = Synthetic oligonucleotide
  source 1..21: mol_type = other RNA
aagcagacacgaaccgaaatt

SEQ ID NO: 805 - RNA, 21 nt, synthetic construct
  misc_feature 1..21: note = Synthetic oligonucleotide
  source 1..21: mol_type = other RNA
agcagacacgaaccgaaattt

SEQ ID NO: 806 - RNA, 21 nt, synthetic construct
  misc_feature 1..21: note = Synthetic oligonucleotide
  source 1..21: mol_type = other RNA
gcagacacgaaccgaaatttt

SEQ ID NO: 807 - RNA, 21 nt, synthetic construct
  misc_feature 1..21: note = Synthetic oligonucleotide
  source 1..21: mol_type = other RNA
cagacacgaaccgaaatttta

SEQ ID NO: 808 - RNA, 21 nt, synthetic construct
  misc_feature 1..21: note = Synthetic oligonucleotide
  source 1..21: mol_type = other RNA
agacacgaaccgaaattttaa

SEQ ID NO: 809 - RNA, 21 nt, synthetic construct
  misc_feature 1..21: note = Synthetic oligonucleotide
  source 1..21: mol_type = other RNA
gacacgaaccgaaattttaaa

SEQ ID NO: 810 - RNA, 21 nt, synthetic construct
  misc_feature 1..21: note = Synthetic oligonucleotide
  source 1..21: mol_type = other RNA
agaaagagaaattgacacccg

SEQ ID NO: 811 - RNA, 21 nt, synthetic construct
  misc_feature 1..21: note = Synthetic oligonucleotide
  source 1..21: mol_type = other RNA
gaaagagaaattgacacccga

SEQ ID NO: 812 - RNA, 21 nt, synthetic construct
  misc_feature 1..21: note = Synthetic oligonucleotide
  source 1..21: mol_type = other RNA
aaagagaaattgacacccgac

SEQ ID NO: 813 - RNA, 21 nt, synthetic construct
  misc_feature 1..21: note = Synthetic oligonucleotide
  source 1..21: mol_type = other RNA
aagagaaattgacacccgact

SEQ ID NO: 814 - RNA, 21 nt, synthetic construct
  misc_feature 1..21: note = Synthetic oligonucleotide
  source 1..21: mol_type = other RNA
agagaaattgacacccgactg

SEQ ID NO: 815 - RNA, 21 nt, synthetic construct
  misc_feature 1..21: note = Synthetic oligonucleotide
  source 1..21: mol_type = other RNA
gagaaattgacacccgactga

SEQ ID NO: 816 - RNA, 21 nt, synthetic construct
  misc_feature 1..21: note = Synthetic oligonucleotide
  source 1..21: mol_type = other RNA
agaaattgacacccgactgaa

SEQ ID NO: 817 - RNA, 21 nt, synthetic construct
  misc_feature 1..21: note = Synthetic oligonucleotide
  source 1..21: mol_type = other RNA
gaaattgacacccgactgaaa

SEQ ID NO: 818 - RNA, 21 nt, synthetic construct
  misc_feature 1..21: note = Synthetic oligonucleotide
  source 1..21: mol_type = other RNA
aaattgacacccgactgaaaa

SEQ ID NO: 819 - RNA, 21 nt, synthetic construct
  misc_feature 1..21: note = Synthetic oligonucleotide
  source 1..21: mol_type = other RNA
aattgacacccgactgaaaac

SEQ ID NO: 820 - RNA, 21 nt, synthetic construct
  misc_feature 1..21: note = Synthetic oligonucleotide
  source 1..21: mol_type = other RNA
attgacacccgactgaaaacg

SEQ ID NO: 821 - RNA, 21 nt, synthetic construct
  misc_feature 1..21: note = Synthetic oligonucleotide
  source 1..21: mol_type = other RNA
ttgacacccgactgaaaacgc

SEQ ID NO: 822 - RNA, 21 nt, synthetic construct
  misc_feature 1..21: note = Synthetic oligonucleotide
  source 1..21: mol_type = other RNA
tgacacccgactgaaaacgcc

SEQ ID NO: 823 - RNA, 21 nt, synthetic construct
  misc_feature 1..21: note = Synthetic oligonucleotide
  source 1..21: mol_type = other RNA
gacacccgactgaaaacgccc

SEQ ID NO: 824 - RNA, 21 nt, synthetic construct
  misc_feature 1..21: note = Synthetic oligonucleotide
  source 1..21: mol_type = other RNA
acacccgactgaaaacgcccc

SEQ ID NO: 825 - RNA, 21 nt, synthetic construct
  misc_feature 1..21: note = Synthetic oligonucleotide
  source 1..21: mol_type = other RNA
cacccgactgaaaacgcccct

SEQ ID NO: 826 - RNA, 21 nt, synthetic construct
  misc_feature 1..21: note = Synthetic oligonucleotide
  source 1..21: mol_type = other RNA
acccgactgaaaacgcccctt

SEQ ID NO: 827 - RNA, 21 nt, synthetic construct
  misc_feature 1..21: note = Synthetic oligonucleotide
  source 1..21: mol_type = other RNA
cccgactgaaaacgcccctta

SEQ ID NO: 828 - RNA, 21 nt, synthetic construct
  misc_feature 1..21: note = Synthetic oligonucleotide
  source 1..21: mol_type = other RNA
ccgactgaaaacgccccttac

SEQ ID NO: 829 - RNA, 21 nt, synthetic construct
  misc_feature 1..21: note = Synthetic oligonucleotide
  source 1..21: mol_type = other RNA
cgactgaaaacgccccttact

SEQ ID NO: 830 - RNA, 21 nt, synthetic construct
  misc_feature 1..21: note = Synthetic oligonucleotide
  source 1..21: mol_type = other RNA
gactgaaaacgccccttactt

SEQ ID NO: 831 - RNA, 21 nt, synthetic construct
  misc_feature 1..21: note = Synthetic oligonucleotide
  source 1..21: mol_type = other RNA
actgaaaacgccccttacttg

SEQ ID NO: 832 - RNA, 21 nt, synthetic construct
  misc_feature 1..21: note = Synthetic oligonucleotide
  source 1..21: mol_type = other RNA
ctgaaaacgccccttacttgt

SEQ ID NO: 833 - RNA, 21 nt, synthetic construct
  misc_feature 1..21: note = Synthetic oligonucleotide
  source 1..21: mol_type = other RNA
tgaaaacgccccttacttgtg

SEQ ID NO: 834 - RNA, 21 nt, synthetic construct
  misc_feature 1..21: note = Synthetic oligonucleotide
  source 1..21: mol_type = other RNA
gaaaacgccccttacttgtgc

SEQ ID NO: 835 - RNA, 21 nt, synthetic construct
  misc_feature 1..21: note = Synthetic oligonucleotide
  source 1..21: mol_type = other RNA
aaaacgccccttacttgtgca

SEQ ID NO: 836 - RNA, 21 nt, synthetic construct
  misc_feature 1..21: note = Synthetic oligonucleotide
  source 1..21: mol_type = other RNA
aaacgccccttacttgtgcaa

SEQ ID NO: 837 - RNA, 21 nt, synthetic construct
  misc_feature 1..21: note = Synthetic oligonucleotide
  source 1..21: mol_type = other RNA
aacgccccttacttgtgcaat

SEQ ID NO: 838 - RNA, 21 nt, synthetic construct
  misc_feature 1..21: note = Synthetic oligonucleotide
  source 1..21: mol_type = other RNA
acgccccttacttgtgcaatc

SEQ ID NO: 839 - RNA, 21 nt, synthetic construct
  misc_feature 1..21: note = Synthetic oligonucleotide
  source 1..21: mol_type = other RNA
cgccccttacttgtgcaatct

SEQ ID NO: 840 - RNA, 21 nt, synthetic construct
  misc_feature 1..21: note = Synthetic oligonucleotide
  source 1..21: mol_type = other RNA
gccccttacttgtgcaatctt

SEQ ID NO: 841 - RNA, 21 nt, synthetic construct
  misc_feature 1..21: note = Synthetic oligonucleotide
  source 1..21: mol_type = other RNA
ccccttacttgtgcaatcttc

SEQ ID NO: 842 - RNA, 21 nt, synthetic construct
  misc_feature 1..21: note = Synthetic oligonucleotide
  source 1..21: mol_type = other RNA
cccttacttgtgcaatcttcc

SEQ ID NO: 843 - RNA, 21 nt, synthetic construct
  misc_feature 1..21: note = Synthetic oligonucleotide
  source 1..21: mol_type = other RNA
ccttacttgtgcaatcttcct

SEQ ID NO: 844 - RNA, 21 nt, synthetic construct
  misc_feature 1..21: note = Synthetic oligonucleotide
  source 1..21: mol_type = other RNA
cttacttgtgcaatcttcctg

SEQ ID NO: 845 - RNA, 21 nt, synthetic construct
  misc_feature 1..21: note = Synthetic oligonucleotide
  source 1..21: mol_type = other RNA
ttacttgtgcaatcttcctgc

SEQ ID NO: 846 - RNA, 21 nt, synthetic construct
  misc_feature 1..21: note = Synthetic oligonucleotide
  source 1..21: mol_type = other RNA
tacttgtgcaatcttcctgcc

SEQ ID NO: 847 - RNA, 21 nt, synthetic construct
  misc_feature 1..21: note = Synthetic oligonucleotide
  source 1..21: mol_type = other RNA
acttgtgcaatcttcctgcct

SEQ ID NO: 848 - RNA, 21 nt, synthetic construct
  misc_feature 1..21: note = Synthetic oligonucleotide
  source 1..21: mol_type = other RNA
cttgtgcaatcttcctgcctg

SEQ ID NO: 849 - RNA, 21 nt, synthetic construct
  misc_feature 1..21: note = Synthetic oligonucleotide
  source 1..21: mol_type = other RNA
ttgtgcaatcttcctgcctgg

SEQ ID NO: 850 - RNA, 21 nt, synthetic construct
  misc_feature 1..21: note = Synthetic oligonucleotide
  source 1..21: mol_type = other RNA
tgtgcaatcttcctgcctggt

SEQ ID NO: 851 - RNA, 21 nt, synthetic construct
  misc_feature 1..21: note = Synthetic oligonucleotide
  source 1..21: mol_type = other RNA
gtgcaatcttcctgcctggta

SEQ ID NO: 852 - RNA, 21 nt, synthetic construct
  misc_feature 1..21: note = Synthetic oligonucleotide
  source 1..21: mol_type = other RNA
tgcaatcttcctgcctggtag

SEQ ID NO: 853 - RNA, 21 nt, synthetic construct
  misc_feature 1..21: note = Synthetic oligonucleotide
  source 1..21: mol_type = other RNA
gcaatcttcctgcctggtagt

SEQ ID NO: 854 - RNA, 21 nt, synthetic construct
  misc_feature 1..21: note = Synthetic oligonucleotide
  source 1..21: mol_type = other RNA
caatcttcctgcctggtagtt

SEQ ID NO: 855 - RNA, 21 nt, synthetic construct
  misc_feature 1..21: note = Synthetic oligonucleotide
  source 1..21: mol_type = other RNA
aatcttcctgcctggtagttc

SEQ ID NO: 856 - RNA, 21 nt, synthetic construct
  misc_feature 1..21: note = Synthetic oligonucleotide
  source 1..21: mol_type = other RNA
atcttcctgcctggtagttcc

SEQ ID NO: 857 - RNA, 21 nt, synthetic construct
  misc_feature 1..21: note = Synthetic oligonucleotide
  source 1..21: mol_type = other RNA
tcttcctgcctggtagttccg

SEQ ID NO: 858 - RNA, 21 nt, synthetic construct
  misc_feature 1..21: note = Synthetic oligonucleotide
  source 1..21: mol_type = other RNA
cttcctgcctggtagttccgc

SEQ ID NO: 859 - RNA, 21 nt, synthetic construct
  misc_feature 1..21: note = Synthetic oligonucleotide
  source 1..21: mol_type = other RNA
ttcctgcctggtagttccgcc

SEQ ID NO: 860 - RNA, 21 nt, synthetic construct
  misc_feature 1..21: note = Synthetic oligonucleotide
  source 1..21: mol_type = other RNA
tcctgcctggtagttccgcca

SEQ ID NO: 861 - RNA, 21 nt, synthetic construct
  misc_feature 1..21: note = Synthetic oligonucleotide
  source 1..21: mol_type = other RNA
cctgcctggtagttccgccaa

SEQ ID NO: 862 - RNA, 21 nt, synthetic construct
  misc_feature 1..21: note = Synthetic oligonucleotide
  source 1..21: mol_type = other RNA
ctgcctggtagttccgccaaa

SEQ ID NO: 863 - RNA, 21 nt, synthetic construct
  misc_feature 1..21: note = Synthetic oligonucleotide
  source 1..21: mol_type = other RNA
tgcctggtagttccgccaaaa

SEQ ID NO: 864 - RNA, 21 nt, synthetic construct
  misc_feature 1..21: note = Synthetic oligonucleotide
  source 1..21: mol_type = other RNA
gcctggtagttccgccaaaaa

SEQ ID NO: 865 - RNA, 21 nt, synthetic construct
  misc_feature 1..21: note = Synthetic oligonucleotide
  source 1..21: mol_type = other RNA
cctggtagttccgccaaaaac

SEQ ID NO: 866 - RNA, 21 nt, synthetic construct
  misc_feature 1..21: note = Synthetic oligonucleotide
  source 1..21: mol_type = other RNA
aaacaaaacagtcaaagcgca

SEQ ID NO: 867 - RNA, 21 nt, synthetic construct
  misc_feature 1..21: note = Synthetic oligonucleotide
  source 1..21: mol_type = other RNA
aacaaaacagtcaaagcgcaa

SEQ ID NO: 868 - RNA, 21 nt, synthetic construct
  misc_feature 1..21: note = Synthetic oligonucleotide
  source 1..21: mol_type = other RNA
acaaaacagtcaaagcgcaaa

SEQ ID NO: 869 - RNA, 21 nt, synthetic construct
  misc_feature 1..21: note = Synthetic oligonucleotide
  source 1..21: mol_type = other RNA
caaaacagtcaaagcgcaaac

SEQ ID NO: 870 - RNA, 21 nt, synthetic construct
  misc_feature 1..21: note = Synthetic oligonucleotide
  source 1..21: mol_type = other RNA
aaaacagtcaaagcgcaaacc

SEQ ID NO: 871 - RNA, 21 nt, synthetic construct
  misc_feature 1..21: note = Synthetic oligonucleotide
  source 1..21: mol_type = other RNA
aaacagtcaaagcgcaaacct

SEQ ID NO: 872 - RNA, 21 nt, synthetic construct
  misc_feature 1..21: note = Synthetic oligonucleotide
  source 1..21: mol_type = other RNA
aacagtcaaagcgcaaacctg

SEQ ID NO: 873 - RNA, 21 nt, synthetic construct
  misc_feature 1..21: note = Synthetic oligonucleotide
  source 1..21: mol_type = other RNA
acagtcaaagcgcaaacctgg

SEQ ID NO: 874 - RNA, 21 nt, synthetic construct
  misc_feature 1..21: note = Synthetic oligonucleotide
  source 1..21: mol_type = other RNA
cagtcaaagcgcaaacctgga

SEQ ID NO: 875 - RNA, 21 nt, synthetic construct
  misc_feature 1..21: note = Synthetic oligonucleotide
  source 1..21: mol_type = other RNA
agtcaaagcgcaaacctggaa

SEQ ID NO: 876 - RNA, 21 nt, synthetic construct
  misc_feature 1..21: note = Synthetic oligonucleotide
  source 1..21: mol_type = other RNA
gtcaaagcgcaaacctggaag

SEQ ID NO: 877 - RNA, 21 nt, synthetic construct
  misc_feature 1..21: note = Synthetic oligonucleotide
  source 1..21: mol_type = other RNA
tcaaagcgcaaacctggaagg

SEQ ID NO: 878 - RNA, 21 nt, synthetic construct
  misc_feature 1..21: note = Synthetic oligonucleotide
  source 1..21: mol_type = other RNA
caaagcgcaaacctggaaggg

SEQ ID NO: 879 - RNA, 21 nt, synthetic construct
  misc_feature 1..21: note = Synthetic oligonucleotide
  source 1..21: mol_type = other RNA
aaagcgcaaacctggaagggg

SEQ ID NO: 880 - RNA, 21 nt, synthetic construct
  misc_feature 1..21: note = Synthetic oligonucleotide
  source 1..21: mol_type = other RNA
aagcgcaaacctggaaggggc

SEQ ID NO: 881 - RNA, 21 nt, synthetic construct
  misc_feature 1..21: note = Synthetic oligonucleotide
  source 1..21: mol_type = other RNA
agcgcaaacctggaaggggcg

SEQ ID NO: 882 - RNA, 21 nt, synthetic construct
  misc_feature 1..21: note = Synthetic oligonucleotide
  source 1..21: mol_type = other RNA
gcgcaaacctggaaggggcgc

SEQ ID NO: 883 - RNA, 21 nt, synthetic construct
  misc_feature 1..21: note = Synthetic oligonucleotide
  source 1..21: mol_type = other RNA
cgcaaacctggaaggggcgcg

SEQ ID NO: 884 - RNA, 21 nt, synthetic construct
  misc_feature 1..21: note = Synthetic oligonucleotide
  source 1..21: mol_type = other RNA
gcaaacctggaaggggcgcgc

SEQ ID NO: 885 - RNA, 21 nt, synthetic construct
  misc_feature 1..21: note = Synthetic oligonucleotide
  source 1..21: mol_type = other RNA
caaacctggaaggggcgcgca

SEQ ID NO: 886 - RNA, 21 nt, synthetic construct
  misc_feature 1..21: note = Synthetic oligonucleotide
  source 1..21: mol_type = other RNA
cgcgcaggcaccaactttgcc

SEQ ID NO: 887 - RNA, 21 nt, synthetic construct
  misc_feature 1..21: note = Synthetic oligonucleotide
  source 1..21: mol_type = other RNA
gcgcaggcaccaactttgcca

SEQ ID NO: 888 - RNA, 21 nt, synthetic construct
  misc_feature 1..21: note = Synthetic oligonucleotide
  source 1..21: mol_type = other RNA
cgcaggcaccaactttgccaa

SEQ ID NO: 889 - RNA, 21 nt, synthetic construct
  misc_feature 1..21: note = Synthetic oligonucleotide
  source 1..21: mol_type = other RNA
gcaggcaccaactttgccaaa

SEQ ID NO: 890 - RNA, 21 nt, synthetic construct
  misc_feature 1..21: note = Synthetic oligonucleotide
  source 1..21: mol_type = other RNA
caggcaccaactttgccaaac

SEQ ID NO: 891 - RNA, 21 nt, synthetic construct
  misc_feature 1..21: note = Synthetic oligonucleotide
  source 1..21: mol_type = other RNA
aggcaccaactttgccaaacg

SEQ ID NO: 892 - RNA, 21 nt, synthetic construct
  misc_feature 1..21: note = Synthetic oligonucleotide
  source 1..21: mol_type = other RNA
ggcaccaactttgccaaacgc

SEQ ID NO: 893 - RNA, 21 nt, synthetic construct
  misc_feature 1..21: note = Synthetic oligonucleotide
  source 1..21: mol_type = other RNA
gcaccaactttgccaaacgct

SEQ ID NO: 894 - RNA, 21 nt, synthetic construct
  misc_feature 1..21: note = Synthetic oligonucleotide
  source 1..21: mol_type = other RNA
caccaactttgccaaacgcta

SEQ ID NO: 895 - RNA, 21 nt, synthetic construct
  misc_feature 1..21: note = Synthetic oligonucleotide
  source 1..21: mol_type = other RNA
accaactttgccaaacgctac

SEQ ID NO: 896 - RNA, 21 nt, synthetic construct
  misc_feature 1..21: note = Synthetic oligonucleotide
  source 1..21: mol_type = other RNA
ccaactttgccaaacgctaca

SEQ ID NO: 897 - RNA, 21 nt, synthetic construct
  misc_feature 1..21: note = Synthetic oligonucleotide
  source 1..21: mol_type = other RNA
caactttgccaaacgctacag

SEQ ID NO: 898 - RNA, 21 nt, synthetic construct
  misc_feature 1..21: note = Synthetic oligonucleotide
  source 1..21: mol_type = other RNA
aactttgccaaacgctacaga

SEQ ID NO: 899 - RNA, 21 nt, synthetic construct
  misc_feature 1..21: note = Synthetic oligonucleotide
  source 1..21: mol_type = other RNA
actttgccaaacgctacagag

SEQ ID NO: 900 - RNA, 21 nt, synthetic construct
  misc_feature 1..21: note = Synthetic oligonucleotide
  source 1..21: mol_type = other RNA
ctttgccaaacgctacagagt

SEQ ID NO: 901 - RNA, 21 nt, synthetic construct
  misc_feature 1..21: note = Synthetic oligonucleotide
  source 1..21: mol_type = other RNA
tttgccaaacgctacagagtt

SEQ ID NO: 902 - RNA, 21 nt, synthetic construct
  misc_feature 1..21: note = Synthetic oligonucleotide
  source 1..21: mol_type = other RNA
ttgccaaacgctacagagttt

SEQ ID NO: 903 - RNA, 21 nt, synthetic construct
  misc_feature 1..21: note = Synthetic oligonucleotide
  source 1..21: mol_type = other RNA
tgccaaacgctacagagtttt

SEQ ID NO: 904 - RNA, 21 nt, synthetic construct
  misc_feature 1..21: note = Synthetic oligonucleotide
  source 1..21: mol_type = other RNA
gccaaacgctacagagttttc

SEQ ID NO: 905 - RNA, 21 nt, synthetic construct
  misc_feature 1..21: note = Synthetic oligonucleotide
  source 1..21: mol_type = other RNA
ccaaacgctacagagttttcc

SEQ ID NO: 906 - RNA, 21 nt, synthetic construct
  misc_feature 1..21: note = Synthetic oligonucleotide
  source 1..21: mol_type = other RNA
caaacgctacagagttttccg

SEQ ID NO: 907 - RNA, 21 nt, synthetic construct
  misc_feature 1..21: note = Synthetic oligonucleotide
  source 1..21: mol_type = other RNA
aaacgctacagagttttccgt

SEQ ID NO: 908 - RNA, 21 nt, synthetic construct
  misc_feature 1..21: note = Synthetic oligonucleotide
  source 1..21: mol_type = other RNA
aacgctacagagttttccgtt

SEQ ID NO: 909 - RNA, 21 nt, synthetic construct
  misc_feature 1..21: note = Synthetic oligonucleotide
  source 1..21: mol_type = other RNA
acgctacagagttttccgttt

SEQ ID NO: 910 - RNA, 21 nt, synthetic construct
  misc_feature 1..21: note = Synthetic oligonucleotide
  source 1..21: mol_type = other RNA
cgctacagagttttccgtttg

SEQ ID NO: 911 - RNA, 21 nt, synthetic construct
  misc_feature 1..21: note = Synthetic oligonucleotide
  source 1..21: mol_type = other RNA
gctacagagttttccgtttgc

SEQ ID NO: 912 - RNA, 21 nt, synthetic construct
  misc_feature 1..21: note = Synthetic oligonucleotide
  source 1..21: mol_type = other RNA
ctacagagttttccgtttgca

SEQ ID NO: 913 - RNA, 21 nt, synthetic construct
  misc_feature 1..21: note = Synthetic oligonucleotide
  source 1..21: mol_type = other RNA
tacagagttttccgtttgcag

SEQ ID NO: 914 - RNA, 21 nt, synthetic construct
  misc_feature 1..21: note = Synthetic oligonucleotide
  source 1..21: mol_type = other RNA
acagagttttccgtttgcaga

SEQ ID NO: 915 - RNA, 21 nt, synthetic construct
  misc_feature 1..21: note = Synthetic oligonucleotide
  source 1..21: mol_type = other RNA
cagagttttccgtttgcagaa

SEQ ID NO: 916 - RNA, 21 nt, synthetic construct
  misc_feature 1..21: note = Synthetic oligonucleotide
  source 1..21: mol_type = other RNA
agagttttccgtttgcagaaa

SEQ ID NO: 917 - RNA, 21 nt, synthetic construct
  misc_feature 1..21: note = Synthetic oligonucleotide
  source 1..21: mol_type = other RNA
gagttttccgtttgcagaaat

SEQ ID NO: 918 - RNA, 21 nt, synthetic construct
  misc_feature 1..21: note = Synthetic oligonucleotide
  source 1..21: mol_type = other RNA
agttttccgtttgcagaaatg

SEQ ID NO: 919 - RNA, 21 nt, synthetic construct
  misc_feature 1..21: note = Synthetic oligonucleotide
  source 1..21: mol_type = other RNA
gttttccgtttgcagaaatga

SEQ ID NO: 920 - RNA, 21 nt, synthetic construct
  misc_feature 1..21: note = Synthetic oligonucleotide
  source 1..21: mol_type = other RNA
ttttccgtttgcagaaatgac

SEQ ID NO: 921 - RNA, 21 nt, synthetic construct
  misc_feature 1..21: note = Synthetic oligonucleotide
  source 1..21: mol_type = other RNA
tttccgtttgcagaaatgacc

SEQ ID NO: 922 - RNA, 21 nt, synthetic construct
  misc_feature 1..21: note = Synthetic oligonucleotide
  source 1..21: mol_type = other RNA
ttccgtttgcagaaatgaccc

SEQ ID NO: 923 - RNA, 21 nt, synthetic construct
  misc_feature 1..21: note = Synthetic oligonucleotide
  source 1..21: mol_type = other RNA
tccgtttgcagaaatgaccca

SEQ ID NO: 924 - RNA, 21 nt, synthetic construct
  misc_feature 1..21: note = Synthetic oligonucleotide
  source 1..21: mol_type = other RNA
ccgtttgcagaaatgacccaa

SEQ ID NO: 925 - RNA, 21 nt, synthetic construct
  misc_feature 1..21: note = Synthetic oligonucleotide
  source 1..21: mol_type = other RNA
cgtttgcagaaatgacccaaa

SEQ ID NO: 926 - RNA, 21 nt, synthetic construct
  misc_feature 1..21: note = Synthetic oligonucleotide
  source 1..21: mol_type = other RNA
gtttgcagaaatgacccaaaa

SEQ ID NO: 927 - RNA, 21 nt, synthetic construct
  misc_feature 1..21: note = Synthetic oligonucleotide
  source 1..21: mol_type = other RNA
tttgcagaaatgacccaaaag

SEQ ID NO: 928 - RNA, 21 nt, synthetic construct
  misc_feature 1..21: note = Synthetic oligonucleotide
  source 1..21: mol_type = other RNA
ttgcagaaatgacccaaaaga

SEQ ID NO: 929 - RNA, 21 nt, synthetic construct
  misc_feature 1..21: note = Synthetic oligonucleotide
  source 1..21: mol_type = other RNA
tgcagaaatgacccaaaagaa

SEQ ID NO: 930 - RNA, 21 nt, synthetic construct
  misc_feature 1..21: note = Synthetic oligonucleotide
  source 1..21: mol_type = other RNA
gcagaaatgacccaaaagaag

SEQ ID NO: 931 - RNA, 21 nt, synthetic construct
  misc_feature 1..21: note = Synthetic oligonucleotide
  source 1..21: mol_type = other RNA
cagaaatgacccaaaagaagg

SEQ ID NO: 932 - RNA, 21 nt, synthetic construct
  misc_feature 1..21: note = Synthetic oligonucleotide
  source 1..21: mol_type = other RNA
agaaatgacccaaaagaaggg

SEQ ID NO: 933 - RNA, 21 nt, synthetic construct
  misc_feature 1..21: note = Synthetic oligonucleotide
  source 1..21: mol_type = other RNA
gaaatgacccaaaagaagggg

SEQ ID NO: 934 - RNA, 21 nt, synthetic construct
  misc_feature 1..21: note = Synthetic oligonucleotide
  source 1..21: mol_type = other RNA
aaatgacccaaaagaaggggg

SEQ ID NO: 935 - RNA, 21 nt, synthetic construct
  misc_feature 1..21: note = Synthetic oligonucleotide
  source 1..21: mol_type = other RNA
aatgacccaaaagaagggggg

SEQ ID NO: 936 - RNA, 21 nt, synthetic construct
  misc_feature 1..21: note = Synthetic oligonucleotide
  source 1..21: mol_type = other RNA
cgctccccgtgattccccaat

SEQ ID NO: 937 - RNA, 21 nt, synthetic construct
  misc_feature 1..21: note = Synthetic oligonucleotide
  source 1..21: mol_type = other RNA
gctccccgtgattccccaatc

SEQ ID NO: 938 - RNA, 21 nt, synthetic construct
  misc_feature 1..21: note = Synthetic oligonucleotide
  source 1..21: mol_type = other RNA
ctccccgtgattccccaatcg

SEQ ID NO: 939 - RNA, 21 nt, synthetic construct
  misc_feature 1..21: note = Synthetic oligonucleotide
  source 1..21: mol_type = other RNA
tccccgtgattccccaatcgg

SEQ ID NO: 940 - RNA, 21 nt, synthetic construct
  misc_feature 1..21: note = Synthetic oligonucleotide
  source 1..21: mol_type = other RNA
ccccgtgattccccaatcggc

SEQ ID NO: 941 - RNA, 21 nt, synthetic construct
  misc_feature 1..21: note = Synthetic oligonucleotide
  source 1..21: mol_type = other RNA
cccgtgattccccaatcggct

SEQ ID NO: 942 - RNA, 21 nt, synthetic construct
  misc_feature 1..21: note = Synthetic oligonucleotide
  source 1..21: mol_type = other RNA
ccgtgattccccaatcggctt

SEQ ID NO: 943 - RNA, 21 nt, synthetic construct
  misc_feature 1..21: note = Synthetic oligonucleotide
  source 1..21: mol_type = other RNA
cgtgattccccaatcggcttc

SEQ ID NO: 944 - RNA, 21 nt, synthetic construct
  misc_feature 1..21: note = Synthetic oligonucleotide
  source 1..21: mol_type = other RNA
gtgattccccaatcggcttct

SEQ ID NO: 945 - RNA, 21 nt, synthetic construct
  misc_feature 1..21: note = Synthetic oligonucleotide
  source 1..21: mol_type = other RNA
tgattccccaatcggcttctg

SEQ ID NO: 946 - RNA, 21 nt, synthetic construct
  misc_feature 1..21: note = Synthetic oligonucleotide
  source 1..21: mol_type = other RNA
gattccccaatcggcttctgg

SEQ ID NO: 947 - RNA, 21 nt, synthetic construct
  misc_feature 1..21: note = Synthetic oligonucleotide
  source 1..21: mol_type = other RNA
attccccaatcggcttctgga

SEQ ID NO: 948 - RNA, 21 nt, synthetic construct
  misc_feature 1..21: note = Synthetic oligonucleotide
  source 1..21: mol_type = other RNA
ttccccaatcggcttctggag

SEQ ID NO: 949 - RNA, 21 nt, synthetic construct
  misc_feature 1..21: note = Synthetic oligonucleotide
  source 1..21: mol_type = other RNA
tccccaatcggcttctggagc

SEQ ID NO: 950 - RNA, 21 nt, synthetic construct
  misc_feature 1..21: note = Synthetic oligonucleotide
  source 1..21: mol_type = other RNA
ccccaatcggcttctggagcc

SEQ ID NO: 951 - RNA, 21 nt, synthetic construct
  misc_feature 1..21: note = Synthetic oligonucleotide
  source 1..21: mol_type = other RNA
cccaatcggcttctggagcca

SEQ ID NO: 952 - RNA, 21 nt, synthetic construct
  misc_feature 1..21: note = Synthetic oligonucleotide
  source 1..21: mol_type = other RNA
ccaatcggcttctggagccag

SEQ ID NO: 953 - RNA, 21 nt, synthetic construct
  misc_feature 1..21: note = Synthetic oligonucleotide
  source 1..21: mol_type = other RNA
caatcggcttctggagccaga

SEQ ID NO: 954 - RNA, 21 nt, synthetic construct
  misc_feature 1..21: note = Synthetic oligonucleotide
  source 1..21: mol_type = other RNA
aatcggcttctggagccagag

SEQ ID NO: 955 - RNA, 21 nt, synthetic construct
  misc_feature 1..21: note = Synthetic oligonucleotide
  source 1..21: mol_type = other RNA
atcggcttctggagccagagg

SEQ ID NO: 956 - RNA, 21 nt, synthetic construct
  misc_feature 1..21: note = Synthetic oligonucleotide
  source 1..21: mol_type = other RNA
tcggcttctggagccagaggc

SEQ ID NO: 957 - RNA, 21 nt, synthetic construct
  misc_feature 1..21: note = Synthetic oligonucleotide
  source 1..21: mol_type = other RNA
cggcttctggagccagaggct

SEQ ID NO: 958 - RNA, 21 nt, synthetic construct
  misc_feature 1..21: note = Synthetic oligonucleotide
  source 1..21: mol_type = other RNA
ggcttctggagccagaggctc

SEQ ID NO: 959 - RNA, 21 nt, synthetic construct
  misc_feature 1..21: note = Synthetic oligonucleotide
  source 1..21: mol_type = other RNA
gcttctggagccagaggctcc

SEQ ID NO: 960 - RNA, 21 nt, synthetic construct
  misc_feature 1..21: note = Synthetic oligonucleotide
  source 1..21: mol_type = other RNA
cttctggagccagaggctcct

SEQ ID NO: 961 - RNA, 21 nt, synthetic construct
  misc_feature 1..21: note = Synthetic oligonucleotide
  source 1..21: mol_type = other RNA
ttctggagccagaggctcctc

SEQ ID NO: 962 - RNA, 21 nt, synthetic construct
  misc_feature 1..21: note = Synthetic oligonucleotide
  source 1..21: mol_type = other RNA
tctggagccagaggctcctcc

SEQ ID NO: 963 - RNA, 21 nt, synthetic construct
  misc_feature 1..21: note = Synthetic oligonucleotide
  source 1..21: mol_type = other RNA
ctggagccagaggctcctcct

SEQ ID NO: 964 - RNA, 21 nt, synthetic construct
  misc_feature 1..21: note = Synthetic oligonucleotide
  source 1..21: mol_type = other RNA
tggagccagaggctcctcctc

SEQ ID NO: 965 - RNA, 21 nt, synthetic construct
  misc_feature 1..21: note = Synthetic oligonucleotide
  source 1..21: mol_type = other RNA
ggagccagaggctcctcctcg

SEQ ID NO: 966 - RNA, 21 nt, synthetic construct
  misc_feature 1..21: note = Synthetic oligonucleotide
  source 1..21: mol_type = other RNA
tgctgcccgcttccctgctcc

SEQ ID NO: 967 - RNA, 21 nt, synthetic construct
  misc_feature 1..21: note = Synthetic oligonucleotide
  source 1..21: mol_type = other RNA
cgcttccctgctccgaggaaa

SEQ ID NO: 968 - RNA, 21 nt, synthetic construct
  misc_feature 1..21: note = Synthetic oligonucleotide
  source 1..21: mol_type = other RNA
gcttccctgctccgaggaaaa

SEQ ID NO: 969 - RNA, 21 nt, synthetic construct
  misc_feature 1..21: note = Synthetic oligonucleotide
  source 1..21: mol_type = other RNA
cttccctgctccgaggaaaaa

SEQ ID NO: 970 - RNA, 21 nt, synthetic construct
  misc_feature 1..21: note = Synthetic oligonucleotide
  source 1..21: mol_type = other RNA
ttccctgctccgaggaaaaac

SEQ ID NO: 971 - RNA, 21 nt, synthetic construct
  misc_feature 1..21: note = Synthetic oligonucleotide
  source 1..21: mol_type = other RNA
aaaacgactccacaactttgt

SEQ ID NO: 972 - RNA, 21 nt, synthetic construct
  misc_feature 1..21: note = Synthetic oligonucleotide
  source 1..21: mol_type = other RNA
aaacgactccacaactttgtg

SEQ ID NO: 973 - RNA, 21 nt, synthetic construct
  misc_feature 1..21: note = Synthetic oligonucleotide
  source 1..21: mol_type = other RNA
aacgactccacaactttgtgc

SEQ ID NO: 974 - RNA, 21 nt, synthetic construct
  misc_feature 1..21: note = Synthetic oligonucleotide
  source 1..21: mol_type = other RNA
acgactccacaactttgtgcg

SEQ ID NO: 975 - RNA, 21 nt, synthetic construct
  misc_feature 1..21: note = Synthetic oligonucleotide
  source 1..21: mol_type = other RNA
cgactccacaactttgtgcgc

SEQ ID NO: 976 - RNA, 21 nt, synthetic construct
  misc_feature 1..21: note = Synthetic oligonucleotide
  source 1..21: mol_type = other RNA
gactccacaactttgtgcgcg

SEQ ID NO: 977 - RNA, 21 nt, synthetic construct
  misc_feature 1..21: note = Synthetic oligonucleotide
  source 1..21: mol_type = other RNA
actccacaactttgtgcgcga

SEQ ID NO: 978 - RNA, 21 nt, synthetic construct
  misc_feature 1..21: note = Synthetic oligonucleotide
  source 1..21: mol_type = other RNA
ctccacaactttgtgcgcgag

SEQ ID NO: 979 - RNA, 21 nt, synthetic construct
  misc_feature 1..21: note = Synthetic oligonucleotide
  source 1..21: mol_type = other RNA
tccacaactttgtgcgcgaga

SEQ ID NO: 980 - RNA, 21 nt, synthetic construct
  misc_feature 1..21: note = Synthetic oligonucleotide
  source 1..21: mol_type = other RNA
ccacaactttgtgcgcgagag

SEQ ID NO: 981 - RNA, 21 nt, synthetic construct
  misc_feature 1..21: note = Synthetic oligonucleotide
  source 1..21: mol_type = other RNA
cacaactttgtgcgcgagaga

SEQ ID NO: 982 - RNA, 21 nt, synthetic construct
  misc_feature 1..21: note = Synthetic oligonucleotide
  source 1..21: mol_type = other RNA
acaactttgtgcgcgagagag

SEQ ID NO: 983 - RNA, 21 nt, synthetic construct
  misc_feature 1..21: note = Synthetic oligonucleotide
  source 1..21: mol_type = other RNA
gagaggctcgctccttcggcg

SEQ ID NO: 984 - RNA, 21 nt, synthetic construct
  misc_feature 1..21: note = Synthetic oligonucleotide
  source 1..21: mol_type = other RNA
agaggctcgctccttcggcgg

SEQ ID NO: 985 - RNA, 21 nt, synthetic construct
  misc_feature 1..21: note = Synthetic oligonucleotide
  source 1..21: mol_type = other RNA
tccttcggcggctcctcctcc

SEQ ID NO: 986 - RNA, 21 nt, synthetic construct
  misc_feature 1..21: note = Synthetic oligonucleotide
  source 1..21: mol_type = other RNA
tgcgggcctgcccctttgttg

SEQ ID NO: 987 - RNA, 21 nt, synthetic construct
  misc_feature 1..21: note = Synthetic oligonucleotide
  source 1..21: mol_type = other RNA
gcgggcctgcccctttgttgg

SEQ ID NO: 988 - RNA, 21 nt, synthetic construct
  misc_feature 1..21: note = Synthetic oligonucleotide
  source 1..21: mol_type = other RNA
cgggcctgcccctttgttgga

SEQ ID NO: 989 - RNA, 21 nt, synthetic construct
  misc_feature 1..21: note = Synthetic oligonucleotide
  source 1..21: mol_type = other RNA
gggcctgcccctttgttggag

SEQ ID NO: 990 - RNA, 21 nt, synthetic construct
  misc_feature 1..21: note = Synthetic oligonucleotide
  source 1..21: mol_type = other RNA
ggcctgcccctttgttggagc

SEQ ID NO: 991 - RNA, 21 nt, synthetic construct
  misc_feature 1..21: note = Synthetic oligonucleotide
  source 1..21: mol_type = other RNA
gcctgcccctttgttggagcc

SEQ ID NO: 992 - RNA, 21 nt, synthetic construct
  misc_feature 1..21: note = Synthetic oligonucleotide
  source 1..21: mol_type = other RNA
cctgcccctttgttggagccg

SEQ ID NO: 993 - RNA, 21 nt, synthetic construct
  misc_feature 1..21: note = Synthetic oligonucleotide
  source 1..21: mol_type = other RNA
ctgcccctttgttggagccga

SEQ ID NO: 994 - RNA, 21 nt, synthetic construct
  misc_feature 1..21: note = Synthetic oligonucleotide
  source 1..21: mol_type = other RNA
tgcccctttgttggagccgag

SEQ ID NO: 995 - RNA, 21 nt, synthetic construct
  misc_feature 1..21: note = Synthetic oligonucleotide
  source 1..21: mol_type = other RNA
gcccctttgttggagccgagc

SEQ ID NO: 996 - RNA, 21 nt, synthetic construct
  misc_feature 1..21: note = Synthetic oligonucleotide
  source 1..21: mol_type = other RNA
cccctttgttggagccgagcg

SEQ ID NO: 997 - RNA, 21 nt, synthetic construct
  misc_feature 1..21: note = Synthetic oligonucleotide
  source 1..21: mol_type = other RNA
ccctttgttggagccgagcgc

SEQ ID NO: 998 - RNA, 21 nt, synthetic construct
  misc_feature 1..21: note = Synthetic oligonucleotide
  source 1..21: mol_type = other RNA
cctttgttggagccgagcgct

SEQ ID NO: 999 - RNA, 21 nt, synthetic construct
  misc_feature 1..21: note = Synthetic oligonucleotide
  source 1..21: mol_type = other RNA
ctttgttggagccgagcgctg

SEQ ID NO: 1000 - RNA, 21 nt, synthetic construct
  misc_feature 1..21: note = Synthetic oligonucleotide
  source 1..21: mol_type = other RNA
tttgttggagccgagcgctgg

SEQ ID NO: 1001 - RNA, 21 nt, synthetic construct
  misc_feature 1..21: note = Synthetic oligonucleotide
  source 1..21: mol_type = other RNA
ttgttggagccgagcgctgga

SEQ ID NO: 1002 - RNA, 21 nt, synthetic construct
  misc_feature 1..21: note = Synthetic oligonucleotide
  source 1..21: mol_type = other RNA
tgttggagccgagcgctggag

SEQ ID NO: 1003 - RNA, 21 nt, synthetic construct
  misc_feature 1..21: note = Synthetic oligonucleotide
  source 1..21: mol_type = other RNA
gttggagccgagcgctggagg

SEQ ID NO: 1004 - RNA, 21 nt, synthetic construct
  misc_feature 1..21: note = Synthetic oligonucleotide
  source 1..21: mol_type = other RNA
ttggagccgagcgctggaggc

SEQ ID NO: 1005 - RNA, 21 nt, synthetic construct
  misc_feature 1..21: note = Synthetic oligonucleotide
  source 1..21: mol_type = other RNA
caggggtccgctctccagatg

SEQ ID NO: 1006 - RNA, 21 nt, synthetic construct
  misc_feature 1..21: note = Synthetic oligonucleotide
  source 1..21: mol_type = other RNA
aggggtccgctctccagatga

SEQ ID NO: 1007 - RNA, 21 nt, synthetic construct
  misc_feature 1..21: note = Synthetic oligonucleotide
  source 1..21: mol_type = other RNA
ggggtccgctctccagatgag

SEQ ID NO: 1008 - RNA, 21 nt, synthetic construct
  misc_feature 1..21: note = Synthetic oligonucleotide
  source 1..21: mol_type = other RNA
gggtccgctctccagatgaga

SEQ ID NO: 1009 - RNA, 21 nt, synthetic construct
  misc_feature 1..21: note = Synthetic oligonucleotide
  source 1..21: mol_type = other RNA
ggtccgctctccagatgagaa

SEQ ID NO: 1010 - RNA, 21 nt, synthetic construct
  misc_feature 1..21: note = Synthetic oligonucleotide
  source 1..21: mol_type = other RNA
gtccgctctccagatgagaag

SEQ ID NO: 1011 - RNA, 21 nt, synthetic construct
  misc_feature 1..21: note = Synthetic oligonucleotide
  source 1..21: mol_type = other RNA
tccgctctccagatgagaagg

SEQ ID NO: 1012 - RNA, 21 nt, synthetic construct
  misc_feature 1..21: note = Synthetic oligonucleotide
  source 1..21: mol_type = other RNA
ccgctctccagatgagaaggg

SEQ ID NO: 1013 - RNA, 21 nt, synthetic construct
  misc_feature 1..21: note = Synthetic oligonucleotide
  source 1..21: mol_type = other RNA
cgctctccagatgagaagggt

SEQ ID NO: 1014 - RNA, 21 nt, synthetic construct
  misc_feature 1..21: note = Synthetic oligonucleotide
  source 1..21: mol_type = other RNA
gctctccagatgagaagggtg

SEQ ID NO: 1015 - RNA, 21 nt, synthetic construct
  misc_feature 1..21: note = Synthetic oligonucleotide
  source 1..21: mol_type = other RNA
ctctccagatgagaagggtgc

SEQ ID NO: 1016 - RNA, 21 nt, synthetic construct
  misc_feature 1..21: note = Synthetic oligonucleotide
  source 1..21: mol_type = other RNA
tctccagatgagaagggtgca

SEQ ID NO: 1017 - RNA, 21 nt, synthetic construct
  misc_feature 1..21: note = Synthetic oligonucleotide
  source 1..21: mol_type = other RNA
ctccagatgagaagggtgcag

SEQ ID NO: 1018 - RNA, 21 nt, synthetic construct
  misc_feature 1..21: note = Synthetic oligonucleotide
  source 1..21: mol_type = other RNA
tccagatgagaagggtgcaga

SEQ ID NO: 1019 - RNA, 21 nt, synthetic construct
  misc_feature 1..21: note = Synthetic oligonucleotide
  source 1..21: mol_type = other RNA
ccagatgagaagggtgcagat

SEQ ID NO: 1020 - RNA, 21 nt, synthetic construct
  misc_feature 1..21: note = Synthetic oligonucleotide
  source 1..21: mol_type = other RNA
cagatgagaagggtgcagatc

SEQ ID NO: 1021 - RNA, 21 nt, synthetic construct
  misc_feature 1..21: note = Synthetic oligonucleotide
  source 1..21: mol_type = other RNA
agatgagaagggtgcagatca

SEQ ID NO: 1022 - RNA, 21 nt, synthetic construct
  misc_feature 1..21: note = Synthetic oligonucleotide
  source 1..21: mol_type = other RNA
gatgagaagggtgcagatcag

SEQ ID NO: 1023 - RNA, 21 nt, synthetic construct
  misc_feature 1..21: note = Synthetic oligonucleotide
  source 1..21: mol_type = other RNA
atgagaagggtgcagatcagc

SEQ ID NO: 1024 - RNA, 21 nt, synthetic construct
  misc_feature 1..21: note = Synthetic oligonucleotide
  source 1..21: mol_type = other RNA
tgagaagggtgcagatcagcg

SEQ ID NO: 1025 - RNA, 21 nt, synthetic construct
  misc_feature 1..21: note = Synthetic oligonucleotide
  source 1..21: mol_type = other RNA
gagaagggtgcagatcagcgc

SEQ ID NO: 1026 - RNA, 21 nt, synthetic construct
  misc_feature 1..21: note = Synthetic oligonucleotide
  source 1..21: mol_type = other RNA
agaagggtgcagatcagcgcc

SEQ ID NO: 1027 - RNA, 21 nt, synthetic construct
  misc_feature 1..21: note = Synthetic oligonucleotide
  source 1..21: mol_type = other RNA
gaagggtgcagatcagcgcca

SEQ ID NO: 1028 - RNA, 21 nt, synthetic construct
  misc_feature 1..21: note = Synthetic oligonucleotide
  source 1..21: mol_type = other RNA
aagggtgcagatcagcgccag

SEQ ID NO: 1029 - RNA, 21 nt, synthetic construct
  misc_feature 1..21: note = Synthetic oligonucleotide
  source 1..21: mol_type = other RNA
agggtgcagatcagcgccagc

SEQ ID NO: 1030 - RNA, 21 nt, synthetic construct
  misc_feature 1..21: note = Synthetic oligonucleotide
  source 1..21: mol_type = other RNA
gggtgcagatcagcgccagcc

SEQ ID NO: 1031 - RNA, 21 nt, synthetic construct
  misc_feature 1..21: note = Synthetic oligonucleotide
  source 1..21: mol_type = other RNA
ggtgcagatcagcgccagccc

SEQ ID NO: 1032 - RNA, 21 nt, synthetic construct
  misc_feature 1..21: note = Synthetic oligonucleotide
  source 1..21: mol_type = other RNA
gtgcagatcagcgccagccct

SEQ ID NO: 1033 - RNA, 21 nt, synthetic construct
  misc_feature 1..21: note = Synthetic oligonucleotide
  source 1..21: mol_type = other RNA
tgcagatcagcgccagccctc

SEQ ID NO: 1034 - RNA, 21 nt, synthetic construct
  misc_feature 1..21: note = Synthetic oligonucleotide
  source 1..21: mol_type = other RNA
gcagatcagcgccagccctcc

SEQ ID NO: 1035 - RNA, 21 nt, synthetic construct
  misc_feature 1..21: note = Synthetic oligonucleotide
  source 1..21: mol_type = other RNA
cagatcagcgccagccctccg

SEQ ID NO: 1036 - RNA, 21 nt, synthetic construct
  misc_feature 1..21: note = Synthetic oligonucleotide
  source 1..21: mol_type = other RNA
agatcagcgccagccctccgg

SEQ ID NO: 1037 - RNA, 21 nt, synthetic construct
  misc_feature 1..21: note = Synthetic oligonucleotide
  source 1..21: mol_type = other RNA
ccgccggcccagcaactttgt

SEQ ID NO: 1038 - RNA, 21 nt, synthetic construct
  misc_feature 1..21: note = Synthetic oligonucleotide
  source 1..21: mol_type = other RNA
cgccggcccagcaactttgtt

SEQ ID NO: 1039 - RNA, 21 nt, synthetic construct
  misc_feature 1..21: note = Synthetic oligonucleotide
  source 1..21: mol_type = other RNA
gccggcccagcaactttgttc

SEQ ID NO: 1040 - RNA, 21 nt, synthetic construct
  misc_feature 1..21: note = Synthetic oligonucleotide
  source 1..21: mol_type = other RNA
ccggcccagcaactttgttcc

SEQ ID NO: 1041 - RNA, 21 nt, synthetic construct
  misc_feature 1..21: note = Synthetic oligonucleotide
  source 1..21: mol_type = other RNA
cggcccagcaactttgttccg

SEQ ID NO: 1042 - RNA, 21 nt, synthetic construct
  misc_feature 1..21: note = Synthetic oligonucleotide
  source 1..21: mol_type = other RNA
ggcccagcaactttgttccgg

SEQ ID NO: 1043 - RNA, 21 nt, synthetic construct
  misc_feature 1..21: note = Synthetic oligonucleotide
  source 1..21: mol_type = other RNA
gcccagcaactttgttccgga

SEQ ID NO: 1044 - RNA, 21 nt, synthetic construct
  misc_feature 1..21: note = Synthetic oligonucleotide
  source 1..21: mol_type = other RNA
cccagcaactttgttccggaa

SEQ ID NO: 1045 - RNA, 21 nt, synthetic construct
  misc_feature 1..21: note = Synthetic oligonucleotide
  source 1..21: mol_type = other RNA
ccagcaactttgttccggaag

SEQ ID NO: 1046 - RNA, 21 nt, synthetic construct
  misc_feature 1..21: note = Synthetic oligonucleotide
  source 1..21: mol_type = other RNA
cagcaactttgttccggaaga

SEQ ID NO: 1047 - RNA, 21 nt, synthetic construct
  misc_feature 1..21: note = Synthetic oligonucleotide
  source 1..21: mol_type = other RNA
agcaactttgttccggaagat

SEQ ID NO: 1048 - RNA, 21 nt, synthetic construct
  misc_feature 1..21: note = Synthetic oligonucleotide
  source 1..21: mol_type = other RNA
gcaactttgttccggaagatc

SEQ ID NO: 1049 - RNA, 21 nt, synthetic construct
  misc_feature 1..21: note = Synthetic oligonucleotide
  source 1..21: mol_type = other RNA
caactttgttccggaagatca

SEQ ID NO: 1050 - RNA, 21 nt, synthetic construct
  misc_feature 1..21: note = Synthetic oligonucleotide
  source 1..21: mol_type = other RNA
aactttgttccggaagatcag

SEQ ID NO: 1051 - RNA, 21 nt, synthetic construct
  misc_feature 1..21: note = Synthetic oligonucleotide
  source 1..21: mol_type = other RNA
actttgttccggaagatcagc

SEQ ID NO: 1052 - RNA, 21 nt, synthetic construct
  misc_feature 1..21: note = Synthetic oligonucleotide
  source 1..21: mol_type = other RNA
ctttgttccggaagatcagcc

SEQ ID NO: 1053 - RNA, 21 nt, synthetic construct
  misc_feature 1..21: note = Synthetic oligonucleotide
  source 1..21: mol_type = other RNA
tttgttccggaagatcagccc

SEQ ID NO: 1054 - RNA, 21 nt, synthetic construct
  misc_feature 1..21: note = Synthetic oligonucleotide
  source 1..21: mol_type = other RNA
ttgttccggaagatcagcccc

SEQ ID NO: 1055 - RNA, 21 nt, synthetic construct
  misc_feature 1..21: note = Synthetic oligonucleotide
  source 1..21: mol_type = other RNA
tgttccggaagatcagcccca

SEQ ID NO: 1056 - RNA, 21 nt, synthetic construct
  misc_feature 1..21: note = Synthetic oligonucleotide
  source 1..21: mol_type = other RNA
gttccggaagatcagccccac

SEQ ID NO: 1057 - RNA, 21 nt, synthetic construct
  misc_feature 1..21: note = Synthetic oligonucleotide
  source 1..21: mol_type = other RNA
ttccggaagatcagccccact

SEQ ID NO: 1058 - RNA, 21 nt, synthetic construct
  misc_feature 1..21: note = Synthetic oligonucleotide
  source 1..21: mol_type = other RNA
tccggaagatcagccccacta

SEQ ID NO: 1059 - RNA, 21 nt, synthetic construct
  misc_feature 1..21: note = Synthetic oligonucleotide
  source 1..21: mol_type = other RNA
ccggaagatcagccccactac

SEQ ID NO: 1060 - RNA, 21 nt, synthetic construct
  misc_feature 1..21: note = Synthetic oligonucleotide
  source 1..21: mol_type = other RNA
cggaagatcagccccactacg

SEQ ID NO: 1061 - RNA, 21 nt, synthetic construct
  misc_feature 1..21: note = Synthetic oligonucleotide
  source 1..21: mol_type = other RNA
ggaagatcagccccactacgt

SEQ ID NO: 1062 - RNA, 21 nt, synthetic construct
  misc_feature 1..21: note = Synthetic oligonucleotide
  source 1..21: mol_type = other RNA
gaagatcagccccactacgtt

SEQ ID NO: 1063 - RNA, 21 nt, synthetic construct
  misc_feature 1..21: note = Synthetic oligonucleotide
  source 1..21: mol_type = other RNA
aagatcagccccactacgttc

SEQ ID NO: 1064 - RNA, 21 nt, synthetic construct
  misc_feature 1..21: note = Synthetic oligonucleotide
  source 1..21: mol_type = other RNA
agatcagccccactacgttcc

SEQ ID NO: 1065 - RNA, 21 nt, synthetic construct
  misc_feature 1..21: note = Synthetic oligonucleotide
  source 1..21: mol_type = other RNA
gatcagccccactacgttccc

SEQ ID NO: 1066 - RNA, 21 nt, synthetic construct
  misc_feature 1..21: note = Synthetic oligonucleotide
  source 1..21: mol_type = other RNA
atcagccccactacgttcccg

SEQ ID NO: 1067 - RNA, 21 nt, synthetic construct
  misc_feature 1..21: note = Synthetic oligonucleotide
  source 1..21: mol_type = other RNA
tcagccccactacgttcccgg

SEQ ID NO: 1068 - RNA, 21 nt, synthetic construct
  misc_feature 1..21: note = Synthetic oligonucleotide
  source 1..21: mol_type = other RNA
cagccccactacgttcccggg

SEQ ID NO: 1069 - RNA, 21 nt, synthetic construct
  misc_feature 1..21: note = Synthetic oligonucleotide
  source 1..21: mol_type = other RNA
agccccactacgttcccgggt

SEQ ID NO: 1070 - RNA, 21 nt, synthetic construct
  misc_feature 1..21: note = Synthetic oligonucleotide
  source 1..21: mol_type = other RNA
ccccactacgttcccgggtca

SEQ ID NO: 1071 - RNA, 21 nt, synthetic construct
  misc_feature 1..21: note = Synthetic oligonucleotide
  source 1..21: mol_type = other RNA
cccactacgttcccgggtcag

SEQ ID NO: 1072 - RNA, 21 nt, synthetic construct
  misc_feature 1..21: note = Synthetic oligonucleotide
  source 1..21: mol_type = other RNA
ccactacgttcccgggtcagc

SEQ ID NO: 1073 - RNA, 21 nt, synthetic construct
  misc_feature 1..21: note = Synthetic oligonucleotide
  source 1..21: mol_type = other RNA
cactacgttcccgggtcagcg

SEQ ID NO: 1074 - RNA, 21 nt, synthetic construct
  misc_feature 1..21: note = Synthetic oligonucleotide
  source 1..21: mol_type = other RNA
actacgttcccgggtcagcgt

SEQ ID NO: 1075 - RNA, 21 nt, synthetic construct
  misc_feature 1..21: note = Synthetic oligonucleotide
  source 1..21: mol_type = other RNA
gttcccgggtcagcgtctgcc

SEQ ID NO: 1076 - RNA, 21 nt, synthetic construct
  misc_feature 1..21: note = Synthetic oligonucleotide
  source 1..21: mol_type = other RNA
ttcccgggtcagcgtctgccg

SEQ ID NO: 1077 - RNA, 21 nt, synthetic construct
  misc_feature 1..21: note = Synthetic oligonucleotide
  source 1..21: mol_type = other RNA
tcgggaacttggcgccccaag

SEQ ID NO: 1078 - RNA, 21 nt, synthetic construct
  misc_feature 1..21: note = Synthetic oligonucleotide
  source 1..21: mol_type = other RNA
cgggaacttggcgccccaagt

SEQ ID NO: 1079 - RNA, 21 nt, synthetic construct
  misc_feature 1..21: note = Synthetic oligonucleotide
  source 1..21: mol_type = other RNA
gggaacttggcgccccaagtc

SEQ ID NO: 1080 - RNA, 21 nt, synthetic construct
  misc_feature 1..21: note = Synthetic oligonucleotide
  source 1..21: mol_type = other RNA
ggaacttggcgccccaagtca

SEQ ID NO: 1081 - RNA, 21 nt, synthetic construct
  misc_feature 1..21: note = Synthetic oligonucleotide
  source 1..21: mol_type = other RNA
gaacttggcgccccaagtcac

SEQ ID NO: 1082 - RNA, 21 nt, synthetic construct
  misc_feature 1..21: note = Synthetic oligonucleotide
  source 1..21: mol_type = other RNA
aacttggcgccccaagtcacc

SEQ ID NO: 1083 - RNA, 21 nt, synthetic construct
  misc_feature 1..21: note = Synthetic oligonucleotide
  source 1..21: mol_type = other RNA
acttggcgccccaagtcacct

SEQ ID NO: 1084 - RNA, 21 nt, synthetic construct
  misc_feature 1..21: note = Synthetic oligonucleotide
  source 1..21: mol_type = other RNA
cttggcgccccaagtcacctg

SEQ ID NO: 1085 - RNA, 21 nt, synthetic construct
  misc_feature 1..21: note = Synthetic oligonucleotide
  source 1..21: mol_type = other RNA
ttggcgccccaagtcacctga

SEQ ID NO: 1086 - RNA, 21 nt, synthetic construct
  misc_feature 1..21: note = Synthetic oligonucleotide
  source 1..21: mol_type = other RNA
tggcgccccaagtcacctgac

SEQ ID NO: 1087 - RNA, 21 nt, synthetic construct
  misc_feature 1..21: note = Synthetic oligonucleotide
  source 1..21: mol_type = other RNA
ggcgccccaagtcacctgact

SEQ ID NO: 1088 - RNA, 21 nt, synthetic construct
  misc_feature 1..21: note = Synthetic oligonucleotide
  source 1..21: mol_type = other RNA
gcgccccaagtcacctgactg

SEQ ID NO: 1089 - RNA, 21 nt, synthetic construct
  misc_feature 1..21: note = Synthetic oligonucleotide
  source 1..21: mol_type = other RNA
cgccccaagtcacctgactgc

SEQ ID NO: 1090 - RNA, 21 nt, synthetic construct
  misc_feature 1..21: note = Synthetic oligonucleotide
  source 1..21: mol_type = other RNA
gccccaagtcacctgactgcg

SEQ ID NO: 1091 - RNA, 21 nt, synthetic construct
  misc_feature 1..21: note = Synthetic oligonucleotide
  source 1..21: mol_type = other RNA
ccccaagtcacctgactgcgc

SEQ ID NO: 1092 - RNA, 21 nt, synthetic construct
  misc_feature 1..21: note = Synthetic oligonucleotide
  source 1..21: mol_type = other RNA
cccaagtcacctgactgcgcg

SEQ ID NO: 1093 - RNA, 21 nt, synthetic construct
  misc_feature 1..21: note = Synthetic oligonucleotide
  source 1..21: mol_type = other RNA
ccaagtcacctgactgcgcgc

SEQ ID NO: 1094 - RNA, 21 nt, synthetic construct
  misc_feature 1..21: note = Synthetic oligonucleotide
  source 1..21: mol_type = other RNA
caagtcacctgactgcgcgcc

SEQ ID NO: 1095 - RNA, 21 nt, synthetic construct
  misc_feature 1..21: note = Synthetic oligonucleotide
  source 1..21: mol_type = other RNA
gtcaatcagcgccggccctgc

SEQ ID NO: 1096 - RNA, 21 nt, synthetic construct
  misc_feature 1..21: note = Synthetic oligonucleotide
  source 1..21: mol_type = other RNA
tcaatcagcgccggccctgcg

SEQ ID NO: 1097 - RNA, 21 nt, synthetic construct
  misc_feature 1..21: note = Synthetic oligonucleotide
  source 1..21: mol_type = other RNA
gcgctccagagtgaaccgtgc

SEQ ID NO: 1098 - RNA, 21 nt, synthetic construct
  misc_feature 1..21: note = Synthetic oligonucleotide
  source 1..21: mol_type = other RNA
cgctccagagtgaaccgtgcg

SEQ ID NO: 1099 - RNA, 21 nt, synthetic construct
  misc_feature 1..21: note = Synthetic oligonucleotide
  source 1..21: mol_type = other RNA
gctccagagtgaaccgtgcgt

SEQ ID NO: 1100 - RNA, 21 nt, synthetic construct
  misc_feature 1..21: note = Synthetic oligonucleotide
  source 1..21: mol_type = other RNA
ctccagagtgaaccgtgcgtg

SEQ ID NO: 1101 - RNA, 21 nt, synthetic construct
  misc_feature 1..21: note = Synthetic oligonucleotide
  source 1..21: mol_type = other RNA
tccagagtgaaccgtgcgtgc

SEQ ID NO: 1102 - RNA, 21 nt, synthetic construct
  misc_feature 1..21: note = Synthetic oligonucleotide
  source 1..21: mol_type = other RNA
ccagagtgaaccgtgcgtgcc

SEQ ID NO: 1103 - RNA, 21 nt, synthetic construct
  misc_feature 1..21: note = Synthetic oligonucleotide
  source 1..21: mol_type = other RNA
cagagtgaaccgtgcgtgccg

SEQ ID NO: 1104 - RNA, 21 nt, synthetic construct
  misc_feature 1..21: note = Synthetic oligonucleotide
  source 1..21: mol_type = other RNA
agagtgaaccgtgcgtgccgg

SEQ ID NO: 1105 - RNA, 21 nt, synthetic construct
  misc_feature 1..21: note = Synthetic oligonucleotide
  source 1..21: mol_type = other RNA
gggagtccggagaccgaacca

SEQ ID NO: 1106 - RNA, 21 nt, synthetic construct
  misc_feature 1..21: note = Synthetic oligonucleotide
  source 1..21: mol_type = other RNA
ggagtccggagaccgaaccag

SEQ ID NO: 1107 - RNA, 21 nt, synthetic construct
  misc_feature 1..21: note = Synthetic oligonucleotide
  source 1..21: mol_type = other RNA
gagtccggagaccgaaccaga

SEQ ID NO: 1108 - RNA, 21 nt, synthetic construct
  misc_feature 1..21: note = Synthetic oligonucleotide
  source 1..21: mol_type = other RNA
agtccggagaccgaaccagaa

SEQ ID NO: 1109 - RNA, 21 nt, synthetic construct
  misc_feature 1..21: note = Synthetic oligonucleotide
  source 1..21: mol_type = other RNA
gtccggagaccgaaccagaat

SEQ ID NO: 1110 - RNA, 21 nt, synthetic construct
  misc_feature 1..21: note = Synthetic oligonucleotide
  source 1..21: mol_type = other RNA
tccggagaccgaaccagaatg

SEQ ID NO: 1111 - RNA, 21 nt, synthetic construct
  misc_feature 1..21: note = Synthetic oligonucleotide
  source 1..21: mol_type = other RNA
ccggagaccgaaccagaatgg

SEQ ID NO: 1112 - RNA, 21 nt, synthetic construct
  misc_feature 1..21: note = Synthetic oligonucleotide
  source 1..21: mol_type = other RNA
cggagaccgaaccagaatgga

SEQ ID NO: 1113 - RNA, 21 nt, synthetic construct
  misc_feature 1..21: note = Synthetic oligonucleotide
  source 1..21: mol_type = other RNA
ggagaccgaaccagaatggat

SEQ ID NO: 1114 - RNA, 21 nt, synthetic construct
  misc_feature 1..21: note = Synthetic oligonucleotide
  source 1..21: mol_type = other RNA
gagaccgaaccagaatggatg

SEQ ID NO: 1115 - RNA, 21 nt, synthetic construct
  misc_feature 1..21: note = Synthetic oligonucleotide
  source 1..21: mol_type = other RNA
agaccgaaccagaatggatgc

SEQ ID NO: 1116 - RNA, 21 nt, synthetic construct
  misc_feature 1..21: note = Synthetic oligonucleotide
  source 1..21: mol_type = other RNA
gaccgaaccagaatggatgcc

SEQ ID NO: 1117 - RNA, 21 nt, synthetic construct
  misc_feature 1..21: note = Synthetic oligonucleotide
  source 1..21: mol_type = other RNA
accgaaccagaatggatgcct

SEQ ID NO: 1118 - RNA, 21 nt, synthetic construct
  misc_feature 1..21: note = Synthetic oligonucleotide
  source 1..21: mol_type = other RNA
ccgaaccagaatggatgcctc

SEQ ID NO: 1119 - RNA, 21 nt, synthetic construct
  misc_feature 1..21: note = Synthetic oligonucleotide
  source 1..21: mol_type = other RNA
cgaaccagaatggatgcctcg

SEQ ID NO: 1120 - RNA, 21 nt, synthetic construct
  misc_feature 1..21: note = Synthetic oligonucleotide
  source 1..21: mol_type = other RNA
gaaccagaatggatgcctcga

SEQ ID NO: 1121 - RNA, 21 nt, synthetic construct
  misc_feature 1..21: note = Synthetic oligonucleotide
  source 1..21: mol_type = other RNA
aaccagaatggatgcctcgag

SEQ ID NO: 1122 - RNA, 21 nt, synthetic construct
  misc_feature 1..21: note = Synthetic oligonucleotide
  source 1..21: mol_type = other RNA
accagaatggatgcctcgagc

SEQ ID NO: 1123 - RNA, 21 nt, synthetic construct
  misc_feature 1..21: note = Synthetic oligonucleotide
  source 1..21: mol_type = other RNA
ccagaatggatgcctcgagca

SEQ ID NO: 1124 - RNA, 21 nt, synthetic construct
  misc_feature 1..21: note = Synthetic oligonucleotide
  source 1..21: mol_type = other RNA
cagaatggatgcctcgagcag

SEQ ID NO: 1125 - RNA, 21 nt, synthetic construct
  misc_feature 1..21: note = Synthetic oligonucleotide
  source 1..21: mol_type = other RNA
agaatggatgcctcgagcaga

SEQ ID NO: 1126 - RNA, 21 nt, synthetic construct
  misc_feature 1..21: note = Synthetic oligonucleotide
  source 1..21: mol_type = other RNA
gaatggatgcctcgagcagac

SEQ ID NO: 1127 - RNA, 21 nt, synthetic construct
  misc_feature 1..21: note = Synthetic oligonucleotide
  source 1..21: mol_type = other RNA
aatggatgcctcgagcagaca

SEQ ID NO: 1128 - RNA, 21 nt, synthetic construct
  misc_feature 1..21: note = Synthetic oligonucleotide
  source 1..21: mol_type = other RNA
atggatgcctcgagcagacac

SEQ ID NO: 1129 - RNA, 21 nt, synthetic construct
  misc_feature 1..21: note = Synthetic oligonucleotide
  source 1..21: mol_type = other RNA
tggatgcctcgagcagacaca

SEQ ID NO: 1130 - RNA, 21 nt, synthetic construct
  misc_feature 1..21: note = Synthetic oligonucleotide
  source 1..21: mol_type = other RNA
ggatgcctcgagcagacacaa

SEQ ID NO: 1131 - RNA, 21 nt, synthetic construct
  misc_feature 1..21: note = Synthetic oligonucleotide
  source 1..21: mol_type = other RNA
gatgcctcgagcagacacaat

SEQ ID NO: 1132 - RNA, 21 nt, synthetic construct
  misc_feature 1..21: note = Synthetic oligonucleotide
  source 1..21: mol_type = other RNA
atgcctcgagcagacacaatt

SEQ ID NO: 1133 - RNA, 21 nt, synthetic construct
  misc_feature 1..21: note = Synthetic oligonucleotide
  source 1..21: mol_type = other RNA
tgcctcgagcagacacaattc

SEQ ID NO: 1134 - RNA, 21 nt, synthetic construct
  misc_feature 1..21: note = Synthetic oligonucleotide
  source 1..21: mol_type = other RNA
gcctcgagcagacacaattcc

SEQ ID NO: 1135 - RNA, 21 nt, synthetic construct
  misc_feature 1..21: note = Synthetic oligonucleotide
  source 1..21: mol_type = other RNA
cctcgagcagacacaattccc

SEQ ID NO: 1136 - RNA, 21 nt, synthetic construct
  misc_feature 1..21: note = Synthetic oligonucleotide
  source 1..21: mol_type = other RNA
ctcgagcagacacaattcccc

SEQ ID NO: 1137 - RNA, 21 nt, synthetic construct
  misc_feature 1..21: note = Synthetic oligonucleotide
  source 1..21: mol_type = other RNA
tcgagcagacacaattcccct

SEQ ID NO: 1138 - RNA, 21 nt, synthetic construct
  misc_feature 1..21: note = Synthetic oligonucleotide
  source 1..21: mol_type = other RNA
cgagcagacacaattcccctt

SEQ ID NO: 1139 - RNA, 21 nt, synthetic construct
  misc_feature 1..21: note = Synthetic oligonucleotide
  source 1..21: mol_type = other RNA
gagcagacacaattcccctta

SEQ ID NO: 1140 - RNA, 21 nt, synthetic construct
  misc_feature 1..21: note = Synthetic oligonucleotide
  source 1..21: mol_type = other RNA
agcagacacaattccccttat

SEQ ID NO: 1141 - RNA, 21 nt, synthetic construct
  misc_feature 1..21: note = Synthetic oligonucleotide
  source 1..21: mol_type = other RNA
gcagacacaattccccttatt

SEQ ID NO: 1142 - RNA, 21 nt, synthetic construct
  misc_feature 1..21: note = Synthetic oligonucleotide
  source 1..21: mol_type = other RNA
cagacacaattccccttattc

SEQ ID NO: 1143 - RNA, 21 nt, synthetic construct
  misc_feature 1..21: note = Synthetic oligonucleotide
  source 1..21: mol_type = other RNA
agacacaattccccttattca

SEQ ID NO: 1144 - RNA, 21 nt, synthetic construct
  misc_feature 1..21: note = Synthetic oligonucleotide
  source 1..21: mol_type = other RNA
gacacaattccccttattcag

SEQ ID NO: 1145 - RNA, 21 nt, synthetic construct
  misc_feature 1..21: note = Synthetic oligonucleotide
  source 1..21: mol_type = other RNA
acacaattccccttattcaga

SEQ ID NO: 1146 - RNA, 21 nt, synthetic construct
  misc_feature 1..21: note = Synthetic oligonucleotide
  source 1..21: mol_type = other RNA
cacaattccccttattcagat

SEQ ID NO: 1147 - RNA, 21 nt, synthetic construct
  misc_feature 1..21: note = Synthetic oligonucleotide
  source 1..21: mol_type = other RNA
acaattccccttattcagatt

SEQ ID NO: 1148 - RNA, 21 nt, synthetic construct
  misc_feature 1..21: note = Synthetic oligonucleotide
  source 1..21: mol_type = other RNA
caattccccttattcagatta

SEQ ID NO: 1149 - RNA, 21 nt, synthetic construct
  misc_feature 1..21: note = Synthetic oligonucleotide
  source 1..21: mol_type = other RNA
tccccttattcagattatccg

SEQ ID NO: 1150 - RNA, 21 nt, synthetic construct
  misc_feature 1..21: note = Synthetic oligonucleotide
  source 1..21: mol_type = other RNA
ccccttattcagattatccga

SEQ ID NO: 1151 - RNA, 21 nt, synthetic construct
  misc_feature 1..21: note = Synthetic oligonucleotide
  source 1..21: mol_type = other RNA
cccttattcagattatccgaa

SEQ ID NO: 1152 - RNA, 21 nt, synthetic construct
  misc_feature 1..21: note = Synthetic oligonucleotide
  source 1..21: mol_type = other RNA
ccttattcagattatccgaaa

SEQ ID NO: 1153 - RNA, 21 nt, synthetic construct
  misc_feature 1..21: note = Synthetic oligonucleotide
  source 1..21: mol_type = other RNA
cagattatccgaaaatgtgag

SEQ ID NO: 1154 - RNA, 21 nt, synthetic construct
  misc_feature 1..21: note = Synthetic oligonucleotide
  source 1..21: mol_type = other RNA
gattatccgaaaatgtgagtc

SEQ ID NO: 1155 - RNA, 21 nt, synthetic construct
  misc_feature 1..21: note = Synthetic oligonucleotide
  source 1..21: mol_type = other RNA
ttatccgaaaatgtgagtcac

SEQ ID NO: 1156 - RNA, 21 nt, synthetic construct
  misc_feature 1..21: note = Synthetic oligonucleotide
  source 1..21: mol_type = other RNA
tatccgaaaatgtgagtcacg

SEQ ID NO: 1157 - RNA, 21 nt, synthetic construct
  misc_feature 1..21: note = Synthetic oligonucleotide
  source 1..21: mol_type = other RNA
atccgaaaatgtgagtcacgt

SEQ ID NO: 1158 - RNA, 21 nt, synthetic construct
  misc_feature 1..21: note = Synthetic oligonucleotide
  source 1..21: mol_type = other RNA
tccgaaaatgtgagtcacgtc

SEQ ID NO: 1159 - RNA, 21 nt, synthetic construct
  misc_feature 1..21: note = Synthetic oligonucleotide
  source 1..21: mol_type = other RNA
ccgaaaatgtgagtcacgtcc

SEQ ID NO: 1160 - RNA, 21 nt, synthetic construct
  misc_feature 1..21: note = Synthetic oligonucleotide
  source 1..21: mol_type = other RNA
cgaaaatgtgagtcacgtccc

SEQ ID NO: 1161 - RNA, 21 nt, synthetic construct
  misc_feature 1..21: note = Synthetic oligonucleotide
  source 1..21: mol_type = other RNA
gaaaatgtgagtcacgtcccc

SEQ ID NO: 1162 - RNA, 21 nt, synthetic construct
  misc_feature 1..21: note = Synthetic oligonucleotide
  source 1..21: mol_type = other RNA
aaaatgtgagtcacgtcccct

SEQ ID NO: 1163 - RNA, 21 nt, synthetic construct
  misc_feature 1..21: note = Synthetic oligonucleotide
  source 1..21: mol_type = other RNA
aaatgtgagtcacgtcccctt

SEQ ID NO: 1164 - RNA, 21 nt, synthetic construct
  misc_feature 1..21: note = Synthetic oligonucleotide
  source 1..21: mol_type = other RNA
aatgtgagtcacgtcccctta

SEQ ID NO: 1165 - RNA, 21 nt, synthetic construct
  misc_feature 1..21: note = Synthetic oligonucleotide
  source 1..21: mol_type = other RNA
atgtgagtcacgtccccttag

SEQ ID NO: 1166 - RNA, 21 nt, synthetic construct
  misc_feature 1..21: note = Synthetic oligonucleotide
  source 1..21: mol_type = other RNA
tgtgagtcacgtccccttagg

SEQ ID NO: 1167 - RNA, 21 nt, synthetic construct
  misc_feature 1..21: note = Synthetic oligonucleotide
  source 1..21: mol_type = other RNA
gtgagtcacgtccccttaggc

SEQ ID NO: 1168 - RNA, 21 nt, synthetic construct
  misc_feature 1..21: note = Synthetic oligonucleotide
  source 1..21: mol_type = other RNA
tgagtcacgtccccttaggca

SEQ ID NO: 1169 - RNA, 21 nt, synthetic construct
  misc_feature 1..21: note = Synthetic oligonucleotide
  source 1..21: mol_type = other RNA
gagtcacgtccccttaggcaa

SEQ ID NO: 1170 - RNA, 21 nt, synthetic construct
  misc_feature 1..21: note = Synthetic oligonucleotide
  source 1..21: mol_type = other RNA
agtcacgtccccttaggcaaa

SEQ ID NO: 1171 - RNA, 21 nt, synthetic construct
  misc_feature 1..21: note = Synthetic oligonucleotide
  source 1..21: mol_type = other RNA
gtcacgtccccttaggcaaaa

SEQ ID NO: 1172 - RNA, 21 nt, synthetic construct
  misc_feature 1..21: note = Synthetic oligonucleotide
  source 1..21: mol_type = other RNA
tcacgtccccttaggcaaaac

SEQ ID NO: 1173 - RNA, 21 nt, synthetic construct
  misc_feature 1..21: note = Synthetic oligonucleotide
  source 1..21: mol_type = other RNA
cacgtccccttaggcaaaaca

SEQ ID NO: 1174 - RNA, 21 nt, synthetic construct
  misc_feature 1..21: note = Synthetic oligonucleotide
  source 1..21: mol_type = other RNA
acgtccccttaggcaaaacag

SEQ ID NO: 1175 - RNA, 21 nt, synthetic construct
  misc_feature 1..21: note = Synthetic oligonucleotide
  source 1..21: mol_type = other RNA
cgtccccttaggcaaaacagg

SEQ ID NO: 1176 - RNA, 21 nt, synthetic construct
  misc_feature 1..21: note = Synthetic oligonucleotide
  source 1..21: mol_type = other RNA
gtccccttaggcaaaacaggt

SEQ ID NO: 1177 - RNA, 21 nt, synthetic construct
  misc_feature 1..21: note = Synthetic oligonucleotide
  source 1..21: mol_type = other RNA
tccccttaggcaaaacaggtc

SEQ ID NO: 1178 - RNA, 21 nt, synthetic construct
  misc_feature 1..21: note = Synthetic oligonucleotide
  source 1..21: mol_type = other RNA
ccccttaggcaaaacaggtct

SEQ ID NO: 1179 - RNA, 21 nt, synthetic construct
  misc_feature 1..21: note = Synthetic oligonucleotide
  source 1..21: mol_type = other RNA
cccttaggcaaaacaggtctc

SEQ ID NO: 1180 - RNA, 21 nt, synthetic construct
  misc_feature 1..21: note = Synthetic oligonucleotide
  source 1..21: mol_type = other RNA
ccttaggcaaaacaggtctct

SEQ ID NO: 1181 - RNA, 21 nt, synthetic construct
  misc_feature 1..21: note = Synthetic oligonucleotide
  source 1..21: mol_type = other RNA
cttaggcaaaacaggtctcta

SEQ ID NO: 1182 - RNA, 21 nt, synthetic construct
  misc_feature 1..21: note = Synthetic oligonucleotide
  source 1..21: mol_type = other RNA
ttaggcaaaacaggtctctag

SEQ ID NO: 1183 - RNA, 21 nt, synthetic construct
  misc_feature 1..21: note = Synthetic oligonucleotide
  source 1..21: mol_type = other RNA
taggcaaaacaggtctctagt

SEQ ID NO: 1184 - RNA, 21 nt, synthetic construct
  misc_feature 1..21: note = Synthetic oligonucleotide
  source 1..21: mol_type = other RNA
aggcaaaacaggtctctagtt

SEQ ID NO: 1185 - RNA, 21 nt, synthetic construct
  misc_feature 1..21: note = Synthetic oligonucleotide
  source 1..21: mol_type = other RNA
ggcaaaacaggtctctagtta

SEQ ID NO: 1186 - RNA, 21 nt, synthetic construct
  misc_feature 1..21: note = Synthetic oligonucleotide
  source 1..21: mol_type = other RNA
gcaaaacaggtctctagttag

SEQ ID NO: 1187 - RNA, 21 nt, synthetic construct
  misc_feature 1..21: note = Synthetic oligonucleotide
  source 1..21: mol_type = other RNA
caaaacaggtctctagttagg

SEQ ID NO: 1188 - RNA, 21 nt, synthetic construct
  misc_feature 1..21: note = Synthetic oligonucleotide
  source 1..21: mol_type = other RNA
aaaacaggtctctagttaggg

SEQ ID NO: 1189 - RNA, 21 nt, synthetic construct
  misc_feature 1..21: note = Synthetic oligonucleotide
  source 1..21: mol_type = other RNA
aaacaggtctctagttagggt

SEQ ID NO: 1190 - RNA, 21 nt, synthetic construct
  misc_feature 1..21: note = Synthetic oligonucleotide
  source 1..21: mol_type = other RNA
aacaggtctctagttagggtg

SEQ ID NO: 1191 - RNA, 21 nt, synthetic construct
  misc_feature 1..21: note = Synthetic oligonucleotide
  source 1..21: mol_type = other RNA
acaggtctctagttagggtga

SEQ ID NO: 1192 - RNA, 21 nt, synthetic construct
  misc_feature 1..21: note = Synthetic oligonucleotide
  source 1..21: mol_type = other RNA
caggtctctagttagggtgat

SEQ ID NO: 1193 - RNA, 21 nt, synthetic construct
  misc_feature 1..21: note = Synthetic oligonucleotide
  source 1..21: mol_type = other RNA
aggtctctagttagggtgata

SEQ ID NO: 1194 - RNA, 21 nt, synthetic construct
  misc_feature 1..21: note = Synthetic oligonucleotide
  source 1..21: mol_type = other RNA
ggtctctagttagggtgatat

SEQ ID NO: 1195 - RNA, 21 nt, synthetic construct
  misc_feature 1..21: note = Synthetic oligonucleotide
  source 1..21: mol_type = other RNA
gtctctagttagggtgatatt

SEQ ID NO: 1196 - RNA, 21 nt, synthetic construct
  misc_feature 1..21: note = Synthetic oligonucleotide
  source 1..21: mol_type = other RNA
tctctagttagggtgatattt

SEQ ID NO: 1197 - RNA, 21 nt, synthetic construct
  misc_feature 1..21: note = Synthetic oligonucleotide
  source 1..21: mol_type = other RNA
ctctagttagggtgatatttt

SEQ ID NO: 1198 - DNA, 21 bp, synthetic construct
  misc_feature 1..19: note = RNA at (1,2,3,4,5,6,7,8,9,10,11,12,13,14,15,16,17,18,19)
  misc_feature 20..21: note = DNA at (20,21)
  misc_feature 1..21: note = Synthetic oligonucleotide
  source 1..21: mol_type = other DNA
tctaaggacatcagcaacctt

SEQ ID NO: 1199 - DNA, 21 bp, synthetic construct
  misc_feature 1..19: note = RNA at (1,2,3,4,5,6,7,8,9,10,11,12,13,14,15,16,17,18,19)
  misc_feature 20..21: note = DNA at (20,21)
  misc_feature 1..21: note = Synthetic oligonucleotide
  source 1..21: mol_type = other DNA
tctactgtcactcagtagttt

SEQ ID NO: 1200 - DNA, 1000 bp, Homo sapiens
  misc_feature 1..1000: note = Synthetic oligonucleotide
  source 1..1000: mol_type = genomic DNA
atttatctcttaaaaaaatatcaccctaactagagacctgttttgcctaaggggacgtgactcacattttcggataatctgaataaggggaattgtgtctgctcgaggcatccattctggttcggtctccggactcccggctcccggcacgcacggttcactctggagcgcgcgccccaggccagccaagcgccgagccgggctgctgcgggctgggagggcgcgcagggccggcgctgattgacggggcgcgcagtcaggtgacttggggcgccaagttcccgacgcggtggccgcggtgaccgccgaggcccggcagacgctgacccgggaacgtagtggggctgatcttccggaacaaagttgctgggccggcggcggcggggcgagagcgccgagggggagccggagcgctgcagaggcgcgggccggagggctggcgctgatctgcacccttctcatctggagagcggacccctggctgcccggaggcgagccccttcccggggggtgggggcggcaacgcgcgacccagcggtcctgcgccccaccctccctcctccgcctccagcgctcggctccaacaaaggggcaggcccgcagcggggaggaggaggaggagccgccgaaggagcgagcctctctcgcgcacaaagttgtggagtcgtttttcctcggagcagggaagcgggcagcagcagccggccgcgggctttctcccgccgaggggcgaggaggagcctctggctccagaagccgattggggaatcacggggagcggcgccccccttcttttgggtcatttctgcaaacggaaaactctgtagcgtttggcaaagttggtgcctgcgcgccccttccaggtttgcgctttgactgttttgtttttggcggaactaccaggcaggaagattgcacaagtaaggggcgttttcagtcgggtgtcaatttctctttctttctttctttttttaaaatttcggttcgtgtctgcttctcc

SEQ ID NO: 1201 - DNA, 20 bp, synthetic construct
  misc_feature 1..20: note = Synthetic primer
  source 1..20: mol_type = other DNA
gccgtggcaaagatccattt

SEQ ID NO: 1202 - DNA, 22 bp, synthetic construct
  misc_feature 1..22: note = Synthetic primer
  source 1..22: mol_type = other DNA
acattattcaggtcagcaaggg

SEQ ID NO: 1203 - DNA, 22 bp, synthetic construct
  misc_feature 1..22: note = Synthetic primer
  source 1..22: mol_type = other DNA
tgctcacccaccaacaatttag

SEQ ID NO: 1204 - DNA, 22 bp, synthetic construct
  misc_feature 1..22: note = Synthetic primer
  source 1..22: mol_type = other DNA
tctgctctgactttagcacctg

SEQ ID NO: 1205 - DNA, 24 bp, synthetic construct
  misc_feature 1..24: note = Synthetic primer
  source 1..24: mol_type = other DNA
gatagttaagtgggatcgagacat

SEQ ID NO: 1206 - DNA, 21 bp, synthetic construct
  misc_feature 1..21: note = Synthetic primer
  source 1..21: mol_type = other DNA
agcaagcaagcagaatttgga

SEQ ID NO: 1207 - DNA, 141 bp, Homo sapiens
  source 1..141: mol_type = genomic DNA
tagagacctgttttgcctaaggggacgtgactcacattttcggataatctgaataaggggaattgtgtctgctcgaggcatccattctggttcggtctccggactcccggctcccggcacgcacggttcactctggagcgc

SEQ ID NO: 1208 - DNA, 58 bp, Homo sapiens
  source 1..58: mol_type = genomic DNA
gcagacgctgacccgggaacgtagtggggctgatcttccggaacaaagttgctgggcc

SEQ ID NO: 1209 - DNA, 50 bp, Homo sapiens
  source 1..50: mol_type = genomic DNA
ggagggctggcgctgatctgcacccttctcatctggagagcggacccctg

SEQ ID NO: 1210 - DNA, 37 bp, Homo sapiens
  source 1..37: mol_type = genomic DNA
ctccagcgctcggctccaacaaaggggcaggcccgca

SEQ ID NO: 1211 - DNA, 21 bp, synthetic construct
  misc_feature 1..21: note = Synthetic primer
  source 1..21: mol_type = other DNA
aaagatggtcaaggtcgcaag

SEQ ID NO: 1212 - DNA, 23 bp, synthetic construct
  misc_feature 1..23: note = Synthetic primer
  source 1..23: mol_type = other DNA
tagtcaagggcatatcctacaac

SEQ ID NO: 1213 - DNA, 18 bp, synthetic construct
  misc_feature 1..16: note = RNA at (1,2,3,4,5,6,7,8,9,10,11,12,13,14,15,16)
  misc_feature 17..18: note = DNA at (17,18)
  misc_feature 1..18: note = Synthetic oligonucleotide
  source 1..18: mol_type = other DNA
gctcggctccaacaaatt

SEQ ID NO: 1214 - DNA, 18 bp, synthetic construct
  misc_feature 1..16: note = RNA at (1,2,3,4,5,6,7,8,9,10,11,12,13,14,15,16)
  misc_feature 17..18: note = DNA at (17,18)
  misc_feature 1..18: note = Synthetic oligonucleotide
  source 1..18: mol_type = other DNA
tttgttggagccgagctt

SEQ ID NO: 1215 - RNA, 18 nt, synthetic construct
  misc_feature 1..18: note = Synthetic oligonucleotide
  source 1..18: mol_type = other RNA
gctcggctccaacaaagg

SEQ ID NO: 1216 - RNA, 18 nt, synthetic construct
  misc_feature 1..18: note = Synthetic oligonucleotide
  source 1..18: mol_type = other RNA
cctttgttggagccgagc

SEQ ID NO: 1217 - RNA, 22 nt, synthetic construct
  misc_feature 1..22: note = Synthetic oligonucleotide
  source 1..22: mol_type = other RNA
gctcggctccaacaaaggggca

SEQ ID NO: 1218 - RNA, 20 nt, synthetic construct
  misc_feature 1..20: note = Synthetic oligonucleotide
  source 1..20: mol_type = other RNA
cccctttgttggagccgagc

SEQ ID NO: 1219 - RNA, 30 nt, synthetic construct
  misc_feature 1..30: note = Synthetic oligonucleotide
  source 1..30: mol_type = other RNA
gctcggctccaacaaaggggcaggcccgca

SEQ ID NO: 1220 - RNA, 28 nt, synthetic construct
  misc_feature 1..28: note = Synthetic oligonucleotide
  source 1..28: mol_type = other RNA
cgggcctgcccctttgttggagccgagc

SEQ ID NO: 1221 - RNA, 21 nt, synthetic construct
  misc_feature 1..21: note = Synthetic oligonucleotide
  source 1..21: mol_type = other RNA
gctcggctccaacaaaggggc

SEQ ID NO: 1222 - RNA, 21 nt, synthetic construct
  misc_feature 1..21: note = Synthetic oligonucleotide
  source 1..21: mol_type = other RNA
ccctttgttggaccggtgggg

SEQ ID NO: 1223 - RNA, 20 nt, synthetic construct
  misc_feature 1..20: note = Synthetic oligonucleotide
  source 1..20: mol_type = other RNA
gctcggctccaacaaagggg

SEQ ID NO: 1224 - RNA, 19 nt, synthetic construct
  misc_feature 1..19: note = Synthetic oligonucleotide
  source 1..19: mol_type = other RNA
ccctttgttggagccgagc

SEQ ID NO: 1225 - RNA, 22 nt, synthetic construct
  misc_feature 1..22: note = Synthetic oligonucleotide
  source 1..22: mol_type = other RNA
gctcggctccaacaaaggggca

SEQ ID NO: 1226 - RNA, 24 nt, synthetic construct
  misc_feature 1..24: note = Synthetic oligonucleotide
  source 1..24: mol_type = other RNA
gctcggctccaacaaaggggcagg

SEQ ID NO: 1227 - RNA, 19 nt, synthetic construct
  misc_feature 1..19: note = Synthetic oligonucleotide
  source 1..19: mol_type = other RNA
ggcacgcacggttcactct

SEQ ID NO: 1228 - RNA, 19 nt, synthetic construct
  misc_feature 1..19: note = Synthetic oligonucleotide
  source 1..19: mol_type = other RNA
agagtgaaccgtgcgtgcc

SEQ ID NO: 1229 - RNA, 23 nt, synthetic construct
  misc_feature 1..23: note = Synthetic oligonucleotide
  source 1..23: mol_type = other RNA
tcccggcacgcacggttcactct

SEQ ID NO: 1230 - RNA, 25 nt, synthetic construct
  misc_feature 1..25: note = Synthetic oligonucleotide
  source 1..25: mol_type = other RNA
agagtgaaccgtgcgtgccgggagc

SEQ ID NO: 1231 - RNA, 33 nt, synthetic construct
  misc_feature 1..33: note = Synthetic oligonucleotide
  source 1..33: mol_type = other RNA
gactcccggctcccggcacgcacggttcactct

SEQ ID NO: 1232 - RNA, 35 nt, synthetic construct
  misc_feature 1..35: note = Synthetic oligonucleotide
  source 1..35: mol_type = other RNA
agagtgaaccgtgcgtgccgggagccgggagtccg

SEQ ID NO: 1233 - RNA, 21 nt, synthetic construct
  misc_feature 1..21: note = Synthetic oligonucleotide
  source 1..21: mol_type = other RNA
ggcacgcacggtactcacagc

SEQ ID NO: 1234 - RNA, 21 nt, synthetic construct
  misc_feature 1..21: note = Synthetic oligonucleotide
  source 1..21: mol_type = other RNA
agagtgaaccgtgcgtgccgg

SEQ ID NO: 1235 - RNA, 19 nt, synthetic construct
  misc_feature 1..19: note = Synthetic oligonucleotide
  source 1..19: mol_type = other RNA
ggcacgcacggttcactct

SEQ ID NO: 1236 - RNA, 23 nt, synthetic construct
  misc_feature 1..23: note = Synthetic oligonucleotide
  source 1..23: mol_type = other RNA
agagtgaaccgtgcgtgccggga

SEQ ID NO: 1237 - RNA, 25 nt, synthetic construct
  misc_feature 1..25: note = Synthetic oligonucleotide
  source 1..25: mol_type = other RNA
agagtgaaccgtgcgtgccgggagc

SEQ ID NO: 1238 - DNA, 18 bp, synthetic construct
  misc_feature 1..16: note = RNA at (1,2,3,4,5,6,7,8,9,10,11,12,13,14,15,16)
  misc_feature 17..18: note = DNA at (17,18)
  misc_feature 1..18: note = Synthetic oligonucleotide
  source 1..18: mol_type = other DNA
gaggcatccattctggtt

SEQ ID NO: 1239 - DNA, 18 bp, synthetic construct
  misc_feature 1..16: note = RNA at (1,2,3,4,5,6,7,8,9,10,11,12,13,14,15,16)
  misc_feature 17..18: note = DNA at (17,18)
  misc_feature 1..18: note = Synthetic oligonucleotide
  source 1..18: mol_type = other DNA
ccagaatggatgcctctt

SEQ ID NO: 1240 - RNA, 29 nt, synthetic construct
  misc_feature 1..29: note = Synthetic oligonucleotide
  source 1..29: mol_type = other RNA
attgtgtctgctcgaggcatccattctgg

SEQ ID NO: 1241 - RNA, 35 nt, synthetic construct
  misc_feature 1..35: note = Synthetic oligonucleotide
  source 1..35: mol_type = other RNA
ccagaatggatgcctcgagcagacacaattcccct